(19) United States
(12) Patent Application Publication
(10) Pub. No.: US 2024/0058414 A1
(43) Pub. Date: Feb. 22, 2024

(54) METHODS OF TREATING MELANOCORTIN-4 RECEPTOR PATHWAY-ASSOCIATED DISORDERS

(71) Applicant: RHYTHM PHARMACEUTICALS, INC., Boston, MA (US)

(72) Inventors: Leonardus H.T. Van Der Ploeg, Newton, MA (US); Alastair Garfield, Allston, MA (US); Bhavik P. Shah, Winchester, MA (US)

(21) Appl. No.: 18/028,117

(22) PCT Filed: Sep. 24, 2021
(86) PCT No.: PCT/US2021/052032

§ 371 Date: Mar. 23, 2023

Related U.S. Application Data
(60) Provisional application No. 63/082,867, filed on Sep. 24, 2020.

(51) Int. Cl. A61K 38/12; A61K 9/00; A61P 3/04

(52) U.S. Cl. CPC A61K 38/12; A61P 3/04; A61K 9/0019

(57) ABSTRACT

The disclosure is related to a method of treating a disorder, disorder, or condition associated with an MC4R pathway agonizable gene, such as obesity or hyperphagia, in a subject using a melanocortin-4 receptor (MC4R) agonist.

### CLAIM OF PRIORITY

This application claims priority to U.S. Application No. 63/082,867, filed on Sep. 24, 2020, the entire contents of which are incorporated herein by reference.

### BACKGROUND

The melanocortin 4 receptor (MC4R) is a heterotrimeric G-protein-coupled receptor that transduces signals by activating adenylate cyclase. MC4R is primarily expressed in neuronal tissue and plays a role in controlling feeding behavior and energy homeostasis by, for example, integrating an agonist signal provided by the α-melanocyte stimulating hormone (α-MSH), and an antagonist signal provided by the agouti-related peptide (AGRP).

MC4R is a part of the leptin-melanocortin pathway, also known as the POMC-MC4R pathway, which includes a number of proteins such as leptin, leptin receptors, pro-opiomelanocortin (POMC), and prohormone convertases including PCSK1, α-MSH, and others. In mammals, the hypothalamic POMC-MC4R pathway is part of the regulatory network of appetite and body weight.

### SUMMARY OF THE INVENTION

The present disclosure features, inter alia, treatments for diseases, disorders, and conditions related to the melanocortin-4 receptor (MC4R) pathway. In one aspect, the present disclosure comprises a method of treating a disease, disorder, or condition in a subject having an MC4R pathway agonizable gene with a compound (e.g., an MC4R agonist) or compositions thereof. In some embodiments, the MC4R agonist is a compound of any one of Formulas (I), (II), (III), (IV), (V), (VI), (VII), (VIII), (IX), (X), (XI), or (XII), (e.g., as described herein) or a pharmaceutically acceptable salt thereof.

In some embodiments, the subject has or is identified as having a mutation (e.g., a substitution mutation, a deletion mutation, or a polymorphism, e.g., a loss of function mutation) or an epigenetic modification in or at an MC4R pathway agonizable gene, e.g., as described herein. In some embodiments, the MC4R pathway agonizable gene is selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, TRPC5, PHIP, and MeCP2. In some embodiments, the MC4R pathway agonizable gene is selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, and MeCP2. In some embodiments, the MC4R pathway agonizable gene is selected from RAI1 and SRC1. In some embodiments, the MC4R pathway agonizable gene is RAI1. In some embodiments, the MC4R pathway agonizable gene is SRC1. In some embodiments, the MC4R pathway agonizable gene is TRPC5. In some embodiments, the MC4R pathway agonizable gene is PHIP. In some embodiments, the MC4R pathway agonizable gene is PCSK1 N221D. In some embodiments, the MC4R pathway agonizable gene is selected from a gene listed in Table 1, e.g., described herein.

In some embodiments, the MC4R pathway agonizable gene is POMC, PCSK1, LEPR, LEP, MC4R, SDCCAG8, SH2B1, CPE, ALMS1, BBS1, BBS2, BBS4, BBS5, BBS6, BBS7, BBS8, BBS9, BBS10, BBS12, BBS18, BBS20, GNAS, MC3R, NHLH2, SIM1, BDNF, NTRK2, MAGEL2, or a 16p11.2 deletion.

In some embodiments, the subject carries, or is identified as carrying, a mutation in a MC4R pathway agonizable gene. In an embodiment, the subject is, or is identified as being, heterozygous for a mutation in the MC4R pathway agonizable gene.

In some embodiments, a heterozygous subject carries, or is identified as carrying, a non-functional, e.g., mutant, e.g., null mutant, allele of the MC4R pathway agonizable gene and a functional or wildtype allele of the MC4R pathway agonizable gene.

In some embodiments, a heterozygous subject carries, or is identified as carrying, a first non-functional, e.g., mutant, e.g., null mutant, allele of the MC4R pathway agonizable gene and a second non-functional or mutant, e.g., null mutant, allele of the MC4R pathway agonizable gene. For example, the subject is a compound heterozygous carrier having two distinct non-functional alleles.

In some embodiments, the subject is, or is identified as, homozygous for a non-functional, e.g., mutant, e.g., null mutant, allele of an MC4R pathway agonizable gene.

In some embodiments, the subject carries, or is identified as carrying, a mutation in a second MC4R pathway agonizable gene. In an embodiment, the subject is, or is identified as being, heterozygous for a mutation in the second MC4R pathway agonizable gene. In some embodiments, a heterozygous subject carries, or is identified as carrying, a non-functional, e.g., mutant, e.g., null mutant, allele of the second MC4R pathway agonizable gene and a functional or wildtype, allele of the second MC4R pathway agonizable gene. In an embodiment, a heterozygous subject carries, or is identified as carrying, a first non-functional, e.g., mutant, e.g., null mutant, allele of the second MC4R pathway agonizable gene and a second non-functional or mutant, e.g., null mutant, allele of second the MC4R pathway agonizable gene. For example, the subject is a compound heterozygous carrier having two distinct non-functional alleles.

In some embodiments, the subject is, or is identified as, homozygous for a non-functional, e.g., mutant, e.g., null mutant, allele of a second MC4R pathway agonizable gene.

The MC4R agonist, e.g., a compound of any one of Formulas (I), (II), (III), (IV), (V), (VI), (VII), (VIII), (IX), (X), (XI), or (XII), or a pharmaceutically acceptable salt thereof, may be provided as a composition (e.g., a pharmaceutical composition) with a pharmaceutically acceptable excipient. In an embodiment, the pharmaceutically acceptable excipient comprises a polyethylene glycol (e.g., a modified polyethylene glycol), a lipid (e.g., a neutral lipid or a phospholipid). In an embodiment, the pharmaceutically acceptable excipient comprises a modified polyethylene glycol. In an embodiment, the pharmaceutically acceptable excipient comprises a lipid, such as a neutral diacyl lipid or a phospholipid.

The MC4R agonist or composition thereof may be provided in a unit dosage form. For example, the unit dosage form may comprise between about 0.01 mg to 100 mg of the MC4R agonist. In an embodiment, the unit dosage form comprises between 0.1 mg and 100 mg, e.g., between 0.1 mg and 50 mg, 0.1 mg and 25 mg, 0.1 mg and 10 mg, 1 mg and 100 mg, 1 mg and 50 mg, 1 mg and 25 mg, 1 mg and 10 mg, 5 mg and 100 mg, 5 mg and 50 mg, 5 mg and 25 mg, 5 mg and 15 mg, or 5 mg and 10 mg.

The MC4R agonist or composition thereof may be administered to a subject daily, weekly or monthly. In an embodiment, the MC4R agonist or composition thereof is administered daily, e.g., once daily, twice daily, or three times daily. In an embodiment, the MC4R agonist or composition thereof is administered weekly, e.g., once every week, once every two weeks, once every three weeks. In embodiments, the MC4R agonist or composition thereof is administered daily over a period of at least 3 weeks, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks or more, or at least 1, 2, 3, 4, 5, 6, 7, 8, 9, 10, 11, or 12 months or more, or at least 1, 2, 3, 4 years or more.

In embodiments, the method comprises administering the MC4R agonist or composition thereof in a unit dosage form suitable for injection, e.g., subcutaneous injection, to the subject. In embodiments, the unit dosage form is disposed within a delivery device, e.g., a syringe (e.g., prefilled syringe), an implantable device, a needleless hypodermic injection device, an infusion pump (e.g., implantable infusion pump), or an osmotic delivery system. In embodiments, the MC4R agonist is administered subcutaneously, e.g., by subcutaneous injection.

In embodiments, the subject is obese, e.g., severely obese. In embodiments, the subject has early onset severe obesity. In embodiments, the subject is hyperphagic. In embodiments, the subject experiences severe hunger. In embodiments, the subject has a body mass index (BMI) greater than 25 kg/m² (e.g., ≥25, 30, 31, 32, 33, 34, 35, 36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration. In embodiments, the subject has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration. In embodiments, the subject has a body mass index (BMI) greater than 40 kg/m² (e.g., ≥41, 42, 43, 44, 45, 46, 47, 48, 49, 50, 51, 52, 53, 54, 55 kg/m² or greater) prior to administration of the MC4R agonist, e.g., at the time the MC4R agonist is prescribed, or at the time of the first administration. In embodiments, the subject has a body mass index (BMI) greater than 45 kg/m² (e.g., ≥46, 47, 48, 49, 50, 51, 52, 53, 54, 55 kg/m² or greater) prior to administration of the MC4R agonist, e.g., at the time the MC4R agonist is prescribed, or at the time of the first administration.

In embodiments, the subject has a BMI higher than the 85-95th percentile prior to administration of the MC4R agonist or composition thereof, e.g., at the time the MC4R agonist is prescribed, or at the time of the first administration.

In embodiments, the subject has failed one or more previous therapies, e.g., exercise, diet, or behavioral therapies, prior to administration of the MC4R agonist or composition thereof, e.g., at the time the agonist is prescribed, or at the time of the first administration.

In embodiments, the subject has a lower body weight after administration of the MC4R agonist or composition thereof than before administration of the agonist.

In embodiments, administration of the MC4R agonist or composition thereof results in a reduction of weight in the subject compared to the weight of the subject before treatment of about 1 kg to 3 kg after 1 week of treatment, or about 1 kg to 6 kg after 2 weeks of treatment, or about 2 kg to 12 kg after 4 weeks of treatment, or about 4 kg to 24 kg after 8 weeks of treatment, or about 8 kg to 48 kg after 16 weeks of treatment. In embodiments, administration of the MC4R agonist or composition thereof results in a reduction of BMI by about 1%, 2%, 3%, 5%, 6%, 7%, 8%, 9%, 10%, or more, e.g., by at least 2, 3, 4, 5, 6, 7, 8, 9, 10, 11, 12, 13, 14, 15, 16, 17, 18, 19, 20 weeks or longer.

In embodiments, administration of the MC4R agonist or composition thereof results in no detectable/significant decrease in resting energy expenditure (REE) in the subject, e.g., over a period of 24 hours, one week, or 30 days or longer, e.g., as compared to a control REE (e.g., the REE in the subject prior to treatment or a predetermined REE, e.g., in subjects of similar pre-treatment BMI, e.g., when expressed as REE per kg of lean body mass).

In embodiments, administration of the MC4R agonist or composition thereof results in a reduction in food intake of at least 5 kcal/kg/day, e.g., 5, 10, 20, 30, 40, 50, 60, 70, 80, or 90 or more kcal/kg/day. In embodiments, the reduction in food intake is relative to the food intake at baseline. In embodiments, the baseline food intake is at least 100 kcal/kg/day, e.g., for a pediatric subject at about 1 year of age. In embodiments, the baseline food intake is at least 40 kcal/kg/day, e.g., for a pediatric subject, e.g., in late adolescence.

In embodiments, administration of the MC4R agonist or composition thereof results in a reduction in waist circumference of the subject compared to a control (e.g., the waist circumference of the subject prior to treatment), as measured 1, 2, 3, 4, 5, 6, 7, 8, 9, 10 weeks or more after initiation of treatment.

In embodiments, administration of the MC4R agonist or composition thereof results in no detectable increase in blood pressure (e.g., diastolic and/or systolic blood pressure) of the subject compared to the blood pressure of the subject prior to treatment, as measured 1, 2, 3, 4, 5, 6, 7, 8, 9, 10 weeks or more after initiation of treatment. In embodiments, administration of the MC4R agonist or composition thereof results in a reduction in blood pressure (e.g., diastolic and/or systolic blood pressure) of the subject compared to the blood pressure of the subject prior to treatment, as measured 1, 2, 3, 4, 5, 6, 7, 8, 9, 10 weeks or more after initiation of treatment. In embodiments, administration of the MC4R agonist or composition thereof results in a reduction in systolic blood of the subject of at least 3 mmHg (e.g., at least 3, 3.5, 4, 4.5, 5, 5.5, 6, 6.5, 7 mmHg or more) compared to the blood pressure of the subject prior to treatment, as measured 1, 2, 3, 4, 5, 6, 7, 8, 9, 10 weeks or more after initiation of treatment. In embodiments, administration of the MC4R agonist or composition thereof results in a reduction in diastolic blood pressure of the subject of at least 4 mmHg (e.g., at least 4, 7, 7.5, 8, 8.5, 9, 9.5, 10 mmHg or more) compared to the blood pressure of the subject prior to treatment, as measured 1, 2, 3, 4, 5, 6, 7, 8, 9, 10 weeks or more after initiation of treatment.

In embodiments, administration of the MC4R agonist or composition thereof results in a reduction of hunger in a subject. The reduction of hunger may result in a reduction of food intake, decrease in resting energy expenditure (REE), reduction of weight, reduction in waist circumference, and/or reduction in blood pressure in the subject.

In embodiments, the subject is a mammal, e.g., a human.

In embodiments, the method further comprises acquiring knowledge of the genotype of the subject, e.g., acquiring knowledge of the genotype of an MC4R pathway agonizable gene, e.g., a gene listed in Table 1. In embodiments, the knowledge is acquired directly, e.g., from a sample (e.g., a blood, serum, urine, or tissue (e.g., biopsy) sample) from the subject.

In embodiments, the MC4R agonist or composition thereof is administered in response to the detection of a predetermined sequence, e.g., a mutation, MC4R pathway agonizable gene, e.g., a gene listed in Table 1. In embodiments, the predetermined sequence, e.g., mutation, is detected in a nucleic acid by a method chosen from one or more of: a nucleic acid hybridization assay, an amplification-based assay, a PCR-RFLP assay, real-time PCR, sequencing (e.g., DNA sequencing, e.g., next generation sequencing or Sanger method sequencing, bisulfite sequencing, or pyrosequencing), screening analysis, FISH, spectral karyotyping or MFISH, comparative genomic hybridization, in situ hybridization, SSP, HPLC, or mass-spectrometric genotyping. In embodiments, the predetermined sequence, e.g., mutation, is detected in the subject. In embodiments, the predetermined sequence, e.g., mutation, is detected in a nucleic acid molecule or a polypeptide in a sample from the subject. In embodiments, the sample comprises cells from a blood, serum, urine, or tissue (e.g., biopsy) from the subject. In embodiments, the method comprises acquiring knowledge of the genotype of the subject, e.g., acquiring knowledge of the genotype of, e.g., of a mutation in a gene listed in

In some embodiments, the compound is a compound of Formula (I) or a pharmaceutically acceptable salt thereof. In some embodiments, the compound of Formula (I) is Ac-Arg-c(Cys-D-Ala-His-D-Phe-Arg-Trp-Cys)-NH₂ (SEQ ID NO: 140) or a pharmaceutically acceptable salt thereof. In some embodiments, the compound is a compound of Formula (II) or a pharmaceutically acceptable salt thereof. In some embodiments, the compound of Formula (II) is Hydantoin(C(O)-(Arg-Gly))-cyclo(Cys-Glu-His-D-Phe-Arg-Trp-Cys)-NH₂ (SEQ ID NO:13) or a pharmaceutically acceptable salt thereof. In some embodiments, the compound of any one of Formulas (I), (II), (III), (IV), (V), (VI), (VII), (VIII), (IX), (X), (XI), or (XII), is formulated as a pharmaceutical composition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Other features, objects, and advantages of the disclosure will be apparent from the description and drawings, and from the claims.

### DETAILED DESCRIPTION

The present disclosure is based at least in part on the discovery that targeting certain defects in the POMC-MC4R pathway, e.g., such as mutations in an MC4R pathway agonizable gene by using a MC4R agonist, may lead to significant weight loss, decrease in hunger, and/or an increase in energy expenditure in obese subjects. The disclosure is also based in part on the discovery that obese subjects having a defect (e.g., genetic defect) in an MC4R pathway agonizable genes are likely to exhibit a significantly greater response (e.g., in decreasing body weight and/or hunger and/or increasing energy expenditure) to an MC4R agonist than obese subjects not having such a defect.

Without being bound by theory, a MC4R agonist, such as a compound of any one of Formulas (I), (II), (III), (IV), (V), (VI), (VII), (VIII), (IX), (X), (XI), or (XII), (e.g., as described herein), e.g., setmelanotide (i.e., Ac-Arg-c(Cys-D-Ala-His-D-Phe-Arg-Trp-Cys)-NH₂, SEQ ID NO: 140) or a pharmaceutically acceptable salt thereof, can act to replace a missing MC4R signaling step in subjects having a genetic defect an MC4R pathway agonizable gene. As such, it is believed that a MC4R agonist, such as setmelanotide, can lead to even greater efficacy in these patient populations than those with general obesity (e.g., wild-type obesity and/or obesity in a subject lacking an identifiable MC4R pathway deficit). Accordingly, the methods and compositions described herein provide an optimized approach to restore MC4R pathway function in subjects with genetic disorders (e.g., genetic deficiencies in one or more genes of the POMC-MC4R pathway) such as Smith-Magenis syndrome, thereby decreasing the extreme hyperphagia and obesity seen in these subjects. Provided herein are methods to treat subjects having a genetic defect in an MC4R pathway agonizable gene, as well as methods to identify/select subjects that have such defects and/or that are likely to respond to a MC4R agonist (e.g., more likely to respond to a MC4R agonist than wild-type obese subjects).

### Definitions

As used herein “about” and “approximately” generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, or knowledge of (e.g., knowledge of the sequence or mutational state of) a genotype or a nucleic acid or polypeptide, by “directly acquiring” or “indirectly acquiring” the physical entity, value, or knowledge. “Directly acquiring” means performing a physical process (e.g., performing a synthetic or analytical method) to obtain the physical entity, value, or knowledge. “Indirectly acquiring” refers to receiving the physical entity, value, or knowledge from another party or source (e.g., a third-party laboratory that directly acquired the physical entity, value, or knowledge). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value or knowledge includes performing a process that includes a physical change in a sample or another substance. Examples include performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the term “functional,” as applied to an allele, e.g., of a MC4R pathway agonizable gene, refers to an allele having, e.g., at least 5, 10, 20, 30, 40, 50, 70, or 80% of the activity of a reference allele, e.g., a wildtype allele.

As used herein, the term “nonfunctional,” as applied to an allele, e.g., a MC4R pathway agonizable gene, refers to an allele which has less than 5, 10, 20, 30, 40, 50, 70, or 80% of the activity of a reference allele, e.g., a wildtype allele. In an embodiment, a nonfunctional allele is an allele of the gene that is other than a functional allele, as the term functional allele is defined herein. By way of example, in an embodiment, if a functional allele has at least 20% of the activity of a reference allele a nonfunctional allele is an allele with less than 20% of the activity.

As used herein, the term “MC4R pathway agonizable gene” refers to a gene associated with a phenotype which can be modulated, e.g., ameliorated or lessened, by modulating MC4R, e.g., agonizing MC4R, e.g., with an MC4R agonist. In an embodiment, the phenotype is hyperphagia, appetite, unwanted appetite, obesity, weight, body mass, or a metabolic syndrome (e.g., diabetes) and the phenotype is, e.g., modulated, e.g., reduced or ameliorated.

In an embodiment, the term “MC4R pathway agonizable gene” does not include the melanocortin 4 receptor (MC4R) gene. In an embodiment, the term “MC4R pathway agonizable gene” does not include POMC. In an embodiment, the MC4R pathway agonizable gene does not comprise any one of POMC, Proprotein Convertase Subtilisin/Kexin Type 1 (PCSK1, also called PC1/3), MAGE-like-2 (MAGEL2), leptin receptor (leptin-R), leptin, 5-hydroxytryptamine (serotonin) receptor 2C, G protein-coupled (5-HT2c receptor), nescient helix loop helix 2 (NhHL2, also called NSCL2), pro-hormone convertase, carboxypeptidase E (CPE), and single-minded 1 (Sim1). In an embodiment, the MC4R pathway agonizable gene does not comprise any gene disclosed in WO2013/102047 or WO 2017/059076, the full contents of each of which is incorporated herein by reference in its entirety.

In an embodiment, at least one of the MC4R alleles is functional, e.g., it has at least 5, 10, 20, 30, 40, 50, 70, or 80% of the activity of a reference allele, e.g., a wildtype allele, e.g., as measured by a functional assay. In an embodiment, one of the MC4R alleles is functional. In an embodiment, both MC4R alleles are functional. In an embodiment, the subject is heterozygous at the MC4R gene and both alleles are functional. In an embodiment, the subject is homozygous at the MC4R gene for a functional allele.

In an embodiment, both MC4R alleles are nonfunctional. (A nonfunctional allele is an allele which is not functional, as functional is defined herein.) In an embodiment, the subject is heterozygous at the MC4R gene and both alleles are nonfunctional. In an embodiment the subject is homozygous at the MC4R gene for a nonfunctional allele.

In an embodiment, at least one allele of an MC4R pathway agonizable gene other than MC4R is functional, e.g., it has at least 5, 10, 20, 30, 40, 50, 70, or 80% of the activity of a reference allele, e.g., a wildtype allele, e.g., as measured by a functional assay. In an embodiment one allele of an MC4R pathway agonizable gene other than MC4R is functional. In an embodiment both alleles of an MC4R pathway agonizable gene other than MC4R are functional. In an embodiment the subject is heterozygous at an MC4R pathway agonizable gene other than MC4R and both alleles are functional. In an embodiment the subject is homozygous at an MC4R pathway agonizable gene other than MC4R for a functional allele.

In an embodiment both MC4R alleles are nonfunctional. (A nonfunctional allele is an allele which is not functional, as functional is defined herein.) In an embodiment the subject is heterozygous at the MC4R gene and both alleles are nonfunctional. In an embodiment the subject is homozygous at the MC4R gene for a nonfunctional allele.

In an embodiment, an epigenetic modification, e.g., a histone modification, e.g., acetylation or nucleobase methylation, e.g., cytosine methylation, is present and is associated with the MC4R pathway agonizable gene phenotype, e.g., hyperphagia, appetite, unwanted appetite, obesity, weight, body mass, or a metabolic syndrome (e.g., diabetes)

In an embodiment, the epigenetic modification is associated with an MC4R pathway agonizable gene. In an embodiment, the epigenetic modification is associated with MC4R.

In an embodiment, the epigenetic modification is associated with an MC4R pathway agonizable gene other than MC4R. In an embodiment, the MC4R pathway agonizable gene does not comprise any one of POMC, Proprotein Convertase Subtilisin/Kexin Type 1 (PCSK1, also called PC1/3), MAGE-like-2 (MAGEL2), leptin receptor (leptin-R), leptin, 5-hydroxytryptamine (serotonin) receptor 2C, G protein-coupled (5-HT2c receptor), nescient helix loop helix 2 (NhHL2, also called NSCL2), pro-hormone convertase, carboxypeptidase E (CPE), and single-minded 1 (Sim1). In an embodiment, the MC4R pathway agonizable gene does not comprise any gene disclosed in WO2013/102047 or WO 2017/059076, the full contents of each of which is incorporated herein by reference in its entirety. As used herein, the term “obese” refers to a subject having a body mass index (BMI) within the ranges defined as “obese” by the Center for Disease Control (see, e.g., URL.cdc.gov/obesity/defining.html and www.cdc.gov/obesity/childhood-/defining.html, last accessed on Aug. 26, 2012) or as defined by “Clinical Guidelines on the Identification, Evaluation, and Treatment of Overweight and Obesity in Adults” from the National Institutes of Health. BMI is obtained by dividing a subject's weight, e.g., in kilograms (kg) by the square of the subject's height, e.g., in meter (m). For example, an adult who has a BMI of 30 kg/m² or higher is considered obese. For example, an adult with a BMI of 25.0 to 29.9 kg/m² is considered overweight; an adult with a BMI of 18.5 to 24.9 kg/m² is considered to have a normal or healthy weight range; and an adult with a BMI of less than 18.5 kg/m² is considered to be underweight. For example, an adult having a height of 5 feet, 9 inches with a body weight of 203 pounds or more is considered obese. For children and teens, obese refers to a subject having a BMI at or above the 85^{th} to 95^{th} percentile for children and teens of the same age and sex.

A “severely obese” subject or a subject having “severe obesity” refers to a subject having a BMI of 35 kg/m² or higher, e.g., 40 kg/m² or higher. For example, a severely obese subject is over 100% over the ideal (normal, healthy) body weight.

As used herein “early onset”, e.g., as in early onset obesity, refers to an onset (e.g., first occurrence of one or more symptoms of a disorder, e.g., a disorder described herein, e.g., obesity) that occurs in a subject before adulthood, e.g., during childhood, e.g., when the subject is less 18 years of age or younger (e.g., 18, 17, 16, 15, 14, 13, 12, 11, 10, 9, 8, 7, 6, 5, 4, 3, 2, or 1 year of age or younger, or during adolescence, e.g., when the child is younger than 12 years of age or when the child is younger than 6 years of age).

As used herein, the term “metabolic syndrome” refers to a group of symptoms that occur together and increase the risk for coronary artery disease, stroke, and type 2 diabetes. According to the American Heart Association and the National Heart, Lung, and Blood Institute, metabolic syndrome also referred to as Syndrome X) is present if a subject has three or more of the following signs: 1) Blood pressure equal to or higher than 130/85 mmHg; 2) Fasting blood sugar (glucose) equal to or higher than 100 mg/dL; 3) Large waist circumference (length around the waist): —Men—40 inches or more; —Women—35 inches or more; 4) Low HDL cholesterol: —Men—under 40 mg/dL; —Women—under 50 mg/dL; 5) Triglycerides equal to or higher than 150 mg/dL. Metabolic syndrome can be diagnosed by testing subject's blood pressure, blood glucose level, HDL cholesterol level, LDL cholesterol level, total cholesterol level, and triglyceride level.

As used herein, the term “agonist” refers to any chemical compound, either naturally occurring or synthetic, that, upon interacting with (e.g., binding to) its target, e.g., MC4R, raises the signaling activity of MC4R above its basal level. An agonist can be a superagonist (i.e. a compound that is capable of producing a greater maximal response than the endogenous agonist for the target receptor, and thus has an efficacy of more than 100%), a full agonist (i.e. a compound that elicits a maximal response following receptor occupation and activation) or a partial agonist (i.e. a compounds that can activate receptors but are unable to elicit the maximal response of the receptor system).

As used herein “treating” includes achieving one or more of the following results: reducing the body weight (as measured, for example, by a body mass index (BMI) and/or body weight), e.g., compared to a control (e.g., body weight before treatment or a predetermined body weight); reducing the waist circumference, e.g., compared to a control (e.g., waist circumference before treatment or a predetermined waist circumference); reducing the hunger level, e.g., compared to a control (e.g., hunger level before treatment or a predetermined hunger level); increasing the resting energy expenditure (REE), e.g., compared to a control (e.g., REE before treatment or a predetermined REE); decreasing the food intake, e.g., compared to a control level (e.g., before treatment or a predetermined food intake); ameliorating or improving a clinical symptom or indicators associated with a disorder described herein such as obesity, Prader Willi Syndrome, Smith-Magenis syndrome, e.g., type-II diabetes, pre-diabetic condition, blood level of hemoglobin A1C (Hb1Ac) above 6%, hyperinsulimenia, hyperlipidemia, insulin insensitivity, or glucose intolerance; delaying, inhibiting or preventing the progression of obesity and/or obesity related indications; or partially or totally delaying, inhibiting or preventing the onset or development of obesity or a obesity related indication. Delaying, inhibiting or preventing the progression of the obesity includes for example, delaying, inhibiting or preventing the progression of a subject having normal weight to obesity. In embodiments, a control is a value of a parameter measured before treatment by a MC4R agonist described herein or a predetermined value. The term “treating” further includes partially or totally reducing the risk for coronary artery disease, stroke, and type 2 diabetes associated with the metabolic syndrome as well as ameliorating or improving a clinical symptom or signs of metabolic syndrome associated with metabolic syndrome, such as any one or more of the five indicators listed above. For example, the term “treating” includes delaying, inhibiting or preventing the progression of parameters associated with the metabolic syndrome, including insulin resistance, glucose clearance and parameters of cardiovascular disease including heart rate and blood pressure.

As used herein “inhibition” or “inhibits” can include a reduction in a certain parameter, such as a parameter described herein. For example, inhibition of a parameter, e.g., activity, can be at least 5%, 10%, 20%, 30%, 40%, or more is included by this term. Thus, inhibition need not be 100%.

“Prophylactic treatment” refers to treatment before onset of obesity to prevent, inhibit or reduce its occurrence.

As used herein, the term “subject” refers to a mammal, e.g., a human. Subject can also refer to an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein, the term “mutation” can refer to an altered nucleic acid sequence of a gene or fragment thereof compared to a wild-type sequence. For example, a mutation can include a point mutation, frame-shift mutation, missense mutation, inversion, deletion, insertion, truncation, chromosomal translocation. In embodiments, a mutation can result in the gene or fragment thereof coding for a non-functional protein, a protein with reduced activity (or a partially functional protein), or a protein with altered activity. For example, a “loss of function” mutation refers to a mutation that results in the gene or fragment thereof coding for a non-functional protein, which has substantially reduced activity compared to its wild-type counterpart (e.g., a non-functional protein has less than 10%, 9%, 8%, 7%, 6%, 5%, 4%, 3%, 2%, 1% or less activity than its wild-type counterpart). For example, “partial loss of function” mutation refers to a mutation that results in the gene or fragment thereof coding for a partially functional protein, which has reduced activity compared to its wild-type counterpart (e.g., a partially functional protein has less than 50% and greater than 10% of the activity of its wild-type counterpart).

As used herein “heterozygous” refers to the presence of two different alleles (having different nucleic acid sequences) for a given gene in a subject. In some embodiments, “heterozygous mutation” can refer to the presence of a mutation on one allele for a given gene and the lack of a mutation on the other allele of the same gene in a subject (e.g., one mutant allele and one wild type allele for a given gene). In other embodiments, a “heterozygous mutation” can be a “compound heterozygous” mutation, which refers to the presence of a mutation (e.g., loss of function mutation or partial loss of function mutation) on one allele for a given gene and a different (e.g., loss of function mutation or partial loss of function mutation) on the other allele for the same gene (e.g., two different alleles that are both mutated, e.g., non-functional or partially functional). In embodiments, where a compound heterozygous mutation includes two non-functional alleles, the genotype can be a null genotype or functionally deficient genotype.

As used herein “homozygous” refers to the presence of two identical alleles for a given gene. In some embodiments, a “homozygous mutation” refers to the presence of two mutant alleles for a given gene, where the two mutant alleles are identical.

As used herein “null genotype” refers to the presence of two non-functional alleles of a gene in a subject.

As used herein “unit dosage form” refers to a physically discrete unit suited as unitary doses for a subject to be treated. Each unit contains a predetermined quantity of active compound calculated to produce the desired therapeutic effect in association with the required pharmaceutical carrier.

As used herein “dosage” refers to a quantity or amount of a therapeutic agent. In some embodiments, a dosage is the amount administered to the subject in a single administration, e.g., in a single injection, a single infusion, or single administration of one or more unit dosages. In embodiments, a dosage is the amount administered to the subject in multiple administrations, e.g., multiple injections, multiple infusions, or multiple administrations of one or more unit dosages. In other embodiments, a dosage can refer to the total amount administered to the subject in a certain time period, e.g., per day. In such examples, the dosage is typically referred to as “daily dosage” or dosage in terms of quantity per day.

As used herein “hunger” or “hunger level” refers to a subject's appetite, desire to consume food, or perceived need for food. In embodiments, the hunger or hunger level of a subject can be quantified by using a scale to obtain a hunger score. In embodiments, the scale for hunger assigns a higher score for a subject that more frequently (e.g., often or always) feels unbearable hunger and a lower score for a subject that less frequently (e.g., sometimes or never) feels unbearable hunger. See, e.g., Sibilia. Psychological Topics 19 (2010), 2, 341-354. For example, a Likert scale for hunger can be used that assigns scores from 0 to 10 points (0=no hunger; 10=severe hunger). In other examples, a Likert scale for hunger can be used that assigns scores from 1 to 4 points, where a subject who never feels unbearable hunger is assigned a score of 1, where a subject who sometimes feels unbearable hunger is assigned a score of 2, where a subject who often feels unbearable hunger is assigned a score of 3, and where a subject who always feels unbearable hunger is assigned a score of 4. See Id.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Unless defined otherwise, all technical and scientific terms used herein have the same meaning as commonly understood by one of ordinary skill in the art. The chemical structures and formulae set forth herein are constructed according to the standard rules of chemical valency known in the chemical arts. Also, all publications, patent applications, patents and other references mentioned herein are incorporated by reference in their entirety.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The compounds useful for practicing the methods described herein may possess one or more chiral centers and so exist in a number of stereoisomeric forms. All stereoisomers and mixtures thereof are included in the scope of the present disclosure. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the disclosure.

The compounds useful for practicing the methods described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” as used herein is meant to include salts of the active compounds that are prepared with relatively nontoxic acids or bases, depending on the particular substituents found on the compounds described herein. When compounds used in the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

The compounds useful for practicing the methods described herein can also exist in unsolvated forms as well as solvated forms, including hydrated forms. In general, the solvated forms are equivalent to unsolvated forms and are encompassed within the scope of the present disclosure. The compounds useful for practicing the methods described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

| Symbol | Meaning |
|---|---|
| Abu | α-aminobutyric acid |
| Ac | acyl group |
| Acc | 1-amino-1-cyclo(C₃-C₉)alkyl carboxylic acid |
| A3c | 1-amino-1 cyclopropanecarboxylic acid |
| A4c | 1-amino-1-cyclobutanecarboxylic acid |
| A5c | 1-amino-1-cyclopentanecarboxylic acid |
| A6c | 1-amino-1-cyclohexanecarboxylic acid |
| Aha | 7-aminoheptanoic acid |
| Ahx | 6-aminohexanoic acid |
| Aib | α-aminoisobutyric acid |
| Aic | 2-aminoindan-2-carboxylic acid |
| Ala or A | Alanine |
| β-Ala | β-alanine |
| Apc | denotes the structure: |
| | 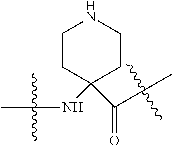 [SMILES: *NC1(C(*)=O)CCNCC1] |
| Apn | 5-aminopentanoic acid (HN—(CH2)₄—C(O) |
| Arg or R | Arginine |
| hArg | Homoarginine |
| Asn or N | Asparagine |
| Asp or D | aspartic acid |
| Bal | 3-benzothienylalanine |
| Bip | 4,4′-biphenylalanine, represented by the structure |
| | 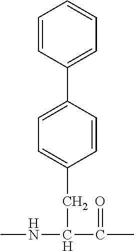 [SMILES: CNC(Cc1ccc(-c2ccccc2)cc1)C(C)=O] |
| Bpa | 4-benzoylphenylalanine |
| 4-Br-Phe | 4-bromo-phenylalanine |
| Cha | β-cyclohexylalanine |
| hCha | homo-cyclohexylalanine |
| Chg | Cyclohexylglycine |
| Cys or C | Cysteine |
| hCys | Homocysteine |
| Dab | 2,4-diaminobutyric acid |
| Dap | 2,3-diaminopropionic acid |
| Dip | β,β-diphenylalanine |
| Doc | 8-amino-3,6-dioxaoctanoic acid with the structure of: |
| | 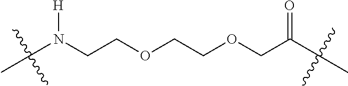 [SMILES: *NCCOCCOCC(*)=O] |
| 2-Fua | β-(2-furyl)-alanine |
| Gaba | 4-aminobutyric acid |
| Gln or Q | Glutamine |
| Glu or E | glutamic acid |
| Gly or G | Glycine |
| His or H | Histidine |
| 3-Hyp | trans-3-hydroxy-L-proline, i.e., (2S,3S)-3-hydroxy- |
| | pyrrolidine-2-carboxylic acid |
| 4-Hyp | 4-hydroxyproline, i.e., (2S,4R)-4-hydorxypyrrolidine- |
| | 2-carboxylic acid |
| Ile or 1 | Isoleucine |
| Leu or L | Leucine |
| hLeu | Homoleucine |
| Lys or K | Lysine |
| Met or M | Methionine |
| β-hMet | β-homomethionine |
| 1-Nal | β-(1-naphthyl)alanine |
| 2-Nal | β-(2-naphthyl)alanine |
| Nip | nipecotic acid |
| Nle | Norleucine |
| Ole | octahydroindole-2-carboxylic acid |
| Orn | Ornithine |
| 2-Pal | β-(2-pyridiyl)alanine |
| 3-Pal | β-(3-pyridiyl)alanine |
| 4-Pal | β-(4-pyridiyl)alanine |
| Pen | Penicillamine |
| Pff | (S)-pentafluorophenylalanine |
| Phe or F | Phenylalanine |
| hPhe | homophenylalanine |
| Pro or P | Proline |
| hProP | Homoproline |
| Ser or S | Serine |
| Tle | tert-Leucine |
| Taz | β-(4-thiazolyl)alanine |
| 2-Thi | β-(2-thienyl)alanine |
| 3-Thi | β-(3-thienyl)alanine |
| Thr or T | Threonine |
| Trp or W | Tryptopham |
| Tyr or Y | Tyrosine |
| D-(Et) Tyr | has a structure of |
| | 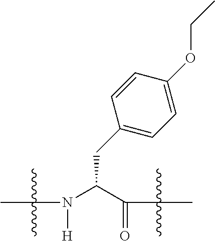 [SMILES: *N[C@H](Cc1ccc(OCC)cc1)C(*)=O] |
| Val or V | Valine |

Certain other abbreviations used herein are defined as follows:
Boc: tert-butyloxycarbonyl
Bzl: Benzyl
DCM: Dichloromethane
DIC: N,N-diisopropylcarbodiimide
DIEA: diisopropylethyl amine
Dmab: 4-{N-(1-(4,4-dimethyl-2,6-dioxocyclohexylidene)-3-methylbutyl)-amino}benzyl
DMAP: 4-(dimethylamino)pyridine
DMF: Dimethylformamide
DNP: 2,4-dinitrophenyl
Fm: Fluorenylmethyl
Fmoc: fluorenylmethyloxycarbonyl
For: Formyl
HBTU: 2-(1H-benzotriazole-1-yl)-1,1,3,3-tetramethyluronium hexafluorophosphate
cHex Cyclohexyl
HOAT: O-(7-azabenzotriazol-1-yl)-1,1,3,3-tetramethyluronium hexafluorophosphate
HOBt: 1-hydroxy-benzotriazole
MBNA 4-methylbenzhydrylamine
Mmt: 4-methoxytrityl
NMP: N-methylpyrrolidone
O-tBu oxy-tert-butyl
Pbf: 2,2,4,6,7-pentamethyldihydrobenzofuran-5-sulfonyl
PyBroP bromo-tris-pyrrolidino-phosphonium hexafluorophosphate
tBu: tert-butyl
TIS: triisopropylsilane
TOS: Tosyl
Trt Trityl
TFA: trifluoro acetic acide
TFFH: tetramethylfluoroforamidiaium hexafluorophosphate
Z: benzyloxycarbonyl

Unless otherwise indicated, with the exception of the N-terminal amino acid, all abbreviations (e.g. Ala) of amino acids in this disclosure stand for the structure of —NH—C(R)(R′)—CO—, wherein R and R′ each is, independently, hydrogen or the side chain of an amino acid (e.g., R═CH₃ and R′═H for Ala), or R and R′ may be joined to form a ring system.

For the N-terminal amino acid, the abbreviation stands for the structure of:

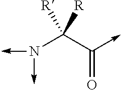

The designation “NH₂” in e.g., as in Ac-Nle-c(Cys-D-Ala-His-D-Phe-Arg-Trp-Cys)-NH₂ (SEQ ID NO:13), indicates that the C-terminus of the peptide is amidated. Ac-Nle-c(Cys-D-Ala-His-D-Phe-Arg-Trp-Cys) (SEQ ID NO:107), or alternatively Ac-Nle-c(Cys-D-Ala-His-D-Phe-Arg-Trp-Cys)-OH (SEQ ID NO:107), indicates that the C-terminus is the free acid.
“-c(Cys-Cys)-” or “-cyclo(Cys-Cys)-” denotes the structure:

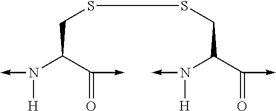

“-c(Cys-Pen)-” or “-cyclo(Cys-Pen)-” denotes the structure:

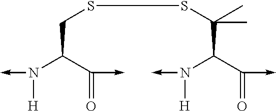

“-c(Asp-Lys)-” or “-cyclo(Asp-Lys)-” denotes the structure:

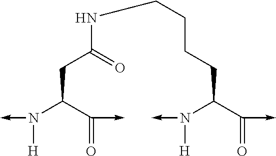

The following abbreviations are used throughout the disclosure:
“Hydantoin-(C(O)-(A^{a}-A^{b}))” denotes the structure:

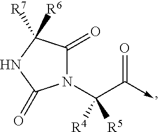

wherein amino acid “A^{a}” has the structure:

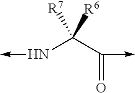

and amino acid “A^{b}” the structure:

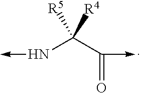

For example, “Hydantoin-(C(O)-Arg-A^{b}))” would have the following structure:

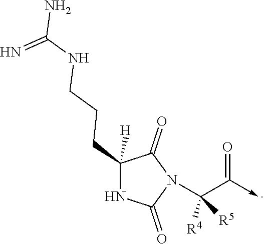

For example, “Hydantoin-(C(O)-(Arg-Gly))” would have the following structure:

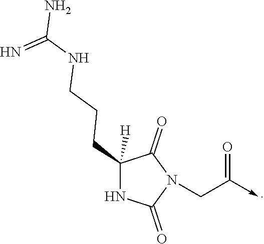

For example, a compound represented as “c[Hydantoin(C(O)-(Cys-A^{b}))-A¹-A²-A³-A⁴-Cys]-” would have the following the structure:

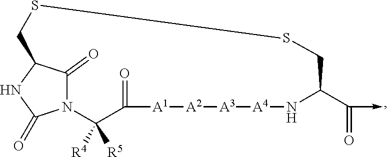

whereas a compound represented as “c[Hydantoin(C(O)-(An-Cys))-A¹-A²-A³-A⁴-Cys]-” would have the structure:

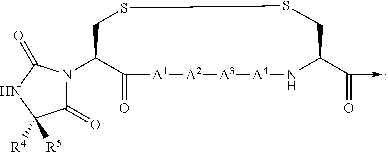

For further guidance, “c[Hydantoin(C(O)-(Asp-A^{b}))-A¹-A²-A³-A⁴-Lys]-” represents the following compound:

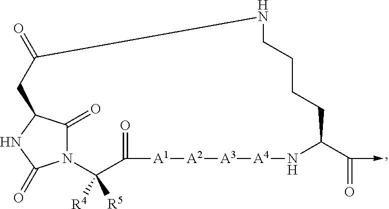

whereas “c[Hydantoin(C(O)-(Dap-A^{b}))-A¹-A²-A³-A⁴-Asp]-” has the following formula:

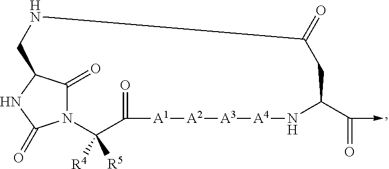

“Acyl” refers to R″—C(O)—, where R″ is H, alkyl, substituted alkyl, heteroalkyl, substituted heteroalkyl, alkenyl, substituted alkenyl, aryl, alkylaryl, or substituted alklyaryl, and is indicated in the general formula of a particular embodiment as “Ac”.
“Alkyl” refers to a hydrocarbon group containing one or more carbon atoms, where multiple carbon atoms if present are joined by single bonds. The alkyl hydrocarbon group may be straight-chain or contain one or more branches or cyclic groups.
“Hydroxyalkyl” refers to an alkyl group wherein one or more hydrogen atoms of the hydrocarbon group are substituted with one or more hydroxy radicals, such as hydroxymethyl, hydroxyethyl, hydroxypropyl, hydroxybutyl, hydroxypentyl, hydroxyhexyl and the like.
“Substituted alkyl” refers to an alkyl wherein one or more hydrogen atoms of the hydrocarbon group are replaced with one or more substituents selected from the group consisting of halogen, (i.e., fluorine, chlorine, bromine, and iodine), —OH, —CN, —SH, amine (e.g., —NH₂, —NHCH₃), —NO₂, guanidine, urea, amidine, and —C₁₋₂₀ alkyl, wherein said —C₁₋₂₀ alkyl optionally may be substituted with one or more substituents selected, independently for each occurrence, from the group consisting of halogens, —CF₃, OCH₃, OCF₃, and —(CH₂)₀₋₂₀—COOH. In different embodiments 1, 2, 3 or 4 substituents are present. The presence of —(CH₂)₀₋₂₀—COOH results in the production of an alkyl acid. Non-limiting examples of alkyl acids containing, or consisting of, —(CH₂)₀₋₂₀—COOH include 2-norbornane acetic acid, tert-butyric acid, 3-cyclopentyl propionic acid, and the like.

The term “halo” encompasses fluoro, chloro, bromo and iodo.

Guanidines are a group of organic compounds that share a common functional group with the general structure (R¹R²N)(R³R⁴N)C═N—R⁵. The central bond within this group is an imine, and the group is related structurally to amidines and ureas.
“Heteroalkyl” refers to an alkyl wherein one of more of the carbon atoms in the hydrocarbon group is replaced with one or more of the following groups: amino, amido, —O—, —S— or carbonyl. In different embodiments 1 or 2 heteroatoms are present.
“Substituted heteroalkyl” refers to a heteroalkyl wherein one or more hydrogen atoms of the hydrocarbon group are replaced with one or more substituents selected from the group consisting of halogen, (i.e., fluorine, chlorine, bromine, and iodine), —OH, —CN, —SH, —NH₂, —NHCH₃, NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.
“Alkenyl” refers to a hydrocarbon group made up of two or more carbons where one or more carbon-carbon double bonds are present. The alkenyl hydrocarbon group may be straight-chain or contain one or more branches or cyclic groups.
“Substituted alkenyl” refers to an alkenyl wherein one or more hydrogens are replaced with one or more substituents selected from the group consisting of halogen (i.e., fluorine, chlorine, bromine, and iodine), —OH, —CN, —SH, —NH₂, —NHCH₃, —NO₂, and —C₁₋₂₀ alkyl, wherein said —C₁₋₂₀ alkyl optionally may be substituted with one or more substituents selected, independently for each occurrence, from the group consisting of halogens, —CF₃, —OCH₃, —OCF₃, and —(CH₂)₀₋₂₀—COOH. In different embodiments 1, 2, 3 or 4 substituents are present.
“Aryl” refers to an optionally substituted aromatic group with at least one ring having a conjugated pi-electron system, containing up to three conjugated or fused ring systems. Aryl includes carbocyclic aryl, heterocyclic aryl and biaryl groups. Preferably, the aryl is a 5- or 6-membered ring. Preferred atoms for a heterocyclic aryl are one or more sulfur, oxygen, and/or nitrogen. Non-limiting examples of aryl include phenyl, 1-naphthyl, 2-naphthyl, indole, quinoline, 2-imidazole, 9-anthracene, and the like. Aryl substituents are selected from the group consisting of —C₁₋₂₀ alkyl, —C₁₋₂₀ alkoxy, halogen (i.e., fluorine, chlorine, bromine, and iodine), —OH, —CN, —SH, —NH₂, —NO₂, —C₁₋₂₀ alkyl substituted with halogens, —CF₃, —OCF₃, and —(CH₂)₀₋₂₀—COH. In different embodiments the aryl contains 0, 1, 2, 3, or 4 substituents.
“Alkylaryl” refers to an “alkyl” joined to an “aryl”.

The term “(C₁₋₁₂)hydrocarbon moiety” encompasses alkyl, alkenyl and alkynyl and in the case of alkenyl and alkynyl there is C₂-C₁₂.

For the avoidance of doubt, unless otherwise indicated, the term substituted means substituted by one or more defined groups. In the case where groups may be selected from a number of alternative groups, the selected groups may be the same or different. For the avoidance of doubt, the term independently means that where more than one substituent is selected from a number of possible substituents, those substituents may be the same or different.

Designation “(amino acid)ₙ” means that an amino acid is repeated n times. For example, designation “(Pro)₂” or “(Arg)₃” mean that proline or arginine residues are repeated, respectively, two or three times.

### MC4R

hMC4R is a protein encoded by a genomic sequence having GenBank accession number CH471077.2. Mutations in the MC4R receptor are an associated cause of severe childhood obesity. The carrier prevalence for MC4R mutations in a juvenile-onset obese population has been noted to be around 2.5% with a highest prevalence of 6% among severely obese children. Humans with MC4R mutations show a more or less similar phenotype as has been described for mice with mutations in the MC4R gene. MC4R deficient patients show hyperphagia, hyperinsulinaemia, increased fat mass, accompanied by lean body mass, bone mineral density and linear growth rate increases, with no changes in cortisol levels, gonadotropin, thyroid and sex steroid levels. In contrast to MC4R deletion, hyperphagia and hyperinsulinaemia tends to subside with age in human subjects. Similar to the MC4R knockout mice, the phenotype in heterozygote carriers is intermediate in comparison to homozygote carriers. The exhibited hyperphagia observed upon a test meal is less severe than that observed in people with a leptin deficiency. The severity of MC4R dysfunction seen in assays in vitro can predict the amount of food ingested at a test meal by the subject harboring that particular mutation and correlates with the onset and severity of the obese phenotype. At least 90 different MC4R mutations have been associated with obesity and additional mutations in the MC4R are likely to be discovered, leading to a similar obesity phenotype.

Examples of the MC4R mutations that cause obesity in humans are described, e.g., in Farooqi et al., The Journal of Clinical Investigation, July 2000, vol. 106 (2), pp. 271-279 and Vaisse et al., The Journal of Clinical Investigation, July 2000, vol. 106(2), pp. 253-262, the relevant portions of which are incorporated herein by reference).

Additional mutations that potentially cause obesity in humans include, R18H, R18L, S36Y, P48S, V50M, F51L, E61K, I69T, D90N, S94R, G98R, I121T, A154D, Y157S, W174C, G181D, F202L, A219 V, 1226T, G231S, G238D, N240S, C271R, S295P, P299L, E308K, I317V, L325F, and 750DelGA, as described in Xiang et al., “Pharmacological characterization of 30 human melanocortin-4 receptor polymorphisms with the endogenous proopiomelanocortin-derived agonists, synthetic agonists, and the endogenous agouti-related protein antagonist.” Biochemistry, 2010 Jun. 8; 49(22):4583-600, the relevant portions of which are incorporated herein by reference.

Further examples of mutations that potentially cause obesity in humans are those listed in Online Mendelian Inheritance in Man (OMIM), a database of human genes and genetic disorders, under the accession number 155541 (MC4R) (more precisely, accession nos. 155541.0001-155541.0023) at the URL http://omim.org/entry/155541. Representative examples include 4-BP DEL, NT631; 4-BP INS, NT732; TYR35TER; ASP37VAL; SER58CYS; ILE102SER; ASN274SER; 1-BP INS, 112A; 4-BP DEL, 211CTCT; ILE125LYS; ALA175THR; ILE316SER; TYR287TER; ASN97ASP; 15-BP DEL (delta88-92 codons); and SER127LEU. The relevant portions of the OMIM database are incorporated herein by reference. Additional exemplary mutations in MC4R are described in Lee. Annals Acad. Med. 38.1(2009):34-44.

In example embodiments, the MC4R mutation results in retention of the MC4R signaling activity. Mutations in the genomic sequence encoding MC4R can be detected by the methods that are known to a person of ordinary skill in the art. For example, the genomic sequence can be cloned using nucleotide primers, such as e.g., the primers described in Farooqi et al., The Journal of Clinical Investigation, July 2000, vol. 106 (2), pp. 271-279 and Vaisse et al., The Journal of Clinical Investigation, July 2000, vol. 106(2), pp. 253-262, and the cloned sequence analyzed using commercially available sequencers and software.

Activity of MC4R can be measured by the methods known to a person of ordinary skill in the art. For example, cells can be transiently transfected with the cloned MC4R DNA, the transfected cells contacted by an agonist of MC4R (e.g. α-MSH), and the intracellular level of cAMP, the secondary messenger of MC4R, measured by an electrochemiluminescence assay described, e.g., in Roubert et al., Journal of Endocrinology (2010) 207, pp. 177-183. A reduction in MC4R signaling can be ascertained by comparing the intracellular level of cAMP produced in response to a given agonist by a wild type MC4R to that produced by a mutant MC4R.

### Melanocortin-4 Receptor (MC4R) Pathway Agonizable Genes

The melanocortin system, which includes melanocortins (MCs), agouti, agouti-related proteins, and their receptors, integrate hormonal, metabolic, and neural signals in order to control energy homeostasis and regulate appetite, energy expenditure, and body weight. The MCs, which include alpha-melanocyte-stimulating hormone (α-MSH), β-MSH, γ-MSH, and ACTH, are a family of peptide hormones that are derived from a precursor protein called pro-opiomelanocortin (POMC). Activation of MC4 receptor (MC4R) in the POMC-MC4R pathway increases energy expenditure and decreases food intake. See, e.g., Fan et al. Nature 1997; 385:165-68. The POMC-MC4R pathway includes a number of proteins, such as melanocortins (MCs), MC4 receptor (MC4R), POMC, Proprotein Convertase Subtilisin/Kexin Type 1 (PCSK1, also called PC1/3), MAGE-like-2 (MAGEL2), leptin receptor (leptin-R), leptin, 5-hydroxytryptamine (serotonin) receptor 2C, G protein-coupled (5-HT2c receptor), nescient helix loop helix 2 (NhHL2, also called NSCL2), pro-hormone convertase, carboxypeptidase E (CPE), and single-minded 1 (Sim1), that together contribute to the regulation of energy homeostasis, e.g., by regulating appetite and energy expenditure. MC4R and other components of the POMC-MC4R pathway have a significant role in weight regulation. A mutation of the MC4R gene was reported to result in early-onset and severe obesity. It is believed that other genetic defects in the POMC-MC4R pathway likely also lead to early-onset and severe obesity. These genes are collectively termed “MC4R pathway agonizable genes” and examples are provided below. In an embodiment, the MC4R pathway agonizable gene does not comprise any one of POMC, Proprotein Convertase Subtilisin/Kexin Type 1 (PCSK1, also called PC1/3), MAGE-like-2 (MAGEL2), leptin receptor (leptin-R), leptin, 5-hydroxytryptamine (serotonin) receptor 2C, G protein-coupled (5-HT2c receptor), nescient helix loop helix 2 (NhHL2, also called NSCL2), pro-hormone convertase, carboxypeptidase E (CPE), and single-minded 1 (Sim1). In an embodiment, the MC4R pathway agonizable gene does not comprise MC4R. In an embodiment, the MC4R pathway agonizable gene does not comprise any gene disclosed in WO2013/102047 or WO 2017/059076, the full contents of each of which is incorporated herein by reference in its entirety.

### ADP Ribosylation Factor-Like GTPase 6 (ARL6)

ADP Ribosylation Factor-like GTPase 6 (ARL6), also known as BBS3, is a member of the ARF-like (ADP ribosylation factor-like) sub-family of the ARF family of GTP-binding proteins, which are involved in the regulation of intracellular traffic. ARL6 is involved in membrane protein trafficking at the base of the ciliary organelle and mediates recruitment onto plasma membrane of the BBSome complex. Together with BBS1, ARL6 is necessary for correct trafficking of PKD1 to primary cilia. Together with the BBSome complex and LTZL1, ARL6 controls SMO ciliary trafficking and contributes to the sonic hedgehog (SHH) pathway regulation. It is believed that ARL6 may regulate cilia assembly and disassembly and subsequent ciliary signaling events such as the Wnt signaling cascade. ARL6 isoform 2 may be required for proper retinal function and organization. A vision-specific transcript, encoding long isoform BBS3L, has also been described.

Mutations in the ARL6 gene are associated with Bardet-Biedl syndrome (BBS), a genetically heterogeneous disorder. BBS is a form of Laurence-Moon-Beidl syndrome and is characterized by obesity, retinopathy, learning disability, polydactyly, hypogenitalism, and retinitis pigmentosa 55. (See, e.g., Young et al. Am. J. Med. Genet. 78(5):461-7 (2002)).

The human ARL6 gene sequence is provided in GenBank Accession No. NG_008119.2, incorporated herein by reference. An exemplary human ARL6 nucleic acid sequence is provided in GenBank Accession No. NM_001278293.3, incorporated herein by reference. An exemplary amino acid sequence of human ARL6 is provided by Q9H0F7, incorporated herein by reference.

### Retinoic Acid Induced 1(RAI1)

Retinoic Acid Induced 1 (RAI1) is a transcription factor that regulates the circadian clock components: CLOCK, ARNTL/BMAL1 ARNTL2/BMAL2, PER1/3, CRY1/2, NR1D1/2, and RORA/C. RAI1 positively regulates the transcriptional activity of CLOCK, a core component of the circadian clock. (See, e.g., Williams et al. Am. J. Hum. Genet. 90(6):941-9 (2012)). RAI1 also regulates transcription through chromatin remodeling by interacting with other proteins in chromatin as well as proteins in the basic transcriptional machinery. It is believed that RAI1 may be important for embryonic and postnatal development and may be involved in neuronal differentiation.

Mutations in RAI1 (e.g., leading to haploinsufficiency) are associated with Smith-Magenis Syndrome, a disorder characterized by cognitive and behavioral abnormalities, including self-injurious behaviors and sleep disturbance, obesity, and distinct craniofacial and skeletal anomalies, that has been associated with deletions involving chromosome 17p11.2. (See, e.g., Slager et al. Nat Genet. 33(4):466-468 (2003)).

The human RAI1 gene sequence is provided in GenBank Accession No. NG_007101.2, incorporated herein by reference. An exemplary human RAI1 nucleic acid sequence is provided in GenBank Accession No. NM_030665.4, incorporated herein by reference. An exemplary amino acid sequence of human RAI1 is provided by Q7Z5J4-1, incorporated herein by reference.

### Steroid Receptor Coactivator 1 (SRC1)

Steroid Receptor Coactivator 1 (SRC1), also known as Nuclear Receptor Coactivator 1 (NCOA1), is a transcriptional coactivator for steroid and nuclear hormone receptors. SRC1 is a member of the p160/SRC family, and like other family members, has histone acetyltransferase activity and contains a nuclear localization signal, as well as bHLH and PAS domains. SRC1 binds nuclear receptors directly and stimulates the transcriptional activities in a hormone-dependent fashion. SRC1 is involved in the coactivation of different nuclear receptors, such as for steroids, retinoids, thyroid hormone, and prostanoids. SRC1 is also involved in coactivation mediated by STAT3, STAT5A, STAT5B, and STAT6 transcription factors. SRC1 plays a central role in creating multi-subunit coactivator complexes that act via remodeling of chromatin, and possibly acts by participating in both chromatin remodeling and recruitment of general transcription factors. It is required with NCOA2 to control energy balance between white and brown adipose tissues and for mediating steroid hormone response. Alternatively spliced transcript variants encoding different isoforms have also been identified.

Mutations in SRC1 has been linked to obesity. Without wishing to be bound by theory, it is believed that SRC-1 modulates the function of hypothalamic Pro-opiomelanocortin (Pomc) neurons, which regulate food intake and body weight. Rare heterozygous variants of SRC1 were found in severely obese individuals that impaired leptin mediated Pomc reporter activity in cells. (See, e.g., Yang et al. Nat. Commun. 10(1):1718 (2019)).

The human SRC1 gene sequence is provided in GenBank Accession No. NG_029014.2, incorporated herein by reference. An exemplary human SRC1 nucleic acid sequence is provided in GenBank Accession No. NM_003743.5, incorporated herein by reference. An exemplary amino acid sequence of human SRC1 is provided by Q15788-1, incorporated herein by reference.

### Bardet-Biedl Syndrome 19 (BBS19)

Bardet-Biedl Syndrome 19 (BBS19), also known as intraflagellar transport protein 27 homolog (IFT27), is a small GTPase-like component of the intraflagellar transport complex B, which is essential for cilia biogenesis and maintenance. BBS19 promotes the exit of the BBSome complex from cilia via its interaction with ARL6. BBS19 forms a subcomplex within the IFT complex B with IFT25 and prevents aggregation of GTP-free ARL6 but is not believed to be involved in entry of the BBSome complex into cilium. (See, e.g., Liew et al. Dev. Cell 31(3):265-278 (2014)). BBS19 is also required for hedgehog signaling. Its role in intraflagellar transport is mainly seen in tissues rich in ciliated cells such as kidney and testis. BBS19 is essential for male fertility, spermiogenesis and sperm flagella formation, plays a role in the early development of the kidney, and may be involved in the regulation of ureteric bud initiation.

Mutations in the BBS19 gene have been associated with Bardet-Biedl syndrome (See, e.g., Aldahmesh et al. Hum. Mol. Genet. 23(12):3307-15 (2014)).

The human BBS19 gene sequence is provided in GenBank Accession No. NG_034205.1, incorporated herein by reference. An exemplary human BBS19 nucleic acid sequence is provided in GenBank Accession No. NM_001177701.3, incorporated herein by reference. An exemplary amino acid sequence of human BBS19 is provided by Q9BW83-1, incorporated herein by reference.

### Bardet-Biedl Syndrome 21 (BBS21)

The Bardet-Biedl syndrome 21 (BBS21) gene, also known as chromosome 8 open reading frame 37 (C8orf37), encodes a broadly expressed protein of unknown function. High levels of BBS21 mRNA can be found in the brain, heart, and retina. The protein has been shown to co-localize with polyglutamylated tubulin at the base of the primary cilium in human retinal pigment epithelial cells. Mutations in the BBS21 gene have been associated with Bardet-Biedl syndrome, autosomal recessive cone-rod dystrophy (arCRD), and retinitis pigmentosa (See, e.g., Heon et al. Hum. Mol. Genet. 25(11):2283-2294 (2016)).

The human BBS21 gene sequence is provided in GenBank Accession No. NG_032804.1, incorporated herein by reference. An exemplary human BBS21 nucleic acid sequence is provided in GenBank Accession No. NM_177965.4, incorporated herein by reference. An exemplary amino acid sequence of human BBS21 is provided by Q96NL8-1, incorporated herein by reference.

### Centrosomal Protein 290 (CEP290)

Centrosomal Protein 290 (CEP290), also known as BBS14, encodes a protein with thirteen putative coiled-coil domains, a region with homology to SMC chromosome segregation ATPases, six KID motifs, three tropomyosin homology domains, and an ATP/GTP binding site motif A. The protein is localized to the centrosome and cilia and has sites for N-glycosylation, tyrosine sulfation, phosphorylation, N-myristoylation, and amidation.

CEP290 is involved in early and late steps in cilia formation and its association with CCP110 is required for inhibition of primary cilia formation by CCP10. CEP290 may play a role in early ciliogenesis in the disappearance of centriolar satellites and in the transition of primary ciliar vesicles (PCVs) to capped ciliary vesicles (CCVs). CEP290 is also required for the centrosomal recruitment of RAB8A and for the targeting of centriole satellite proteins to centrosomes such as of PCM1. It is required for the correct localization of ciliary and phototransduction proteins in retinal photoreceptor cells and may play a role in ciliary transport processes. Required for efficient recruitment of RAB8A to primary cilium. In the ciliary transition zone, CEP290 is part of the tectonic-like complex, which is required for tissue-specific ciliogenesis and may regulate ciliary membrane composition. CEP290 is involved in regulation of the BBSome complex integrity, specifically for presence of BBS2, BBS5, and BBS8/TTC8 in the complex, and in ciliary targeting of selected BBSome cargos. CEP290 may play a role in controlling entry of the BBSome complex to cilia.

Mutations in this gene have been associated with several ciliopathies including Bardet-Biedl syndrome, isolated retinal degeneration, nephronophthisis (NPHP), Joubert syndrome, Senior-Loken syndrome (SLSN), and neonatal lethal Meckel-Gruber syndrome (MKS). (See, e.g., Zhang et al. Hu. Mol. Genet. 23(1):40-51 (2014) and Leitch et al. Nat. Genet. 40(4):443-448 (2008)).

The human CEP290 gene sequence is provided in GenBank Accession No. NG_008417.2, incorporated herein by reference. An exemplary human CEP290 nucleic acid sequence is provided in GenBank Accession No. NM_025114.4, incorporated herein by reference. An exemplary amino acid sequence of human CEP290 is provided by 015078-1, incorporated herein by reference.

### Intraflagellar Transport 74 (IFT74)

Intraflagellar Transport 74 (IFT74) is a core component of the intraflagellar transport (IFT), a multi-protein complex involved in the transport of ciliary proteins along axonemal microtubules. IFT proteins are found at the base of the cilium as well as inside the cilium, where they assemble into long arrays between the ciliary base and tip. Specifically, IFT74, together with IFT81, forms a tubulin-binding module that specifically mediates transport of tubulin within the cilium. IFT74 binds beta-tubulin via its basic region and is required for ciliogenesis.

Naturally occurring mutations in this gene are associated with Bardet-Biedl Syndrome and amyotrophic lateral sclerosis—frontotemporal dementia. (See, e.g., Lindstrand et al. Am. J. Hum. Genet. 99(2):318-336 (2016)).

The human IFT74 gene sequence is provided in GenBank Accession No. NG_053083.1, incorporated herein by reference. An exemplary human IFT74 nucleic acid sequence is provided in GenBank Accession No. NM_001099222.2, incorporated herein by reference. An exemplary amino acid sequence of human IFT74 is provided by Q96LB3-1, incorporated herein by reference.

### Leucine Zipper Transcription Factor Like 1 (LZTFL1)

Leucine Zipper Transcription Factor Like 1 (LZTFL1), also known as BBS17, encodes a ubiquitously expressed protein that localizes to the cytoplasm. The protein interacts with Bardet-Biedl Syndrome (BBS) proteins and, through its interaction with BBS protein complexes, regulates protein trafficking to the ciliary membrane. LZTFL1 regulates ciliary localization of the BBSome complex and, together with the BBSome complex, controls SMO ciliary trafficking and contributes to the sonic hedgehog (SHH) pathway regulation.

Nonsense mutations in this gene are associated with a form of Bardet-Biedl Syndrome. (See, e.g., Deffert et al. Am. J. Med. Genet. A. 143A(2):208-213 (2007)). LZTFL1 may also function as a tumor suppressor; possibly by interacting with E-cadherin and the actin cytoskeleton and thereby regulating the transition of epithelial cells to mesenchymal cells. Alternative splicing of LZTFL1 results in multiple transcript variants.

The human LZTFL1 gene sequence is provided in GenBank Accession No. NG_033917.1, incorporated herein by reference. An exemplary human LZTFL1 nucleic acid sequence is provided in GenBank Accession No. NM_020347.4, incorporated herein by reference. An exemplary amino acid sequence of human LZTFL1 is provided by Q9NQ48-1, incorporated herein by reference.

### MKS Transition Zone Complex Subunit 1 (MKS1)

MKS Transition Zone Complex Subunit 1 (MKS1), also known as BBS13, is a component of the tectonic-like complex, a complex localized at the transition zone of primary cilia and acting as a barrier that prevents diffusion of transmembrane proteins between the cilia and plasma membranes. MKS1 localizes to the basal body and is involved in centrosome migration to the apical cell surface during early ciliogenesis, is required for formation of the primary cilium in ciliated epithelial cells, and is required for ciliary structure and function, including a role in regulating length and appropriate number through modulating centrosome duplication. MKS1 is also required for cell branching morphology.

Mutations in this gene result in Meckel syndrome type 1 and in Bardet-Biedl syndrome type 13. (See, e.g., Xing et al. PLoS One 9(3):e90599 (2014)). Multiple transcript variants encoding different isoforms have been identified for this gene.

The human MKS1 gene sequence is provided in GenBank Accession No. NG_013032.1, incorporated herein by reference. An exemplary human MKS1 nucleic acid sequence is provided in GenBank Accession No. NM_017777.4, incorporated herein by reference. An exemplary amino acid sequence of human MKS1 is provided by Q9NXB0-1, incorporated herein by reference.

### Tripartite Motif Containing 32 (TRIM32)

Tripartite Motif Containing 32 (TRIM32), also known as BBS11, is a member of the tripartite motif (TRIM) family. The protein encoded by the TRIM32 gene contains three zinc-binding domains, a RING, a B-box type 1 and a B-box type 2, and a coiled-coil region. The protein encoded by TRIM32 localizes to cytoplasmic bodies and to the nucleus, where it interacts with the activation domain of the HIV-1 Tat protein. The TRIM32 protein also has E3 ubiquitin ligase activity and has been shown to ubiquitinate DTNBP1 (dysbindin) and promotes its degradation. It may also ubiquitinate BBS2.

Mutations in TRIM32 have been associated with muscular dystrophy, limb-girdle, autosomal recessive 8, and Bardet-Biedl syndrome (See, e.g., Chiang et al. Proc. Natl. Acad. Sci. U.S.A. 103(16):3287-92 (2006)).

The human TRIM32 gene sequence is provided in GenBank Accession No. NG_011619.1, incorporated herein by reference. An exemplary human TRIM32 nucleic acid sequence is provided in GenBank Accession No. NM_012210.4, incorporated herein by reference. An exemplary amino acid sequence of human TRIM32 is provided by Q13049-1, incorporated herein by reference.

### WD Repeat Containing Planar Cell Polarity Effector (WDPCP)

WD Repeat Containing Planar Cell Polarity Effector (WDPCP), also known as BBS15, is a cytoplasmic WD40 repeat protein. WDPCP is proposed to act as a planar cell polarity protein, which plays a critical role in collective cell movement and ciliogenesis by mediating septin localization. Together with FUZ, WDPCP is proposed to function as core component of the CPLANE (ciliogenesis and planar polarity effectors) complex involved in the recruitment of peripheral IFT-A proteins to basal bodies.

Mutations in this gene are associated with Bardet-Biedl syndrome and may also play a role in Meckel-Gruber syndrome. (See, e.g., Kim et al. Science 329(5997):1337-40 (2010)). Alternative splicing results in multiple transcript variants.

The human WDPCP gene sequence is provided in GenBank Accession No. NG_028144.2, incorporated herein by reference. An exemplary human WDPCP nucleic acid sequence is provided in GenBank Accession No. NM_001042692.3, incorporated herein by reference. An exemplary amino acid sequence of human WDPCP is provided by 095876-1, incorporated herein by reference.

### Ribosomal Protein S6 Kinase A3 (RPS6KA3)

Ribosomal Protein S6 Kinase A3 (RPS6KA3) is a member of the RSK (ribosomal S6 kinase) family of serine/threonine kinases that acts downstream of ERK (MAPK1/ERK2 and MAPK3/ERK1) signaling and mediates mitogenic and stress-induced activation of the transcription factors CREB1, ETV1/ER81, and NR4A1/NUR77, regulates translation through RPS6 and EIF4B phosphorylation, and mediates cellular proliferation, survival, and differentiation by modulating mTOR signaling and repressing pro-apoptotic function of BAD and DAPK1. In fibroblasts, RPS6KA3 is required for EGF-stimulated phosphorylation of CREB1 and histone H3 at Ser-10□which results in the subsequent transcriptional activation of several immediate-early genes. In response to mitogenic stimulation (EGF and PMA), RPS6KA3 phosphorylates and activates NR4A1/NUR77 and ETV1/ER81 transcription factors and the cofactor CREBBP. Upon insulin-derived signal, RPS6KA3 acts indirectly on the transcription regulation of several genes by phosphorylating GSK3B at Ser-9□and inhibiting its activity. RPS6KA3 also phosphorylates RPS6 in response to serum or EGF via an mTOR-independent mechanism and promotes translation initiation by facilitating assembly of the preinitiation complex. In response to insulin, RPS6KA3 phosphorylates EIF4B, enhancing EIF4B affinity for the EIF3 complex and stimulating cap-dependent translation. RPS6KA3 is involved in the mTOR nutrient-sensing pathway by directly phosphorylating TSC2 at Ser-1798□which potently inhibits TSC2 ability to suppress mTOR signaling, and mediates phosphorylation of RPTOR, which regulates mTORC1 activity and may promote rapamycin-sensitive signaling independently of the PI3K/AKT pathway. RPS6KA3 mediates cell survival by phosphorylating the pro-apoptotic proteins BAD and DAPK1 and suppressing their pro-apoptotic function. RPS6KA3 promotes the survival of hepatic stellate cells by phosphorylating CEBPB in response to the hepatotoxin carbon tetrachloride (CCl4). RPS6KA3 is also involved in cell cycle regulation by phosphorylating the CDK inhibitor CDKN1B, which promotes CDKN1B association with 14-3-3 proteins and prevents its translocation to the nucleus and inhibition of G1 progression. In LPS-stimulated dendritic cells, RPS6KA3 is involved in TLR4-induced macropinocytosis, and in myeloma cells, it acts as effector of FGFR3-mediated transformation signaling, after direct phosphorylation at Tyr-529 by FGFR3. RPS6KA3 negatively regulates EGF-induced MAPK1/3 phosphorylation via phosphorylation of SOS1. RPS6KA3 phosphorylates SOS1 at Ser-1134□and Ser-1161□ that create YWHAB and YWHAE binding sites and which contribute to the negative regulation of MAPK1/3 phosphorylation and phosphorylates EPHA2 at Ser-897□ the RPS6KA-EPHA2 signaling pathway controls cell migration.

Mutations in this gene have been associated with Coffin-Lowry syndrome (CLS), a rare X-linked semi-dominant syndrome characterized by severe psychomotor retardation, facial dysmorphism, digit abnormalities, and progressive skeletal deformations. (See, e.g., Delaunoy et al. Clin. Genet. 70(2): 161-6 (2006)).

The human RPS6KA3 gene sequence is provided in GenBank Accession No. NG_007488.1, incorporated herein by reference. An exemplary human RPS6KA3 nucleic acid sequence is provided in GenBank Accession No. NM_004586.3, incorporated herein by reference. An exemplary amino acid sequence of human RPS6KA3 is provided by P51812-1, incorporated herein by reference.

### 5-Hydroxytryptamine Receptor 2C (HTR2C)

5-Hydroxytryptamine Receptor 2C (HTR2C) is a seven-transmembrane G-protein-coupled receptor for 5-hydroxytryptamine (serotonin). HTR2C also functions as a receptor for various drugs and psychoactive substances, including ergot alkaloid derivatives, 1-2,5,-dimethoxy-4-iodophenyl-2-aminopropane (DOI) and lysergic acid diethylamide (LSD). Ligand binding causes a conformational change that triggers signaling via guanine nucleotide-binding proteins (G proteins) and modulates the activity of down-stream effectors, Beta-arrestin family members inhibit signaling via G proteins and mediate activation of alternative signaling pathways. Signaling activates a phosphatidylinositol-calcium second messenger system that modulates the activity of phosphatidylinositol 3-kinase and down-stream signaling cascades and promotes the release of Ca²⁺ ions from intracellular stores. HTR2C also regulates neuronal activity via the activation of short transient receptor potential calcium channels in the brain, and thereby modulates the activation of pro-opiomelacortin neurons and the release of CRH that then regulates the release of corticosterone. HTR2C plays a role in the regulation of appetite and eating behavior, responses to anxiogenic stimuli and stress, and also plays a role in insulin sensitivity and glucose homeostasis.

The mRNA of HTR2C is subject to multiple RNA editing events, where adenosine residues encoded by the genome are converted to inosines. RNA editing is predicted to alter the structure of the second intracellular loop, thereby generating alternate protein forms with decreased ability to interact with G proteins. Abnormalities in RNA editing of HTR2C have been detected in victims of suicide that suffer from depression. In addition, naturally occurring variation in the promoter and 5 □hon-coding and coding regions of HTR2C may show statistically significant association with mental illness and behavioral disorders. Alternative splicing results in multiple different transcript variants. Mutations in HTR2C have been linked to hyperphagia, hyperactivity, and obesity. (See, e.g., Xu et al. Neuron. 60(4):582-9 (2008)).

The human HTR2C gene sequence is provided in GenBank Accession No. NG_012082.2, incorporated herein by reference. An exemplary human HTR2C nucleic acid sequence is provided in GenBank Accession No. NM_001256760.2, incorporated herein by reference. An exemplary amino acid sequence of human HTR2C is provided by P28335-1, incorporated herein by reference.

### Kinase Suppressor of Ras 2 (KSR2)

Kinase Suppressor of Ras 2 (KSR2) is an intracellular scaffolding protein involved in multiple signaling pathways. In particular, KSR2 is a location-regulated scaffold connecting MEK to RAF. KSR2has been shown to have very low protein kinase activity and can phosphorylate MAP2K1 at several Ser and Thr residues with very low efficiency in vitro. KSR2acts as MAP2K1/MEK1-dependent allosteric activator of BRAF; upon binding to MAP2K1/MEK1, KSR2dimerizes with BRAF and promotes BRAF-mediated phosphorylation of MAP2K1/MEK1 (See, e.g., Lavoie et al. Nature 554:549-553(2018)). Interaction with BRAF enhances KSR2-mediated phosphorylation of MAP2K1 in vitro. KSR2blocks MAP3K8 kinase activity and MAP3K8-mediated signaling. KSR2also acts as a negative regulator of MAP3K3-mediated activation of ERK, JNK and NF-kappa-B pathways, inhibiting MAP3K3-mediated interleukin-8 production.

Mutations in KSR2are linked to hyperphagia in childhood, low heart rate, reduced basal metabolic rate and severe insulin resistance, suggesting that KSR2 is an important regulator of energy intake, energy expenditure, and substrate utilization in humans. (See, e.g., Pearce et al. Cell. 155(4):765-77 (2013)).

The human KSR2 gene sequence is provided within GenBank Accession No. NC_000012.12, incorporated herein by reference. An exemplary human KSR2 nucleic acid sequence is provided in GenBank Accession No. NM_173598.6, incorporated herein by reference. An exemplary amino acid sequence of human KSR2 is provided by Q6VAB6-1, incorporated herein by reference.

### Prokineticin 2 (PROK2)

The prokineticin 2 (PROK2) gene encodes a protein expressed in the suprachiasmatic nucleus (SCN) circadian clock that may function as the output component of the circadian clock. The secreted form of the encoded protein may also serve as a chemoattractant for neuronal precursor cells in the olfactory bulb. Proteins from other vertebrates which are similar to the PROK2 gene product were isolated based on homology to snake venom; secretions from frog skin and have been shown to have diverse functions.

Mutations in PROK2 are associated with hypogonadotropic hypogonadism 4 with or without anosmia and Kallmann syndrome. Multiple transcript variants encoding different isoforms have been found for this gene. (See, e.g., Dode et al. PLoS Genet. 2(10):e175 (2006)).

The human PROK2 gene sequence is provided in GenBank Accession No. NG_008275.1, incorporated herein by reference. An exemplary human PROK2 nucleic acid sequence is provided in GenBank Accession No. NM_001126128.2, incorporated herein by reference. An exemplary amino acid sequence of human PROK2 is provided by Q9HC23-1, incorporated herein by reference.

### Ras-Related Protein Rab-23 (RAB23)

Ras-Related Protein Rab-23 (RAB23) is a small GTPase of the Ras superfamily. The small GTPases Rab are involved in the regulation of diverse cellular functions associated with intracellular membrane trafficking, including autophagy and immune response to bacterial infection. Rabs cycle between an inactive GDP-bound form and an active GTP-bound form that is able to recruit to membranes different set of downstream effectors directly responsible for vesicle formation, movement, tethering, and fusion. Together with SUFU, the protein encoded by RAB23 prevents nuclear import of GLI1 and thereby inhibits GLI1 transcription factor activity. RAVB23 also regulates GLI1 in differentiating chondrocytes, regulates GLI3 proteolytic processing, and modulates GLI2 and GLI3 transcription factor activity. RAB23 also plays a role in autophagic vacuole assembly, and mediates defense against pathogens, such as S. aureus, by promoting their capture by autophagosomes that then merge with lysosomes. RAB23 may play a role in central nervous system development by antagonizing sonic hedgehog signaling.

Mutations in RAB23 have been associated with cancer and Carpenter syndrome, a pleiotropic disorder with autosomal recessive inheritance, the cardinal features of which include craniosynostosis, polysyndactyly, obesity, and cardiac defects. (See, e.g., Jenkins et al. Am. J. Hum. Genet. 80(6):1162-70 (2007)). Alternative splicing results in multiple transcript variants.

The human RAB23 gene sequence is provided in GenBank Accession No. NG_012170.1, incorporated herein by reference. An exemplary human RAB23 nucleic acid sequence is provided in GenBank Accession No. NM_016277.5, incorporated herein by reference. An exemplary amino acid sequence of human RAB23 is provided by Q9ULC3-1, incorporated herein by reference.

### Melanocortin 2 Receptor Accessory Protein 2 (MRAP2)

Melanocortin 2 Receptor Accessory Protein 2 (MRAP2) is a G-protein-coupled receptor accessory protein that modulates melanocortin receptor signaling and is involved in energy homeostasis. The encoded protein has been shown to interact with all known melanocortin receptors and may regulate both receptor trafficking and activation in response to ligands. MRAP2 is thought to play a central role in the control of energy homeostasis and body weight regulation by increasing ligand-sensitivity of MC4R and MC4R-mediated generation of cAMP. MRAP2 may also act as a negative regulator of MC2R (e.g., by competing with MRAP for binding to MC2R and impairs the binding of corticotropin (ACTH) to MC2R). MRAP2 may also regulate activity of other melanocortin receptors (MC1R, MC3R and MC5R). MRAP2 has been implicated in energy control in rodents, notably via the melanocortin-4 receptor.

Deficiencies in MRAP2 have been associated with obesity (e.g., monogenic hyperphagic obesity, hyperglycemia, and hypertension) in both children and adults. (See, e.g., Baron et al. Nat. Med. 25(11):1733-1738 (2019)).

The human MRAP2 gene sequence is provided in GenBank Accession No. NG_051944.1, incorporated herein by reference. An exemplary human MRAP2 nucleic acid sequence is provided in GenBank Accession No. NM_138409.4, incorporated herein by reference. An exemplary amino acid sequence of human MRAP2 is provided by Q96G30-1, incorporated herein by reference.

### AF4/FMR2 Family Member 4 (AFF4)

AF4/FMR2 family member 4 (AFF4) is a component of the positive transcription elongation factor b (P-TEFb) complex, a core component of the super elongation complex (SEC), which is required to increase the catalytic rate of RNA polymerase II transcription by suppressing transient pausing by the polymerase at multiple sites along the DNA. In the SEC complex, AFF4 acts as a central scaffold that recruits other factors through direct interactions with ELL proteins (e.g., ELL, ELL2, or ELL3) and the P-TEFb complex. In case of infection by HIV-1 virus, the SEC complex is recruited by the viral Tat protein to stimulate viral gene expression.

Chromosomal aberrations involving ATF4 have been found in acute lymphoblastic leukemia (ALL). Missense mutations in AFF4 have been associated with CHOPS syndrome (C for cognitive impairment and coarse facies, H for heart defects, O for obesity, P for pulmonary involvement and S for short stature and skeletal dysplasia). (See, e.g., Izumi et al. Nat. Genet. 47(4):338-44 (2015)).

The human AFF4 gene sequence is provided in GenBank Accession No. NG_030340.1, incorporated herein by reference. An exemplary human AFF4 nucleic acid sequence is provided in GenBank Accession No. NM_014423.4, incorporated herein by reference. An exemplary amino acid sequence of human AFF4 is provided by Q9UHB7-1, incorporated herein by reference.

### Adenylate Cyclase 3 (ADCY3)

Adenylate cyclase 3 (ADCY3) is a membrane-associated enzyme and catalyzes the formation of the secondary messenger cyclic adenosine monophosphate (cAMP). ADCY3 catalyzes the formation of the signaling molecule cAMP in response to G-protein signaling and participates in signaling cascades triggered by odorant receptors via its function in cAMP biosynthesis. ADCY3 is required for the perception of odorants, for normal sperm motility, and normal male fertility. ADCY3 also plays a role in regulating insulin levels and body fat accumulation in response to a high fat diet. ADCY3 is widely expressed in various human tissues and may be involved in a number of physiological and pathophysiological metabolic processes. Two transcript variants encoding different isoforms have been identified for ADCY3.

Loss of function mutations in ADCY4 have been associated with monogenic severe obesity. (See, e.g., Saeed et al. Nat. Genet. 50(2):175-179 (2018)).

The human ADCY3 gene sequence is provided within GenBank Accession No. NC_000002.12, incorporated herein by reference. An exemplary human ADCY3 nucleic acid sequence is provided in GenBank Accession No. NM_001320613.2, incorporated herein by reference. An exemplary amino acid sequence of human ADCY3 is provided by 060266-1, incorporated herein by reference.

### TUB Bipartite Transcription Factor (TUB)

TUB Bipartite Transcription Factor (TUB) is a member of the Tubby family of bipartite transcription factors that functions in signal transduction from heterotrimeric G protein-coupled receptors. The crystal structure has been determined for a similar protein in mouse, which functions as a membrane-bound transcription regulator that translocates to the nucleus in response to phosphoinositide hydrolysis. TUB binds to membranes containing phosphatidylinositol 4,5-bisphosphate and has been shown to bind DNA in vitro. TUB may contribute to the regulation of transcription in the nucleus and could be involved in the hypothalamic regulation of body weight. TUB contributes to stimulation of phagocytosis of apoptotic retinal pigment epithelium (RPE) cells and macrophages. Two transcript variants encoding distinct isoforms have been identified for this gene.

Mutations in TUB have been associated with obesity and retinal dystrophy (e.g., characterized by obesity, night blindness, decreased visual acuity, and electrophysiological features of a rod cone dystrophy). (See, e.g., Borman et al. Hum. Mutat. 35(3):289-93 (2014)).

The human TUB gene sequence is provided in GenBank Accession No. NG_029912.1, incorporated herein by reference. An exemplary human TUB nucleic acid sequence is provided in GenBank Accession No. NM_003320.4, incorporated herein by reference. An exemplary amino acid sequence of human TUB is provided by P50607-1, incorporated herein by reference.

### Orthopedia Homeobox (OTP)

Orthopedia Homeobox (OTP) is a member of the homeodomain (HD) family. HD family proteins are helix-turn-helix transcription factors that play key roles in the specification of cell fates. OTP may function during brain development, specifically in the differentiation of hypothalamic neuroendocrine cells. OTP is also believed to be involved in mammalian energy homeostasis and behavior.

Disruption of OTP has been associated with obesity, marasmus, Kwashiorkor, and anxiety (See, e.g., Moir et al. Mol. Metab. 6(11):1419-1428 (2017)).

The human OTP gene sequence is provided within GenBank Accession No. NC_000005.10, incorporated herein by reference. An exemplary human OTP nucleic acid sequence is provided in GenBank Accession No. NM_032109.3, incorporated herein by reference. An exemplary amino acid sequence of human OTP is provided by Q5XKR4-1, incorporated herein by reference.

### G-Protein Coupled Receptor 101 (GPR101)

G-Protein Coupled Receptor 101 (GPR101) is an orphan G protein-coupled receptor of largely unknown function. The encoded protein is a member of a family of proteins that contain seven transmembrane domains and transduce extracellular signals through heterotrimeric G proteins.

Diseases associated with GPR101 include Pituitary Adenoma 2, Growth Hormone-Secreting and Chromosome Xq26.3 Duplication Syndrome. Neuronal (GLP1Rs has been shown to mediate body weight and anorectic effects of liraglutide but are not required for glucose-lowering effects. (See, e.g., Sisley et al. J. Clin. Invest. 124(6):2456-63 (2014)).

The human GPR101 gene sequence is provided in GenBank Accession No. NG_016367.1, incorporated herein by reference. An exemplary human GPR101 nucleic acid sequence is provided in GenBank Accession No. NM_054021.2, incorporated herein by reference. An exemplary amino acid sequence of human GPR101 is provided by Q96P66-1, incorporated herein by reference.

### T-Box Transcription Factor 3 (TBX3)

T-Box Transcription Factor 3 (TBX3) is a member of a phylogenetically conserved family of genes that share a common DNA-binding domain, the T-box. T-box genes encode transcription factors involved in the regulation of developmental processes. TBX3 is a transcriptional repressor and is thought to play a role in the anterior/posterior axis of the tetrapod forelimb. TBX3 acts as a negative regulator of PML function in cellular senescence. TBX3 may also play a role in limb pattern formation. Alternative splicing of this gene results in three transcript variants encoding different isoforms.

Mutations that disrupt the DNA-binding domain of TBX3 have been associated with Ulnar-mammary syndrome (UMS), a pleiotropic disorder affecting limb, apocrine-gland, tooth, hair, and genital development. (See, e.g., Bamshad et al. Am. J. Hum. Genet. 64(6):1550-62 (1999)).

The human TBX3 gene sequence is provided in GenBank Accession No. NG_008315.1, incorporated herein by reference. An exemplary human TBX3 nucleic acid sequence is provided in GenBank Accession No. NM_016569.4, incorporated herein by reference. An exemplary amino acid sequence of human TBX3 is provided by 015119-1, incorporated herein by reference.

In embodiments of any method described herein, the method comprises treating a subject having a mutation in a gene listed in Table 1 below. In embodiments, a method described herein comprises use of a MC4R agonist described herein to treat a subject having a mutation in an MC4R pathway agonizable gene, e.g., as listed in Table 1. Table 1 describes exemplary genes, alleles, transcripts, and proteins, though other genes, alleles, transcripts, and proteins may be included.

TABLE 1
Exemplary MC4R pathway agonizable
genes, alleles, and transcripts

| | NCBI Reference (exemplary | UniProt Reference |
|---|---|---|
| Gene Name | transcripts) | (Amino Acid) |
| ARL6 | NM_001278293.3 | Q9H0F7 |
| RAI1 | NM_030665.4 | Q7Z5J4-1 |
| SRC1 | NM_003743.5 | Q15788-1 |
| BBS19 | NM_001177701.3 | Q9BW83-1 |
| BBS21 | NM_177965.4 | Q96NL8-1 |
| CEP290 | NM_025114.4 | O15078-1 |
| IFT74 | NM_001099222.2 | Q96LB3-1 |
| LZTFL1 | NM_020347.4 | Q9NQ48-1 |
| MKS1 | NM_017777.4 | Q9NXB0-1 |
| TRIM32 | NM_012210.4 | Q13049-1 |
| WDPCP | NM_001042692.3 | O95876-1 |
| RPS6KA3 | NM_004586.3 | P51812-1 |
| HTR2C | NM_001256760.2 | P28335-1 |
| KSR2 | NM_173598.6 | Q6VAB6-1 |
| PROK2 | NM_001126128.2 | Q9HC23-1 |
| RAB23 | NM_016277.5 | Q9ULC3-1 |
| MRAP2 | NM_138409.4 | Q96G30-1 |
| AFF4 | NM_014423.4 | Q9UHB7-1 |
| ADCY3 | NM_001320613.2 | O60266-1 |
| TUB | NM_003320.4 | P50607-1 |
| OTP | NM_032109.3 | Q5XKR4-1 |
| GPR101 | NM_054021.2 | Q96P66-1 |
| TBX3 | NM_016569.4 | O15119-1 |

### Additional MC4R Pathway Agonizable Genes

Additional MC4R pathway agonizable genes useful in the methods disclosed herein are described as follows:
Acyl-CoA Binding Domain Containing 7 (ACBD7), also known as BA455B2.2, has been associated with food intake, energy expenditure, and body weight in preclinical models. (See, e.g., Lanfray et al. Elife. 15; 5:e11742 (2016)).
Agouti Related Neuropeptide (AGRP), also known as ASIP2, has been associated with hyperphagia and obesity. (See, e.g., Carroll et al. Clin. Dermatol. 22(4):345-9 (2004)).
Cell Adhesion Molecule 1 (CADM1), also known as TSLC1 or IGSF4, has been associated with obesity. (See, e.g., Rathjen et al. Nat. Neurosci. 20(8):1096-1103 (2017)).
Cell Adhesion Molecule 2 (CADM2), also known as IGSF4D, has been associated with obesity. (See e.g., Li et al. Hum. Genet. 132(7):793-801 (2013)).
Cocaine and Amphetamine-Regulated Transcript Protein (CARTPT), also known as CART, has been associated with obesity. (See, e.g., Asnicar et al. Endocrinology. 42(10):4394-400 (2001)).
Coiled-Coil Domain Containing 28B (CCDC28B) has been associated with Bardet-Biedl syndrome. (See, e.g., Novas et al. Sic. Rep. 14; 8(1):3019 (2018)).
Cholecystokinin (CCK), also known as Prepro-Cholecystokinin, has been associated with obesity and body mass index. (See, e.g., Namjou et al. Front. Genet. 3; 4:268 (2013)).
Cannabinoid Receptor 1 (CNR1), also known as CNR, has been associated with obesity and body fat mass and distribution. (See, e.g., Russo et al. J. Endocrinol. Metab. 92(6):2382-6 (2007)).
CREB Binding Protein (CREBBP), also known as RSTS, has been associated with Rubinstein-Taybi syndrome. (See, e.g., Stevens et al. Am. J. Med. Genet. A. 155A(7):1680-4 (2011)).
CREB3 Regulatory Factor (CREBRF), also known as C5orf41, has been associated with obesity and diabetes. (See, e.g., Hanson et al. Diabetologia. 62(9):1647-1652 (2019)).
Cullin 4B (CUL4B), also known as KIAA0695, MRXHF2, MRXS15, MRXSC, and SFM2, has been associated with mental retardation, X-linked, syndromic 15 (Cabezas type). (See, e.g., Tarpey et al. Am. J. Hum. Genet. 80(2):345-52 (2007)).
DNA Methyltransferase 3 Alpha (DNMT3A), also known as HESJAS and TBRS, encodes a protein involved in de novo methylation. (See, e.g., Xie S. et al. Gene 236(1):87-95 (1999)).
Dual Specificity Tyrosine Phosphorylation Regulated Kinase 1B (DYRK1B), also known as Minibrain-related kinase, has been associated with abdominal obesity-metabolic syndrome 3. (See, e.g., Keramati et al. N. Engl. J. Med. 15; 370(20):1909-1919 (2014)).
Ectonucleotide Pyrophosphatase/Phosphodiesterase 1 (ENPP1), also known as NPPS, M6S1, and PDNP1, has been associated with obesity. (See, e.g., Valli-Jaakola et al. Obesity. 16(9):2113-9 (2008)).
E1A Binding Protein P300 (EP300), also known as Histone Acetyltransferase P300, has been associated with Rubinstein-Taybi syndrome. (See, e.g., Stevens et al. Am. J. Med. Genet. A. 155A(7):1680-4 (2011)).
FMRP Translational Regulator 1 (FMR1), also known as POF1 and POF, has been associated with Fragile X Syndrome. (See, e.g., Raspa et al. Am. J. Intelelct. Devv. Disabil. 115(6):482-95 (2010)).
FTO Alpha-Ketoglutarate Dependent Dioxygenase (FTO), also known as FTO Alpha-Ketoglutarate Dependent Dioxygenase, has been associated with obesity-related traits including body mass index, hip circumference, and weight. (See e.g., Scuteri et al. PLoS. Genet. 3(7):e115 (2007)).
Ghrelin and Obestatin Prepropeptide (GHRL), also known as Prepro-Appetite Regulatory Hormone, has been associated with obesity. (See, e.g., J. Clin. Endocrinol. Metab. 87(8):4005-8 (2002)).
Gastric Inhibitory Polypeptide Receptor (GIPR), also known as GIP-R and PGQTL2, has been associated with body mass index and energy intake and expenditure pathways in obesity. (See, e.g., Turcot et al. Nat. Genet. 50(1):26-41 (2018)).
Glucagon Like Peptide 1 Receptor (GLP1R), also known as GLP-1, has been associated with food intake and body weight regulation. (See, e.g., Sisley et al. J. Clin. Invest. 124(6):2456-63 (2014).
Inositol Polyphosphate-5-Phosphatase E (INPP5E), also known as JBTS1, has been associated with Jourbert syndrome and MORM syndrome, an autosomal recessive congenital disorder characterized by mental retardation, truncal obesity, retinal dystrophy, and micropenis. (See, e.g., Jacoby et al. Nat. Genet. 41(9):1027-31 (2009)).
Insulin (INS), also known as IDDM2 and IDDM1, has been associated with body mass index and obesity. (See, e.g., Antúnez-Ortiz et al. Biomed. Res. Int. 2017:2432957 (2017)).
Insulin Induced Gene 2 (INSIG2), also known as Insulin Induced Protein 2, has been associated with feedback control of lipid synthesis and obesity in children. (See, e.g., Kaulfers et al. PLoS One 10(1):e0116340 (2015)).
Insulin Receptor Substrate 1 (IRS1), also known as HIRS-1, has been associated with obesity, type II diabetes, and susceptibility to insulin resistance. (See, e.g., Clausen et al. Lancet. 346(8972):397-402 (1995)).
Insulin Receptor Substrate 4 (IRS4), also known as Pp160, CHNG9, PY160, and Py160, has been associated with obesity, hyperglycemia, and insulin resistance. (See, e.g., Sadagurski et al. Mol. Metab. 23; 3(1):55-63 (2013)).
Insulin Gene Enhancer Protein (ISL1), also known as Islet-1 and Isl-1, is a member of the LIM/homeodomain family of transcription factors, and mutations in this gene have been associated with, inter alia, maturity-onset diabetes. (See, e.g., Tanizawa Y et al. Diabetes (1994)).
Methyl-CpG Binding Protein 2 (MeCP2), also known as AUTSX3, MRXS13, MRX16, RTS, and RTT, encodes a nuclear protein related to onset of Rett syndrome, a progressive neurologic developmental disorder. Amir, R. E. et al. Nat Genet 23(2):185-8 (1999)
Neuropilin 1 (NRP1), also known as CD304 and BDCA4, encodes one of two neuropilins involved in signaling pathways that control cell migration. NRP1 is associated with cerebral arteriopathy, autosomal dominant, and neuroma. (See, e.g., Soker, S. et al Cell 92(6):735-745).
Neuropilin 2 (NRP2), also known as NPN2, NP2, and PR02714, may play a role in cardiovascular development, axon guidance, and tumorigenesis. (See, e.g., Chen, H. et al. Neuron 19(3):547-549 (1997)).
RPGRIP1L Like (RPGRIP1L), also known as FTM, PPP1R134, CORS3, MKS5, JBTS7, and KIAA1005, has been found to interact with neprocystin-4. Defects in this gene have been associated with Joubert syndrome type 7 and Meckel syndrome type 5 (Nagase, T et al DNA Res 6(1):63-70 (1999)).
Plexin A1 (PLXNA1), also known as NOV and PLXN1, is associated with hereditary congenital facial paresis and nephronophthisis 4. (See, e.g., Maestrini, E. et al. Proc Natl Acad Sci USA 93(2):674-678 (1996)).
Plexin A2 (PLXNA2), also known as OCT, KIAA0463, and FLJ11751, is a plexin-A family member believed to be related to signal transduction from semaphorin-3A and semaphorin-3B. (See, e.g., also Coric, V. et al. Depress Anxiety 27(5):417-425 (2010)).
Plexin A3 (PLXNA3), also known as XAP-6, is involved in cytoskeletal remodeling and apoptosis. This gene has been shown to be important in axon pathfinding in developing nervous systems and is associated with tumor progression. (See, e.g., Maestrini, L. et al. Proc Natl Acad Sci USA 93(2):674-678 (1996)).
Plexin A4 (PLXNA4), also known as FAYV2820, KIAA1550, and PR034003, is associated with various signal transduction pathways, particularly involving semaphorin-3A and semaphorin-3B. (See, e.g., Imboden, M. J Allergy Clin Immunol 129(5):1218-1228 (2012)).
Potassium Channel Tetramerization Domain Containing 15 (KCTD15), also known as BTB/POZ Domain-Containing Protein KCTD15, has been associated with body mass index and obesity in children. (See, e.g., Zhao et al. Obesity 17(12):2254-7 (2009)).
Kinase D Interacting Substrate 220 (KIDINS220), also known as ARMS, KIAA1250, and SINO, has been associated with spastic paraplegia, intellectual disability, nystagmus, and obesity. (See, e.g., Josifova et al. Hum. Mol. Genet. 25(11):2158-2167 (2016)).
Melanin Concentrating Hormone Receptor 1 (MCHR1), also known as GPR24, has been associated with regulation of food intake and body weight. (See, e.g., Marsh et al. Proc. Natl. Acad. Sci. U.S.A. 5; 99(5):3240-5 (2002)).
Methionine Sulfoxide Reductase A (MSRA), also known as PMSR, has been associated with several obesity-related traits in children. (See, e.g., Albuquerque et al. J. Hum. Genet. 59(6):307-13 (2014)).
Necdin, MAGE Family Member (NDN), also known as PWCR, has been associated with Prader-Willi syndrome. (See, e.g., Jay et al. Nat. Genet. 17(3):357-61 (1997)).
Neuronal Growth Regulator 1 (NEGR1), also known as Neurotractin, IGLON4, DMML2433, KILON, and Ntra, has been associated with body mass index. (See, e.g., Zhao et al. Obesity. 17(12):2254-7 (2009)).
Neuroligin 2 (NLGN2), also known as KIAA1366, has been associated with anxiety, autism, intellectual disability, hyperphagia, and obesity. (See, e.g., Am. J. Med. Genet. A. 173(1):213-216 (2017)).
Neuropeptide Y (NPY), also known as PYY4, has been associated with obesity. (See, e.g., van Rossum et al. Int. J. Obes. 30(10):1522-8 (2006)).
Nuclear Receptor Subfamily 0 Group B Member 2 (NR0B2), also known as SHP1, has been associated with mild and early-onset obesity. (See, e.g., Nishigori et al. PNAS. 16; 98(2):575-80 (2001)).
Neurotrophic Receptor Tyrosine Kinase 2 (NTRK2), also known as Trk-B, has been associated with severe obesity and developmental delay (e.g., NTRK2 deficiency obesity). (See, e.g., Yeo et al. Nat. Neurosci. 7(11):1187-9 (2004)).
Opioid Receptor Mu 1 (OPRM1), also known as MOR1, MOP, LMOR, OPRM, and HMOP, has been associated with associated with metabolism and the MC4R pathway (See, e.g., Olszewski et al. Neuroreport 12(8):1727-1730 (2001)).
Pericentrin (PCNT), also known as Kendrin and PCNT2, has been associated with Majewski osteodysplastic primordial dwarfism type II. (See, e.g., Rauch et al. Science. 8; 319(5864):816-9 (20008)).
Pleckstrin Homology Domain Interacting Protein (PHIP), also known as WDR11, Ndrp, DCAF14, BRWD2. (See, e.g., Webster et al. Cold Spring Harb Mol Case Stud 2(6):a001172 (2016).
Proprotein Convertase Subtilisin/Kexin Type 2 (PCSK2), also known as NEC2, has been associated with glucose homeostasis, food intake, ultimately body mass. (See, e.g., Anini et al. Int. J. Obes. 34(11):1599-607 (2010)).
PHD Finger Protein 6 (PHF6), also known as BFLS and BORJ, has been associated with Börjeson-Forssman-Lehman syndrome, a syndrome characterized by moderate to severe mental retardation, epilepsy, hypogonadism, hypometabolism, obesity with marked gynecomastia, swelling of subcutaneous tissue of the face, narrow palpebral fissure, and large but not deformed ears. (See, e.g., Lower et al. Nat. Genet. 32(4):661-5 (2002)).
Pro-Melanin Concentrating Hormone (PMCH), also known as MCH and PpMCH, has been associated with regulation of food intake and body weight. (See, e.g., Shimada et al. Nature. 396(6712):670-4 (1998)).
Peroxisome Proliferator Activated Receptor Gamma (PPARG), also known as NR1C3, PPARG1, PPARG2, CIMT1, and GLM1, has been associated with obesity in children and adolescents. (See, e.g., Ochoa et al. Int. J. Obes. Relat. Metab. Disord. 28 Suppl 3:S37-41 (2004)).
Peptide YY (PYY), also known as Peptide Tyrosine Tyrosine, has been associated with regulation of food intake and obesity. (See, e.g., Ahituv et al. Hum. Mol. Genet. 1; 15(3):387-91 (2006)).
Syndecan 3 (SDC3), also known as SDCN, has been associated with energy balance, obesity, body mass index, and LHDL cholesterol. (See, e.g., Chang et al. Int. J. Endocrinol. 30; 2018:9282598 (2018)).
SEC16 Homolog B, Endoplasmic Reticulum Export Factor (SEC16B), also known as LZTR2, has been associated with body mass index. (See, e.g., Felix et al. Hum. Mol. Genet. 15; 25(2):389-403 (2016)).
Solute Carrier Family 6 Member 14 (SLC6A14), also known as BMIQ11, has been associated with body mass index and obesity. (See, e.g., Suviolahti et al. J. Clin. Invest. 112(11):1762-72 (2003)).
Small Nuclear Ribonucleoprotein Polypeptide N (SNRPN), also known as PWCR, has been associated with Prader-Willi Syndrome. (See, e.g., Kuslich et al. Am. J. Hum. Genet. 64(1):70-6 (1999)).
Thyroid Hormone Receptor Beta (THRB), also known as ERBA2 and PRTH, has been associated with regulation of food intake and body weight. (See e.g., Amorim et al. J. Endocrinol. 203(2):291-9 (2009)).
Transient Receptor Potential Cation Channel Subfamily C Member 5 (TRPC5), also known as PPP1R159, TRP-5, HTRP5. (see, e.g., Sossey-Alaoui, K et al. Genomics 60(3):330-3340 (1999)).
Transmembrane Protein 18 (TMEM18), also known as LncND, has been associated with body mass index and body weight regulation. (See, e.g., Willer et al. Nat. Genet. 41(1):25-34 (2009)).
Transmembrane Protein 67 (TMEM67), also known as MKS3, has been associated with Bardet-Biedl Syndrome. (See, e.g., Leitch et al. Nat. Genet. 40(4):443-8 (2008)).
Trafficking Protein Particle Complex 9 (TRAPPC9), also known as NIBP, has been associated with mental retardation, autosomal recessive 13. (See, e.g., Marangi et al. Eur. J. Hum. Genet. 21(2):229-32 (2013)).
Uncoupling Protein 1 (UCP1), also known as thermogenin, SLC25A7, and UCP, has been associated with obesity. (See, e.g., Ramos et al. BMC Med. Genet. 7; 13:101 (2012)).
Uncoupling Protein 3 (UCP3), also known as SLC25A9, has been associated with metabolic fuel partitioning and obesity. (See, e.g., Argyropoulos et al. J. Clin. Invest. 1; 102(7):1345-51 (1998)).
Vacuolar Protein Sorting 13 Homolog B (VPS13B), also known as CHS1 and COH1, has been associated with Cohen syndrome, an autosomal recessive disorder with variability in the clinical manifestations, characterized by mental retardation, postnatal microcephaly, facial dysmorphism, pigmentary retinopathy, myopia, and intermittent neutropenia. (See, e.g., Seifert et al. J. Med. Genet. 43(5):e22 (2006)).

In an embodiment, the MC4R pathway agonizable gene comprises POMC, PCSK1, LEPR, LEP, SDCCAG8, SH2B1, CPE, ALMS1, BBS1, BBS2, BBS4, BBS5, BBS6, BBS7, BBS8, BBS9, BBS10, BBS12, BBS18, BBS20, GNAS, MC3R, NHLH2, SIM1, BDNF, NTRK2, MAGEL2, or a 16p11.2 deletion.

### Disorders

The present disclosure features methods for treating a subject having a disease, disorder, or condition relating to an MC4R pathway agonizable gene. In an embodiment, the disease, disorder, or condition is characterized by a mutation (e.g., a substitution mutation, a deletion mutation, or a polymorphism) in the MC4R pathway agonizable gene. In embodiments, the methods comprise administering to the subject an MC4R agonist or compositions described herein, e.g., a compound of any one of Formulas (I), (II), (III), (IV), (V), (VI), (VII), (VIII), (IX), (X), (XI), or (XII), (e.g., as described herein) or a pharmaceutically acceptable salt thereof. In an embodiment, the MC4R agonist is setmelanotide (i.e., Ac-Arg-c(Cys-D-Ala-His-D-Phe-Arg-Trp-Cys)-NH₂ (SEQ ID NO: 140))

In embodiments, a MC4R agonist, e.g., MC4R agonist described herein, e.g., setmelanotide, is used to treat a genetic disorder caused by a deficiency an MC4R pathway agonizable gene, wherein the MC4R pathway agonizable gene is selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, or MeCP2. In embodiments, a MC4R agonist, e.g., MC4R agonist described herein, e.g., setmelanotide, is used to treat a genetic disorder caused by a deficiency an MC4R pathway agonizable gene, wherein the MC4R pathway agonizable gene is selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, TRPC5, PHIP, or MeCP2.

In embodiments, the genetic disorder is associated with obesity, e.g., severe obesity, and/or hyperphagia. In embodiments, the genetic disorder is BBS. In some embodiments, the genetic disorder is Alström syndrome. In embodiments, the genetic disorder is Smith-Magenis syndrome. In embodiments, the genetic disorder is hypothalamic obesity.

### Bardet-Biedl Syndrome (BBS)

In embodiments, a MC4R agonist described herein is used to treat Bardet-Biedl syndrome (BBS). BBS is a genetically heterogeneous disorder. BBS is a form of Laurence-Moon-Beidl syndrome and is characterized by obesity, retinopathy, learning disability, polydactyly, and hypogenitalism. See, e.g., Green et al. New Engl. J. Med. 321(1989):1002-9. Without wishing to be bound by theory, it is believed that BBS is characterized by one or more mutation(s) in one or more of 20 genes (BBS1-BBS20). Most of the BBS genes encode proteins thought to be important for the function, formation, and stability of cilia. It is believed that eight BBS proteins (BBS1, BBS2, BBS4, BBS5, BBS7, BBS8, BBS9, and BBS18) form a complex called the BBSome that mediates trafficking to the ciliary membrane. BBS6, BBS10, and BBS12 are believed to form a complex with the CCT/TRiC family of group II chaperonins.

Mutation(s) in the BBS gene(s) are thought to lead to defective cilia, e.g., neuronal cilia, or dysfunctional ciliary regulation. Ciliary dysfunction is believed to cause impaired leptin signaling and hyperleptinemia. The role of primary cilia and cilia proteins in energy homeostasis and obesity-related disorders is described, e.g., in., Gupta et al. J. Endocrinol. 203(2009):327-36; and Oh et al. Cell Metab. 21.1(2015):21-31. Patients with BBS have been found to have hyperleptinemia that is suggestive of leptin resistance, with triglycerides, leptin, diastolic BP-Z, and intra-abdominal fat mass significantly greater in BBS patients than in controls. See, e.g., Feuillan et al. J. Clin. Endocrinol. Metab. 96.3(2011). Obesity in BBS mutant mice, for example, is thought to be caused by leptin resistance and defects in leptin receptor trafficking. See, e.g., Berbari et al. Proc. Natl. Acad. Sci. USA 110.19(2013):7796-7801. BBS2, BB4, and BB6 mutant mice have been shown to be hyperleptinemic and failed to reduce their food intake in response to leptin. See, e.g., Berbari et al. Proc. Natl. Acad. Sci. USA 110.19(2013):7796-7801.

Alström syndrome

Alström syndrome (ALMS) is an autosomal recessive disease with clinical symptoms that include severe obesity, hyperinsulinemia, and altered glucose metabolism that can lead to the development of type 2 diabetes at a young age in afflicted subjects. ALMS is caused by mutations in ALMS1, a gene that has been mapped to chromosome 2p13.

The progression from early onset obesity toward the impaired fasting glucose or impaired glucose tolerance and overt diabetes is believed to occur mostly because of a progressive failure of 3-cell insulin secretion without any further worsening of insulin resistance with age, even in the presence of weight reduction (Bettini et al. Pediatr. Diabetes 13:59-67, 2012).

### Prader Willi Syndrome (PWS)

Prader Willi Syndrome (PWS) is a rare genetic disease with a prevalence ranging from approximately one in 8,000 to one in 25,000 patients in the U.S. A hallmark of PWS is severe hyperphagia—an overriding physiological drive to eat—leading to severe obesity and other complications. Obesity is one of the greatest health threats to PWS patients, and hyperphagia impairs the ability of PWS patients to live independently, requiring costly and constant supervision to prevent overeating. Without supervision, these patients are likely to die prematurely as a result of choking, stomach rupture, or from complications caused by morbid obesity. Currently, there are no approved treatments for the obesity and hyperphagia associated with PWS. Symptoms of PWS include infantile hypotonia with failure to thrive, rapid weight gain and overeating during childhood, as well as intellectual disability, developmental delay, short stature, hypogonadism. Diagnostic criteria for PWS are described, e.g., in Holm et al. Pediatrics 91(1993):398-402.

It is believed that the genetics underlying PWS involve a loss of function of several genes on chromosome 15 in humans, in particular, at 15q11-q13. See, e.g., Schaaf et al. Nat. Genet. 45.11(2013):1405-09. Without wishing to be bound by theory, it is believed that the MC4R agonists described herein, e.g., setmelanotide, may reestablish weight and appetite control in PWS subjects by bypassing the defective POMC neurons and activating the MC4 pathways below the block in the pathway. For example, the melanocortin receptor agonists described herein, e.g., setmelanotide, can act as a replacement therapy for MSH.

### Smith-Magenis Syndrome

Smith-Magenis syndrome is a neurobehavioral disorder characterized by a recognizable pattern of physical, behavioral, and developmental features. Common features of the disease include hypotonia, poor gross motor and fine motor skills, feeding problems in infancy, speech delay, developmental delay, intellectual disability, scoliosis, short fingers and toes, vision problems, middle ear abnormalities, sleep disturbances, hearing impairment, decreased sensitivity to pain, and constipation. It is a rare disorder, occurring in between 1 out of every 15,000 to 25,000 individuals. Smith-Magenis syndrome is caused by mutations in the gene RA11, in particular on chromosomal region 17p11.2. While Smith-Magenis syndrome disease is genetic, it is often not familial, and often not inherited from either parent (see, e.g., Falco et al. Appl Clin Genet (2017) 10:85-94).

### Hypothalamic Obesity

Hypothalamic obesity is a form of obesity caused by physical or inherited damage to the hypothalamus, resulting in symptoms such as uncontrollable hunger, rapid and/or excessive weight gain, and a low metabolic rate. Causes for this condition include the presence of a tumor, swelling in the brain, head trauma, radiotherapy, brain surgery, or the presence of certain genetic mutations. For example, hypothalamic obesity may be caused by craniopharyngioma, a rare non-cancerous tumor. Removal of this tumor can result in damage to the hypothalamus, leading to symptoms of hypothalamic obesity. Genetic mutations in the LEP, LEPR, POMC, MC4R, and CART genes may also lead to this disease (see, e.g., Kim et al. Ann Pediatr Endocrinol Metab (2013) 18(4): 161-167). Hypothalamic obesity has also been linked to diminished α-MSH levels (see, e.g., Roth et al. Metabol Clin Exper (2010) 59:186-194).

Additional diseases, disorders, or conditions that may be treated by administration of an MC4R agonist or compositions described herein, e.g., a compound of any one of Formulas (I), (II), (III), (IV), (V), (VI), (VII), (VIII), (IX), (X), (XI), or (XII), (e.g., as described herein) or a pharmaceutically acceptable salt thereof include 5p3 microduplication syndrome, Angelman syndrome, Chudley Lowry syndrome, Cornelia de Lange syndrome, Laron syndrome, Kleefstra syndrome/9q34.3, Camera-Marugo-Cohen syndrome, Clark and Baraitser XLMR syndrome, DiGeorge syndrome, velocardiofacial syndrome, conotruncal anomaly face syndrome, 22q11.2 deletion syndrome, rapid onset obesity with hypothalamic dysfunction (ROHHAD), rapid onset obesity with hypothalamic dysfunction, hypoventilation, autonomic dysregulation and neural crest tumor (ROHHAD NET), Shashi XLMR syndrome, mental retardation, epileptic seizures, hypogonadism and -genitalism, microcephaly, obesity (MEHMO) syndrome, mandibular prognathism with eye and skin anomalies (MOMES) syndrome, and MOMO syndrome. Additional diseases, disorders, or conditions that may be treated by administration of an MC4R agonist, e.g., an MC4R agonist described herein, include those summarized in Kaur et al (2017) Obesity Reviews 18:603-634.

### Outcomes

In embodiments, methods described herein result in one or more outcomes, including a reduction of weight (e.g., body weight), a reduction in hunger level, no detectable decrease in energy expenditure (e.g., resting energy expenditure), an increase in energy expenditure (e.g., resting energy expenditure), a reduction in daily/weekly/monthly food intake, a reduction in waist circumference, no detectable increase in blood pressure, or a reduction in blood pressure in a subject, e.g., relative to a control.

In embodiments, the control is the measurement of the parameter in the subject prior to administration of (treatment with) a MC4R agonist. In embodiments, the control is a predetermined value, e.g., the value of the parameter in an average obese human population, e.g., of like age and gender as the subject; or the value of the parameter measured in the subject at a previous time point (e.g., at a previous visit, e.g., to a physician, medical facility or laboratory).

In embodiments, the outcome (e.g., the reduction, increase, no detectable decrease, or no detectable increase in a given parameter) is measured in the subject 1, 2, 3, 4, 5, 6, 7, 8, 9, 10 weeks or more after initiation of treatment with a MC4R agonist. In other embodiments, the outcome (e.g., the reduction, increase, no detectable decrease, or no detectable increase in a given parameter) is measured in the subject over a period of time (e.g., over a period of 1-2 weeks, 2-4 weeks, 4-6 weeks, 6-8 weeks, 8-12 weeks, or 12-16 weeks) during a course of treatment.

In embodiments, methods described herein result in a reduction of weight (e.g., body weight) in the subject compared to a control (e.g., weight of the subject before treatment or a predetermined value, e.g., average weight of an obese human population of like age and gender as the subject not subjected to therapeutic intervention, or the weight of the subject at a previous measurement, e.g., at a previous visit). In embodiments, the reduction is about 1 kg to 3 kg after 1 week of treatment, about 1 kg to 6 kg after 2 weeks of treatment, about 2 kg to 12 kg after 4 weeks of treatment, about 4 kg to 24 kg after 8 weeks of treatment, or about 8 kg to 48 kg after 16 weeks of treatment. In embodiments, the reduction is at a rate of loss of about 1-2 kg/week, e.g., about 2 kg/week, e.g., over a period of 1-2 weeks of treatment or longer, 2-4 weeks of treatment or longer, 4-8 weeks of treatment or longer, 8-16 weeks of treatment, or 16-32 weeks of treatment, or longer.

Measurement of weight, e.g., body weight, can be performed using standard methods in the art.

In embodiments, methods described herein result in a reduction in hunger level in the subject compared to a control (e.g., hunger level of the subject before treatment or a predetermined hunger level, e.g., average hunger level of an obese human population of like age and gender as the subject or the hunger level of the subject at a previous measurement, e.g., at a previous visit). In embodiments, the methods described herein result in abolishment of hunger in the subject.

In embodiments, hunger is measured by a scale, such as a Likert hunger scale, which ranges from 0 to 10 and is described herein. In embodiments, methods described herein result in a reduction in hunger score in the subject compared to a control (e.g., hunger level of the subject before treatment or a predetermined hunger level, e.g., average hunger level of an obese human population of like age and gender as the subject or the hunger level of the subject at a previous measurement, e.g., at a previous visit). In embodiments, methods described herein result in a lower score on the Likert hunger scale, e.g., a lower score by at least 1, 2, 3, 4, 5, 6, 7, 8, 9 or 10 points, compared to the control (e.g., hunger level of the subject before treatment or a predetermined hunger level, e.g., average hunger level of an obese human population of like age and gender as the subject or the hunger level of the subject at a previous measurement, e.g., at a previous visit). In embodiments, methods described herein result in a score of 0 on the Likert hunger scale after treatment.

In embodiments, the reduction in hunger level is measured/observed after 1 to 2 weeks of treatment or longer, 2-4 weeks of treatment or longer, 4-8 weeks of treatment or longer, or 8-16 weeks of treatment or longer.

REE is a measure of the basal metabolic rate of the subject and can be determined using methods such as those described in Chen et al. J. Clin. Endocrinol. Metab. 100.4(2015):1639-45. In embodiments, the REE can be determined by placing the subject in a whole-room indirect calorimeter (also called a metabolic chamber) at a certain time after treatment (e.g., after 3, 4, 5, 6, 7 days, or 1, 2, 3, 4, or more weeks). In embodiments, the REE is measured in 30-minute measurements periods, and in some cases, REE values from several 30-minute periods are averaged to generate an average REE. In embodiments, the REE can be determined after a 10-12 hour fasting period, at thermoneutrality (e.g., around 25 deg C), where the subject is awake without psychological or physical stress. In embodiments, REE is measured in units of energy per unit time (e.g., kcal/h or kcal/day). In embodiments, the REE is measured relative to kg lean body mass in a subject (e.g., REE/kg lean mass), e.g., as described in the Examples.

In embodiments, methods described herein result in no change or no decrease in energy expenditure, e.g., resting energy expenditure (REE), in the subject over an hourly, daily (e.g., in 24 hours), weekly (e.g., in 7 days), or monthly (e.g., in 30 days) period compared to a control REE (e.g., the REE in the subject prior to treatment or a predetermined REE, e.g., average REE of an obese human population of like age and gender and normalized for weight as the subject or the REE of the subject at a previous measurement, e.g., previous visit), e.g., as measured after 3, 4, 5, 6, 7 days, or 1, 2, 3, 4, or more weeks of treatment.

In embodiments, methods described herein result in no detectable change or no detectable decrease in energy expenditure, e.g., resting energy expenditure (REE) per kg lean body mass, in the subject over an hourly, daily (e.g., in 24 hours), weekly (e.g., in 7 days), or monthly (e.g., in 30 days) period compared to the control REE (e.g., the REE in the subject prior to treatment or a predetermined REE, e.g., average REE of an obese human population of like age and gender as the subject or the REE of the subject at a previous measurement, e.g., previous visit), e.g., as measured after 3, 4, 5, 6, 7 days, or 1, 2, 3, 4, or more weeks of treatment.

In embodiments, methods described herein result in an increase in energy expenditure, e.g., resting energy expenditure (REE), in the subject over a hourly, daily (e.g., in 24 hours), weekly (e.g., in 7 days), or monthly (e.g., in 30 days) period compared to a control REE (e.g., the REE in the subject prior to treatment or a predetermined REE, e.g., average REE of an obese human population of like age and gender and normalized for weight as the subject or the REE of the subject at a previous measurement, e.g., previous visit), e.g., as measured after 3, 4, 5, 6, 7 days, or 1, 2, 3, 4, or more weeks of treatment.

In embodiments, the increase in REE in the subject is at least 20 kcal/day (e.g., at least 20, 30, 40, 50, 60, 70, 80, 90, 100, 110, 120, 130, 140, 150 kcal/day or more), e.g., as measured after 3, 4, 5, 6, 7 days, or 1, 2, 3, 4, or more weeks of treatment.

In embodiments, the increase in REE in the subject is at least 2% (e.g., at least 2%, 3%, 4%, 5%, 6%, 7% 8%, 9%, 10%, 11%, 12%, 13%, 14%, 15% or more), e.g., as measured after 3, 4, 5, 6, 7 days, or 1, 2, 3, 4, or more weeks of treatment, compared to the REE in the subject prior to treatment.

In embodiments, the REE in the subject (e.g., adult subject) after treatment with a MC4R agonist (e.g., after 3, 4, 5, 6, 7 days, or 1, 2, 3, 4, or more weeks of treatment) is at least 1800 kcal/day (e.g., at least 1800, 1825, 1850, 1875, 1900, 1925, 1950, 1975, 2000, 2025, 2050, 2100, 2150, 2200, 2250, 2300, 2400 kcal/day, or more), e.g., for an adult subject. In embodiments, the REE in the subject (e.g., pediatric subject) after treatment with a MC4R agonist (e.g., after 3, 4, 5, 6, 7 days, or 1, 2, 3, 4, or more weeks of treatment) is at least 200 kcal/day (e.g., at least 200, 225, 250, 275, 300, 325, 350, 375, 400, 450, 500 kcal/day or more), e.g., for pediatric patients.

In embodiments, methods described herein result in a reduction in food intake by the subject compared to a control (e.g., the food intake of the subject prior to treatment or a predetermined food intake level, e.g., the food intake of an average human obese population or the food intake of the subject at a previous measurement, e.g., at a previous visit), e.g., where the food intake is measured as daily food intake or food intake over a period of 24 hours, or one week. In embodiments, the reduction is at least 100 kilocalories, e.g., at least 100, 125, 150, 175, 200, 225, 250, 275, 300, 325, 350, 375, 400, 425, 450, 475, 500, 525, 550, 575, 600, 1000 kilocalories or more, e.g., for daily food intake or food intake over a period of 24 hours, or one week, or 30 days or for longer time periods, e.g., for an adult subject. In embodiments, mean food intake can decrease from a baseline at or above about 100 kcal/kg/day to about 90, 80, 70, 60, 50, 40, 30, 20 or 10 kcal/kg/day or lower after treatment with a MC4R agonist, e.g., setmelanotide, e.g., in a pediatric subject at about 1 year of age. In embodiments, mean food intake can decrease from a baseline at or above about 40 kcal/kg/day to about 35, 30, 20 or 10 kcal/kg/day or lower after treatment with a MC4R agonist, e.g., setmelanotide, e.g., in a pediatric subject in late adolescence.

Food intake can be determined by standard methods, e.g., as described in Rutishauser. Pub. Health Nutr. 8.7A(2005):1100-07.

In embodiments, methods described herein result in a reduction in waist circumference of the subject compared to a control (e.g., the waist circumference of the subject prior to treatment or the waist circumference of the subject at a previous measurement, e.g., previous visit), as measured 1, 2, 3, 4, 5, 6, 7, 8, 9, 10 weeks or more after initiation of treatment.

In embodiments, the reduction in waist circumference is at least 2 cm (e.g., at least 2, 3, 4, 5, 6, 7, 8, 9, 10 cm or more) in the subject (e.g., adult subject) compared to a control (e.g., the waist circumference of the subject prior to treatment or a predetermined waist circumference, e.g., the waist circumference of an average obese human population of like age and gender or the waist circumference of the subject at a previous measurement, e.g., previous visit), as measured 1, 2, 3, 4, 5, 6, 7, 8, 9, 10 weeks or more after initiation of treatment.

In embodiments, the waist circumference is measured using standard methods. In embodiments, the waist circumference is the largest circumference around a subject's mid-section, e.g., around a subject's abdomen. In other embodiments, the waist circumference is measured around the natural waist (e.g., in between the lowest rib and the top of the hip bone), the umbilicus, or at the narrowest point of the midsection.

In embodiments, methods described herein result in no detectable increase in blood pressure (e.g., diastolic and/or systolic blood pressure) of the subject compared to a control blood pressure (e.g., the blood pressure of the subject prior to treatment or a predetermined blood pressure, e.g., the blood pressure of an average obese human population of like age and gender or the blood pressure of the subject at a previous measurement, e.g., previous visit), as measured 1, 2, 3, 4, 5, 6, 7, 8, 9, 10 weeks or more after initiation of treatment.

In embodiments, methods described herein result in a reduction in blood pressure (e.g., diastolic and/or systolic blood pressure) of the subject a control blood pressure (e.g., the blood pressure of the subject prior to treatment or a predetermined blood pressure, e.g., the blood pressure of an average obese human population of like age and gender or the blood pressure of the subject at a previous measurement, e.g., previous visit), as measured 1, 2, 3, 4, 5, 6, 7, 8, 9, 10 weeks or more after initiation of treatment.

In embodiments, the reduction in blood pressure, e.g., systolic blood pressure, is at least 3 mmHg (e.g., at least 3, 3.5, 4, 4.5, 5, 5.5, 6, 6.5, 7 mmHg or more) compared to the blood pressure of the subject prior to treatment, as measured 1, 2, 3, 4, 5, 6, 7, 8, 9, 10 weeks or more after initiation of treatment.

In embodiments, the reduction in blood pressure, e.g., diastolic blood pressure, is at least 4 mmHg (e.g., at least 4, 7, 7.5, 8, 8.5, 9, 9.5, 10 mmHg or more) compared to the blood pressure of the subject prior to treatment, as measured 1, 2, 3, 4, 5, 6, 7, 8, 9, 10 weeks or more after initiation of treatment.

In embodiments, the methods described herein do not result in an adverse effect on heart rate or blood pressure.

### Patient Selection

In accordance with any method described herein, in certain embodiments, the subject is obese, e.g., prior to administration of an MC4R agonist described herein, e.g., at the time the MC4R agonist is prescribed, or at the time of the first administration of the MC4R agonist. In embodiments, the subject is a severely obese, pediatric or adult patient e.g., prior to administration of an MC4R agonist described herein, e.g., at the time the MC4R agonist is prescribed or at the time of the first administration of the MC4R agonist. In embodiments, the subject is hyperphagic, e.g., prior to administration of an MC4R agonist described herein, e.g., at the time the MC4R agonist is prescribed, or at the time of the first administration of the MC4R agonist.

In embodiments, the subject (e.g., adult subject) has a body mass index (BMI) greater than 25 kg/m² or 30 kg/m² (e.g., ≥25, 26, 27, 28, 29, 30, 31, 32, 33, 34, 35, 36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration.

In embodiments, the subject (e.g., pediatric subject) has a body mass index (BMI) higher than 85-95 percentile prior to administration of the MC4R agonist, e.g., at the time the MC4R agonist is prescribed, or at the time of the first administration.

In embodiments, the subject has a body weight of at least about 5 kg, e.g., at least about 5 kg, 10 kg, 20 kg, 30, 40, 50, 60, 70, 80, 90, 100, 110, 120, 130, 140,145, 150, 155, 160, 165, 170, 175, 180, 185, 190, 200, 205, 210, 215, 220 kg or greater, e.g., prior to administration of the MC4R agonist, e.g., at the time the MC4R agonist is prescribed, or at the time of the first administration. In embodiments, the subject has a body weight of a least 20 kg, at least 60 kg, or at least 100 kg, e.g., prior to administration of the MC4R agonist, e.g., at the time the MC4R agonist is prescribed, or at the time of the first administration.

In embodiments, the subject has received intervention in the gastrointestinal system. For example, the subject may have received a gallbladder surgery, an intestinal surgery, a gastric surgery (e.g., a bariatric surgery), or other survival procedure. In an embodiment, the subject has received a gastric bypass surgery. In an embodiment, the subject has received a surgery resulting in a restriction of the total amount of food capable of being held or processed at one time, e.g., the stomach, small intestine, large intestine, or colon.

In embodiments, the subject is an adult, e.g., 18 years of age or older, e.g., 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or older.

In embodiments, the subject is a pediatric subject, e.g., less 18 years of age or younger (e.g., 18, 17, 16, 15, 14, 13, 12, 11, 10, 9, 8, 7, 6, 5, 4, 3, 2, or 1 year of age or younger.

In embodiments, the subject has or is identified as having a defect, e.g., genetic defect, or a mutation, in an MC4R pathway agonizable gene. In embodiments, the subject has or is identified as having a mutation a gene selected from n the ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, or MeCP2 genes. In embodiments, the subject has a disease or disorder associated with a gene in Table 1. In embodiments, the subject has or is identified as having a loss of function mutation in one or more genes in Table 1.

In embodiments, methods herein can comprise identifying or selecting a subject having a defect e.g., genetic defect, or a mutation, in one or more genes listed in Table 1. In embodiments, methods herein can comprise acquiring knowledge of the genotype, predetermined sequence, or mutation. In embodiments, the methods herein can comprise acquiring knowledge of the genotype of, e.g., of a mutation in one or more of ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, or VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, or MeCP2 genes. In embodiments, the MC4R agonist is administered in response to acquiring knowledge, e.g., detection or identification, of a predetermined sequence, e.g., a mutation, in a gene described herein, one or more of ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, or VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, or MeCP2 genes.

In embodiments, identification or selection of a subject as having a certain genotype or predetermined sequence, e.g., mutation, in a gene, can comprise acquiring knowledge of the certain genotype or predetermined sequence, e.g., mutation. Knowledge of the sort can be acquired in a number of ways, as described in detail in the Definitions section.

In some embodiments, a sequence is acquired, e.g., by obtaining possession of a nucleotide sequence, by “directly acquiring” or “indirectly acquiring” the sequence. “Directly acquiring a sequence” means performing a process (e.g., performing a synthetic or analytical method) to obtain the sequence, such as performing a sequencing method (e.g., a Next Generation Sequencing (NGS) method). “Indirectly acquiring a sequence” refers to receiving information or knowledge of, or receiving, the sequence from another party or source (e.g., a third-party laboratory that directly acquired the sequence). The sequence acquired need not be a full sequence, e.g., sequencing of at least one nucleotide, or obtaining information or knowledge, that identifies a genotype or predetermined sequence, e.g., mutation, disclosed herein as being present in a subject constitutes acquiring a sequence.

In embodiments, the sequence can be directly acquired. Directly acquiring a sequence includes performing a process that includes a physical change in a physical substance, e.g., a starting material, such as a tissue sample, e.g., a blood sample or tissue biopsy, or analysis of an isolated nucleic acid (e.g., DNA or RNA) sample. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above.

In some embodiments, acquiring knowledge of the certain genotype or predetermined sequence, e.g., mutation, can comprise acquiring a sample, e.g., from which the genotype or predetermined sequence, e.g., mutation, is determined. “Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third-party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

In some aspects, provided herein is also a method of evaluating a subject, e.g., for likely responsiveness to a MC4R agonist, e.g., a MC4R agonist described herein, e.g., setmelanotide. In some embodiments, the method comprises acquiring information about the genotype of the subject. In embodiments, the method comprises acquiring information about the presence or absence of a defect, e.g., genetic defect, in one or more genes listed in Table 1 in the subject.

In embodiments, the subject can be identified as having a defect, e.g., genetic defect, e.g., mutation, in one or more genes listed in Table 1, using methods described herein.

In embodiments, the identification of the subject having a defect, e.g., genetic defect, e.g., mutation, indicates that the subject is likely to respond (e.g., with an improvement in one or more symptoms) to a MC4R agonist, e.g., a MC4R agonist described herein, e.g., setmelanotide. In embodiments, an improvement in a symptom can include an outcome described herein. For example, an improvement in a symptom can include a reduction of weight (e.g., body weight), a reduction in hunger level, no detectable decrease in energy expenditure (e.g., resting energy expenditure), an increase in energy expenditure (e.g., resting energy expenditure), a reduction in daily/weekly/monthly food intake, or a reduction in waist circumference, e.g., relative to a control.

In embodiments, the identification of the subject having the defect, e.g., genetic defect, e.g., mutation, indicates that the subject is more likely to respond to (or is likely to have a greater response to) a MC4R agonist, e.g., a MC4R agonist described herein, e.g., setmelanotide, than a subject (e.g., obese subject, e.g., of like age and/or pre-treatment weight) lacking a genetic defect in one or more genes listed in Table 1, e.g., a wild-type obese subject. In embodiments, a subject that is more likely to respond is more likely to have one or more improved symptoms, such as symptoms described herein, e.g., compared to a control, e.g., a subject (e.g., obese subject, e.g., of like age and/or pre-treatment weight) lacking a genetic defect in one or more genes listed in Table 1, e.g., a wild-type obese subject. In embodiments, a subject that is likely to have a greater response is likely to have a greater improvement in symptoms, e.g., symptoms described herein, e.g., greater weight loss, greater decrease in waist circumference, greater increase in resting energy expenditure, greater decrease in food intake, greater decrease in hunger level, e.g., compared to a control, e.g., a subject (e.g., obese subject, e.g., of like age and/or pre-treatment weight) lacking a genetic defect in one or more genes listed in Table 1, e.g., a wild-type obese subject.

In embodiments, methods described herein further comprise providing a report that identifies the presence or absence of the genetic defect and in some cases an identifier for the subject. In embodiments, the report provides a recommendation on potential therapeutic options, likely effectiveness of a therapeutic option, and/or recommendations/instructions for administration of the therapeutic option (e.g., MC4R agonist, e.g., MC4R agonist described herein, e.g., setmelanotide).

### MC4R Agonists

Described herein are methods for the treating a relating to an MC4R pathway agonizable gene, comprising administering to a subject a melanocorin 4 receptor (MC4R) agonist. Examples of naturally occurring MC4R agonists include α-MSH, β-MSH, γ-MSH and adenocorticitropic hormone (ACTH) or a functional fragment thereof. Examples of synthetic MC4R agonists are described in detail below.

In some embodiments, an MC4R agonist can be any known agonist of MC4R. In some example embodiment, the MC4R agonist is not an adrenocorticotropic hormone (ACTH) or a fragment thereof. Exemplary MC4R agonists include those described in WO2011104378; WO2011104379; WO201060901; WO200887189, WO200887188, WO200887187, WO200887186; US20110065652; WO2010144341; WO2010144344; WO201065799; WO201065800; WO201065801; WO201065802; WO201037081; WO2009152079; WO2009151383; US20100311648; US20100280079; WO201081666; WO201034500; WO200910299; WO2008116665; WO201052256; WO201052255; WO201126015; US20100120783; WO201096854; US20100190793; WO201025142; WO2014144260; WO2017059075; and WO201015972. Further examples of MC4R agonists are found in U.S. Pat. Nos. 8,263,608; 8,247,530; 8,114,844; and 7,968,548. The entire teachings of these publications are incorporated herein by reference.

In some embodiments, the MC4R agonist is a compound of any one of Formulas (I), (II), (III), (IV), (V), (VI), (VII), (VIII), (IX), (X), (XI), or (XII), or a pharmaceutically acceptable salt thereof as described herein. In some embodiments, the MC4R agonist is a compound of any one of Formulas (I) or (II), or a pharmaceutically acceptable salt thereof as described herein. In one embodiment, the MC4R agonist is a compound of Formula (I). In one embodiment, the MC4R agonist is a compound of Formula (II).

In one example embodiment, the agonist of MC4R is a tripeptide D-Phe-Arg-Trp (SEQ ID NO: 560) or a pharmaceutical salt thereof. In another example, the agonist is any peptide that includes SEQ ID NO: 560 or a pharmaceutical salt thereof. In yet another example, the MC4R agonist is an acetylated tripeptide Ac-D-Phe-Arg-Trp-NH₂ (SEQ ID NO: 561) or a pharmaceutical salt thereof.

In some embodiments, the MC4R agonist is a compound of Formula (I):

(R²R³)-A¹-c(A²-A³-A⁴-A⁵-A⁶-A⁷-A⁸-A⁹)-A¹⁰-R¹ (I)

or a pharmaceutically acceptable salt thereof, wherein A¹ is Acc, HN—(CH₂)ₘ—C(O), L- or D-amino acid, or deleted; A² is Cys, D-Cys, hCys, D-hCys, Pen, D-Pen, Asp, or Glu; A³ is Gly, Ala, β-Ala, Gaba, Aib, D-amino acid, or deleted; A⁴ is His, 2-Pal, 3-Pal, 4-Pal, Taz, 2-Thi, 3-Thi, or (X¹, X², X³, X⁴, X⁵)Phe; A⁵ is D-Phe, D-1-Nal, D-2-Nal, D-Trp, D-Bal, D-(X¹, X², X³, X⁴, X⁵)Phe, L-Phe or D-(Et)Tyr; A⁶ is Arg, hArg, Dab, Dap, Lys, Orn, or HN—CH((CH₂)ₙ—N(R⁴R⁵))—C(O); A⁷ is Trp, 1-Nal, 2-Nal, Bal, Bip, D-Trp, D-2-Nal, D-Bal or D-Bip; A⁸ is Gly, D-Ala, Acc, Ala, 13-Ala, Gaba, Apn, Ahx, Aha, HN—(CH₂)ₛ—C(O), or deleted; A⁹ is Cys, D-Cys, hCys, D-hCys, Pen, D-Pen, Dab, Dap, Orn, or Lys; A¹⁰ is Acc, HN—(CH₂)ₜ—C(O), L- or D-amino acid, or deleted; R¹ is OH or NH₂; each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl; each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂; m is, independently for each occurrence, 1, 2, 3, 4, 5, 6 or 7; n is, independently for each occurrence, 1, 2, 3, 4 or 5; s is, independently for each occurrence, 1, 2, 3, 4, 5, 6, or 7; t is, independently for each occurrence, 1, 2, 3, 4, 5, 6, or 7; X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

In some embodiments, for Formula (I), when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or —C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl.

In some embodiments, for Formula (I), when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;

In some embodiments, for Formula (I), either A³ or A⁸ or both must be present in said compound.

In some embodiments, for Formula (I) when A² is Cys, D-Cys, hCys, D-hCys, Pen, or D-Pen, then A⁹ is Cys, D-Cys, hCys, D-hCys, Pen, or D-Pen.

In some embodiments, for Formula (I), when A² is Asp or Glu, then A⁹ is Dab, Dap, Orn, or Lys.

In some embodiments, for Formula (I), when A⁸ is Ala or Gly, then A¹ is not NIe.

In some embodiments, for Formula (I), when A¹ is deleted, then R² and R³ cannot both be H.

In some embodiments, for Formula (I): A¹ is A⁶c, Arg, D-Arg, Cha, D-Cha, hCha, Chg, D-Chg, Gaba, Ile, Leu, hLeu, Met, β-hMet, 2-Nal, D-2-Nal, Nip, Nle, Oic, Phe, D-Phe, hPhe, hPro, Val, or deleted; A² is Asp, Cys, D-Cys, hCys, D-hCys, Glu, Pen, or D-Pen; A³ is D-Abu, Aib, Ala, β-Ala, D-Ala, D-Cha, Gaba, D-Glu, Gly, D-Ile, D-Leu, D-Tle, D-Val, or deleted; A⁴ is His or 3-Pal; A⁵ is D-Bal, D-1-Nal, D-2-Nal, D-Phe, D-Trp, or D-(Et)Tyr; A⁶ is Arg, or hArg; A⁷ is Bal, Bip, 1-Nal, 2-Nal, Trp, D-Trp; A⁸ is A⁶c, D-Ala, Aha, Ahx, Ala, 0-Ala, Apn, Gaba, Gly or deleted; A⁹ is Cys, D-Cys, hCys, D-hCys, Lys, Pen, or D-Pen; and A¹⁰ is Thr, or deleted, wherein at least one of A³ or A⁸ is deleted, but not both.

In some embodiments, the compound of Formula (I) is a compound disclosed in International Patent Application Publication Number WO 2007/008704, which is incorporated herein by reference in its entirety.

In some embodiments, the compound of Formula (I) is selected from:

SEQ ID NO: 1
Ac-Nle-c(Asp-His-D-Phe-Arg-Trp-β-Ala-Lys)-NH₂;
SEQ ID NO: 2
Ac-Nle-c(Asp-His-D-Phe-Arg-Trp-A6c-Lys)-NH₂;
SEQ ID NO: 3
Ac-Nle-c(Cys-His-D-Phe-Arg-Trp-Ahx-Cys)-NH₂;
SEQ ID NO: 4
D-Phe-c(Cys-His-D-Phe-Arg-Trp-Ala-D-Cys)-Thr-NH₂;
SEQ ID NO: 5
D-Phe-c(Cys-His-D-Phe-Arg-Trp-β-Ala-D-Cys)-Thr-NH₂;
SEQ ID NO: 6
D-Phe-c(Cys-His-D-Phe-Arg-Trp-Gaba-D-Cys)-Thr-NH₂;
SEQ ID NO: 7
Ac-Nle-c(Cys-His-D-Phe-Arg-Trp-Apn-Cys)-NH₂;
SEQ ID NO: 8
Ac-Nle-c(Asp-His-D-Phe-Arg-Trp-Apn-Lys)-NH₂;
SEQ ID NO: 9
Ac-A6c-c(Asp-His-D-Phe-Arg-Trp-Gaba-Lys)-NH₂;
SEQ ID NO: 10
Ac-D-2-Nal-c(Asp-His-D-Phe-Arg-Trp-Gaba-Lys)-NH₂;
SEQ ID NO: 11
Ac-Cha-c(Asp-His-D-Phe-Arg-Trp-Gaba-Lys)-NH₂;
SEQ ID NO: 12
Ac-Nle-c(Asp-His-D-Phe-Arg-Trp-Gaba-Lys)-NH₂;
SEQ ID NO: 13
Ac-Nle-c(Cys-D-Ala-His-D-Phe-Arg-Trp-Cys)-NH₂;
SEQ ID NO: 14
Ac-Nle-c(Cys-β-Ala-His-D-Phe-Arg-Trp-Cys)-NH₂;
SEQ ID NO: 15
Ac-Nle-c(Cys-Gaba-His-D-Phe-Arg-Trp-Cys)-NH₂;
SEQ ID NO: 16
Ac-Nle-c(Cys-Aib-His-D-Phe-Arg-Trp-Cys)-NH₂;
SEQ ID NO: 17
Ac-Nle-c(Cys-Gly-His-D-Phe-Arg-Trp-Cys)-NH₂;
SEQ ID NO: 18
Ac-Nle-c(D-Cys-Ala-His-D-Phe-Arg-Trp-Cys)-NH₂;
SEQ ID NO: 19
Ac-Nle-c(D-Cys-D-Ala-His-D-Phe-Arg-Trp-Cys)-NH₂;
SEQ ID NO: 20
Ac-Nle-c(D-Cys-β-Ala-His-D-Phe-Arg-Trp-Cys)-NH₂;
SEQ ID NO: 21
Ac-Nle-c(D-Cys-Gaba-His-D-Phe-Arg-Trp-Cys)-NH₂;
SEQ ID NO: 22
Ac-Nle-c(D-Cys-Aib-His-D-Phe-Arg-Trp-Cys)-NH₂;
SEQ ID NO: 23
Ac-Nle-c(D-Cys-Gly-His-D-Phe-Arg-Trp-Cys)-NH₂;
SEQ ID NO: 24
Ac-Nle-c(Cys-D-Ala-His-D-Phe-Arg-Trp-D-Cys)-NH₂;
SEQ ID NO: 25
Ac-Nle-c(Cys-β-Ala-His-D-Phe-Arg-Trp-D-Cys)-NH₂;
SEQ ID NO: 26
Ac-Nle-c(Cys-Gaba-His-D-Phe-Arg-Trp-D-Cys)-NH₂;
SEQ ID NO: 27
Ac-Nle-c(Cys-Aib-His-D-Phe-Arg-Trp-D-Cys)-NH₂;
SEQ ID NO: 28
Ac-Nle-c(Cys-Gly-His-D-Phe-Arg-Trp-D-Cys)-NH₂;
SEQ ID NO: 29
Ac-Nle-c(D-Cys-Ala-His-D-Phe-Arg-Trp-D-Cys)-NH₂;
SEQ ID NO: 30
Ac-Nle-c(D-Cys-D-Ala-His-D-Phe-Arg-Trp-D-Cys)-NH₂;
SEQ ID NO: 31
Ac-Nle-c(D-Cys-β-Ala-His-D-Phe-Arg-Trp-D-Cys)-NH₂;
SEQ ID NO: 32
Ac-Nle-c(D-Cys-Gaba-His-D-Phe-Arg-Trp-D-Cys)-NH₂;
SEQ ID NO: 33
Ac-Nle-c(D-Cys-Aib-His-D-Phe-Arg-Trp-D-Cys)-NH₂;
SEQ ID NO: 34
Ac-Oic-c(Asp-His-D-Phe-Arg-Trp-Gaba-Lys)-NH₂;
SEQ ID NO: 35
Ac-Chg-c(Asp-His-D-Phe-Arg-Trp-Gaba-Lys)-NH₂;
SEQ ID NO: 36
Ac-hCha-c(Asp-His-D-Phe-Arg-Trp-Gaba-Lys)-NH₂;
SEQ ID NO: 37
Ac-D-Cha-c(Asp-His-D-Phe-Arg-Trp-Gaba-Lys)-NH₂;
SEQ ID NO: 38
Ac-D-hCha-c(Asp-His-D-Phe-Arg-Trp-Gaba-Lys)-NH₂;
SEQ ID NO: 39
Ac-Nip-c(Asp-His-D-Phe-Arg-Trp-Gaba-Lys)-NH₂;
SEQ ID NO: 40
Ac-hPro-c(Asp-His-D-Phe-Arg-Trp-Gaba-Lys)-NH₂;
SEQ ID NO: 41
Ac-hLeu-c(Asp-His-D-Phe-Arg-Trp-Gaba-Lys)-NH₂;
SEQ ID NO: 42
Ac-Phe-c(Asp-His-D-Phe-Arg-Trp-Gaba-Lys)-NH₂;
SEQ ID NO: 43
Ac-D-Phe-c(Asp-His-D-Phe-Arg-Trp-Gaba-Lys)-NH₂;
SEQ ID NO: 44
Ac-D-Chg-c(Asp-His-D-Phe-Arg-Trp-Gaba-Lys)-NH₂;
SEQ ID NO: 45
n-butanoyl-Cha-c(Asp-His-D-Phe-Arg-Trp-Gaba-Lys)-NH₂;
SEQ ID NO: 46
n-butyryl-Cha-c(Asp-His-D-Phe-Arg-Trp-Gaba-Lys)-NH₂;
SEQ ID NO: 47
Ac-hPhe-c(Asp-His-D-Phe-Arg-Trp-Gaba-Lys)-NH₂;
SEQ ID NO: 48
Ac-β-hMet-c(Asp-His-D-Phe-Arg-Trp-Gaba-Lys)-NH₂;
SEQ ID NO: 49
Ac-Gaba-c(Asp-His-D-Phe-Arg-Trp-Gaba-Lys)-NH₂;
SEQ ID NO: 50
Ac-Cha-c(Asp-His-D-Phe-Arg-D-Trp-Ala-Lys)-NH₂;
SEQ ID NO: 51
Ac-hCha-c(Asp-His-D-Phe-Arg-D-Trp-Ala-Lys)-NH₂;
SEQ ID NO: 52
Ac-Leu-c(Asp-His-D-Phe-Arg-D-Trp-Ala-Lys)-NH₂;
SEQ ID NO: 53
Ac-hLeu-c(Asp-His-D-Phe-Arg-D-Trp-Ala-Lys)-NH₂;
SEQ ID NO: 54
Ac-Phe-c(Asp-His-D-Phe-Arg-D-Trp-Ala-Lys)-NH₂;
SEQ ID NO: 55
Ac-Nle-c(Asp-His-D-Phe-Arg-D-Trp-D-Ala-Lys)-NH₂;
SEQ ID NO: 56
Ac-Nle-c(Asp-His-D-Phe-Arg-D-Trp-β-Ala-Lys)-NH₂;
SEQ ID NO: 57
Ac-Nle-c(Asp-His-D-Phe-Arg-D-Trp-Gaba-Lys)-NH₂;
SEQ ID NO: 58
Ac-Nle-c(Asp-His-D-Phe-Arg-D-Trp-Aha-Lys)-NH₂;
SEQ ID NO: 59
Ac-Nle-c(Asp-His-D-Phe-Arg-D-Trp-Apn-Lys)-NH₂;
SEQ ID NO: 60
Ac-Nle-c(Cys-His-D-Phe-Arg-D-Trp-Apn-Cys)-NH₂;
SEQ ID NO: 61
Ac-Nle-c(Cys-His-D-Phe-Arg-D-Trp-Gaba-Cys)-NH₂;
SEQ ID NO: 62
Ac-Nle-c(Cys-His-D-Phe-Arg-D-Trp-Ahx-Cys)-NH₂;
SEQ ID NO: 63
Ac-Nle-c(Cys-His-D-Phe-Arg-D-Trp-β-Ala-Cys)-NH₂;
SEQ ID NO: 64
Ac-Nle-c(Cys-His-D-Phe-Arg-D-Trp-D-Ala-Cys)-NH₂;
SEQ ID NO: 65
Ac-Nle-c(Cys-D-Ala-His-D-2-Nal-Arg-Trp-Cys)-NH₂;
SEQ ID NO: 66
Ac-Nle-c(Cys-D-Ala-His-D-2-Nal-Arg-2-Nal-Cys)-NH₂;
SEQ ID NO: 67
Ac-Nle-c(Cys-D-Ala-His-D-2-Nal-Arg-1-Nal-Cys)-NH₂;
SEQ ID NO: 68
n-butanoyl-Nle-c(Cys-D-Ala-His-D-Phe-Arg-2-Nal-Cys)-NH₂;
SEQ ID NO: 69
n-butanoyl-Nle-c(Cys-D-Ala-His-D-Phe-Arg-Trp-Cys)-NH₂;
SEQ ID NO: 70
Ac-Nle-c(Cys-D-Ala-His-D-Phe-Arg-2-Nal-Cys)-NH₂;
SEQ ID NO: 71
Ac-Nle-c(Cys-D-Ala-His-D-Phe-Arg-1-Nal-Cys)-NH₂;
SEQ ID NO: 72
Ac-Nle-c(Cys-D-Ala-His-D-Phe-Arg-Bal-Cys)-NH₂;
SEQ ID NO: 73
Ac-Nle-c(Cys-D-Glu-His-D-Phe-Arg-Trp-Cys)-NH₂;
SEQ ID NO: 74
Ac-Nle-c(Asp-His-D-Phe-Arg-Trp-D-Ala-Lys)-NH₂;
SEQ ID NO: 75
Ac-Nle-c(Cys-D-Ala-His-D-2-Nal-Arg-Bal-Cys)-NH₂;
SEQ ID NO: 76
Ac-Nle-c(Pen-D-Ala-His-D-Phe-Arg-Trp-Cys)-NH₂;
SEQ ID NO: 77
Ac-Nle-c(Cys-D-Ala-His-D-Phe-Arg-Trp-Pen)-NH₂;
SEQ ID NO: 78
Ac-Nle-c(Pen-D-Ala-His-D-Phe-Arg-Trp-Pen)-NH₂;
SEQ ID NO: 79
D-Phe-c(Cys-His-D-Phe-hArg-Trp-β-Ala-D-Cys)-Thr-NH₂;
SEQ ID NO: 80
D-Phe-c(Cys-His-D-(Et)Tyr-Arg-Trp-β-Ala-D-Cys)-Thr-NH₂;
SEQ ID NO: 81
D-Phe-c(Cys-His-D-Phe-Arg-Bip-β-Ala-D-Cys)-Thr-NH₂;
SEQ ID NO: 82
D-Phe-c(Cys-His-D-(Et)Tyr-hArg-Trp-β-Ala-D-Cys)-Thr-NH₂;
SEQ ID NO: 83
D-Phe-c(Cys-His-D-Phe-hArg-Bip-β-Ala-D-Cys)-Thr-NH₂;
SEQ ID NO: 84
D-Phe-c(Cys-His-D-(Et)Tyr-hArg-Bip-β-Ala-D-Cys)-Thr-NH₂;
SEQ ID NO: 85
Nle-c(Cys-His-D-Phe-Arg-Trp-Apn-Cys)-NH₂;
SEQ ID NO: 86
Ac-Nle-c(Asp-D-Ala-His-D-Phe-Arg-Trp-Lys)-NH₂;
SEQ ID NO: 87
Ac-Nle-c(Asp-D-Ala-His-D-Phe-Arg-Bal-Lys)-NH₂;
SEQ ID NO: 88
Ac-Nle-c(Cys-D-Ala-His-D-Phe-Arg-Trp-Pen)-OH;
SEQ ID NO: 89
Ac-Nle-c(Cys-D-Abu-His-D-Phe-Arg-Trp-Cys)-NH₂;
SEQ ID NO: 90
Ac-Nle-c(Cys-D-Val-His-D-Phe-Arg-Trp-Cys)-NH₂;
SEQ ID NO: 91
Ac-Nle-c(Cys-D-Ile-His-D-Phe-Arg-Trp-Cys)-NH₂;
SEQ ID NO: 92
Ac-Nle-c(Cys-D-Leu-His-D-Phe-Arg-Trp-Cys)-NH₂;
SEQ ID NO: 93
Ac-Nle-c(Cys-D-Tle-His-D-Phe-Arg-Trp-Cys)-NH₂;
SEQ ID NO: 94
Ac-Nle-c(Cys-D-Cha-His-D-Phe-Arg-Trp-Cys)-NH₂;
SEQ ID NO: 95
Ac-Nle-c(Pen-His-D-Phe-Arg-Trp-Gaba-Cys)-NH₂;
SEQ ID NO: 96
Ac-Nle-c(Cys-His-D-Phe-Arg-Trp-Gaba-Pen)-NH₂;
SEQ ID NO: 97
Ac-Nle-c(Pen-His-D-Phe-Arg-Trp-Gaba-Pen)-NH₂;
SEQ ID NO: 98
Ac-Leu-c(Cys-His-D-Phe-Arg-Trp-Gaba-Cys)-NH₂;
SEQ ID NO: 99
Ac-Cha-c(Cys-His-D-Phe-Arg-Trp-Gaba-Cys)-NH₂;
SEQ ID NO: 100
Ac-Ile-c(Cys-His-D-Phe-Arg-Trp-Gaba-Cys)-NH₂;
SEQ ID NO: 101
Ac-Phe-c(Cys-His-D-Phe-Arg-Trp-Gaba-Cys)-NH₂;
SEQ ID NO: 102
Ac-Val-c(Cys-His-D-Phe-Arg-Trp-Gaba-Cys)-NH₂;
SEQ ID NO: 103
Ac-2-Nal-c(Cys-His-D-Phe-Arg-Trp-Gaba-Cys)-NH₂;
SEQ ID NO: 104
Nle-c(Cys-His-D-Phe-Arg-Trp-Gaba-Cys)-NH₂;
SEQ ID NO: 105
Phe-c(Cys-His-D-Phe-Arg-Trp-Gaba-Cys)-NH₂;
SEQ ID NO: 106
Ac-Nle-c(Cys-3-Pal-D-Phe-Arg-Trp-Gaba-Cys)-NH₂;
SEQ ID NO: 107
Ac-Nle-c(Cys-D-Ala-His-D-Phe-Arg-Trp-Cys)-OH;
SEQ ID NO: 108
Ac-Nle-c(Cys-His-Phe-Arg-D-Trp-Gaba-Cys)-NH₂;
SEQ ID NO: 109
Ac-Nle-c(Asp-His-D-2-Nal-Arg-Trp-Ala-Lys)-NH₂;
SEQ ID NO: 110
Ac-Nle-c(Asp-His-D-2-Nal-Arg-Trp-β-Ala-Lys)-NH₂;
SEQ ID NO: 111
Ac-Nle-c(Cys-His-D-2-Nal-Arg-Trp-Gaba-Cys)-NH₂;
SEQ ID NO: 112
Ac-Nle-c(Cys-His-D-2-Nal-Arg-Trp-Ahx-Cys)-NH₂;
SEQ ID NO: 113
Ac-hPhe-c(Asp-His-D-2-Nal-Arg-Trp-Gaba-Lys)-NH₂;
SEQ ID NO: 114
Ac-Cha-c(Asp-His-D-2-Nal-Arg-Trp-Gaba-Lys)-NH₂;
SEQ ID NO: 115
Ac-Nle-c(Asp-His-D-Phe-Arg-Trp-β-Ala-Lys)-OH;
SEQ ID NO: 116
Ac-Nle-c(Cys-His-D-Phe-Arg-Trp-Ahx-Cys)-OH;
SEQ ID NO: 117
D-Phe-c(Cys-His-D-Phe-Arg-Trp-Ala-D-Cys)-Thr-OH;
SEQ ID NO: 118
D-Phe-c(Cys-His-D-Phe-Arg-Trp-β-Ala-D-Cys)-Thr-OH;
SEQ ID NO: 119
D-Phe-c(Cys-His-D-Phe-Arg-Trp-Gaba-D-Cys)-Thr-OH;
SEQ ID NO: 120
Ac-Nle-c(Cys-His-D-Phe-Arg-Trp-Apn-Cys)-OH;
SEQ ID NO: 121
Ac-Nle-c(Asp-His-D-Phe-Arg-Trp-Apn-Lys)-OH;
SEQ ID NO: 122
Ac-Cha-c(Asp-His-D-Phe-Arg-Trp-Gaba-Lys)-OH;
SEQ ID NO: 123
Ac-Nle-c(Asp-His-D-Phe-Arg-Trp-Gaba-Lys)-OH;
SEQ ID NO: 124
Ac-Chg-c(Asp-His-D-Phe-Arg-Trp-Gaba-Lys)-OH;
SEQ ID NO: 125
Ac-D-Cha-c(Asp-His-D-Phe-Arg-Trp-Gaba-Lys)-OH;
SEQ ID NO: 126
Ac-hCha-c(Asp-His-D-Phe-Arg-Trp-Gaba-Lys)-OH;
SEQ ID NO: 127
Ac-D-Chg-c(Asp-His-D-Phe-Arg-Trp-Gaba-Lys)-OH;
SEQ ID NO: 128
Ac-hPhe-c(Asp-His-D-Phe-Arg-Trp-Gaba-Lys)-OH;
SEQ ID NO: 129
Ac-Nle-c(Cys-His-D-Phe-Arg-D-Trp-Gaba-Cys)-OH;
SEQ ID NO: 130
Ac-Nle-c(Cys-His-D-Phe-Arg-D-Trp-Ahx-Cys)-OH;
SEQ ID NO: 131
Ac-Nle-c(Cys-His-D-Phe-Arg-D-Trp-β-Ala-Cys)-OH;
SEQ ID NO: 132
Ac-Nle-c(Cys-His-D-Phe-Arg-D-Trp-D-Ala-Cys)-OH;
SEQ ID NO: 133
Ac-Nle-c(Cys-D-Ala-His-D-2-Nal-Arg-Trp-Cys)-OH;
SEQ ID NO: 134
Ac-Nle-c(Cys-D-Ala-His-D-2-Nal-Arg-2-Nal-Cys)-OH;
SEQ ID NO: 135
Ac-Nle-c(Cys-D-Ala-His-D-2-Nal-Arg-1-Nal-Cys)-OH;
SEQ ID NO: 136
Ac-Nle-c(Cys-D-Ala-His-D-2-Nal-Arg-Bal-Cys)-OH;
SEQ ID NO: 137
Ac-Nle-c(Pen-D-Ala-His-D-Phe-Arg-Trp-Cys)-OH;
SEQ ID NO: 138
Ac-Nle-c(Cys-His-D-Phe-Arg-Trp-Gaba-Pen)-OH;
SEQ ID NO: 139
Ac-Arg-c(Cys-D-Ala-His-D-2-Nal-Arg-Trp-Cys)-NH₂;
SEQ ID NO: 140
Ac-Arg-c(Cys-D-Ala-His-D-Phe-Arg-Trp-Cys)-NH₂;
SEQ ID NO: 141
Ac-D-Arg-c(Cys-D-Ala-His-D-Phe-Arg-Trp-Cys)-NH₂;
SEQ ID NO: 142
Ac-D-Arg-c(Cys-D-Ala-His-D-Phe-Arg-Trp-Pen)-NH₂;
SEQ ID NO: 143
Ac-D-Arg-c(Cys-His-D-Phe-Arg-Trp-Gaba-Pen)-NH₂;
SEQ ID NO: 144
Ac-Arg-c(Cys-His-D-Phe-Arg-Trp-Gaba-Pen)-NH₂;
SEQ ID NO: 145
Ac-Arg-c(Cys-D-Ala-His-D-Phe-Arg-Trp-Pen)-NH₂;
SEQ ID NO: 146
Ac-D-Arg-c(Asp-His-D-Phe-Arg-Trp-Ala-Lys)-NH₂;
and
SEQ ID NO: 147
Ac-Arg-c(Asp-His-D-Phe-Arg-Trp-Ala-Lys)-NH₂;

or a pharmaceutically acceptable salt thereof.

In embodiments, the compound of Formula (I) is Ac-Arg-c(Cys-D-Ala-His-D-Phe-Arg-Trp-Cys)-NH₂ (SEQ ID NO: 140) or a pharmaceutically acceptable salt thereof. Ac-Arg-c(Cys-D-Ala-His-D-Phe-Arg-Trp-Cys)-NH₂ (SEQ ID NO: 140), also known as RM-493 and setmelanotide, is a peptide that retains the specificity and functionality of the naturally occurring hormone that activates MC4R and has not been shown to adversely affect blood pressure in clinical trials (see, e.g., Chen et al. J. Clin. Endocrinol. Metab. 2015; 100(4):1639-45. The structure of Ac-Arg-c(Cys-D-Ala-His-D-Phe-Arg-Trp-Cys)-NH₂ (SEQ ID NO: 140) is shown below:

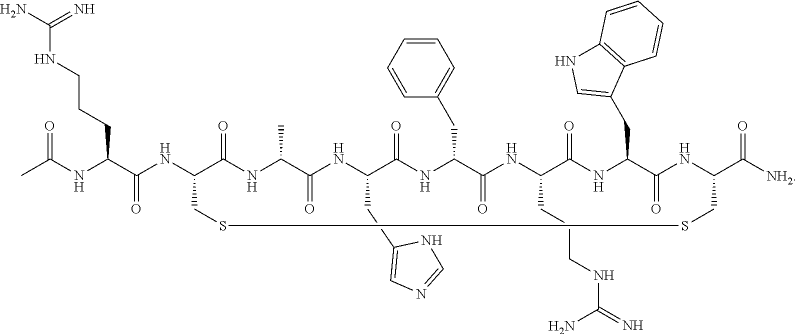 [SMILES: CC(=O)N[C@@H](CCCNC(=N)N)C(=O)N[C@H]1CSSC[C@@H](C(N)=O)NC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC(=O)[C@@H](C)NC1=O]

In some embodiments, the MC4R agonist is a compound of Formula (II):

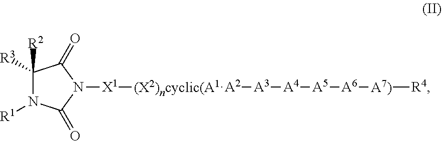

or a pharmaceutically acceptable salt thereof, wherein:

### X¹ is

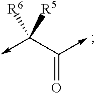

### X² is

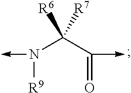

A is Asp, Cys, D-Cys, Dab, Dap, Glu, Lys, Orn, Pen or D-Pen; A² is an L- or D-amino acid; A³ is H is, 2-Pal, 3-Pal, 4-Pal, (X¹, X², X³, X⁴, X⁵)Phe, Taz, 2-Thi or 3-Thi; A⁴ is D-Bal, D-1-Nal, D-2-Nal, D-Phe or D-(X¹, X², X³, X⁴, X⁵)Phe; A⁵ is Arg, hArg, Dab, Dap, Lys or Orn; A⁶ is Bal, 1-Nal, 2-Nal, (X¹, X², X³, X⁴, X⁵)Phe or Trp; A⁷ is Asp, Cys, D-Cys, Dab, Dap, Glu, Lys, Orn, Pen or D-Pen; R¹ is H, (C₁-C₁₀)alkyl or substituted (C₁-C₁₀)alkyl; R² and R³ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together form a cyclic moiety; R⁴ is OH, NH₂, CO₂H or C(O)NH₂; R⁵ and R⁶ each is, independently, H, (C₁-00)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R⁵ and R⁶ may be fused together form a cyclic moiety; R⁷ and R⁸ each is, independently, H, (C₁-C₁₀)alkyl, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl; or R⁷ and R⁸ may be fused together form a cyclic moiety; R⁹ is H, (C₁-C₁₀)alkyl or substituted (C₁-C₁₀)alkyl; and n is, independently for each occurrence thereof, 0, 1, 2, 3, 4, 5, 6 or 7; or a pharmaceutically acceptable salt thereof.

In some embodiments of Formula (II), A¹ is Cys; A² is D-Ala, Asn, Asp, Gln, Glu or D-Phe; A³ is H is; A⁴ is D-2-Nal or D-Phe; A⁵ is Arg; A⁶ is Trp; and A⁷ is Cys or Pen; each of R¹, R², R³, and R⁹ is, independently, H; R⁴ is C(O)NH₂; each of R⁵ and R⁶ is, independently, H, (C₁-C₁₀)heteroalkyl, substituted (C₁-C₁₀)alkyl or substituted (C₁-C₁₀)heteroalkyl or R⁵ and R⁶ may be fused together form a cyclic moiety; and each of R⁷ and R⁸ is, independently, H, (C₁-C₁₀)alkyl, (C₁-C₁₀)heteroalkyl, substituted (C₁-C₁₀)alkyl or substituted (C₁-C₁₀)heteroalkyl;

or pharmaceutically acceptable salts thereof.

In some embodiments, the compound of Formula (II) is selected from:

(SEQ ID NO: 500)
Hydantoin(C(O)-(Arg-Gly))-c(Cys-Glu-His-D-Phe-Arg-Trp-Cys)-NH₂;
(SEQ ID NO: 501)
Hydantoin(C(O)-(Nle-Gly))-c(Cys-Glu-His-D-Phe-Arg-Trp-Cys)-NH₂;
(SEQ ID NO: 502)
Hydantoin(C(O)-(Gly-Gly))-c(Cys-Glu-His-D-Phe-Arg-Trp-Cys)-NH₂;
(SEQ ID NO: 503)
Hydantoin(C(O)-(Nle-Gly))-c(Cys-D-Ala-His-D-Phe-Arg-Trp-Cys)-NH₂;
(SEQ ID NO: 504)
Hydantoin(C(O)-(Gly-Gly))-c(Cys-D-Ala-His-D-Phe-Arg-Trp-Cys)-NH₂;
(SEQ ID NO: 505)
Hydantoin(C(O)-(Nle-Gly))-c(Cys-D-Ala-His-D-Phe-Arg-Trp-Pen)-NH₂;
(SEQ ID NO: 506)
Hydantoin(C(O)-(Gly-Gly))-c(Cys-D-Ala-His-D-Phe-Arg-Trp-Pen)-NH₂;
(SEQ ID NO: 507)
Hydantoin(C(O)-(Ala-Gly))-c(Cys-D-Ala-His-D-Phe-Arg-Trp-Cys)-NH₂;
(SEQ ID NO: 508)
Hydantoin(C(O)-(D-Ala-Gly))-c(Cys-D-Ala-His-D-Phe-Arg-Trp-Cys)-NH₂;
(SEQ ID NO: 509)
Hydantoin(C(O)-(Aib-Gly))-c(Cys-D-Ala-His-D-Phe-Arg-Trp-Cys)-NH₂;
(SEQ ID NO: 510)
Hydantoin(C(O)-(Val-Gly))-c(Cys-D-Ala-His-D-Phe-Arg-Trp-Cys)-NH₂;
(SEQ ID NO: 511)
Hydantoin(C(O)-(Ile-Gly))-c(Cys-D-Ala-His-D-Phe-Arg-Trp-Cys)-NH₂;
(SEQ ID NO: 512)
Hydantoin(C(O)-(Leu-Gly))-c(Cys-D-Ala-His-D-Phe-Arg-Trp-Cys)-NH₂;
(SEQ ID NO: 513)
Hydantoin(C(O)-(Gly-Gly))-c(Cys-Glu-His-D-2-Nal-Arg-Trp-Cys)-NH₂;
(SEQ ID NO: 514)
Hydantoin(C(O)-(Nle-Gly))-c(Cys-Glu-His-D-2-Nal-Arg-Trp-Cys)-NH₂;
(SEQ ID NO: 515)
Hydantoin(C(O)-(D-Arg-Gly))-c(Cys-Glu-His-D-Phe-Arg-Trp-Cys)-NH₂;
(SEQ ID NO: 516)
Hydantoin(C(O)-(D-Arg-Gly))-c(Cys-D-Ala-His-D-Phe-Arg-Trp-Cys)-NH₂;
(SEQ ID NO: 517)
Hydantoin(C(O)-(Arg-Gly))-c(Cys-D-Ala-His-D-Phe-Arg-Trp-Cys)-NH₂;
(SEQ ID NO: 518)
Hydantoin(C(O)-(D-Arg-Gly))-c(Cys-D-Ala-His-D-2-Nal-Arg-Trp-Cys)-NH₂;
(SEQ ID NO: 519)
Hydantoin(C(O)-(Arg-Gly))-c(Cys-D-Ala-His-D-2-Nal-Arg-Trp-Cys)-NH₂;
(SEQ ID NO: 520)
Hydantoin(C(O)-(Ala-Nle))-c(Cys-Glu-His-D-Phe-Arg-Trp-Cys)-NH₂;
(SEQ ID NO: 521)
Hydantoin(C(O)-(Val-Nle))-c(Cys-Glu-His-D-Phe-Arg-Trp-Cys)-NH₂;
(SEQ ID NO: 522)
Hydantoin(C(O)-(Gly-Nle))-c(Cys-Glu-His-D-Phe-Arg-Trp-Cys)-NH₂;
(SEQ ID NO: 523)
Hydantoin(C(O)-(A6c-Nle))-c(Cys-D-Ala-His-D-Phe-Arg-Trp-Cys)-NH₂;
(SEQ ID NO: 524)
Hydantoin(C(O)-(Gly-Nle))-c(Cys-D-Ala-His-D-Phe-Arg-Trp-Cys)-NH₂;
(SEQ ID NO: 525)
Hydantoin(C(O)-(Ala-Nle))-c(Cys-D-Ala-His-D-Phe-Arg-Trp-Cys)-NH₂;
(SEQ ID NO: 526)
Hydantoin(C(O)-(D-Ala-Nle))-c(Cys-D-Ala-His-D-Phe-Arg-Trp-Cys)-NH₂;
(SEQ ID NO: 527)
Hydantoin(C(O)-(Val-Nle))-c(Cys-D-Ala-His-D-Phe-Arg-Trp-Cys)-NH₂;
(SEQ ID NO: 528)
Hydantoin(C(O)-(Leu-Nle))-c(Cys-D-Ala-His-D-Phe-Arg-Trp-Cys)-NH₂;
(SEQ ID NO: 529)
Hydantoin(C(O)-(Cha-Nle))-c(Cys-D-Ala-His-D-Phe-Arg-Trp-Cys)-NH₂;
(SEQ ID NO: 530)
Hydantoin(C(O)-(Aib-Nle))-c(Cys-D-Ala-His-D-Phe-Arg-Trp-Cys)-NH₂;
(SEQ ID NO: 531)
Hydantoin(C(O)-(Gly-Arg))-c(Cys-Glu-His-D-Phe-Arg-Trp-Cys)-NH₂;
(SEQ ID NO: 532)
Hydantoin(C(O)-(Gly-Arg))-c(Cys-Glu-His-D-2-Nal-Arg-Trp-Cys)-NH₂;
(SEQ ID NO: 533)
Hydantoin(C(O)-(Gly-Arg))-c(Cys-D-Ala-His-D-Phe-Arg-Trp-Cys)-NH₂;
(SEQ ID NO: 534)
Hydantoin(C(O)-(Gly-Arg))-c(Cys-D-Ala-His-D-2-Nal-Arg-Trp-Cys)-NH₂;
(SEQ ID NO: 535)
Hydantoin(C(O)-(Gly-D-Arg))-c(Cys-Glu-His-D-Phe-Arg-Trp-Cys)-NH₂;
(SEQ ID NO: 536)
Hydantoin(C(O)-(Gly-D-Arg))-c(Cys-D-Ala-His-D-Phe-Arg-Trp-Cys)-NH₂;
(SEQ ID NO: 537)
Hydantoin(C(O)-(Gly-D-Arg))-c(Cys-D-Ala-His-D-2-Nal-Arg-Trp-Cys)-NH₂;
and
(SEQ ID NO: 538)
Hydantoin(C(O)-(Nle-Ala))-c(Cys-Glu-His-D-Phe-Arg-Trp-Cys)-NH₂;

or a pharmaceutically acceptable salt thereof.

In some embodiments, the compound of Formula (II) is described in WO2008/147556 or International Patent Application Number PCT/US08/06675, each of which is incorporated herein by reference in its entirety.

In embodiments, the compound of Formula (II) is hydantoin(C(O)-(Arg-Gly))-c(Cys-Glu-His-D-Phe-Arg-Trp-Cys)-NH₂ (SEQ ID NO: 500) or a pharmaceutically acceptable salt thereof, also known as RM-511. The structure of hydantoin(C(O)-(Arg-Gly))-c(Cys-Glu-His-D-Phe-Arg-Trp-Cys)-NH₂ (SEQ ID NO: 500) is shown below:

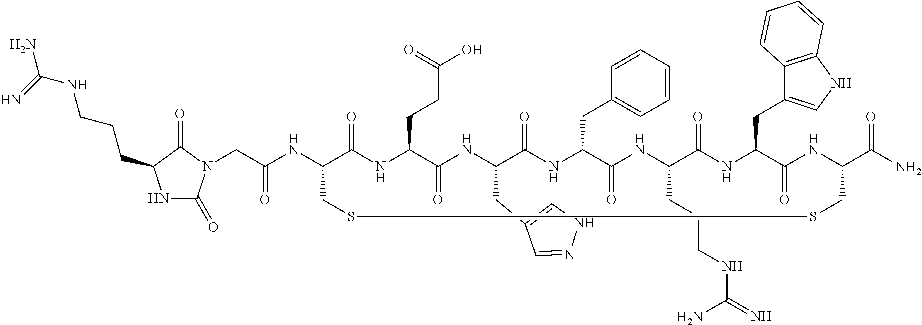 [SMILES: N=C(N)NCCC[C@@H]1NC(=O)[C@@H](Cc2ccccc2)NC(=O)[C@H](Cc2cn[nH]c2)NC(=O)[C@H](CCC(=O)O)NC(=O)[C@@H](NC(=O)CN2C(=O)N[C@@H](CCCNC(=N)N)C2=O)CSSC[C@@H](C(N)=O)NC(=O)[C@H](Cc2c[nH]c3ccccc23)NC1=O]

In some embodiments, the MC4R agonist is a compound of Formula (III):

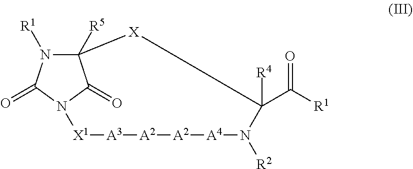

or a pharmaceutically acceptable salt thereof, wherein X is selected from the group consisting of —CH₂S—S—CH₂—, —C(CH₃)₂S—S—CH₂—, CH₂—S—S—C(CH₃)₂, C(CH₃)₂—S—S—C(CH₃)₂—, (CH₂)₂S—S—CH₂—, CH₂—S—S—(CH₂)₂—, (CH₂)₂—S—S—(CH₂)₂—, C(CH₃)₂—S—S—(CH₂)₂—, (CH₂)₂—S—S—C(CH₃)₂, (CH₂)ₜ—C(O)—NR⁸—(CH₂)ᵣ— and (CH₂)ᵣ—NR⁸—C(O)—(CH₂)ₜ; R² each is, independently, H, (C₁-C₁₀)alkyl or substituted (C₁-C₁₀)alkyl; R³ is OH or —NH₂; R⁴ and R⁵ each is, independently, H, (C₁-C₁₀)alkyl or substituted (C₁-C₁₀)alkyl; X¹ is

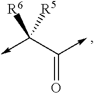

A¹ is H is, 2-Pal, 3-Pal, 4-Pal, (X¹, X², X³, X⁴, X⁵)Phe, Taz, 2-Thi, 3-Thi or is deleted; A² is D-Bal, D-1-Nal, D-2-Nal, D-Phe or D-(X¹, X², X³, X⁴, X⁵)Phe; A³ is Arg, hArg, Dab, Dap, Lys or Orn; A⁴ is Bal, 1-Nal, 2-Nal, (X¹, X², X³, X⁴, X⁵)Phe or Trp; R⁶ and R⁷ each is, independently for each occurrence thereof, H, (C₁-C₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl provided that R⁶ and R⁷ may be joined together to form a ring; R⁸ is H, (C₁-C₁₀)alkyl or substituted (C₁-C₁₀)alkyl; r is, independently for each occurrence thereof, 1, 2, 3, 4 or 5; and t is, independently for each occurrence thereof, 1 or 2.

Compounds according the foregoing formula can include compounds wherein X 1 is selected from the group consisting of:

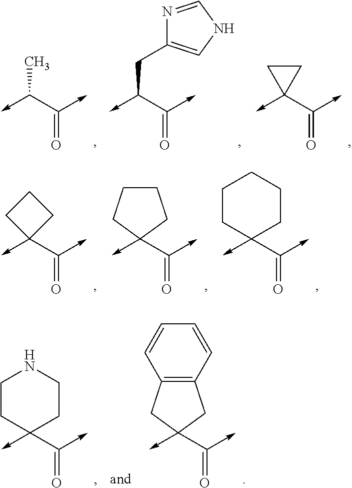

Compounds of Formula (III) are disclosed in International Patent Publication WO 2008/147556 or International Patent Application Number PCT/US08/06675, each of which is incorporated herein by reference in its entirety.

In some embodiments, the compound of Formula (III) is selected from:

SEQ ID NO: 474)
c[Hydantoin(C(O)-(Cys-D-Ala))-His-D-Phe-Arg-Trp-Cys]-NH₂;
(SEQ ID NO: 475)
c[Hydantoin(C(O)-(hCys-D-Ala))-His-D-Phe-Arg-Trp-Cys]-NH₂;
(SEQ ID NO: 476)
c[Hydantoin(C(O)-(Cys-D-Ala))-His-D-2-Nal-Arg-Trp-Cys]-NH₂;
(SEQ ID NO: 477)
c[Hydantoin(C(O)-(hCys-D-Ala))-His-D-2-Nal-Arg-Trp-Cys]-NH₂;
(SEQ ID NO: 478)
c[Hydantoin(C(O)-(Asp-D-Ala))-His-D-Phe-Arg-Trp-Lys]-NH₂;
(SEQ ID NO: 479)
c[Hydantoin(C(O)-(Asp-D-Ala))-His-D-Phe-Arg-Trp-Orn]-NH₂;
(SEQ ID NO: 480)
c[Hydantoin(C(O)-(Asp-D-Ala))-His-D-Phe-Arg-Trp-Dab]-NH₂;
(SEQ ID NO: 481)
c[Hydantoin(C(O)-(Asp-D-Ala))-His-D-Phe-Arg-Trp-Dap]-NH₂;
(SEQ ID NO: 482)
c[Hydantoin(C(O)-(Asp-His))-D-2-Nal-Arg-Trp-Lys]-NH₂;
(SEQ ID NO: 483)
c[Hydantoin(C(O)-(Asp-His))-D-Phe-Arg-Trp-Lys]-NH₂;
(SEQ ID NO: 484)
c[Hydantoin(C(O)-(Asp-A3c))-D-Phe-Arg-Trp-Lys]-NH₂;
(SEQ ID NO: 485)
c[Hydantoin(C(O)-(Asp-A5c))-D-Phe-Arg-Trp-Lys]-NH₂;
(SEQ ID NO: 486)
c[Hydantoin(C(O)-(Asp-A6c))-D-Phe-Arg-Trp-Lys]-NH₂;
(SEQ ID NO: 487)
c[Hydantoin(C(O)-(Asp-A3c))-D-2-Nal-Arg-Trp-Lys]-NH₂;
(SEQ ID NO: 488)
c[Hydantoin(C(O)-(Asp-A5c))-D-2-Nal-Arg-Trp-Lys]-NH₂;
(SEQ ID NO: 489)
c[Hydantoin(C(O)-(Asp-A6c))-D-2-Nal-Arg-Trp-Lys]-NH₂;
(SEQ ID NO: 490)
c[Hydantoin(C(O)-(Asp-Aic))-D-Phe-Arg-Trp-Lys]-NH₂;
(SEQ ID NO: 491)
c[Hydantoin(C(O)-(Asp-Apc))-D-Phe-Arg-Trp-Lys]-NH₂;
(SEQ ID NO: 492)
c[Hydantoin(C(O)-(Asp-Aic))-D-2-Nal-Arg-Trp-Lys]-NH₂;
(SEQ ID NO: 493)
c[Hydantoin(C(O)-(Asp-Apc))-D-2-Nal-Arg-Trp-Lys]-NH₂;
(SEQ ID NO: 494)
c[Hydantoin(C(O)-(Glu-D-Ala))-His-D-Phe-Arg-Trp-Orn]-NH₂;
(SEQ ID NO: 495)
c[Hydantoin(C(O)-(Glu-D-Ala))-His-D-Phe-Arg-Trp-Dab]-NH₂;
(SEQ ID NO: 496)
c[Hydantoin(C(O)-(Glu-D-Ala))-His-D-Phe-Arg-Trp-Dap]-NH₂;
(SEQ ID NO: 497)
c[Hydantoin(C(O)-(Glu-D-Ala))-His-D-Phe-Arg-Trp-Lys]-NH₂;
(SEQ ID NO: 498)
c[Hydantoin(C(O)-(Glu-His))-D-Phe-Arg-Trp-Dap]-NH₂;
and
(SEQ ID NO: 499)
c[Hydantoin(C(O)-(Glu-His))-D-Phe-Arg-Trp-Lys]-NH₂,

or a pharmaceutically acceptable salt thereof.

In some embodiments, the MC4R agonist is a compound of Formula (IV):

(R²R³)-A¹-c(A²-A³-A⁴-A⁵-A⁶-A⁷-A⁸-A⁹)-NH₂ (IV)

or a pharmaceutically acceptable salt thereof, wherein A¹ is Nle or deleted; A² is Cys or Asp; A³ is Glu or D-Ala; A⁴ is His; A⁵ is D-Phe; A⁶ is Arg; A⁷ is Trp, 2-Nal or Bal; A⁸ is Gly, Ala, D-Ala, (β-Ala, Gaba or Apn; A⁹ is Cys or Lys; each of R² and R³ is independently selected from the group consisting of H or (C₁-C₆)acyl.

In exemplary embodiments of Formula (IV): (I) when R² is (C₁-C₆)acyl, then R³ is H; and (II) when A² is Cys, then A⁹ is Cys.

Exemplary MC4R agonists of Formula (IV) are disclosed in International Patent Application Publication Number WO 2007/008704, which is incorporated herein by reference in its entirety.

In some embodiments, the compound of Formula (IV) is selected from:

SEQ ID NO: 148
Ac-Nle-c(Cys-D-Ala-His-D-Phe-Arg-Trp-Gly-Cys)-NH₂;
SEQ ID NO: 149
Ac-Nle-c(Cys-D-Ala-His-D-Phe-Arg-Trp-D-Ala-Cys)-NH₂;
SEQ ID NO: 150
Ac-Nle-c(Cys-D-Ala-His-D-Phe-Arg-Trp-β-Ala-Cys)-NH₂;
SEQ ID NO: 151
Ac-Nle-c(Cys-D-Ala-His-D-Phe-Arg-Trp-Gaba-Cys)-NH₂;
SEQ ID NO: 152
Ac-Nle-c(Cys-D-Ala-His-D-Phe-Arg-Trp-Apn-Cys)-NH₂;
SEQ ID NO: 153
Ac-c(Cys-Glu-His-D-Phe-Arg-Trp-Ala-Cys)-NH₂;
SEQ ID NO: 154
Ac-c(Cys-Glu-His-D-Phe-Arg-2-Nal-Ala-Cys)-NH₂;
SEQ ID NO: 155
Ac-c(Cys-D-Ala-His-D-Phe-Arg-Trp-Ala-Cys)-NH₂;
SEQ ID NO: 156
Ac-c(Cys-D-Ala-His-D-Phe-Arg-2-Nal-Ala-Cys)-NNH₂;
SEQ ID NO: 157
Ac-Nle-c(Cys-D-Ala-His-D-Phe-Arg-Trp-Ala-Cys)-NH₂;
or
SEQ ID NO: 158
Ac-Nle-c(Asp-D-Ala-His-D-Phe-Arg-Bal-Ala-Lys)-NH₂;

or a pharmaceutically acceptable salt thereof.

In some embodiments, the MC4R agonist is a compound of Formula (V):

(R²R³)—B¹-A¹-c(A²-A³-A⁴-A⁵-A⁶-A⁷-A⁸-A⁹)-A¹⁰-A¹¹-A¹²-A¹³-B²-B³-R¹ (V)

or a pharmaceutically acceptable salt thereof: B¹ is a peptide moiety which contains 5, 6, 7, 8, 9, 10, 11, 12, 13, 14, or 15 amino acids, wherein at least 5 amino acids are independently selected from the group consisting of L-Arg, D-Arg, L-hArg and D-hArg, or B¹ is optionally deleted; A¹ is Acc, HN—(CH₂)ₘ—C(O), L- or D-amino acid or deleted; A² is Cys, D-Cys, hCys, D-hCys, Pen, D-Pen, Asp or Glu; A³ is Gly, Glu, Ala, β-Ala, Gaba, Aib, D-amino acid or deleted; A⁴ is H is, 2-Pal, 3-Pal, 4-Pal, Taz, 2-Thi, 3-Thi or (X′, X², X³, X⁴, X⁵)Phe; A⁵ is D-Phe, D-1-Nal, D-2-Nal, D-Trp, D-Bal, D-(X¹, X², X³, X⁴, X⁵)Phe, D-(Et)Tyr, D-Dip, D-Bip or D-Bpa; A⁶ is Arg, hArg, Dab, Dap, Lys, Orn or HN-CH((CH₂)ₙ—N(R⁴R⁵))—C(O); A⁷ is Trp, 1-Nal, 2-Nal, Bal, Bip, Dip, Bpa, D-Trp, D-1-Nal, D-2-Nal, D-Bal, D-Bip, D-Dip or D-Bpa; A⁸ is Gly, D-Ala, Acc, Ala, β-Ala, Gaba, Apn, Ahx, Aha, HN—(CH₂)ₛ—C(O) or deleted; A⁹ is Cys, D-Cys, hCys, D-hCys, Pen, D-Pen, Dab, Dap, Orn or Lys; A¹⁰ is Acc, HN—(CH₂)ᵣ—C(O), Pro, hPro, 3-Hyp, 4-Hyp, Thr, an L- or D-amino acid or deleted; A¹¹ is Pro, hPro, 3-Hyp, 4-Hyp or deleted; A¹² is Lys, Dab, Dap, Arg, hArg or deleted; A¹³ is Asp, Glu or deleted; B² is a peptide moiety containing 1, 2, 3, 4, or 5 amino acids or deleted, B³ is a peptide moiety which contains 5, 6, 7, 8, 9, 10, 11, 12, 13, 14 or 15 amino acids wherein at least 5 amino acids are independently selected from the group consisting of L-Arg, D-Arg, L-hArg and D-hArg, or is deleted; R¹ is OH or NH₂; R² and R³ each is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl and substituted aryl(C₁-C₃₀)acyl; R⁴ and R⁵ each is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl or C(NH)—NH₂; n is, independently for each occurrence, 1, 2, 3, 4 or 5; m is, independently for each occurrence, 1, 2, 3, 4, 5, 6 or 7; s is, independently for each occurrence, 1, 2, 3, 4, 5, 6 or 7; t is, independently for each occurrence, 1, 2, 3, 4, 5, 6 or 7; X¹, X², X³, X⁴ and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂ or CN.

In some embodiments of Formula (V):
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl or C(NH)—NH₂, then R⁵ is H, (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl or substituted aryl(C₁-C₄₀)alkyl;
(II) when R² is (C₁-C₃₀)acyl, aryl(C₁-C₃₀)acyl, substituted (C₁-C₃₀)acyl or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl or substituted aryl(C₁-C₃₀)alkyl;
(III) neither B¹ nor B² contains one or more of the following amino acid sequences: Arg-(Lys)₂-(Arg)₂-Gln-(Arg)₃, Tyr-Ala-Arg-Lys-Ala-(Arg)₂-Gln-Ala-(Arg)₂, Tyr-Ala-Arg-(Ala)₂-(Arg)₂-(Ala)₂-(Arg)₂, Tyr-Ala-(Arg)₉, Tyr-(Ala)₃-(Arg)₇, Tyr-Ala-Arg-Ala-Pro-(Arg)₂-Ala-(Arg)₃ or Tyr-Ala-Arg-Ala-Pro-(Arg)₂-Pro-(Arg)₂;
(IV) either B¹ or B² or both must be present in said compound;
(V) when A² is Cys, D-Cys, hCys, D-hCys, Pen or D-Pen, then A⁹ is Cys, D-Cys, hCys, D-hCys, Pen or D-Pen; and
(VI) when A² is Asp or Glu, then A⁹ is Dab, Dap, Orn or Lys.

In some embodiments of Formula (V):
B¹ is Arg-Lys-Gln-Lys-(Arg)₅, Arg-(Lys)₂-Arg-Gln-(Arg)₄, Arg-(Lys)₂-(Arg)₃-Gln-(Arg)₂, Arg-(Lys)₂-(Arg)₄-Gln-Arg, Arg-(Lys)₂-(Arg)₅-Gln, Arg-(Lys)₂-Gln-(Arg)₅, Arg-Gln-(Lys)₂-(Arg)₅, Arg-Gln-(Arg)₇, Arg-Gln-(Arg)₅, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg)₅-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₈, (Arg)s-Gln-Arg, (Arg)₉, (Arg)₉-Gln, (D-Arg)₅, (D-Arg)₆, (D-Arg)₇, (D-Arg)₈, (D-Arg)₉, Gln-Arg-(Lys)₂-(Arg)₅, Gln-(Arg)₅, Gln-(Arg)₉, Tyr-Gly-Arg-(Lys)₂-(Arg)₂-Gln-(Arg)₃, Tyr-Gly-Arg-(Lys)₂-(Arg)₂-Gln-(Arg)₃-Doc; or deleted;
B² is β-Ala, β-Ala-Gly, β-Ala-Tyr, β-Ala-Tyr-Gly, (β-Ala)₂, (β-Ala)₂-Gly, (β-Ala)₂-Tyr, (β-Ala)₂-Tyr-Gly, Doc, Doc-Gly, Doc-Tyr, Doc-Tyr-Gly, (DoC)₂, (Doc)₂-Gly, (DoC)₂-Tyr, Doc)₂-Tyr-Gly, or deleted;
B³ is Arg-Lys-Gln-Lys-(Arg)₅, Arg-Lys-(Arg)₃-Gln-(Arg)₃, Arg-(Lys)₂-Arg-Gln-(Arg)₄, Arg-(Lys)₂-Gln-(Arg)₅, Arg-(Lys)₂-(Arg)₂-Gln-(Arg)₃, Arg-(Lys)₂-(Arg)₃-Gln-(Arg)₂, Arg-(Lys)₂-(Arg)₄-Gln-Arg, Arg-(Lys)₂-(Arg)₅-Gln, Arg-Gln-(Lys)₂-(Arg)₅, Arg-Gln-(Arg)₇, Arg-Gln-(Arg)₅, (Arg)₂-Lys-(Arg)₂-Gln-(Arg)₃, (Arg)₂-Gln-(Arg)₆, (Arg)₂-Gln-(Arg)₇, (Arg)₃-Gln-(Arg)₅, (Arg)₃-Gln-(Arg)₆, (Arg)₄-Gln-(Arg)₄, (Arg)₄-Gln-(Arg)₅, (Arg)₅, (Arg)₅-Gln-(Arg)₃, (Arg)₅-Gln-(Arg)₄, (Arg)₆, (Arg)₆-Gln-(Arg)₃, (Arg)₇, (Arg)₇-Gln-(Arg)₂, (Arg)₅, (Arg)₅-Gln-Arg, (Arg)₉, (Arg)₉-Gln, (D-Arg)₅, (D-Arg)₆, (D-Arg)₇, (D-Arg)₅, (D-Arg)₉, Gln-Arg-(Lys)₂-(Arg)₅, Gln-(Arg)₅, Gln-(Arg)₉, or deleted;
A¹ is A6c, Cha, hCha, Chg, D-Chg, hChg, Gaba, hLeu, Met, β-hMet, D-2-Nal, Nip, Nle, Oic, Phe, D-Phe, hPhe, hPro, or deleted;
A² is Cys;
A³ is D-Abu, Aib, Ala, 1-Ala, D-Ala, D-Cha, Gaba, Glu, Gly, D-Ile, D-Leu, D-Met, D-Nle, D-Phe, D-Tle, D-Trp, D-Tyr, D-Val, or deleted;
A⁴ is H;
A⁵ is D-Bal, D-1-Nal, D-2-Nal, D-Phe, D-(X¹, X², X³, X⁴, X⁵)Phe, D-Trp, or D-(Et)Tyr;
A⁶ is Arg or hArg;
A⁷ is Bal, Bip, 1-Nal, 2-Nal, Trp, or D-Trp;
A⁸ is A⁵c, A⁶c, Aha, Ahx, Ala, 1-Ala, Apn, Gaba, Gly, or deleted;
A⁹ is Cys, D-Cys, hCys, D-hCys, Lys, Pen, or D-Pen;
A¹⁰ is Pro, Thr or deleted;
A¹¹ is Pro or deleted;
A¹² is arg, Lys, or deleted;
A¹³ is Asp or deleted;
each of R² and R³ is, independently, H or acyl;
   or pharmaceutically acceptable salts thereof.

In some embodiments, the compound of Formula (V) is selected from:

(SEQ ID NO: 159)
Tyr-Gly-Arg-(Lys)₂-(Arg)₂-Gln-(Arg)₃-Nle-c(Asp-His-D-2-Nal-Arg-Trp-Lys)-NH₂;
(SEQ ID NO: 160)
Tyr-Gly-Arg-(Lys)₂-(Arg)₂-Gln-(Arg)₃-Doc-Nle-c(Asp-His-D-2-Nal-Arg-Trp-Lys)-
NH₂;
(SEQ ID NO: 161)
Nle-c(Asp-His-D-2-Nal-Arg-Trp-Lys)-β-Ala-Tyr-Gly-Arg-(Lys)₂-(Arg)₂-Gln-(Arg)₃-
NH₂;
(SEQ ID NO: 162)
Ac-Nle-c(Asp-His-D-2-Nal-Arg-Trp-Lys)-β-Ala-Tyr-Gly-Arg-(Lys)₂-(Arg)₂-Gln-
(Arg)₃-NH₂;
(SEQ ID NO: 163)
Nle-c(Asp-His-D-2-Nal-Arg-Trp-Lys)-(Doc)₂-Tyr-Gly-Arg-(Lys)₂-(Arg)₂-Gln-
(Arg)₃-NH₂;
(SEQ ID NO: 164)
Ac-Nle-c(Asp-His-D-2-Nal-Arg-Trp-Lys)-(Pro)₂-Lys-Asp-Tyr-Gly-Arg-(Lys)₂ -
(Arg)₂-Gln-(Arg)₃-NH₂;
(SEQ ID NO: 165)
Ac-c(Cys-Glu-His-D-2-Nal-Arg-Trp-Gly-Cys)-(Pro)₂-Lys-Asp-Tyr-Gly-Arg-(Lys)₂-
(Arg)₂-Gln-(Arg)₃-NH₂;
(SEQ ID NO: 166)
Ac-Nle-c(Asp-His-D-2-Nal-Arg-Trp-Lys)-(β-Ala)₂-Tyr-Gly-Arg-(Lys)₂-(Arg)₂-Gln-
(Arg)₃-NH₂;
(SEQ ID NO: 167)
Ac-Nle-c(Asp-His-D-2-Nal-Arg-Trp-Lys)-(Pro)₂-Lys-Asp-Doc-Tyr-Gly-Arg-(Lys)₂-
(Arg)₂-Gln-(Arg)₃-NH₂;
(SEQ ID NO: 168)
Ac-c(Cys-Glu-His-D-2-Nal-Arg-Trp-Gly-Cys)-(Pro)₂-Lys-Asp-Doc-Tyr-Gly-Arg-
(Lys)₂-(Arg)₂-Gln-(Arg)₃-NH₂;
(SEQ ID NO: 169)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 170)
Ac-c(Cys-Glu-His-D-2-Nal-Arg-Trp-Ala-Cys)-(Pro)₂-Lys-Asp-Doc-Tyr-Gly-Arg-
(Lys)₂-(Arg)₂-Gln-(Arg)₃-NH₂;
(SEQ ID NO: 171)
Ac-Nle-c(Asp-His-D-2-Nal-Arg-Trp-Lys)-(Doc)₂-Tyr-Gly-Arg-(Lys)₂-(Arg)₂-Gln-
(Arg)₃-NH₂;
(SEQ ID NO: 172)
Ac-c(Cys-Glu-His-D-2-Nal-Arg-2-Nal-Ala-Cys)-(Pro)₂-Lys-Asp-β-Ala-Tyr-Gly-Arg-
(Lys)₂-(Arg)₂-Gln-(Arg)₃-NH₂;
(SEQ ID NO: 173)
Ac-c(Cys-Glu-His-D-2-Nal-Arg-Bal-Ala-Cys)-(Pro)₂-Lys-Asp-β-Ala-(Arg)₅-Gln-
(Arg)₃-NH₂;
(SEQ ID NO: 174)
Ac-c(Cys-Glu-His-D-2-Nal-Arg-Bal-Ala-Cys)-(Pro)₂-Lys-Asp-β-Ala-Gly-(Arg)₅-
Gln-(Arg)₃-NH₂;
(SEQ ID NO: 175)
Ac-c(Cys-Glu-His-D-2-Nal-Arg-Bal-Ala-Cys)-(Pro)₂-Lys-Asp-β-Ala-Tyr-Gly-
(Arg)₅-Gln-(Arg)₃-NH₂;
(SEQ ID NO: 176)
Ac-c(Cys-Glu-His-D-2-Nal-Arg-Trp-Ala-Cys)-(Pro)₂-Lys-Asp-β-Ala-Tyr-Gly-Arg-
(Lys)₂-Arg-Gln-(Arg)₄-NH₂;
(SEQ ID NO: 177)
Ac-c(Cys-Glu-His-D-2-Nal-Arg-Trp-Ala-Cys)-(Pro)₂-Lys-Asp-β-Ala-Tyr-Gly-Arg-
(Lys)₂-Gln-(Arg)₅-NH₂;
(SEQ ID NO: 178)
Ac-c(Cys-Glu-His-D-2-Nal-Arg-Trp-Ala-Cys)-(Pro)₂-Lys-Asp-β-Ala-Tyr-Gly-Arg-
Lys-Gln-Lys-(Arg)₅-NH₂;
(SEQ ID NO: 179)
Ac-c(Cys-Glu-His-D-2-Nal-Arg-Trp-Ala-Cys)-(Pro)₂-Lys-Asp-β-Ala-Tyr-Gly-Arg-
(Lys)₂-(Arg)₄-Gln-Arg-NH₂;
(SEQ ID NO: 180)
Ac-c(Cys-Glu-His-D-2-Nal-Arg-Bal-Ala-Cys)-(Pro)₂-Lys-Asp-β-Ala-Tyr-Aib-Arg-
(Lys)₂-(Arg)₂-Gln-(Arg)₃-NH₂;
(SEQ ID NO: 181)
Ac-c(Cys-Glu-His-D-2-Nal-Arg-1-Nal-Ala-Cys)-(Pro)₂-Arg-Asp-β-Ala-(Arg)₅ -Gln-
(Arg)₃-NH₂;
(SEQ ID NO: 182)
Ac-c(Cys-Glu-His-D-2-Nal-Arg-1-Nal-Ala-Cys)-(Pro)₂-Lys-Asp-β-Ala-(Arg)₅-Gln-
(Arg)₃-NH₂;
(SEQ ID NO: 183)
Ac-c(Cys-Glu-His-D-2-Nal-Arg-1-Nal-Ala-Cys)-(Pro)₂-Lys-Asp-β-Ala-(Arg)₆-Gln-
(Arg)₃-NH₂;
(SEQ ID NO: 184)
Ac-c(Cys-Glu-His-D-2-Nal-Arg-2-Nal-Ala-Cys)-(Pro)₂-Arg-Asp-β-Ala-(Arg)₅-Gln-
(Arg)₃-NH₂;
(SEQ ID NO: 185)
Ac-c(Cys-Glu-His-D-2-Nal-Arg-2-Nal-Ala-Cys)-(Pro)₂-Lys-Asp-β-Ala-(Arg)₅ -Gln-
(Arg)₃-NH₂;
(SEQ ID NO: 186)
Ac-c(Cys-Glu-His-D-2-Nal-Arg-2-Nal-Ala-Cys)-(Pro)₂-Lys-Asp-β-Ala-(Arg)₆ -Gln-
(Arg)₃-NH₂;
(SEQ ID NO: 187)
Ac-c(Cys-Glu-His-D-2-Nal-Arg-Bal-Ala-Cys)-(Pro)₂-Arg-Asp-ß-Ala-(Arg)₆-Gln-
(Arg)₃-NH₂;
(SEQ ID NO: 188)
Ac-c(Cys-Glu-His-D-2-Nal-Arg-Bal-Ala-Cys)-(Pro)₂-Arg-Asp-ß-Ala-(Arg)₅-Gln-
(Arg)₃-NH₂;
(SEQ ID NO: 189)
Ac-c(Cys-Glu-His-D-2-Nal-Arg-Bal-Ala-Cys)-(Pro)₂-Lys-Asp-ß-Ala-(Arg)₆-Gln-
(Arg)₃-NH₂;
(SEQ ID NO: 190)
Ac-c(Cys-Glu-His-D-2-Nal-Arg-Trp-Ala-Cys)-(Pro)₂-Lys-Asp-ß-Ala-Tyr-Gly-Arg-
(Lys)₂-(Arg)₃-Gln-(Arg)₂-NH₂;
(SEQ ID NO: 191)
Ac-c(Cys-Glu-His-D-2-Nal-Arg-Trp-Ala-Cys)-(Pro)₂-Lys-Asp-ß-Ala-Tyr-Gly-Arg-
Gln-(Lys)₂-(Arg)₅-NH₂;
(SEQ ID NO: 192)
Ac-c(Cys-Glu-His-D-2-Nal-Arg-Trp-Ala-Cys)-(Pro)₂-Lys-Asp-ß-Ala-Tyr-Gly-Arg-
(Lys)₂-(Arg)₅-Gln-NH₂;
(SEQ ID NO: 193)
Ac-c(Cys-Glu-His-D-2-Nal-Arg-1-Nal-Ala-Cys)-(Pro)₂-Lys-Asp-ß-Ala-Tyr-Gly-Arg-
(Lys)₂-(Arg)₂-Gln-(Arg)₃-NH₂;
(SEQ ID NO: 194)
Ac-c(Cys-Glu-His-D-2-Nal-Arg-Bal-Ala-Cys)-(Pro)₂-Lys-Asp-ß-Ala-Tyr-Gly-Arg-
(Lys)₂₋(Arg)₂-Gln-(Arg)₃-NH₂;
(SEQ ID NO: 195)
Ac-c(Cys-Glu-His-D-2-Nal-Arg-1-Nal-Ala-Cys)-(Pro)₂-Lys-Asp-ß-Ala-(Arg)₂-Lys-
(Arg)₂-Gln-(Arg)₃-NH₂;
(SEQ ID NO: 196)
Ac-c(Cys-Glu-His-D-2-Nal-Arg-1-Nal-Ala-Cys)-(Pro)₂-Lys-Asp-ß-Ala-Arg-Lys-
(Arg)₃-Gln-(Arg)₃-NH₂;
(SEQ ID NO: 197)
Ac-c(Cys-Glu-His-D-2-Nal-Arg-2-Nal-Ala-Cys)-(Pro)₂-Lys-Asp-ß-Ala-(Arg)₂-Lys-
(Arg)₂-Gln-(Arg)₃-NH₂;
(SEQ ID NO: 198)
Ac-c(Cys-Glu-His-D-2-Nal-Arg-2-Nal-Ala-Cys)-(Pro)₂-Lys-Asp-ß-Ala-Tyr-Gly-
(Arg)₂-Lys-(Arg)₂-Gln-(Arg)₃-NH₂;
(SEQ ID NO: 199)
Ac-c(Cys-Glu-His-D-2-Nal-Arg-2-Nal-Ala-Cys)-(Pro)₂-Lys-Asp-ß-Ala-Gly-(Arg)₂-
Lys-(Arg)₂-Gln-(Arg)₃-NH₂;
(SEQ ID NO: 200)
Ac-c(Cys-Glu-His-D-2-Nal-Arg-2-Nal-Ala-Cys)-(Pro)₂-Lys-Asp-ß-Ala-Gly-Arg-
Lys-(Arg)₃-Gln-(Arg)₃-NH₂;
(SEQ ID NO: 201)
Ac-c(Cys-Glu-His-D-2-Nal-Arg-1-Nal-Ala-Cys)-(Pro)₂-Lys-Asp-ß-Ala-Tyr-Gly-
(Arg)₂-Lys-(Arg)₂-Gln-(Arg)₃-NH₂;
(SEQ ID NO: 202)
Ac-c(Cys-Glu-His-D-2-Nal-Arg-1-Nal-Ala-Cys)-(Pro)₂-Lys-Asp-ß-Ala-Tyr-Gly-Arg-
Lys-(Arg)₃-Gln-(Arg)₃-NH₂;
(SEQ ID NO: 203)
Ac-c(Cys-Glu-His-D-2-Nal-Arg-1-Nal-Ala-Cys)-(Pro)₂-Lys-Asp-ß-Ala-Gly-(Arg)₂-
Lys-(Arg)₂-Gln-(Arg)₃-NH₂;
(SEQ ID NO: 204)
Ac-c(Cys-Glu-His-D-2-Nal-Arg-1-Nal-Ala-Cys)-(Pro)₂-Lys-Asp-ß-Ala-Gly-Arg-
Lys-(Arg)₃-Gln-(Arg)₃-NH₂;
(SEQ ID NO: 205)
Ac-c(Cys-Glu-His-D-2-Nal-Arg-2-Nal-Ala-Cys)-(Pro)₂-Lys-Asp-ß-Ala-(Arg)₂-Lys-
(Arg)₂-Gln-(Arg)₃-NH₂;
(SEQ ID NO: 206)
Ac-c(Cys-Glu-His-D-2-Nal-Arg-2-Nal-Ala-Cys)-(Pro)₂-Lys-Asp-ß-Ala-Arg-Lys-
(Arg)₃-Gln-(Arg)₃-NH₂;
(SEQ ID NO: 207)
Ac-c(Cys-Glu-His-D-2-Nal-Arg-2-Nal-Ala-Cys)-(Pro)₂-Lys-Asp-ß-Ala-Tyr-Gly-Arg-
Lys-(Arg)₃-Gln-(Arg)₃-NH₂;
(SEQ ID NO: 208)
Ac-c(Cys-Glu-His-D-2-Nal-Arg-Bal-Ala-Cys)-(Pro)₂-Lys-Asp-ß-Ala-(Arg)₂-Lys-
(Arg)₂-Gln-(Arg)₃-NH₂;
(SEQ ID NO: 209)
Ac-c(Cys-Glu-His-D-2-Nal-Arg-Bal-Ala-Cys)-(Pro)₂-Lys-Asp-ß-Ala-Arg-Lys-
(Arg)₃-Gln-(Arg)₃-NH₂;
(SEQ ID NO: 210)
Ac-c(Cys-Glu-His-D-2-Nal-Arg-Bal-Ala-Cys)-(Pro)₂-Lys-Asp-ß-Ala-Tyr-Gly-
(Arg)₂-Lys-(Arg)₂-Gln-(Arg)₃-NH₂;
(SEQ ID NO: 211)
Ac-c(Cys-Glu-His-D-2-Nal-Arg-Bal-Ala-Cys)-(Pro)₂-Lys-Asp-ß-Ala-Tyr-Gly-Arg-
Lys-(Arg)₃-Gln-(Arg)₃-NH₂;
(SEQ ID NO: 212)
Ac-c(Cys-Glu-His-D-2-Nal-Arg-Bal-Ala-Cys)-(Pro)₂-Lys-Asp-ß-Ala-Gly-(Arg)₂-
Lys-(Arg)₂-Gln-(Arg)₃-NH₂;
(SEQ ID NO: 213)
Ac-c(Cys-Glu-His-D-2-Nal-Arg-Bal-Ala-Cys)-(Pro)₂-Lys-Asp-ß-Ala-Gly-Arg-Lys-
(Arg)₃-Gln-(Arg)₃-NH₂;
(SEQ ID NO: 214)
Ac-c(Cys-Glu-His-D-2-Nal-Arg-Trp-Ala-Cys)-(Pro)₂-Lys-Asp-ß-Ala-(Arg)₅-Gln-
(Arg)₃-NH₂;
(SEQ ID NO: 215)
Ac-c(Cys-Glu-His-D-2-Nal-Arg-Trp-Ala-Cys)-(Pro)₂-Arg-Asp-ß-Ala-(Arg)₅-Gln-
(Arg)₃-NH₂;
(SEQ ID NO: 216)
Ac-c(Cys-Glu-His-D-2-Nal-Arg-Trp-Ala-Cys)-(Pro)₂-Lys-Asp-ß-Ala-Tyr-Gly-
(Arg)₅-Gln-(Arg)₃-NH₂;
(SEQ ID NO: 217)
Ac-c(Cys-Glu-His-D-2-Nal-Arg-Trp-Ala-Cys)-(Pro)₂-Arg-Asp-ß-Ala-Tyr-Gly-
(Arg)₅-Gln-(Arg)₃-NH₂;
(SEQ ID NO: 218)
Ac-c(Cys-Glu-His-D-2-Nal-Arg-Trp-Ala-Cys)-(Pro)₂-Lys-Asp-ß-Ala-(Arg)₅-Gln-
(Arg)₄-NH₂;
(SEQ ID NO: 219)
Ac-c(Cys-Glu-His-D-2-Nal-Arg-Trp-Ala-Cys)-(Pro)₂-Arg-Asp-ß-Ala-(Arg)₅-Gln-
(Arg)₄-NH₂;
(SEQ ID NO: 220)
Ac-c(Cys-Glu-His-D-2-Nal-Arg-Trp-Ala-Cys)-(Pro)₂-Lys-Asp-ß-Ala-Tyr-Gly-
(Arg)₅-Gln-(Arg)₄-NH₂;
(SEQ ID NO: 221)
Ac-c(Cys-Glu-His-D-2-Nal-Arg-Trp-Ala-Cys)-(Pro)₂-Arg-Asp-ß-Ala-Tyr-Gly-
(Arg)₅-Gln-(Arg)₄-NH₂;
(SEQ ID NO: 222)
Ac-c(Cys-Glu-His-D-2-Nal-Arg-1-Nal-Ala-Cys)-(Pro)₂-Lys-Asp-ß-Ala-(Arg)₅-Gln-
(Arg)₄-NH₂;
(SEQ ID NO: 223)
Ac-c(Cys-Glu-His-D-2-Nal-Arg-1-Nal-Ala-Cys)-(Pro)₂-Arg-Asp-ß-Ala-(Arg)₅-Gln-
(Arg)₄-NH₂;
(SEQ ID NO: 224)
Ac-c(Cys-Glu-His-D-2-Nal-Arg-1-Nal-Ala-Cys)-(Pro)₂-Arg-Asp-ß-Ala-(Arg)₆-Gln-
(Arg)₃-NH₂;
(SEQ ID NO: 225)
Ac-c(Cys-Glu-His-D-2-Nal-Arg-1-Nal-Ala-Cys)-(Pro)₂-Lys-Asp-ß-Ala-Tyr-Gly-
(Arg)₅-Gln-(Arg)₃-NH₂;
(SEQ ID NO: 226)
Ac-c(Cys-Glu-His-D-2-Nal-Arg-1-Nal-Ala-Cys)-(Pro)₂-Arg-Asp-ß-Ala-Tyr-Gly-
(Arg)₅-Gln-(Arg)₃-NH₂;
(SEQ ID NO: 227)
Ac-c(Cys-Glu-His-D-2-Nal-Arg-1-Nal-Ala-Cys)-(Pro)₂-Lys-Asp-ß-Ala-Tyr-Gly-
(Arg)₅-Gln-(Arg)₄-NH₂;
(SEQ ID NO: 228)
Ac-c(Cys-Glu-His-D-2-Nal-Arg-1-Nal-Ala-Cys)-(Pro)₂-Arg-Asp-ß-Ala-Tyr-Gly-
(Arg)₅-Gln-(Arg)₄-NH₂;
(SEQ ID NO: 229)
Ac-c(Cys-Glu-His-D-2-Nal-Arg-1-Nal-Ala-Cys)-(Pro)₂-Lys-Asp-ß-Ala-Tyr-Gly-
(Arg)₆-Gln-(Arg)₃-NH₂;
(SEQ ID NO: 230)
Ac-c(Cys-Glu-His-D-2-Nal-Arg-1-Nal-Ala-Cys)-(Pro)₂-Arg-Asp-ß-Ala-Tyr-Gly-
(Arg)₆-Gln-(Arg)₃-NH₂;
(SEQ ID NO: 231)
Ac-c(Cys-Glu-His-D-2-Nal-Arg-2-Nal-Ala-Cys)-(Pro)₂-Arg-Asp-ß-Ala-(Arg)₆-Gln-
(Arg)₃-NH₂;
(SEQ ID NO: 232)
Ac-c(Cys-Glu-His-D-2-Nal-Arg-2-Nal-Ala-Cys)-(Pro)₂-Lys-Asp-ß-Ala-(Arg)₅-Gln-
(Arg)₄-NH₂;
(SEQ ID NO: 233)
Ac-c(Cys-Glu-His-D-2-Nal-Arg-2-Nal-Ala-Cys)-(Pro)₂-Arg-Asp-ß-Ala-(Arg)₅-Gln-
(Arg)₄-NH₂;
(SEQ ID NO: 234)
Ac-c(Cys-Glu-His-D-2-Nal-Arg-2-Nal-Ala-Cys)-(Pro)₂-Lys-Asp-ß-Ala-Tyr-Gly-
(Arg)₅-Gln-(Arg)₃-NH₂;
(SEQ ID NO: 235)
Ac-c(Cys-Glu-His-D-2-Nal-Arg-2-Nal-Ala-Cys)-(Pro)₂-Arg-Asp-ß-Ala-Tyr-Gly-
(Arg)₅-Gln-(Arg)₃-NH₂;
(SEQ ID NO: 236)
Ac-c(Cys-Glu-His-D-2-Nal-Arg-2-Nal-Ala-Cys)-(Pro)₂-Lys-Asp-ß-Ala-Tyr-Gly-
(Arg)₆-Gln-(Arg)₃-NH₂;
(SEQ ID NO: 237)
Ac-c(Cys-Glu-His-D-2-Nal-Arg-2-Nal-Ala-Cys)-(Pro)₂-Arg-Asp-ß-Ala-Tyr-Gly-
(Arg)₆-Gln-(Arg)₃-NH₂;
(SEQ ID NO: 238)
Ac-c(Cys-Glu-His-D-2-Nal-Arg-2-Nal-Ala-Cys)-(Pro)₂-Lys-Asp-ß-Ala-Tyr-Gly-
(Arg)₅-Gln-(Arg)₄-NH₂;
(SEQ ID NO: 239)
Ac-c(Cys-Glu-His-D-2-Nal-Arg-2-Nal-Ala-Cys)-(Pro)₂-Arg-Asp-ß-Ala-Tyr-Gly-
(Arg)₅-Gln-(Arg)₄-NH₂;
(SEQ ID NO: 240)
Ac-c(Cys-Glu-His-D-2-Nal-Arg-Bal-Ala-Cys)-(Pro)₂-Lys-Asp-ß-Ala-(Arg)₅-Gln-
(Arg)₄-NH₂;
(SEQ ID NO: 241)
Ac-c(Cys-Glu-His-D-2-Nal-Arg-Bal-Ala-Cys)-(Pro)₂-Arg-Asp-ß-Ala-(Arg)₅-Gln-
(Arg)₄-NH₂;
(SEQ ID NO: 242)
Ac-c(Cys-Glu-His-D-2-Nal-Arg-Bal-Ala-Cys)-(Pro)₂-Arg-Asp-ß-Ala-Tyr-Gly-
(Arg)₅-Gln-(Arg)₃-NH₂;
(SEQ ID NO: 243)
Ac-c(Cys-Glu-His-D-2-Nal-Arg-Bal-Ala-Cys)-(Pro)₂-Lys-Asp-ß-Ala-Tyr-Gly-
(Arg)₅-Gln-(Arg)₄-NH₂;
(SEQ ID NO: 244)
Ac-c(Cys-Glu-His-D-2-Nal-Arg-Bal-Ala-Cys)-(Pro)₂-Arg-Asp-ß-Ala-Tyr-Gly-
(Arg)₅-Gln-(Arg)₄-NH₂;
(SEQ ID NO: 245)
Ac-c(Cys-Glu-His-D-2-Nal-Arg-Bal-Ala-Cys)-(Pro)₂-Lys-Asp-ß-Ala-Tyr-Gly-
(Arg)₆-Gln-(Arg)₃-NH₂;
(SEQ ID NO: 246)
Ac-c(Cys-Glu-His-D-2-Nal-Arg-Bal-Ala-Cys)-(Pro)₂-Arg-Asp-ß-Ala-Tyr-Gly-
(Arg)₆-Gln-(Arg)₃-NH₂;
(SEQ ID NO: 247)
Ac-Nle-c(Cys-D-Ala-His-D-Phe-Arg-Trp-Cys)-(Doc)₂-Tyr-Gly-Arg-(Lys)₂-(Arg)₂-
Gln-(Arg)₃-NH₂;
(SEQ ID NO: 248)
Ac-Nle-c(Cys-D-Ala-His-D-Phe-Arg-Trp-Cys)-ß-Ala-Tyr-Gly-Arg-(Lys)₂-Arg-Gln-
(Arg)₄-NH₂;
(SEQ ID NO: 249)
Ac-Nle-c(Cys-D-Ala-His-D-Phe-Arg-Trp-Cys)-Doc-Tyr-Gly-Arg-(Lys)₂-(Arg)₂-
Gln-(Arg)₃-NH₂;
(SEQ ID NO: 250)
Ac-Nle-c(Cys-D-Ala-His-D-Phe-Arg-Trp-Cys)-ß-Ala-(Arg)₅-Gln-(Arg)₃-NH₂;
(SEQ ID NO: 251)
Ac-Nle-c(Cys-D-Ala-His-D-Phe-Arg-Trp-Cys)-ß-Ala-Gly-(Arg)₅-Gln-(Arg)₃-NH₂;
(SEQ ID NO: 252)
Ac-Nle-c(Cys-D-Ala-His-D-Phe-Arg-Trp-Cys)-ß-Ala-Tyr-Gly-(Arg)₅-Gln-(Arg)₃-
NH₂;
(SEQ ID NO: 253)
Ac-Nle-c(Cys-D-Ala-His-D-Phe-Arg-Trp-Cys)-ß-Ala-Gly-(Arg)₅-Gln-(Arg)₄-NH₂;
(SEQ ID NO: 254)
Ac-Nle-c(Cys-D-Ala-His-D-Phe-Arg-Trp-Cys)-ß-Ala-Tyr-Gly-(Arg)₂-Lys-(Arg)₂-
Gln-(Arg)₃-NH₂;
(SEQ ID NO: 255)
Ac-Nle-c(Cys-D-Ala-His-D-Phe-Arg-Trp-Cys)-ß-Ala-Tyr-Gly-Arg-Lys-(Arg)₃-Gln-
(Arg)₃-NH₂;
(SEQ ID NO: 256)
Ac-Nle-c(Cys-D-Ala-His-D-Phe-Arg-Trp-Cys)-ß-Ala-Gly-(Arg)₂-Lys-(Arg)₂-Gln-
(Arg)₃-NH₂;
(SEQ ID NO: 257)
Ac-Nle-c(Cys-D-Ala-His-D-Phe-Arg-Trp-Cys)-ß-Ala-Gly-Arg-Lys-(Arg)₃-Gln-
(Arg)₃-NH₂;
(SEQ ID NO: 258)
Ac-Nle-c(Cys-D-Ala-His-D-Phe-Arg-Trp-Cys)-ß-Ala-(Arg)₂-Lys-(Arg)₂-Gln-(Arg)₃-
NH₂;
(SEQ ID NO: 259)
Ac-Nle-c(Cys-D-Ala-His-D-Phe-Arg-Trp-Cys)-ß-Ala-Arg-Lys-(Arg)₃-Gln-(Arg)₃-
NH₂;
(SEQ ID NO: 260)
Ac-Nle-c(Cys-D-Ala-His-D-Phe-Arg-Trp-Cys)-(ß-Ala)₂-(Arg)₅-Gln-(Arg)₃-NH₂;
(SEQ ID NO: 261)
Ac-Nle-c(Cys-D-Ala-His-D-Phe-Arg-Trp-Cys)-(ß-Ala)₂-Gly-(Arg)₅-Gln-(Arg)₃-
NH₂;
(SEQ ID NO: 262)
Ac-Nle-c(Cys-D-Ala-His-D-Phe-Arg-Trp-Cys)-(ß-Ala)₂-Tyr-Gly-(Arg)₅-Gln-(Arg)₃-
NH₂;
(SEQ ID NO: 263)
Ac-Nle-c(Cys-D-Ala-His-D-Phe-Arg-Trp-Cys)-Doc-(Arg)₅-Gln-(Arg)₃-NH₂;
(SEQ ID NO: 264)
Ac-Nle-c(Cys-D-Ala-His-D-Phe-Arg-Trp-Cys)-Doc-Gly-(Arg)₅-Gln-(Arg)₃-NH₂;
(SEQ ID NO: 265)
Ac-Nle-c(Cys-D-Ala-His-D-Phe-Arg-Trp-Cys)-Doc-Tyr-Gly-(Arg)₅-Gln-(Arg)₃-
NH₂;
(SEQ ID NO: 266)
Ac-Nle-c(Cys-D-Ala-His-D-Phe-Arg-Trp-Cys)-(Doc)2-(Arg)₅-Gln-(Arg)₃-NH₂;
(SEQ ID NO: 267)
Ac-Nle-c(Cys-D-Ala-His-D-Phe-Arg-Trp-Cys)-(Doc)₂-Gly-(Arg)₅-Gln-(Arg)₃-NH₂;
(SEQ ID NO: 268)
Ac-Nle-c(Cys-D-Ala-His-D-Phe-Arg-Trp-Cys)-(Doc)₂-Tyr-Gly-(Arg)₅-Gln-(Arg)₃-
NH₂;
(SEQ ID NO: 269)
Ac-Nle-c(Cys-D-Ala-His-D-Phe-Arg-Trp-Cys)-ß-Ala-(Arg)₅-Gln-(Arg)₄-NH₂;
(SEQ ID NO: 270)
Ac-Nle-c(Cys-D-Ala-His-D-Phe-Arg-Trp-Cys)-ß-Ala-Tyr-Gly-(Arg)₅-Gln-(Arg)₄-
NH₂;
(SEQ ID NO: 271)
Ac-Nle-c(Cys-D-Ala-His-D-Phe-Arg-Trp-Cys)-(ß-Ala)₂-(Arg)₅-Gln-(Arg)₄-NH₂;
(SEQ ID NO: 272)
Ac-Nle-c(Cys-D-Ala-His-D-Phe-Arg-Trp-Cys)-(ß-Ala)₂-Gly-(Arg)₅-Gln-(Arg)₄-NH₂;
(SEQ ID NO: 273)
Ac-Nle-c(Cys-D-Ala-His-D-Phe-Arg-Trp-Cys)-(ß-Ala)₂-Tyr-Gly-(Arg)₅-Gln-(Arg)₄-
NH₂;
(SEQ ID NO: 274)
Ac-Nle-c(Cys-D-Ala-His-D-Phe-Arg-Trp-Cys)-Doc-(Arg)₅-Gln-(Arg)₄-NH₂;
(SEQ ID NO: 275)
Ac-Nle-c(Cys-D-Ala-His-D-Phe-Arg-Trp-Cys)-Doc-Gly-(Arg)₅-Gln-(Arg)₄-NH₂;
(SEQ ID NO: 276)
Ac-Nle-c(Cys-D-Ala-His-D-Phe-Arg-Trp-Cys)-Doc-Tyr-Gly-(Arg)₅-Gln-(Arg)₄-
NH₂;
(SEQ ID NO: 277)
Ac-Nle-c(Cys-D-Ala-His-D-Phe-Arg-Trp-Cys)-(Doc)₂-(Arg)₅-Gln-(Arg)₄-NH₂;
(SEQ ID NO: 278)
Ac-Nle-c(Cys-D-Ala-His-D-Phe-Arg-Trp-Cys)-(Doc)₂-Gly-(Arg)₅-Gln-(Arg)₄-NH₂;
(SEQ ID NO: 279)
Ac-Nle-c(Cys-D-Ala-His-D-Phe-Arg-Trp-Cys)-(Doc)₂-Tyr-Gly-(Arg)₅-Gln-(Arg)₄-
NH₂;
(SEQ ID NO: 280)
Ac-Nle-c(Cys-His-D-Phe-Arg-Trp-Gaba-Cys)-ß-Ala-Tyr-Gly-(Arg)₅-Gln-(Arg)₃-
NH₂;
(SEQ ID NO: 281)
Ac-Nle-c(Cys-His-D-Phe-Arg-Trp-Gaba-Cys)-ß-Ala-(Arg)₅-Gln-(Arg)₃-NH₂;
(SEQ ID NO: 282)
Ac-Nle-c(Asp-His-D-Phe-Arg-Trp-Ala-Lys)-ß-Ala-Tyr-Gly-(Arg)₅-Gln-(Arg)₃-
NH₂;
(SEQ ID NO: 283)
Ac-Nle-c(Asp-His-D-Phe-Arg-Trp-Ala-Lys)-ß-Ala-(Arg)₅-Gln-(Arg)₃-NH₂;
(SEQ ID NO: 284)
Ac-Nle-c(Asp-His-D-Phe-Arg-Trp-Lys)-ß-Ala-Tyr-Gly-(Arg)₅-Gln-(Arg)₃-NH₂;
(SEQ ID NO: 285)
Ac-Nle-c(Asp-His-D-Phe-Arg-Trp-Lys)-ß-Ala-Gly-(Arg)₅-Gln-(Arg)₃-NH₂;
(SEQ ID NO: 286)
Ac-Nle-c(Asp-His-D-Phe-Arg-Trp-Lys)-ß-Ala-(Arg)₅-Gln-(Arg)₃-NH₂;
(SEQ ID NO: 287)
Ac-Nle-c(Asp-His-D-Phe-Arg-Trp-Lys)-(ß-Ala)₂-Tyr-Gly-(Arg)₅-Gln-(Arg)₃-NH₂;
(SEQ ID NO: 288)
Ac-Nle-c(Asp-His-D-Phe-Arg-Trp-Lys)-(ß-Ala)₂-Gly-(Arg)₅-Gln-(Arg)₃-NH₂;
(SEQ ID NO: 289)
Ac-Nle-c(Asp-His-D-Phe-Arg-Trp-Lys)-(ß-Ala)₂-(Arg)₅-Gln-(Arg)₃-NH₂;
(SEQ ID NO: 290)
Ac-Nle-c(Asp-His-D-Phe-Arg-Trp-Lys)-Doc-Tyr-Gly-(Arg)₅-Gln-(Arg)₃-NH₂;
(SEQ ID NO: 291)
Ac-Nle-c(Asp-His-D-Phe-Arg-Trp-Lys)-Doc-Gly-(Arg)₅-Gln-(Arg)₃-NH₂;
(SEQ ID NO: 292)
Ac-Nle-c(Asp-His-D-Phe-Arg-Trp-Lys)-Doc-(Arg)₅-Gln-(Arg)₃-NH₂;
(SEQ ID NO: 293)
Ac-Nle-c(Asp-His-D-Phe-Arg-Trp-Lys)-(Doc)₂-Tyr-Gly-(Arg)₅-Gln-(Arg)₃-NH₂;
(SEQ ID NO: 294)
Ac-Nle-c(Asp-His-D-Phe-Arg-Trp-Lys)-(Doc)₂-Gly-(Arg)₅-Gln-(Arg)₃-NH₂;
(SEQ ID NO: 295)
Ac-Nle-c(Asp-His-D-Phe-Arg-Trp-Lys)-(Doc)₂-(Arg)₅-Gln-(Arg)₃-NH₂;
(SEQ ID NO: 296)
Ac-Nle-c(Asp-His-D-Phe-Arg-Trp-Lys)-ß-Ala-Tyr-Gly-(Arg)₅-Gln-(Arg)₄-NH₂;
(SEQ ID NO: 297)
Ac-Nle-c(Asp-His-D-Phe-Arg-Trp-Lys)-ß-Ala-Gly-(Arg)₅-Gln-(Arg)₄-NH₂;
(SEQ ID NO: 298)
Ac-Nle-c(Asp-His-D-Phe-Arg-Trp-Lys)-ß-Ala-(Arg)₅-Gln-(Arg)₄-NH₂;
(SEQ ID NO: 299)
Ac-Nle-c(Asp-His-D-Phe-Arg-Trp-Lys)-(ß-Ala)₂-Tyr-Gly-(Arg)₅-Gln-(Arg)₄-NH₂;
(SEQ ID NO: 300)
Ac-Nle-c(Asp-His-D-Phe-Arg-Trp-Lys)-(ß-Ala)₂-Gly-(Arg)₅-Gln-(Arg)₄-NH₂;
(SEQ ID NO: 301)
Ac-Nle-c(Asp-His-D-Phe-Arg-Trp-Lys)-(ß-Ala)₂-(Arg)₅-Gln-(Arg)₄-NH₂;
(SEQ ID NO: 302)
Ac-Nle-c(Asp-His-D-Phe-Arg-Trp-Lys)-Doc-Tyr-Gly-(Arg)₅-Gln-(Arg)₄-NH₂;
(SEQ ID NO: 303)
Ac-Nle-c(Asp-His-D-Phe-Arg-Trp-Lys)-Doc-Gly-(Arg)₅-Gln-(Arg)₄-NH₂;
(SEQ ID NO: 304)
Ac-Nle-c(Asp-His-D-Phe-Arg-Trp-Lys)-Doc-(Arg)₅-Gln-(Arg)₄-NH₂;
(SEQ ID NO: 305)
Ac-Nle-c(Asp-His-D-Phe-Arg-Trp-Lys)-(Doc)₂-Tyr-Gly-(Arg)₅-Gln-(Arg)₄-NH₂;
(SEQ ID NO: 306)
Ac-Nle-c(Asp-His-D-Phe-Arg-Trp-Lys)-(Doc)₂-Gly-(Arg)₅-Gln-(Arg)₄-NH₂;
(SEQ ID NO: 307)
Ac-Nle-c(Asp-His-D-Phe-Arg-Trp-Lys)-(Doc)₂-(Arg)₅-Gln-(Arg)₄-NH₂;
(SEQ ID NO: 308)
Ac-Nle-c(Asp-His-D-Phe-Arg-Trp-ß-Ala-Lys)-ß-Ala-Tyr-Gly-(Arg)₅-Gln-(Arg)₃-
NH₂;
(SEQ ID NO: 309)
Ac-Nle-c(Asp-His-D-Phe-Arg-Trp-ß-Ala-Lys)-ß-Ala-(Arg)₅-Gln-(Arg)₃-NH₂;
(SEQ ID NO: 310)
Ac-Nle-c(Cys-His-D-Phe-Arg-Trp-Ahx-Cys)-ß-Ala-Tyr-Gly-(Arg)5-Gln-(Arg)₃-
NH₂;
(SEQ ID NO: 311)
Ac-Nle-c(Cys-His-D-Phe-Arg-Trp-Ahx-Cys)-ß-Ala-(Arg)₅-Gln-(Arg)₃-NH₂;
(SEQ ID NO: 312)
D-Phe-c(Cys-His-D-Phe-Arg-Trp-ß-Ala-D-Cys)-Thr-ß-Ala-Tyr-Gly-(Arg)₅-Gln-
(Arg)₃-NH₂;
(SEQ ID NO: 313)
D-Phe-c(Cys-His-D-Phe-Arg-Trp-ß-Ala-D-Cys)-Thr-ß-Ala-(Arg)₅-Gln-(Arg)₃-NH₂;
(SEQ ID NO: 314)
Ac-Nle-c(Cys-His-D-Phe-Arg-Trp-Apn-Cys)-ß-Ala-Tyr-Gly-(Arg)₅-Gln-(Arg)₃-
NH₂;
(SEQ ID NO: 315)
Ac-Nle-c(Cys-His-D-Phe-Arg-Trp-Apn-Cys)-ß-Ala-(Arg)₅-Gln-(Arg)₃-NH₂;
(SEQ ID NO: 316)
Ac-Cha-c(Asp-His-D-Phe-Arg-Trp-Gaba-Lys)-ß-Ala-Tyr-Gly-(Arg)₅-Gln-(Arg)₃-
NH₂;
(SEQ ID NO: 317)
Ac-Cha-c(Asp-His-D-Phe-Arg-Trp-Gaba-Lys)-ß-Ala-(Arg)₅-Gln-(Arg)₃-NH₂;
(SEQ ID NO: 318)
Ac-Nle-c(Asp-His-D-Phe-Arg-Trp-Gaba-Lys)-ß-Ala-Tyr-Gly-(Arg)₅-Gln-(Arg)₃-
NH₂;
(SEQ ID NO: 319)
Ac-Nle-c(Asp-His-D-Phe-Arg-Trp-Gaba-Lys)-ß-Ala-(Arg)₅-Gln-(Arg)₃-NH₂;
(SEQ ID NO: 320)
Ac-Chg-c(Asp-His-D-Phe-Arg-Trp-Gaba-Lys)-ß-Ala-Tyr-Gly-(Arg)₅-Gln-(Arg)₃-
NH₂;
(SEQ ID NO: 321)
Ac-Chg-c(Asp-His-D-Phe-Arg-Trp-Gaba-Lys)-ß-Ala-(Arg)₅-Gln-(Arg)₃-NH₂;
(SEQ ID NO: 322)
Ac-hCha-c(Asp-His-D-Phe-Arg-Trp-Gaba-Lys)-ß-Ala-Tyr-Gly-(Arg)₅-Gln-(Arg)₃-
NH₂;
(SEQ ID NO: 323)
Ac-hCha-c(Asp-His-D-Phe-Arg-Trp-Gaba-Lys)-ß-Ala-(Arg)₅-Gln-(Arg)₃-NH₂;
(SEQ ID NO: 324)
Ac-hCha-c(Asp-His-D-Phe-Arg-Trp-Gaba-Lys)-(ß-Ala)₂-Tyr-Gly-(Arg)₅-Gln-
(Arg)₃-NH₂;
(SEQ ID NO: 325)
Ac-hCha-c(Asp-His-D-Phe-Arg-Trp-Gaba-Lys)-(ß-Ala)₂-(Arg)₅-Gln-(Arg)₃-NH₂;
(SEQ ID NO: 326)
Ac-hCha-c(Asp-His-D-Phe-Arg-Trp-Gaba-Lys)-Doc-Tyr-Gly-(Arg)₅-Gln-(Arg)₃-
NH₂;
(SEQ ID NO: 327)
Ac-hCha-c(Asp-His-D-Phe-Arg-Trp-Gaba-Lys)-Doc-(Arg)₅-Gln-(Arg)₃-NH₂;
(SEQ ID NO: 328)
Ac-hCha-c(Asp-His-D-Phe-Arg-Trp-Gaba-Lys)-(Doc)₂-Tyr-Gly-(Arg)₅-Gln-(Arg)₃-
NH₂;
(SEQ ID NO: 329)
Ac-hCha-c(Asp-His-D-Phe-Arg-Trp-Gaba-Lys)-(Doc)₂-(Arg)₅-Gln-(Arg)₃-NH₂;
(SEQ ID NO: 330)
Ac-hCha-c(Asp-His-D-Phe-Arg-Trp-Gaba-Lys)-ß-Ala-Tyr-Gly-(Arg)₅-Gln-(Arg)₄-
NH₂;
(SEQ ID NO: 331)
Ac-hCha-c(Asp-His-D-Phe-Arg-Trp-Gaba-Lys)-ß-Ala-(Arg)₅-Gln-(Arg)₄-NH₂;
(SEQ ID NO: 332)
Ac-hCha-c(Asp-His-D-Phe-Arg-Trp-Gaba-Lys)-(ß-Ala)₂-Tyr-Gly-(Arg)₅-Gln-
(Arg)₄-NH₂;
(SEQ ID NO: 333)
Ac-hCha-c(Asp-His-D-Phe-Arg-Trp-Gaba-Lys)-(ß-Ala)₂-(Arg)₅-Gln-(Arg)₄-NH₂;
(SEQ ID NO: 334)
Ac-hCha-c(Asp-His-D-Phe-Arg-Trp-Gaba-Lys)-Doc-Tyr-Gly-(Arg)₅-Gln-(Arg)₄-
NH₂;
(SEQ ID NO: 335)
Ac-hCha-c(Asp-His-D-Phe-Arg-Trp-Gaba-Lys)-Doc-(Arg)₅-Gln-(Arg)₄-NH₂;
(SEQ ID NO: 336)
Ac-hCha-c(Asp-His-D-Phe-Arg-Trp-Gaba-Lys)-(Doc)₂-Tyr-Gly-(Arg)₅-Gln-(Arg)₄-
NH₂;
(SEQ ID NO: 337)
Ac-hCha-c(Asp-His-D-Phe-Arg-Trp-Gaba-Lys)-(Doc)₂-(Arg)₅-Gln-(Arg)₄-NH₂;
(SEQ ID NO: 338)
Ac-D-Chg-c(Asp-His-D-Phe-Arg-Trp-Gaba-Lys)-ß-Ala-Tyr-Gly-(Arg)₅-Gln-(Arg)₃-
NH₂;
(SEQ ID NO: 339)
Ac-D-Chg-c(Asp-His-D-Phe-Arg-Trp-Gaba-Lys)-ß-Ala-(Arg)₅-Gln-(Arg)₃-NH₂;
(SEQ ID NO: 340)
Ac-hPhe-c(Asp-His-D-Phe-Arg-Trp-Gaba-Lys)-ß-Ala-Tyr-Gly-(Arg)₅-Gln-(Arg)₃-
NH₂;
(SEQ ID NO: 341)
Ac-hPhe-c(Asp-His-D-Phe-Arg-Trp-Gaba-Lys)-ß-Ala-(Arg)₅-Gln-(Arg)₃-NH₂;
(SEQ ID NO: 342)
Ac-Nle-c(Cys-His-D-Phe-Arg-D-Trp-Apn-Cys)-ß-Ala-Tyr-Gly-(Arg)₅-Gln-(Arg)₃-
NH₂;
(SEQ ID NO: 343)
Ac-Nle-c(Cys-His-D-Phe-Arg-D-Trp-Apn-Cys)-ß-Ala-(Arg)₅-Gln-(Arg)₃-NH₂;
(SEQ ID NO: 344)
Ac-Nle-c(Cys-His-D-Phe-Arg-D-Trp-Ahx-Cys)-ß-Ala-Tyr-Gly-(Arg)₅-Gln-(Arg)₃-
NH₂;
(SEQ ID NO: 345)
Ac-Nle-c(Cys-His-D-Phe-Arg-D-Trp-Ahx-Cys)-ß-Ala-(Arg)₅-Gln-(Arg)₃-NH₂;
(SEQ ID NO: 346)
Ac-Nle-c(Cys-His-D-Phe-Arg-D-Trp-ß-Ala-Cys)-ß-Ala-Tyr-Gly-(Arg)₅-Gln-(Arg)₃-
NH₂;
(SEQ ID NO: 347)
Ac-Nle-c(Cys-His-D-Phe-Arg-D-Trp-ß-Ala-Cys)-ß-Ala-(Arg)₅-Gln-(Arg)₃-NH₂;
(SEQ ID NO: 348)
Ac-Nle-c(Cys-D-Ala-His-D-Phe-Arg-Trp-Pen)-ß-Ala-Tyr-Gly-(Arg)₅-Gln-(Arg)₃-
NH₂;
(SEQ ID NO: 349)
Ac-Nle-c(Cys-D-Ala-His-D-Phe-Arg-Trp-Pen)-ß-Ala-Gly-(Arg)₅-Gln-(Arg)₃-NH₂;
(SEQ ID NO: 350)
Ac-Nle-c(Cys-D-Ala-His-D-Phe-Arg-Trp-Pen)-ß-Ala-(Arg)₅-Gln-(Arg)₃-NH₂;
(SEQ ID NO: 351)
Ac-Nle-c(Cys-D-Ala-His-D-Phe-Arg-Trp-Pen)-(ß-Ala)₂-Tyr-Gly-(Arg)₅-Gln-(Arg)₃-
NH₂;
(SEQ ID NO: 352)
Ac-Nle-c(Cys-D-Ala-His-D-Phe-Arg-Trp-Pen)-(ß-Ala)₂-Gly-(Arg)₅-Gln-(Arg)₃-
NH₂;
(SEQ ID NO: 353)
Ac-Nle-c(Cys-D-Ala-His-D-Phe-Arg-Trp-Pen)-(ß-Ala)₂-(Arg)₅-Gln-(Arg)₃-NH₂;
(SEQ ID NO: 354)
Ac-Nle-c(Cys-D-Ala-His-D-Phe-Arg-Trp-Pen)-Doc-Tyr-Gly-(Arg)₅-Gln-(Arg)₃-
NH₂;
(SEQ ID NO: 355)
Ac-Nle-c(Cys-D-Ala-His-D-Phe-Arg-Trp-Pen)-Doc-Gly-(Arg)₅-Gln-(Arg)₃-NH₂;
(SEQ ID NO: 356)
Ac-Nle-c(Cys-D-Ala-His-D-Phe-Arg-Trp-Pen)-Doc-(Arg)₅-Gln-(Arg)₃-NH₂;
(SEQ ID NO: 357)
Ac-Nle-c(Cys-D-Ala-His-D-Phe-Arg-Trp-Pen)-(Doc)₂-Tyr-Gly-(Arg)₅-Gln-(Arg)₃-
NH₂;
(SEQ ID NO: 358)
Ac-Nle-c(Cys-D-Ala-His-D-Phe-Arg-Trp-Pen)-(Doc)₂-Gly-(Arg)₅-Gln-(Arg)₃-NH₂;
(SEQ ID NO: 359)
Ac-Nle-c(Cys-D-Ala-His-D-Phe-Arg-Trp-Pen)-(Doc)₂-(Arg)₅-Gln-(Arg)₃-NH₂;
(SEQ ID NO: 360)
D-Phe-c(Cys-His-D-(Et)Tyr-Arg-Trp-ß-Ala-D-Cys)-ß-Ala-Tyr-Gly-(Arg)₅-Gln-
(Arg)₃-NH₂;
(SEQ ID NO: 361)
D-Phe-c(Cys-His-D-(Et)Tyr-Arg-Trp-ß-Ala-D-Cys)-ß-Ala-(Arg)₅-Gln-(Arg)₃-NH₂;
(SEQ ID NO: 362)
D-Phe-c(Cys-His-D-(Et)Tyr-Arg-Trp-ß-Ala-D-Cys)-ß-Ala-Gly-(Arg)₅-Gln-(Arg)₃-
NH₂;
(SEQ ID NO: 363)
D-Phe-c(Cys-His-D-(Et)Tyr-Arg-Trp-ß-Ala-D-Cys)-ß-Ala-(Arg)₅-Gln-(Arg)₄-NH₂;
(SEQ ID NO: 364)
D-Phe-c(Cys-His-D-(Et)Tyr-Arg-Trp-ß-Ala-D-Cys)-(ß-Ala)₂-Tyr-Gly-(Arg)₅-Gln-
(Arg)₃-NH₂;
(SEQ ID NO: 365)
D-Phe-c(Cys-His-D-(Et)Tyr-Arg-Trp-ß-Ala-D-Cys)-(ß-Ala)₂-(Arg)₅-Gln-(Arg)₃-
NH₂;
(SEQ ID NO: 366)
D-Phe-c(Cys-His-D-(Et)Tyr-Arg-Trp-ß-Ala-D-Cys)-(ß-Ala)₂-Gly-(Arg)₅-Gln-(Arg)₃-
NH₂;
(SEQ ID NO: 367)
D-Phe-c(Cys-His-D-(Et)Tyr-Arg-Trp-ß-Ala-D-Cys)-(ß-Ala)₂-(Arg)₅-Gln-(Arg)₄-
NH₂;
(SEQ ID NO: 368)
D-Phe-c(Cys-His-D-(Et)Tyr-Arg-Trp-ß-Ala-D-Cys)-Doc-Tyr-Gly-(Arg)₅-Gln-
(Arg)₃-NH₂;
(SEQ ID NO: 369)
D-Phe-c(Cys-His-D-(Et)Tyr-Arg-Trp-ß-Ala-D-Cys)-Doc-(Arg)₅-Gln-(Arg)₃-NH₂;
(SEQ ID NO: 370)
D-Phe-c(Cys-His-D-(Et)Tyr-Arg-Trp-ß-Ala-D-Cys)-Doc-Gly-(Arg)₅-Gln-(Arg)₃-
NH₂;
(SEQ ID NO: 371)
D-Phe-c(Cys-His-D-(Et)Tyr-Arg-Trp-ß-Ala-D-Cys)-Doc-(Arg)₅-Gln-(Arg)₄-NH₂;
(SEQ ID NO: 372)
D-Phe-c(Cys-His-D-(Et)Tyr-Arg-Trp-ß-Ala-D-Cys)-(Doc)₂-Tyr-Gly-(Arg)₅-Gln-
(Arg)₃-NH₂;
(SEQ ID NO: 373)
D-Phe-c(Cys-His-D-(Et)Tyr-Arg-Trp-ß-Ala-D-Cys)-(Doc)₂-(Arg)₅-Gln-(Arg)₃-
NH₂;
(SEQ ID NO: 374)
D-Phe-c(Cys-His-D-(Et)Tyr-Arg-Trp-ß-Ala-D-Cys)-(Doc)₂-Gly-(Arg)₅-Gln-(Arg)₃-
NH₂;
(SEQ ID NO: 375)
D-Phe-c(Cys-His-D-(Et)Tyr-Arg-Trp-ß-Ala-D-Cys)-(Doc)₂-(Arg)₅-Gln-(Arg)₄-NH₂;
(SEQ ID NO: 376)
D-Phe-c(Cys-His-D-(Et)Tyr-hArg-Trp-ß-Ala-D-Cys)-Thr-ß-Ala-Tyr-Gly-(Arg)₅-
Gln-(Arg)₃-NH₂;
(SEQ ID NO: 377)
D-Phe-c(Cys-His-D-(Et)Tyr-hArg-Trp-ß-Ala-D-Cys)-Thr-ß-Ala-(Arg)₅-Gln-(Arg)₃-
NH₂;
(SEQ ID NO: 378)
D-Phe-c(Cys-His-D-(Et)Tyr-hArg-Trp-ß-Ala-D-Cys)-Thr-(ß-Ala)₂-Tyr-Gly-(Arg)₅-
Gln-(Arg)₃-NH₂;
(SEQ ID NO: 379)
D-Phe-c(Cys-His-D-(Et)Tyr-hArg-Trp-ß-Ala-D-Cys)-Thr-(ß-Ala)₂-(Arg)₅-Gln-
(Arg)₃-NH₂;
(SEQ ID NO: 380)
D-Phe-c(Cys-His-D-(Et)Tyr-hArg-Trp-ß-Ala-D-Cys)-Thr-Doc-Tyr-Gly-(Arg)₅-Gln-
(Arg)₃-NH₂;
(SEQ ID NO: 381)
D-Phe-c(Cys-His-D-(Et)Tyr-hArg-Trp-ß-Ala-D-Cys)-Thr-Doc-(Arg)₅-Gln-(Arg)₃-
NH₂;
(SEQ ID NO: 382)
D-Phe-c(Cys-His-D-(Et)Tyr-hArg-Trp-ß-Ala-D-Cys)-Thr-(Doc)₂-Tyr-Gly-(Arg)₅-
Gln-(Arg)₃-NH₂;
(SEQ ID NO: 383)
D-Phe-c(Cys-His-D-(Et)Tyr-hArg-Trp-ß-Ala-D-Cys)-Thr-ß-Ala-Tyr-Gly-(Arg)₅-
Gln-(Arg)₄-NH₂;
(SEQ ID NO: 384)
D-Phe-c(Cys-His-D-(Et)Tyr-hArg-Trp-ß-Ala-D-Cys)-Thr-ß-Ala-(Arg)₅-Gln-(Arg)₄-
NH₂;
(SEQ ID NO: 385)
D-Phe-c(Cys-His-D-(Et)Tyr-hArg-Trp-ß-Ala-D-Cys)-Thr-(ß-Ala)₂-Tyr-Gly-(Arg)₅-
Gln-(Arg)₄-NH₂;
(SEQ ID NO: 386)
D-Phe-c(Cys-His-D-(Et)Tyr-hArg-Trp-ß-Ala-D-Cys)-Thr-(ß-Ala)₂-(Arg)₅-Gln-
(Arg)₄-NH₂;
(SEQ ID NO: 387)
D-Phe-c(Cys-His-D-(Et)Tyr-hArg-Trp-ß-Ala-D-Cys)-Thr-Doc-Tyr-Gly-(Arg)₅-Gln-
(Arg)₄-NH₂;
(SEQ ID NO: 388)
D-Phe-c(Cys-His-D-(Et)Tyr-hArg-Trp-ß-Ala-D-Cys)-Thr-Doc-(Arg)₅-Gln-(Arg)₄-
NH₂;
(SEQ ID NO: 389)
D-Phe-c(Cys-His-D-(Et)Tyr-hArg-Trp-ß-Ala-D-Cys)-Thr-(Doc)₂-Tyr-Gly-(Arg)₅-
Gln-(Arg)₄-NH₂;
(SEQ ID NO: 390)
D-Phe-c(Cys-His-D-(Et)Tyr-hArg-Trp-ß-Ala-D-Cys)-Thr-(Doc)₂-(Arg)₅-Gln-(Arg)₄-
NH₂;
(SEQ ID NO: 391)
D-Phe-c(Cys-His-D-(Et)Tyr-hArg-Bip-ß-Ala-D-Cys)-Thr-ß-Ala-Tyr-Gly-(Arg)₅-
Gln-(Arg)₃-NH₂;
(SEQ ID NO: 392)
D-Phe-c(Cys-His-D-(Et)Tyr-hArg-Bip-ß-Ala-D-Cys)-Thr-ß-Ala-Tyr-Gly-(Arg)₅-Gln-
(Arg)₄-NH₂;
(SEQ ID NO: 393)
D-Phe-c(Cys-His-D-(Et)Tyr-hArg-Bip-ß-Ala-D-Cys)-Thr-ß-Ala-(Arg)₅-Gln-(Arg)₃-
NH₂;
(SEQ ID NO: 394)
D-Phe-c(Cys-His-D-(Et)Tyr-hArg-Bip-ß-Ala-D-Cys)-Thr-(ß-Ala)₂-Tyr-Gly-(Arg)₅-
Gln-(Arg)₃-NH₂;
(SEQ ID NO: 395)
D-Phe-c(Cys-His-D-(Et)Tyr-hArg-Bip-ß-Ala-D-Cys)-Thr-(ß-Ala)₂-(Arg)₅-Gln-
(Arg)₃-NH₂;
(SEQ ID NO: 396)
D-Phe-c(Cys-His-D-(Et)Tyr-hArg-Bip-ß-Ala-D-Cys)-Thr-Doc-Tyr-Gly-(Arg)₅-Gln-
(Arg)₃-NH₂;
(SEQ ID NO: 397)
D-Phe-c(Cys-His-D-(Et)Tyr-hArg-Bip-ß-Ala-D-Cys)-Thr-Doc-Tyr-Gly-(Arg)₅-Gln-
(Arg)₄-NH₂;
(SEQ ID NO: 398)
D-Phe-c(Cys-His-D-(Et)Tyr-hArg-Bip-ß-Ala-D-Cys)-Thr-Doc-(Arg)₅-Gln-(Arg)₃-
NH₂;
(SEQ ID NO: 399)
D-Phe-c(Cys-His-D-(Et)Tyr-hArg-Bip-ß-Ala-D-Cys)-Thr-(Doc)₂-Tyr-Gly-(Arg)₅-
Gln-(Arg)₃-NH₂;
(SEQ ID NO: 400)
D-Phe-c(Cys-His-D-(Et)Tyr-hArg-Bip-ß-Ala-D-Cys)-Thr-(Doc)₂-(Arg)₅-Gln-
(Arg)₃-NH₂;
(SEQ ID NO: 401)
Ac-Nle-c(Cys-D-Ala-His-D-Phe-Arg-Trp-Gly-Cys)-ß-Ala-Tyr-Gly-(Arg)₅-Gln-
(Arg)₃-NH₂;
(SEQ ID NO: 402)
Ac-Nle-c(Cys-D-Ala-His-D-Phe-Arg-Trp-Gly-Cys)-ß-Ala-(Arg)₅-Gln-(Arg)₃-NH₂;
(SEQ ID NO: 403)
Nle-c(Cys-His-D-Phe-Arg-Trp-Apn-Cys)-ß-Ala-Tyr-Gly-(Arg)₅-Gln-(Arg)₃-NH₂;
(SEQ ID NO: 404)
Nle-c(Cys-His-D-Phe-Arg-Trp-Apn-Cys)-ß-Ala-(Arg)₅-Gln-(Arg)₃-NH₂;
(SEQ ID NO: 405)
Nle-c(Cys-His-D-Phe-Arg-Trp-Apn-Cys)-(ß-Ala)₂-Tyr-Gly-(Arg)₅-Gln-(Arg)₃-NH₂;
(SEQ ID NO: 406)
Nle-c(Cys-His-D-Phe-Arg-Trp-Apn-Cys)-(ß-Ala)₂-(Arg)₅-Gln-(Arg)₃-NH₂;
(SEQ ID NO: 407)
Nle-c(Cys-His-D-Phe-Arg-Trp-Apn-Cys)-ß-Ala-Tyr-Gly-(Arg)₅-Gln-(Arg)₄-NH₂;
(SEQ ID NO: 408)
Nle-c(Cys-His-D-Phe-Arg-Trp-Apn-Cys)-ß-Ala-(Arg)₅-Gln-(Arg)₄-NH₂;
(SEQ ID NO: 409)
Nle-c(Cys-His-D-Phe-Arg-Trp-Apn-Cys)-(ß-Ala)₂-Tyr-Gly-(Arg)₅-Gln-(Arg)₄-NH₂;
(SEQ ID NO: 410)
Nle-c(Cys-His-D-Phe-Arg-Trp-Apn-Cys)-(ß-Ala)₂-(Arg)₅-Gln-(Arg)₄-NH₂;
(SEQ ID NO: 411)
Nle-c(Cys-His-D-Phe-Arg-Trp-Apn-Cys)-Doc-Tyr-Gly-(Arg)₅-Gln-(Arg)₃-NH₂;
(SEQ ID NO: 412)
Nle-c(Cys-His-D-Phe-Arg-Trp-Apn-Cys)-Doc-(Arg)₅-Gln-(Arg)₃-NH₂;
(SEQ ID NO: 413)
Nle-c(Cys-His-D-Phe-Arg-Trp-Apn-Cys)-(Doc)₂-Tyr-Gly-(Arg)₅-Gln-(Arg)₃-NH₂;
(SEQ ID NO: 414)
Nle-c(Cys-His-D-Phe-Arg-Trp-Apn-Cys)-(Doc)₂-(Arg)₅-Gln-(Arg)₃-NH₂;
(SEQ ID NO: 415)
Nle-c(Cys-His-D-Phe-Arg-Trp-Apn-Cys)-Doc-Tyr-Gly-(Arg)₅-Gln-(Arg)₄-NH₂;
(SEQ ID NO: 416)
Nle-c(Cys-His-D-Phe-Arg-Trp-Apn-Cys)-Doc-(Arg)₅-Gln-(Arg)₄-NH₂;
(SEQ ID NO: 417)
Nle-c(Cys-His-D-Phe-Arg-Trp-Apn-Cys)-(Doc)₂-Tyr-Gly-(Arg)₅-Gln-(Arg)₄-NH₂;
(SEQ ID NO: 418)
Nle-c(Cys-His-D-Phe-Arg-Trp-Apn-Cys)-(Doc)₂-(Arg)₅-Gln-(Arg)₄-NH₂;
(SEQ ID NO: 419)
Ac-Nle-c(Cys-D-Leu-His-D-Phe-Arg-Trp-Cys)-ß-Ala-Tyr-Gly-(Arg)₅-Gln-(Arg)₃-
NH₂;
(SEQ ID NO: 420)
Ac-Nle-c(Cys-D-Leu-His-D-Phe-Arg-Trp-Cys)-ß-Ala-(Arg)₅-Gln-(Arg)₃-NH₂;
(SEQ ID NO: 421)
Ac-Nle-c(Cys-D-Leu-His-D-Phe-Arg-Trp-Cys)-(ß-Ala)₂-Tyr-Gly-(Arg)₅-Gln-(Arg)₃-
NH₂;
(SEQ ID NO: 422)
Ac-Nle-c(Cys-D-Leu-His-D-Phe-Arg-Trp-Cys)-(ß-Ala)₂-(Arg)₅-Gln-(Arg)₃-NH₂;
(SEQ ID NO: 423)
Ac-Nle-c(Cys-D-Leu-His-D-Phe-Arg-Trp-Cys)-Doc-Tyr-Gly-(Arg)₅-Gln-(Arg)₃-
NH₂;
(SEQ ID NO: 424)
Ac-Nle-c(Cys-D-Leu-His-D-Phe-Arg-Trp-Cys)-Doc-(Arg)₅-Gln-(Arg)₃-NH₂;
(SEQ ID NO: 425)
Ac-Nle-c(Cys-D-Leu-His-D-Phe-Arg-Trp-Cys)-(Doc)₂-Tyr-Gly-(Arg)₅-Gln-(Arg)₃-
NH₂;
(SEQ ID NO: 426)
Ac-Nle-c(Cys-D-Leu-His-D-Phe-Arg-Trp-Cys)-(Doc)₂-(Arg)₅-Gln-(Arg)₃-NH₂;
(SEQ ID NO: 427)
Ac-Nle-c(Cys-D-Leu-His-D-Phe-Arg-Trp-Cys)-ß-Ala-Tyr-Gly-(Arg)₅-Gln-(Arg)₄-
NH₂;
(SEQ ID NO: 428)
Ac-Nle-c(Cys-D-Leu-His-D-Phe-Arg-Trp-Cys)-ß-Ala-(Arg)₅-Gln-(Arg)₄-NH₂;
(SEQ ID NO: 429)
Ac-Nle-c(Cys-D-Leu-His-D-Phe-Arg-Trp-Cys)-(ß-Ala)2-Tyr-Gly-(Arg)₅-Gln-(Arg)₄-
NH₂;
(SEQ ID NO: 430)
Ac-Nle-c(Cys-D-Leu-His-D-Phe-Arg-Trp-Cys)-(ß-Ala)₂-(Arg)₅-Gln-(Arg)₄-NH₂;
(SEQ ID NO: 431)
Ac-Nle-c(Cys-D-Leu-His-D-Phe-Arg-Trp-Cys)-Doc-Tyr-Gly-(Arg)₅-Gln-(Arg)₄-
NH₂;
(SEQ ID NO: 432)
Ac-Nle-c(Cys-D-Leu-His-D-Phe-Arg-Trp-Cys)-Doc-(Arg)₅-Gln-(Arg)₄-NH₂;
(SEQ ID NO: 433)
Ac-Nle-c(Cys-D-Leu-His-D-Phe-Arg-Trp-Cys)-(Doc)₂-Tyr-Gly-(Arg)₅-Gln-(Arg)₄-
NH₂;
(SEQ ID NO: 434)
Ac-Nle-c(Cys-D-Leu-His-D-Phe-Arg-Trp-Cys)-(Doc)₂-(Arg)₅-Gln-(Arg)₄-NH₂;
(SEQ ID NO: 435)
Ac-Nle-c(Cys-D-Cha-His-D-Phe-Arg-Trp-Cys)-ß-Ala-Tyr-Gly-(Arg)₅-Gln-(Arg)₃-
NH₂;
(SEQ ID NO: 436)
Ac-Nle-c(Cys-D-Cha-His-D-Phe-Arg-Trp-Cys)-ß-Ala-(Arg)₅-Gln-(Arg)₃-NH₂;
(SEQ ID NO: 437)
Ac-Nle-c(Cys-D-Cha-His-D-Phe-Arg-Trp-Cys)-(ß-Ala)₂-Tyr-Gly-(Arg)₅-Gln-
(Arg)₃-NH₂;
(SEQ ID NO: 438)
Ac-Nle-c(Cys-D-Cha-His-D-Phe-Arg-Trp-Cys)-(ß-Ala)₂-(Arg)₅-Gln-(Arg)₃-NH₂;
(SEQ ID NO: 439)
Ac-Nle-c(Cys-D-Cha-His-D-Phe-Arg-Trp-Cys)-Doc-Tyr-Gly-(Arg)₅-Gln-(Arg)₃-
NH₂;
(SEQ ID NO: 440)
Ac-Nle-c(Cys-D-Cha-His-D-Phe-Arg-Trp-Cys)-Doc-(Arg)₅-Gln-(Arg)₃-NH₂;
(SEQ ID NO: 441)
Ac-Nle-c(Cys-D-Cha-His-D-Phe-Arg-Trp-Cys)-(Doc)₂-Tyr-Gly-(Arg)₅-Gln-(Arg)₃-
NH₂;
(SEQ ID NO: 442)
Ac-Nle-c(Cys-D-Cha-His-D-Phe-Arg-Trp-Cys)-(Doc)₂-(Arg)₅-Gln-(Arg)₃-NH₂;
(SEQ ID NO: 443)
Ac-Nle-c(Cys-D-Cha-His-D-Phe-Arg-Trp-Cys)-ß-Ala-Tyr-Gly-(Arg)₅-Gln-(Arg)₄-
NH₂;
(SEQ ID NO: 444)
Ac-Nle-c(Cys-D-Cha-His-D-Phe-Arg-Trp-Cys)-ß-Ala-(Arg)₅-Gln-(Arg)₄-NH₂;
(SEQ ID NO: 445)
Ac-Nle-c(Cys-D-Cha-His-D-Phe-Arg-Trp-Cys)-(ß-Ala)₂-Tyr-Gly-(Arg)₅-Gln-
(Arg)₄-NH₂;
(SEQ ID NO: 446)
Ac-Nle-c(Cys-D-Cha-His-D-Phe-Arg-Trp-Cys)-(ß-Ala)₂-(Arg)₅-Gln-(Arg)₄-NH₂;
(SEQ ID NO: 447)
Ac-Nle-c(Cys-D-Cha-His-D-Phe-Arg-Trp-Cys)-Doc-Tyr-Gly-(Arg)₅-Gln-(Arg)₄-
NH₂;
(SEQ ID NO: 448)
Ac-Nle-c(Cys-D-Cha-His-D-Phe-Arg-Trp-Cys)-Doc-(Arg)₅-Gln-(Arg)₄-NH₂;
(SEQ ID NO: 449)
Ac-Nle-c(Cys-D-Cha-His-D-Phe-Arg-Trp-Cys)-(Doc)₂-Tyr-Gly-(Arg)₅-Gln-(Arg)₄-
NH₂;
(SEQ ID NO: 450)
Ac-Nle-c(Cys-D-Cha-His-D-Phe-Arg-Trp-Cys)-(Doc)₂-(Arg)₅-Gln-(Arg)₄-NH₂;
(SEQ ID NO: 451)
Nle-c(Cys-His-D-Phe-Arg-Trp-Gaba-Cys)-ß-Ala-Tyr-Gly-(Arg)₅-Gln-(Arg)₃-NH₂;
(SEQ ID NO: 452)
Nle-c(Cys-His-D-Phe-Arg-Trp-Gaba-Cys)-ß-Ala-(Arg)₅-Gln-(Arg)₃-NH₂;
(SEQ ID NO: 453)
Nle-c(Cys-His-D-Phe-Arg-Trp-Gaba-Cys)-(ß-Ala)₂-Tyr-Gly-(Arg)₅-Gln-(Arg)₃-NH₂;
(SEQ ID NO: 454)
Nle-c(Cys-His-D-Phe-Arg-Trp-Gaba-Cys)-(ß-Ala)₂-(Arg)₅-Gln-(Arg)₃-NH₂;
(SEQ ID NO: 455)
Nle-c(Cys-His-D-Phe-Arg-Trp-Gaba-Cys)-ß-Ala-Tyr-Gly-(Arg)₅-Gln-(Arg)₄-NH₂;
(SEQ ID NO: 456)
Nle-c(Cys-His-D-Phe-Arg-Trp-Gaba-Cys)-ß-Ala-(Arg)₅-Gln-(Arg)₄-NH₂;
(SEQ ID NO: 457)
Nle-c(Cys-His-D-Phe-Arg-Trp-Gaba-Cys)-(ß-Ala)₂-Tyr-Gly-(Arg)₅-Gln-(Arg)₄-NH₂;
(SEQ ID NO: 458)
Nle-c(Cys-His-D-Phe-Arg-Trp-Gaba-Cys)-(ß-Ala)₂-(Arg)₅-Gln-(Arg)₄-NH₂;
(SEQ ID NO: 459)
Nle-c(Cys-His-D-Phe-Arg-Trp-Gaba-Cys)-Doc-Tyr-Gly-(Arg)₅-Gln-(Arg)₃-NH₂;
(SEQ ID NO: 460)
Nle-c(Cys-His-D-Phe-Arg-Trp-Gaba-Cys)-Doc-(Arg)₅-Gln-(Arg)₃-NH₂;
(SEQ ID NO: 461)
Nle-c(Cys-His-D-Phe-Arg-Trp-Gaba-Cys)-(Doc)₂-Tyr-Gly-(Arg)₅-Gln-(Arg)₃-NH₂;
(SEQ ID NO: 462)
Nle-c(Cys-His-D-Phe-Arg-Trp-Gaba-Cys)-(Doc)₂-(Arg)₅-Gln-(Arg)₃-NH₂;
(SEQ ID NO: 463)
Nle-c(Cys-His-D-Phe-Arg-Trp-Gaba-Cys)-Doc-Tyr-Gly-(Arg)₅-Gln-(Arg)₄-NH₂;
(SEQ ID NO: 464)
Nle-c(Cys-His-D-Phe-Arg-Trp-Gaba-Cys)-Doc-(Arg)₅-Gln-(Arg)₄-NH₂;
(SEQ ID NO: 465)
Nle-c(Cys-His-D-Phe-Arg-Trp-Gaba-Cys)-(Doc)₂-Tyr-Gly-(Arg)₅-Gln-(Arg)₄-NH₂;
or
(SEQ ID NO: 466)
Nle-c(Cys-His-D-Phe-Arg-Trp-Gaba-Cys)-(Doc)₂-(Arg)₅-Gln-(Arg)₄-NH₂,

or pharmaceutically acceptable salts thereof.

In some embodiments, a compound of Formula (V) is disclosed in International Application Publication Number WO 2007/008684, which is incorporated herein by reference in its entirety.

In some embodiments, the MC4R agonist is a compound of Formula (VI):

Ac-c(Cys-Glu-His-A¹-Arg-A²-A³-Cys)-(Pro)₂-Lys-Asp-NH₂ (VI)

or pharmaceutically acceptable salts thereof. In Formula (IV):
A¹ is the D-isomer of X-Phe or 2-Nal where X is halogen;
A² is Bal, 1-Nal, 2-Nal, or Trp; and
A³ is Aib, Ala, β-Ala or Gly,

In some embodiments, the compound of Formula (VI) is selected from:

| (SEQ ID NO: 467) | |
|---|---|
| | Ac-c(Cys-Glu-His-D-4-Br-Phe-Arg-Trp-Gly-Cys)- |
| | (Pro)₂-Lys-Asp-NH₂; |
| (SEQ ID NO: 468) | |
| | Ac-c(Cys-Glu-His-D-2-Nal-Arg-Trp-Ala-Cys)- |
| | (Pro)₂-Lys-Asp-NH₂; |
| (SEQ ID NO: 469) | |
| | Ac-c(Cys-Glu-His-D-2-Nal-Arg-2-Nal-Ala-Cys)- |
| | (Pro)₂-Lys-Asp-NH₂; |
| (SEQ ID NO: 470) | |
| | Ac-c(Cys-Glu-His-D-2-Nal-Arg-1-Nal-Ala-Cys)- |
| | (Pro)₂-Lys-Asp-NH₂; |
| (SEQ ID NO: 471) | |
| | Ac-c(Cys-Glu-His-D-2-Nal-Arg-Bal-Ala-Cys)- |
| | (Pro)₂-Lys-Asp-NH₂; |
| (SEQ ID NO: 47₂) | |
| | Ac-c(Cys-Glu-His-D-2-Nal-Arg-2-Nal-B-Ala-Cys)- |
| | (Pro)₂-Lys-Asp-NH₂; |
| | or |
| (SEQ ID NO: 473) | |
| | Ac-c(Cys-Glu-His-D-2-Nal-Arg-2-Nal-Aib-Cys)- |
| | (Pro)₂-Lys-Asp-NH₂; |

or pharmaceutically acceptable salts thereof.

In an example embodiment, the MC4R agonist is a compound of Formula (VII):

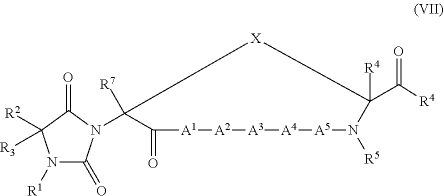

or a pharmaceutically acceptable salt thereof wherein:
X is selected from the group consisting of —CH₂—S—S—CH₂—, —C(CH₃)₂SSCH₂—, —CH₂—S—S—C(CH₃)₂—, —C(CH₃)₂—S—S—C(CH₃)_{z}—, —(CH₂)₂—S—S—CH₂—, —CH₂—S—S—(CH₂)₂, —(CH₂)₂—S—S—(CH₂)₂—, —C(CH₃)₂—S—S—(CH₂)₂—, —(CH₂)₂—S—S—C(CH₃)₂—, —(CH₂)ₜ—C(O)—NR⁸—(CH₂)ᵣ and —(CH₂)ᵣ—NR⁸—C(O)—(CH₂)ₜ—;
each of R¹ and R⁵ is, independently, H, (C₁-C₁₀)alkyl or substituted (C₁-C₁₀)alkyl;
each of R² and R³ is, independently, H, (C₁-C₁₀)alkyl, (C₁₋₁₀)heteroalkyl, aryl(C₁-C₅)alkyl, substituted (C₁-C₁₀)alkyl, substituted (C₁-C₁₀)heteroalkyl or substituted aryl(C₁-C₅)alkyl or R² and R³ may be fused together to form a ring;
R⁴ is OH or NH₂;
each of R⁶ and R⁷ is, independently, H, (C₁-C₁₀)alkyl or substituted (C₁-C₁₀)alkyl;
A¹ is an L- or D-amino acid or deleted;
A² is H is, 2-Pal, 3-Pal, 4-Pal, (X¹, X², X³, X⁴, X⁵)Phe, Taz, 2-Thi or 3-Thi;
A³ is D-Bal, D-1-Nal, D-2-Nal, D-Phe or D-(X¹, X², X³, X⁴, X⁵)Phe;
A⁴ is Arg, hArg, Dab, Dap, Lys or Orn;
A⁵ is Bal, 1-Nal, 2-Nal, (X¹, X², X³, X⁴, X⁵)Phe or Trp;
r is, independently for each occurrence thereof, 1, 2, 3, 4 or 5; and
t is, independently for each occurrence thereof, 1 or 2;
or pharmaceutically acceptable salts thereof.

In an example embodiment of the compounds of Formula (VII),
A¹ is Ala, D-Ala, Asn, Asp, Gln, Glu or Gly.

Example compounds according to Formula (VII) include:

(SEQ ID NO: 539)

c[Hydantoin(C(O)-(Nle-Cys))-D-Ala-His-D-Phe-Arg-
Trp-Cys]-NH₂;

(SEQ ID NO: 540)

c[Hydantoin(C(O)-(Ala-Cys))-D-Ala-His-D-Phe-Arg-
Trp-Cys]-NH₂;

(SEQ ID NO: 541)

c[Hydantoin(C(O)-(D-Ala-Cys))-D-Ala-His-D-Phe-
Arg-Trp-Cys]-NH₂;

(SEQ ID NO: 542)

c[Hydantoin(C(O)-(Aib-Cys))-D-Ala-His-D-Phe-Arg-
Trp-Cys]-NH₂;

(SEQ ID NO: 543)

c[Hydantoin(C(O)-(Val-Cys))-D-Ala-His-D-Phe-Arg-
Trp-Cys]-NH₂;

(SEQ ID NO: 544)

c[Hydantoin(C(O)-(Abu-Cys))-D-Ala-His-D-Phe-Arg-
Trp-Cys]-NH₂;

(SEQ ID NO: 545)

c[Hydantoin(C(O)-(Leu-Cys))-D-Ala-His-D-Phe-Arg-
Trp-Cys]-NH₂;

(SEQ ID NO: 546)

c[Hydantoin(C(O)-(Ile-Cys))-D-Ala-His-D-Phe-Arg-
Trp-Cys]-NH₂;

(SEQ ID NO: 547)

c[Hydantoin(C(O)-(Cha-Cys))-D-Ala-His-D-Phe-Arg-
Trp-Cys]-NH₂;

(SEQ ID NO: 548)

c[Hydantoin(C(O)-(A6c-Cys))-D-Ala-His-D-Phe-Arg-
Trp-Cys]-NH₂;

(SEQ ID NO: 549)

c[Hydantoin(C(O)-(Phe-Cys))-D-Ala-His-D-Phe-Arg-
Trp-Cys]-NH₂;

(SEQ ID NO: 550)

c[Hydantoin(C(O)-(Gly-Cys))-D-Ala-His-D-Phe-Arg-
Trp-Cys]-NH₂;
or

(SEQ ID NO: 551)

c[Hydantoin(C(O)-(Gly-Cys))-Glu-His-D-Phe-Arg-
Trp-Cys]-NH₂;

or pharmaceutically acceptable salts thereof.

In some embodiments, a compound of Formula (VII) is disclosed in International Application Publication Number WO2008/147556, which is incorporated herein by reference in its entirety.

In some embodiments, the MC4R agonist is a compound of Formula (VIII):

(R²R³)-A⁰-A¹-c(A²-A³-A⁴-A⁵-A⁶-A⁷-A⁸-A⁹)-A¹⁰-R¹ (VIII)

or a pharmaceutically acceptable salt thereof wherein:
A⁰ is an aromatic amino acid
A¹ is Acc, HN—(CH₂)ₘ—C(O), an L- or D-amino acid;
A² is Asp, Cys, D-Cys, hCys, D-hCys, Glu, Pen, or D-Pen;
A³ is Aib, Ala, β-Ala, Gaba, Gly or a D-amino acid;
A⁴ is H is, 2-Pal, 3-Pal, 4-Pal, (X¹, X², X³, X⁴, X⁵)Phe, Taz, 2-Thi, or 3-Thi;
A⁵ is D-Bal, D-1-Nal, D-2-Nal, D-Phe, L-Phe, D-(X¹, X², X³, X⁴, X⁵)Phe, L-Phe, D-Trp or D-(Et)Tyr;
A⁶ is Arg, hArg, Dab, Dap, Lys, Orn, or HN CH((CH₂)ₙ N(R⁴R⁵)) C(O);
A⁷ is Bal, D-Bal, Bip, D-Bip, 1-Nal, D-1-Nal, 2-Nal, D-2-Nal, or D-Trp;
A⁸ is Acc, Aha, Ahx, Ala, D-Ala, β-Ala, Apn, Gaba, Gly, HN—(CH₂)ₛ—C(O), or deleted;
A⁹ is Cys, D-Cys, hCys, D-hCys, Dab, Dap, Lys, Orn, Pen, or D-Pen;
A¹⁰ is Acc, HN—(CH₂)ₜ—C(O), L- or D-amino acid, or deleted;
R¹ is OH, or NH₂;
each of R² and R³ is, independently for each occurrence selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)allyl, substituted aryl(C₁-C₄₀)acyl, (C₁-C₄₀)alkylsulfonyl, or C(NH) NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X¹, X², X³, X⁴, and X⁵ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

In example embodiments of Formula (VIII),
(I) when R⁴ is (C₁-C₄₀)acyl, aryl(C₁-C₄₀)acyl, substituted (C₁-C₄₀)acyl, substituted aryl(C₁-C₄₀)acyl, (C₁-C₄₀)alkylsulfonyl, or C(NH)—NH₂, then R⁵ is H or (C₁-C₄₀)alkyl, (C₁-C₄₀)heteroalkyl, (C₂-C₄₀)alkenyl, (C₂-C₄₀)alkynyl, aryl(C₁-C₄₀)alkyl, substituted (C₁-C₄₀)alkyl, substituted (C₁-C₄₀)heteroalkyl, substituted (C₂-C₄₀)alkenyl, substituted (C₂-C₄₀)alkynyl, or substituted aryl(C₁-C₄₀)alkyl;
(II) when R² is (C₁-C₃₀)acyl, aryl(C₁-C₃₀)acyl, substituted (C₁-C₃₀)acyl, or substituted aryl(C₁-C₃₀)acyl, then R³ is H, (C₁-C₃₀)alkyl, (C₁-C₃₀)heteroalkyl, (C₂-C₃₀)alkenyl, (C₂-C₃₀)alkynyl, aryl(C₁-C₃₀)alkyl, substituted (C₁-C₃₀)alkyl, substituted (C₁-C₃₀)heteroalkyl, substituted (C₂-C₃₀)alkenyl, substituted (C₂-C₃₀)alkynyl, or substituted aryl(C₁-C₃₀)alkyl;
(III) when A² is Cys, D-Cys, hCys, D-hCys, Pen, or D-Pen, then A⁹ is Cys, D-Cys, hCys, D-hCys, Pen, or D-Pen;
(IV) when A² is Asp or Glu, then A⁹ is Dab, Dap, Orn, or Lys;
(V) when A⁸ is Ala or Gly, then A¹ is not Nle; or pharmaceutically acceptable salts thereof.

In example embodiments of compounds of Formula (VIII):
A⁰ is 1-Nal, 2-Nal, H is, Pff, Phe, Trp, or Tyr; A¹ is Arg; A² is Cys; A³ is D-Ala; A⁴ is H; A⁵ is D-Phe; A⁶ is Arg; A⁷ is Trp; A⁸ is deleted; A⁹ is Cys; and A¹⁰ is deleted; or
   pharmaceutically acceptable salts thereof.
   Particular compounds of the immediately foregoing group of compounds include:

(SEQ ID NO: 552)
Ac-Tyr-Arg-c(Cys-D-Ala-His-D-Phe-Arg-Trp-Cys)-NH₂;
(SEQ ID NO: 553)
Ac-2-Nal-Arg-c(Cys-D-Ala-His-D-Phe-Arg-Trp-Cys)-
NH₂;
(SEQ ID NO: 554)
Ac-1-Nal-Arg-c(Cys-D-Ala-His-DPhe-Arg-Trp-Cys)-
NH₂;
(SEQ ID NO: 555)
Ac-Phe-Arg-c(Cys-D-Ala-His-D-Phe-Arg-Trp-Cys)-NH₂;
(SEQ ID NO: 556)
Ac-Trp-Arg-c(Cys-D-Ala-His-D-Phe-Arg-Trp-Cys)-NH₂;
(SEQ ID NO: 557)
Ac-Pff-Arg-c(Cys-D-Ala-His-D-Phe-Arg-Trp-Cys)-NH₂;
(SEQ ID NO: 558)
H-His-Arg-c(Cys-D-Ala-His-D-Phe-Arg-Trp-Cys)-NH₂;
or
(SEQ ID NO: 559)
Ac-His-Arg-c(Cys-D-Ala-His-D-Phe-Arg-Trp-Cys)-NH₂;

or a pharmaceutically acceptable salt thereof.

In some embodiments, the MC4R agonist is an agonist described in WO2014/144260 A1, incorporated herein by reference.

In one example embodiment, an MC4R agonist is a compound represented by structural formula (IX):

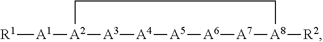

or a pharmaceutically acceptable salt thereof, wherein:
R¹ is H, or a C₁-C₆ acyl;
R² is, —NR³R⁴, or —OR⁵ wherein R³, R⁴, and R⁵ are each independently is H or a C₁-C₆ alkyl;
A¹ is an amino acid residue selected from Arg, Lys, Orn, His, Nle, Phe, Val, Leu, Trp, Tyr, Ala, Ser, Thr, Gln, Asn, Asp, Glu, or TzAla; or
A¹ is a moiety selected from an optionally substituted C₁-C₁₂ alkyl, an optionally substituted C₆-C₁₈ aryl, an optionally substituted C₅-C₁₈ heteroaryl, an aralkyl wherein the aryl portion is an optionally substituted C₆-C₁₈ aryl, and the alkyl portion is an optionally substituted C₁-C₁₂ alkyl, or a heteroaralkyl, wherein the heteroaryl portion is an optionally substituted C₅-C₁₈ heteroaryl, and the alkyl portion is an optionally substituted C₁-C₁₂ alkyl;
A² and A⁸ is each independently an amino acid residue selected from Cys, hCys, Pen, Asp, Glu, Lys, Orn, Dbu, or Dpr, wherein A² and A⁸ are pairwise selected so as to be able to form covalent bond between their respective side chains;
A³ is absent or is an amino acid residue selected from Ala, Tle, Val, Leu, Ile, Cha, Pro, Ser, Thr, Lys, Arg, His, Phe, Gln, Sar, Gly, Asn, Aib, or residue Y, wherein Y is an amino acid selected from amino acids represented by the following structural formulas

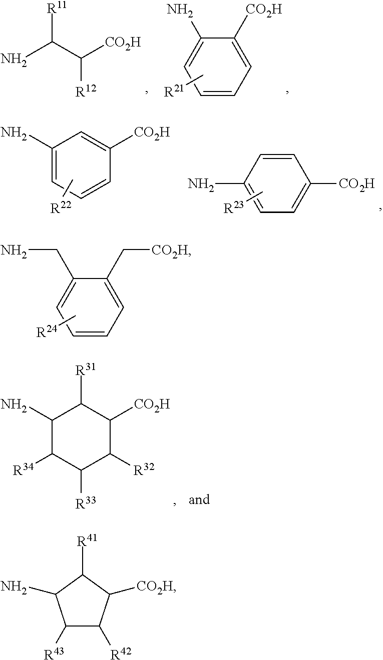

wherein:
R¹¹ and R¹², each independently, is H, —CH₃, phenyl, or benzyl;
R²¹, R²², R²³, and R²⁴, each independently is H, —CH₃, —CF₃, phenyl, benzyl, F, Cl, Br, I, —OCH₃, or —OH;
R³¹, R³², R³³, R³⁴, R⁴¹, R⁴², and R⁴³, each independently is H, —CH₃, —CF₃, phenyl, benzyl, F, Cl, Br, I, —OCH₃, or —OH;
A⁴ is absent or is an amino acid residue selected from Atc, Ala, QAla, Aib, Sar, Ser, Thr, Pro, Hyp, Asn, Gln, an optionally substituted His, Trp, Tyr, Lys, Arg, sChp, or residue X, where the X is an amino acid selected from amino acids represented by the following structural formulas:

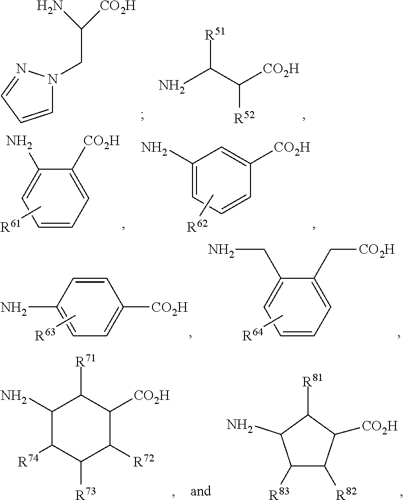

wherein:
R⁵¹ and R⁵², each independently, is H, —CH₃, phenyl, or benzyl;
R⁶¹, R⁶², R⁶³, and R⁶⁴, each independently is H, —CH₃, —CF₃, phenyl, benzyl, F, Cl, Br, I, —OCH₃, or —OH;
R⁷¹, R⁷², R⁷³, R⁷⁴, R⁸¹, R⁸², and R⁸³, each independently is H, —CH₃, —CF₃, phenyl, benzyl, F, Cl, Br, I, —OCH₃, or —OH;
A⁵ is an optionally substituted Phe, an optionally substituted 1-Nal, or an optionally substituted 2-Nal;
A⁶ is Arg; and
A⁷ is Trp,
   wherein any amino acid residue is either in L- or in D-configuration.

Exemplary compound of Formula (IX) include:

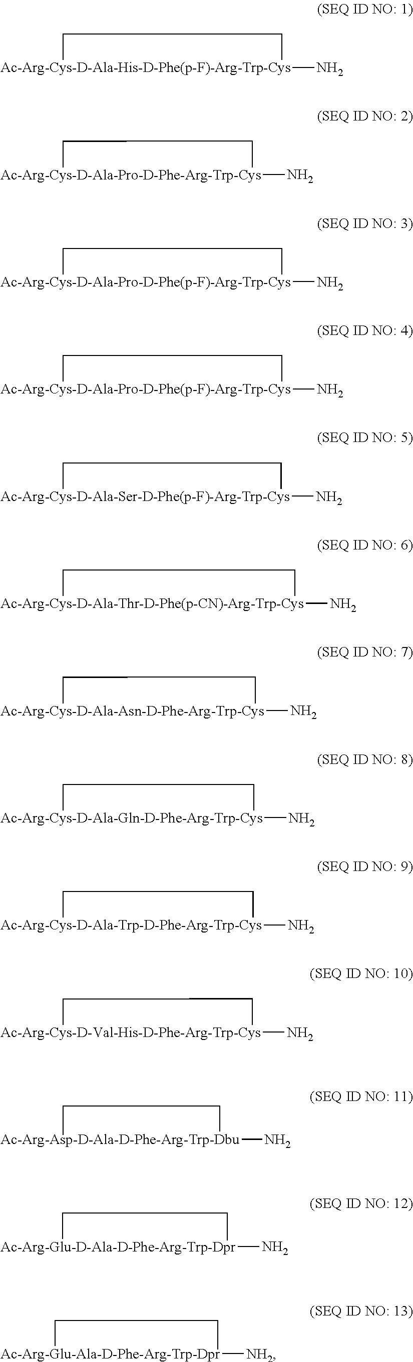

pharmaceutically acceptable salt thereof.

In yet another embodiment, the polypeptides of the present invention include any one of the following structural formulas:

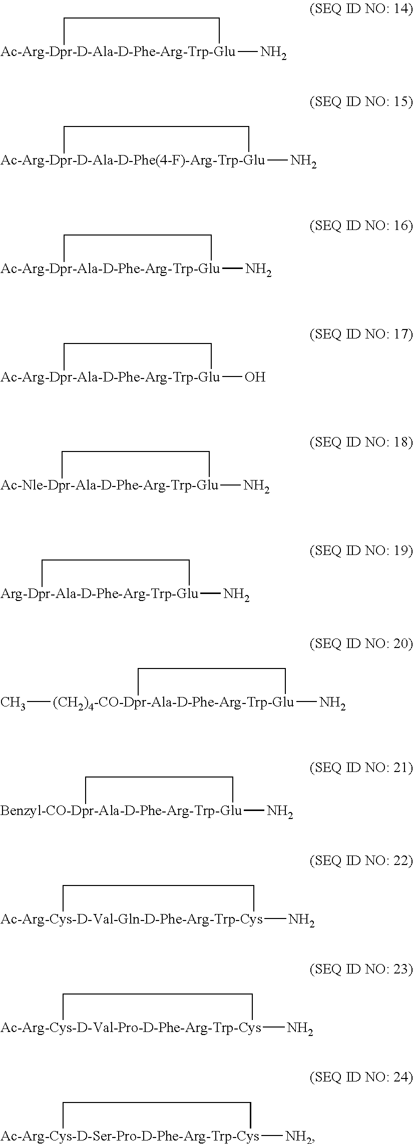

or a pharmaceutically acceptable salt thereof.

In a further embodiment, the polypeptides of the present invention include the polypeptide represented by any one of the following structural formulas:

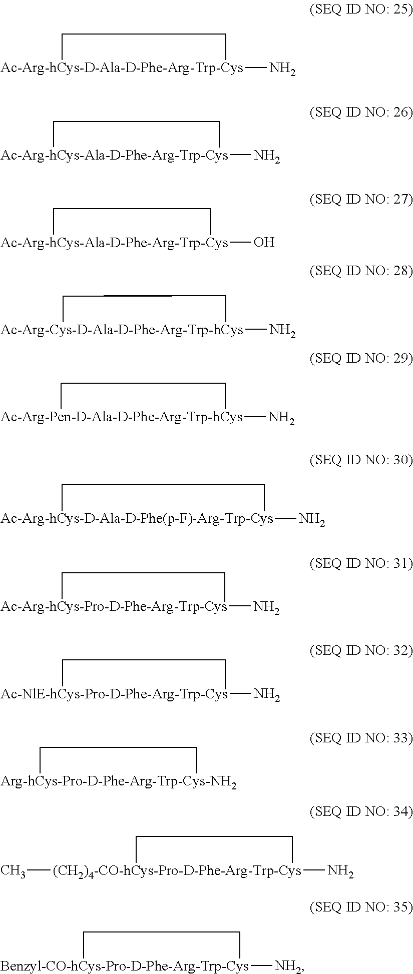

or a pharmaceutically acceptable salt thereof.

In yet another embodiment, the polypeptides of the present invention include a polypeptide represented by formula (I), wherein A⁴ is an amino acid residue selected from Atc, Ala, QAla, Aib, Sar, Ser, Thr, Pro, Hyp, Asn, Gln, a substituted His, Trp, Tyr, Lys, Arg, sChp, or residue X. Examples of such peptides include peptides represented by any one of the following structural formulas:

(SEQ ID NO: 36)
Ac-Arg-cyclo[Cys-D-Ala-His(3-Me)-D-Phe-Arg-Trp-
Cys]-NH₂;
(SEQ ID NO: 37)
Ac-Arg-cyclo[Cys-D-Ala-His(1-Me)-D-Phe-Arg-Trp-
Cys]-NH₂;
(SEQ ID NO: 9)
Ac-Arg-cyclo[Cys-D-Ala-Trp-D-Phe-Arg-Trp-Cys]-NH₂;
(SEQ ID NO: 8)
Ac-Arg-cyclo[Cys-D-Ala-Gln-D-Phe-Arg-Trp-Cys]-NH₂;
(SEQ ID NO: 7)
Ac-Arg-cyclo[Cys-D-Ala-Asn-D-Phe-Arg-Trp-Cys]-NH₂;
(SEQ ID NO: 38)
Ac-Arg-cyclo[Cys-D-Ala-Arg-D-Phe-Arg-Trp-Cys]-NH₂;
(SEQ ID NO: 39)
Ac-Arg-cyclo[Cys-D-Ala-Tyr-D-Phe-Arg-Trp-Cys]-NH₂;
(SEQ ID NO: 40)
Ac-Arg-cyclo[Cys-D-Ala-D-Pro-D-Phe-Arg-Trp-Cys]-
NH₂;
(SEQ ID NO: 2)
Ac-Arg-cyclo[Cys-D-Ala-Pro-D-Phe-Arg-Trp-Cys]-NH₂;
(SEQ ID NO: 4)
Ac-Arg-cyclo[Cys-D-Ala-Pro-D-Phe(p-F)-Arg-Trp-
Cys]-NH₂;
(SEQ ID NO: 41)
Ac-Arg-cyclo[Cys-D-Ala-Atc-D-Phe-Arg-Trp-Cys]-NH₂;
(SEQ ID NO: 42)
Ac-Arg-cyclo[Cys-D-Ala-QAla-D-Phe-Arg-Trp-Cys]-
NH₂;
(SEQ ID NO: 43)
Ac-Arg-cyclo[Cys-D-Ala-sChp-D-Phe-Arg-Trp-Cys]-
NH₂;
or
(SEQ ID NO: 44)
Ac-Arg-cyclo[Cys-D-Ala-X-D-Phe-Arg-Trp-Cys]-NH₂,

or a pharmaceutically acceptable salt thereof.

In example embodiments, the polypeptides of the present invention include a polypeptide represented by any one of the following structural formulas:

(SEQ ID NO: 15)
Ac-Arg-cyclo[hCys-Ala-D-Phe-Arg-Trp-Cys]-NH₂;
(SEQ ID NO: 14)
Ac-Arg-cyclo[hCys-D-Ala-D-Phe-Arg-Trp-Cys]-NH₂;
(SEQ ID NO: 45)
Ac-Arg-cyclo[hCys-D-Ala-D-Phe-Arg-Trp-Pen]-NH₂;
(SEQ ID NO: 26)
Ac-Arg-cyclo[Glu-D-Ala-D-Phe-Arg-Trp-Dpr]-NH₂;
(SEQ ID NO: 27)
Ac-Arg-cyclo[Glu-Ala-D-Phe-Arg-Trp-Dpr]-NH₂;
(SEQ ID NO: 46)
Ac-Arg-cyclo[hCys-Aib-D-Phe-Arg-Trp-Cys]-NH₂;
(SEQ ID NO: 47)
Ac-Arg-cyclo[hCys-Sar-D-Phe-Arg-Trp-Cys]-NH₂;
(SEQ ID NO: 48)
Ac-Arg-cyclo[hCys-Val-D-Phe-Arg-Trp-Cys]-NH₂;
(SEQ ID NO: 49)
Ac-Arg-cyclo[hCys-D-Val-D-Phe-Arg-Trp-Cys]-NH₂;
(SEQ ID NO: 50)
Ac-Arg-cyclo[hCys-Gln-D-Phe-Arg-Trp-Cys]-NH₂;
(SEQ ID NO: 51)
Ac-Arg-cyclo[hCys-D-Gln-D-Phe-Arg-Trp-Cys]-NH₂;
(SEQ ID NO: 52)
Ac-Arg-cyclo[hCys-Ala-D-Phe-Arg-Trp-Pen]-NH₂;
(SEQ ID NO: 53)
Ac-Arg-cyclo[D-Pen-D-Ala-D-Phe-Arg-Trp-hCys]-NH₂;
(SEQ ID NO: 17)
Ac-Arg-cyclo[Cys-D-Ala-D-Phe-Arg-Trp-hCys]-NH₂;
(SEQ ID NO: 54)
Ac-Arg-cyclo[Pen-D-Ala-D-Phe-Arg-Trp-hCys]-NH₂;
(SEQ ID NO: 55)
Ac-Arg-cyclo[D-hCys-D-Ala-D-Phe-Arg-Trp-Cys]-NH₂;
(SEQ ID NO: 20)
Ac-Arg-cyclo[hCys-Pro-D-Phe-Arg-Trp-Cys]-NH₂;
or
(SEQ ID NO: 56)
Ac-Arg-cyclo[hCys-D-Pro-D-Phe-Arg-Trp-Cys]-NH₂,

or a pharmaceutically acceptable salt thereof.

In another embodiment, the polypeptides of the present invention include polypeptides represented by formula (I), wherein A³ is an amino acid residue selected from Tle, Val, Leu, Ile, Cha, Pro, Ser, Thr, Lys, Arg, His, Phe, Gln, Sar, Gly, Asn, or Aib; and A⁴ is an amino acid residue selected from Atc, Ala, QAla, Aib, Sar, Ser, Thr, Pro, Hyp, Asn, Gln, a substituted His, Trp, Tyr, Lys, Arg, sChp, or residue X. Examples of such polypeptides are polypeptides represented by any one of the following structural formulas:

(SEQ ID NO: 57)
Ac-Arg-cyclo[Cys-Val-Gln-D-Phe-Arg-Trp-Cys]-NH₂;
(SEQ ID NO: 11)
Ac-Arg-cyclo[Cys-D-Val-Gln-D-Phe-Arg-Trp-Cys]-NH₂;
or
(SEQ ID NO: 58)
Ac-Arg-cyclo[Cys-D-Val-His(1-Me)-D-Phe-Arg-Trp-
Cys]-NH₂,

or a pharmaceutically acceptable salt thereof.

In a further embodiment, the polypeptides of the present invention include a polypeptide represented by any one of the following structural formulas:

(SEQ ID NO: 59)
Ac-TzAla-cyclo[Cys-Ala-Gln-D-Phe-Arg-Trp-Cys]-NH₂;
or
(SEQ ID NO: 60)
Ac-Glu-cyclo[Cys-Ala-His-D-Phe-Arg-Trp-Cys]-NH₂,

or a pharmaceutically acceptable salt thereof.

In yet another embodiment, the polypeptides of the present invention include a polypeptide represented by any one of the following structural formulas:

(SEQ ID NO: 37)
Ac-Arg-cyclo[Cys-D-Ala-His(1-Me)-D-Phe-Arg-Trp-
Cys]-NH₂;
(SEQ ID NO: 8)
Ac-Arg-cyclo[Cys-D-Ala-Gln-D-Phe-Arg-Trp-Cys]-NH₂;
or
(SEQ ID NO: 7)
Ac-Arg-cyclo[Cys-D-Ala-Asn-D-Phe-Arg-Trp-Cys]-NH₂,

or a pharmaceutically acceptable salt thereof.

In a further embodiment, the polypeptides of the present invention include a polypeptide represented by any one of the following structural formulas:

(SEQ ID NO: 61)
Ac-Arg-cyclo[Cys-D-Leu-His-D-Phe-Arg-Trp-Cys]-NH₂;
(SEQ ID NO: 62)
Ac-Arg-cyclo[Cys-D-Ile-His-D-Phe-Arg-Trp-Cys]-NH₂;
(SEQ ID NO: 63)
Ac-Arg-cyclo[Cys-D-Tle-His-D-Phe-Arg-Trp-Cys]-NH₂;
or
(SEQ ID NO: 10)
Ac-Arg-cyclo[Cys-D-Val-His-D-Phe-Arg-Trp-Cys]-NH₂,

or a pharmaceutically acceptable salt thereof.

In a further embodiment, the polypeptides of the present invention include a polypeptide represented by any one of the following structural formulas:

(SEQ ID NO: 64)

Ac-Arg-cyclo[Cys-D-Ala-His(1-Me)-D-2-Nal-Arg-
Trp-Cys]-NH₂;

(SEQ ID NO: 65)

Ac-Arg-cyclo[Cys-D-Ala-Gln-D-2-Nal-Arg-Trp-Cys]-
NH₂;
or

(SEQ ID NO: 66)

Ac-Arg-cyclo[Cys-D-Ala-Asn-D-2-Nal-Arg-Trp-Cys]-
NH₂,

or a pharmaceutically acceptable salt thereof.

In a further embodiment, the polypeptides of the present invention include a polypeptide represented by any one of the following structural formulas:

Ac-Arg-cyclo[Cys-D-Ala-His(1-Me)-D-Phe-Arg-Trp-Cys]-OH; (SEQ ID NO: 67)

Ac-Arg-cyclo[Cys-D-Ala-Gln-D-Phe-Arg-Trp-Cys]-OH; (SEQ ID NO: 68) or

Ac-Arg-cyclo[Cys-D-Ala-Asn-D-Phe-Arg-Trp-Cys]-OH, (SEQ ID NO: 69)

or a pharmaceutically acceptable salt thereof.

In one example embodiment, an MC4R agonist is a compound represented by structural formula (X):

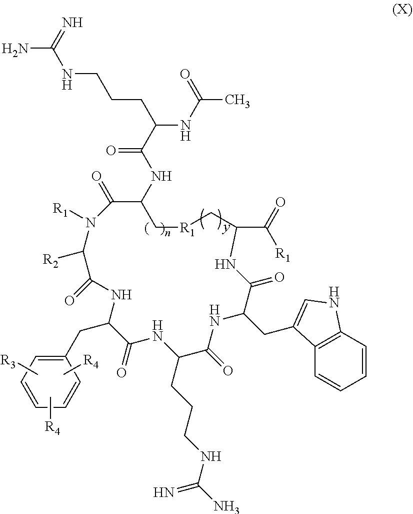

or a pharmaceutically acceptable salt thereof. In structural formula (X), the chemical substituents are defined as follows:
R₁ is —NH—C(O)— or —C(O)—NH—;
R₂ is —H, —CH₂—, or, R₂, together with R₃, forms a pyrrolidine ring optionally substituted with —OH;
R₃ is —(CH₂)₂— if R₂ is —CH₂—, and otherwise R₃ is selected from

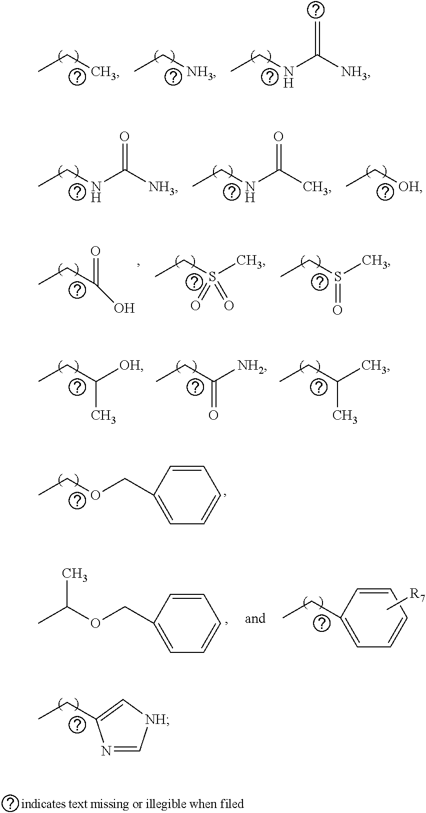

R₄ₐ, R_{4b}, and R_{4c} are each independently selected from hydrogen, halo, (C₁-C₁₀)alkyl-halo, (C₁-C₁₀)alkyl-dihalo, (C₁-C₁₀)alkyl-trihalo, (C₁-C₁₀)alkyl, (C₁-C₁₀)alkoxy, (C₁-C₁₀)alkylthio, aryl, aryloxy, nitro, nitrile, sulfoniamide, amino, hydroxyl, carboxy, and alkoxy-carbonyl. In one example embodiment, R₄ₐ, R_{4b}, and R_{4c} is not hydrogen.
R₅ is —OH or —N(R₆ₐ)(R_{6b});
R₆ₐ and R_{6b} are each independently H or C₁ to C₄ linear, branched or cyclic alkyl chain;
R₇ is —H or —C(O)—NH₂;
w is in each instance independently 0 to 5;
x is 1 to 5;
y is 1 to 5;
z is in each instance independently 1 to 5.

An example of a compound of structural formula (X) is a cyclic peptide defined by structural formula (XI).

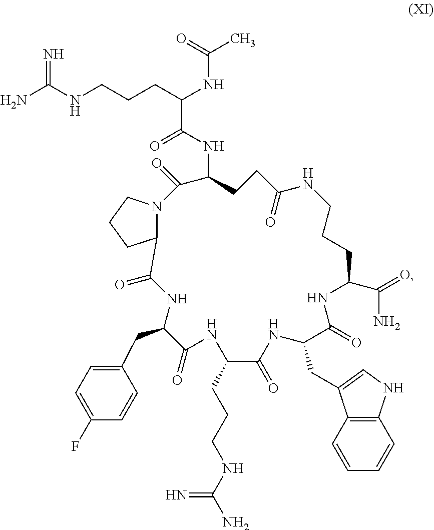 [SMILES: CC(=O)NC(CCCNC(=N)N)C(=O)N[C@H]1CCC(=O)NCCC[C@@H](C(N)=O)NC(=O)[C@H](Cc2c[nH]c3ccccc23)NC(=O)[C@H](CCCNC(=N)N)NC(=O)[C@@H](Cc2ccc(F)cc2)NC(=O)C2CCCN2C1=O]

or a pharmaceutically acceptable salt thereof.

In one example embodiment, the MC4R agonist is Ac-Arg-c(Cys-D-Ala-His-D-Phe-Arg-Trp-Cys)-NH₂ (SEQ ID NO: 140) or a pharmaceutically acceptable salt thereof. In another example embodiment, the MC4R agonist is Hydantoin(C(O)-(Arg-Gly))-c(Cys-Glu-His-D-Phe-Arg-Trp-Cys)-NH₂ (SEQ ID NO: 500) or a pharmaceutically acceptable salt thereof.

In some embodiments, the MC4R agonist is an agonist described in WO2014/144260 A1, incorporated herein by reference.

In one example embodiment, the MC4 agonist is a compound represented by Formula (XII):

General Structure:

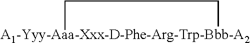

Aaa, Bbb=Selected from Cys, hCys, Pen; capable of establishing a disulfide bridge or
   Glu, Asp, Lys, Orn, Dpr, Dbu capable of establishing a lactam bridge
Xxx=Asn, Gln, Ser, Thr
Yyy=Lys, Arg, D-Lys, D-Arg
A₁=H, Ac
A₂=OH, NH₂

In embodiments, the MC4R agonist is chosen from one or more of the following compounds, (or pharmaceutically acceptable salt thereof):

| (SEQ ID NO: 1) | |
|---|---|
| | Ac-Arg-(hCys-Asn-D-Phe-Arg-Trp-Pen)-NH₂ |
| (SEQ ID NO: 2) | |
| | Ac-Arg-(hCys-Gln-D-Phe-Arg-Trp-Pen)-NH₂ |
| (SEQ ID NO: 3) | |
| | Ac-Arg-(hCys-Ser-D-Phe-Arg-Trp-Pen)-NH₂ |
| (SEQ ID NO: 4) | |
| | Ac-Arg-(hCys-Thr-D-Phe-Arg-Trp-Pen)-NH₂ |
| (SEQ ID NO: 5) | |
| | Ac-Arg-(Glu-Gln-D-Phe-Arg-Trp-Apr)-NH₂ |
| (SEQ ID NO: 6) | |
| | Ac-Arg-(hCys-Asn-D-Phe-Arg-Trp-Pen)-OH |
| (SEQ ID NO: 7) | |
| | H-Arg-(hCys-Asn-D-Phe-Arg-Trp-Pen)-NH₂ |
| (SEQ ID NO: 8) | |
| | H-Arg-(hCys-Asn-D-Phe-Arg-Trp-Pen)-OH |
| (SEQ ID NO: 9) | |
| | Ac-D-Arg-(hCys-Asn-D-Phe-Arg-Trp-Pen)-NH₂ |
| (SEQ ID NO: 10) | |
| | H-D-Arg-(hCys-Asn-D-Phe-Arg-Trp-Pen)-NH₂ |
| (SEQ ID NO: 11) | |
| | Ac-D-Arg-(hCys-Asn-D-Phe-Arg-Trp-Pen)-OH |
| (SEQ ID NO: 12) | |
| | H-D-Arg-(hCys-Asn-D-Phe-Arg-Trp-Pen)-OH |
| (SEQ ID NO: 13) | |
| | Ac-Lys-(hCys-Asn-D-Phe-Arg-Trp-Pen)-NH₂ |
| (SEQ ID NO: 14) | |
| | Ac-Lys-(hCys-Asn-D-Phe-Arg-Trp-Pen)-OH |
| (SEQ ID NO: 15) | |
| | H-Lys-(hCys-Asn-D-Phe-Arg-Trp-Pen)-NH₂ |
| (SEQ ID NO: 16) | |
| | H-Lys-(hCys-Asn-D-Phe-Arg-Trp-Pen)-OH |
| (SEQ ID NO: 17) | |
| | Ac-D-Lys-(hCys-Asn-D-Phe-Arg-Trp-Pen)-NH₂ |
| (SEQ ID NO: 18) | |
| | H-D-Lys-(hCys-Asn-D-Phe-Arg-Trp-Pen)-NH₂ |
| (SEQ ID NO: 19) | |
| | Ac-D-Lys-(hCys-Asn-D-Phe-Arg-Trp-Pen)-OH |
| (SEQ ID NO: 20) | |
| | H-D-Lys-(hCys-Asn-D-Phe-Arg-Trp-Pen)-OH |
| (SEQ ID NO: 21) | |
| | Ac-Arg-(hCys-Gln-D-Phe-Arg-Trp-Pen)-OH |
| (SEQ ID NO: 22) | |
| | H-Arg-(hCys-Gln-D-Phe-Arg-Trp-Pen)-NH₂ |
| (SEQ ID NO: 23) | |
| | H-Arg-(hCys-Gln-D-Phe-Arg-Trp-Pen)-OH |
| (SEQ ID NO: 24) | |
| | Ac-D-Arg-(hCys-Gln-D-Phe-Arg-Trp-Pen)-NH₂ |
| (SEQ ID NO: 25) | |
| | H-D-Arg-(hCys-Gln-D-Phe-Arg-Trp-Pen)-NH₂ |
| (SEQ ID NO: 26) | |
| | Ac-D-Arg-(hCys-Gln-D-Phe-Arg-Trp-Pen)-OH |
| (SEQ ID NO: 27) | |
| | H-D-Arg-(hCys-Gln-D-Phe-Arg-Trp-Pen)-OH |
| (SEQ ID NO: 28) | |
| | Ac-Lys-(hCys-Gln-D-Phe-Arg-Trp-Pen)-NH₂ |
| (SEQ ID NO: 29) | |
| | Ac-Lys-(hCys-Gln-D-Phe-Arg-Trp-Pen)-OH |
| (SEQ ID NO: 30) | |
| | H-Lys-(hCys-Gln-D-Phe-Arg-Trp-Pen)-NH₂ |
| (SEQ ID NO: 31) | |
| | H-Lys-(hCys-Gln-D-Phe-Arg-Trp-Pen)-OH |
| (SEQ ID NO: 32) | |
| | Ac-D-Lys-(hCys-Gln-D-Phe-Arg-Trp-Pen)-NH₂ |
| (SEQ ID NO: 33) | |
| | H-D-Lys-(hCys-Gln-D-Phe-Arg-Trp-Pen)-NH₂ |
| (SEQ ID NO: 34) | |
| | Ac-D-Lys-(hCys-Gln-D-Phe-Arg-Trp-Pen)-OH |
| (SEQ ID NO: 35) | |
| | H-D-Lys-(hCys-Gln-D-Phe-Arg-Trp-Pen)-OH |
| (SEQ ID NO: 36) | |
| | Ac-Arg-(hCys-Ser-D-Phe-Arg-Trp-Pen)-OH |
| (SEQ ID NO: 37) | |
| | H-Arg-(hCys-Ser-D-Phe-Arg-Trp-Pen)-NH₂ |
| (SEQ ID NO: 38) | |
| | H-Arg-(hCys-Ser-D-Phe-Arg-Trp-Pen)-OH |
| (SEQ ID NO: 39) | |
| | Ac-D-Arg-(hCys-Ser-D-Phe-Arg-Trp-Pen)-NH₂ |
| (SEQ ID NO: 40) | |
| | H-D-Arg-(hCys-Ser-D-Phe-Arg-Trp-Pen)-NH₂ |
| (SEQ ID NO: 41) | |
| | Ac-D-Arg-(hCys-Ser-D-Phe-Arg-Trp-Pen)-OH |
| (SEQ ID NO: 42) | |
| | H-D-Arg-(hCys-Ser-D-Phe-Arg-Trp-Pen)-OH |
| (SEQ ID NO: 43) | |
| | Ac-Lys-(hCys-Ser-D-Phe-Arg-Trp-Pen)-NH₂ |
| (SEQ ID NO: 44) | |
| | Ac-Lys-(hCys-Ser-D-Phe-Arg-Trp-Pen)-OH |
| (SEQ ID NO: 45) | |
| | H-Lys-(hCys-Ser-D-Phe-Arg-Trp-Pen)-NH₂ |
| (SEQ ID NO: 46) | |
| | H-Lys-(hCys-Ser-D-Phe-Arg-Trp-Pen)-OH |
| (SEQ ID NO: 47) | |
| | Ac-D-Lys-(hCys-Ser-D-Phe-Arg-Trp-Pen)-NH₂ |
| (SEQ ID NO: 48) | |
| | H-D-Lys-(hCys-Ser-D-Phe-Arg-Trp-Pen)-NH₂ |
| (SEQ ID NO: 49) | |
| | Ac-D-Lys-(hCys-Ser-D-Phe-Arg-Trp-Pen)-OH |
| (SEQ ID NO: 50) | |
| | H-D-Lys-(hCys-Ser-D-Phe-Arg-Trp-Pen)-OH |
| (SEQ ID NO: 51) | |
| | Ac-Arg-(hCys-Thr-D-Phe-Arg-Trp-Pen)-OH |
| (SEQ ID NO: 52) | |
| | H-Arg-(hCys-Thr-D-Phe-Arg-Trp-Pen)-NH₂ |
| (SEQ ID NO: 53) | |
| | H-Arg-(hCys-Thr-D-Phe-Arg-Trp-Pen)-OH |
| (SEQ ID NO: 54) | |
| | Ac-D-Arg-(hCys-Thr-D-Phe-Arg-Trp-Pen)-NH₂ |
| (SEQ ID NO: 55) | |
| | H-D-Arg-(hCys-Thr-D-Phe-Arg-Trp-Pen)-NH₂ |
| (SEQ ID NO: 56) | |
| | Ac-D-Arg-(hCys-Thr-D-Phe-Arg-Trp-Pen)-OH |
| (SEQ ID NO: 57) | |
| | H-D-Arg-(hCys-Thr-D-Phe-Arg-Trp-Pen)-OH |
| (SEQ ID NO: 58) | |
| | Ac-Lys-(hCys-Thr-D-Phe-Arg-Trp-Pen)-NH₂ |
| (SEQ ID NO: 59) | |
| | Ac-Lys-(hCys-Thr-D-Phe-Arg-Trp-Pen)-OH |
| (SEQ ID NO: 60) | |
| | H-Lys-(hCys-Thr-D-Phe-Arg-Trp-Pen)-NH₂ |
| (SEQ ID NO: 61) | |
| | H-Lys-(hCys-Thr-D-Phe-Arg-Trp-Pen)-OH |
| (SEQ ID NO: 62) | |
| | Ac-D-Lys-(hCys-Thr-D-Phe-Arg-Trp-Pen)-NH₂ |
| (SEQ ID NO: 63) | |
| | H-D-Lys-(hCys-Thr-D-Phe-Arg-Trp-Pen)-NH₂ |
| (SEQ ID NO: 64) | |
| | Ac-D-Lys-(hCys-Thr-D-Phe-Arg-Trp-Pen)-NH₂ |
| | or |
| (SEQ ID NO: 65) | |
| | H-D-Lys-(hCys-Thr-D-Phe-Arg-Trp-Pen)-NH₂. |

In some embodiments, the MC4R agonist is an agonist described in WO2014/144260 or WO2017/059075, each of which is incorporated herein by reference. Administration of a compound or pharmaceutically acceptable salt thereof or a composition comprising a compound or pharmaceutical salt of a compound of the disclosure useful to practice the methods described herein, can be continuous, hourly, four times daily, three time daily, twice daily, once daily, once every other day, twice weekly, once weekly, once every two weeks, once a month, or once every two months, or longer or some other intermittent dosing regimen.

Examples of administration of a compound or composition comprising a compound or pharmaceutical salt of a compound of the disclosure include peripheral administration. Examples of peripheral administration include oral, subcutaneous, intraperitoneal, intramuscular, intravenous, rectal, transdermal or intranasal forms of administration.

As used herein, peripheral administration can include all forms of administration of a compound or a composition comprising a compound of the instant disclosure which excludes intracranial administration. Examples of peripheral administration include, but are not limited to, oral, parenteral (e.g., intramuscular, intraperitoneal, intravenous or subcutaneous injection, extended release, slow release implant, depot and the like), nasal, vaginal, rectal, sublingual or topical routes of administration, including transdermal patch applications and the like.

The nomenclature used to define the peptides is that typically used in the art wherein the amino group at the N-terminus appears to the left and the carboxyl group at the C-terminus appears to the right. Where the amino acid has D and L isomeric forms, it is the L form of the amino acid that is represented unless otherwise explicitly indicated.

The compounds of the disclosure useful for practicing the methods described herein may possess one or more chiral centers and so exist in a number of stereoisomeric forms. All stereoisomers and mixtures thereof are included in the scope of the present disclosure. Racemic compounds may either be separated using preparative HPLC and a column with a chiral stationary phase or resolved to yield individual enantiomers utilizing methods known to those skilled in the art. In addition, chiral intermediate compounds may be resolved and used to prepare chiral compounds of the disclosure.

The compounds described herein may exist in one or more tautomeric forms. All tautomers and mixtures thereof are included in the scope of the present disclosure. For example, a claim to 2-hydroxypyridinyl would also cover its tautomeric form, α-pyridonyl.

Unless defined otherwise, all technical and scientific terms used herein have the same meaning as commonly understood by one of ordinary skill in the art to which this disclosure belongs. Also, all publications, patent applications, patents and other references mentioned herein are incorporated by reference in their entirety.

### Pharmaceutical Compositions/Administration

In accordance with any method or composition described herein, in embodiments, provided herein is a unit dosage of a MC4R agonist described herein, e.g., setmelanotide. In embodiments, the unit dosage contains 0.1-10 mg, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, or 10 mg of the MC4R agonist. In embodiments, the unit dosage contains about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2 mg of the agonist. In embodiments, the unit dosage is suitable for injection, e.g., subcutaneous injection. In embodiments, the unit dosage is disposed in a delivery device suitable for injection, e.g., subcutaneous injection. In embodiments, the unit dosage is disposed in a syringe suitable for injection, e.g., subcutaneous injection, or a pen-type injector. Exemplary pen-type injectors are described, e.g., in U.S. Pat. Nos. 8,512,297B2, 5,688,251A, 5,820,602A, US2014/0163526A1, and U.S. Pat. No. 5,226,895A, incorporated herein by reference.

In embodiments, also provided herein is a pharmaceutical composition comprising a MC4R agonist described herein, e.g., setmelanotide. In embodiments, the pharmaceutical composition includes a therapeutically effective amount of a MC4R agonist described herein, e.g., setmelanotide. A therapeutically effective amount of the agonist can vary according to factors such as the disease state, age, sex, and weight of the individual, and the ability of the agonist to elicit a desired response in the individual, e.g., amelioration of at least one disorder parameter, e.g., a parameter of obesity or hyperphagia, or amelioration of at least one symptom of the disorder, e.g., obesity, hyperphagia, a disease or disorder associated with a gene in Table 1, or other obesity-associated genetic disorder. In embodiments, a therapeutically effective amount is also one in which any toxic or a detrimental effect of the composition is outweighed by the therapeutically beneficial effects.

In certain embodiments, the agonist may be prepared with a carrier that will protect it against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In other embodiments, the MC4R agonist can be prepared as described in WO2014/144842, incorporated herein by reference. In embodiments, the MC4R agonist is prepared in a formulation comprising an anionic excipient, e.g., PEG-carboxylic acid, fatty acid having 10 or more carbon atoms, and/or anionic phospholipid. In embodiments, the anionic phospholipid is described in WO2014/144842 (e.g., at pages 7-9). In some embodiments, the anionic phospholipid is 1,2-distearoyl-sn-Glycero-3-Phosphoethanolamine (DSPE), optionally conjugated to polyethylene glycol (PEG), the structure of which is:

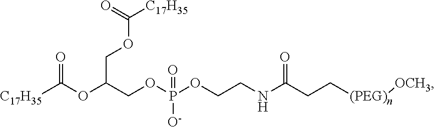

with the value of “n” varying with molecular weight. In embodiments, the fatty acid is described in WO2014/144842 (e.g., at page 9). In embodiments, the PEG-carboxylic acid is described in WO2014/144842 (e.g., at pages 9-11). In embodiments, the molar ratio of the agonist to the anionic excipient ranges from about 1:1 to about 1:10.

In embodiments, the MC4R agonist forms an ionic complex with the other components of the formulation, and e.g., provides a desirable pharmacokinetic profile for the agonist (e.g., extend duration of drug action and/or minimize adverse effects). In embodiments, the formulation is a sustained release formulation. In embodiments, the formulation provides reduced fluctuations in concentration of the agonist after administration.

In other embodiments, the MC4R agonist can be prepared as described in WO 2019/099735, incorporated herein by reference. In embodiments, the MC4R agonist is prepared in a formulation comprising a neutral diacyl lipid and/or a tocopherol; a phospholipid: an alcohol; and optionally, a polar solvent, e.g., a buffer, optionally comprising an antioxidant. In an embodiment, the neutral diacyl lipid comprises glycerol dioleate (GDO). In an embodiment, the phospholipid comprises phosphatidylcholine (e.g., soybean phosphatidylcholine). In an embodiment, the alcohol comprises ethanol. In an embodiment, the formulation is an injectable formulation.

A MC4R agonist described herein, e.g., setmelanotide, can be administered to a subject, e.g., human subject, by various methods. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and/or to other mucosal surfaces. In embodiments, the route of administration is one of: intravenous injection or infusion, subcutaneous injection, or intramuscular injection. In embodiments, the route of administration is subcutaneous injection.

In embodiments, pharmaceutical compositions, e.g., comprising a MC4R agonist described herein, can be administered with medical devices. For example, compositions comprising the agonist can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Other such implants, delivery systems, and modules can also be used.

In embodiments, continuous administration can be indicated, e.g., via subcutaneous pump. In embodiments, the agonist is administered via a syringe (e.g., prefilled syringe), an implantable device, a needleless hypodermic injection device, an infusion pump (e.g., implantable infusion pump), or an osmotic delivery system.

In embodiments, the agonist is administered at a unit dosage, e.g., comprising 0.1-10 mg, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, or 10 mg of the agonist, e.g., subcutaneously.

In embodiments, the MC4R agonist is administered in a bolus at a dose of between 0.1-10 mg, e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, or 10 mg of the MC4R agonist, e.g., subcutaneously.

In embodiments, the MC4R agonist is administered continuously, e.g., via a pump, e.g., subcutaneous pump.

In embodiments, the MC4R agonist, e.g., a unit dosage of the MC4R agonist, is disposed within a delivery device, e.g., a syringe (e.g., prefilled syringe), an implantable device, a needleless hypodermic injection device, an infusion pump (e.g., implantable infusion pump), or an osmotic delivery system.

In embodiments, a daily dosage of the MC4R agonist is administered, e.g., subcutaneously, to a subject. In embodiments, the daily dosage of the MC4R agonist is about 0.1 mg to about 10 mg, e.g., 0.1-0.2, 0.2-0.4, 0.4-0.6, 0.6-0.8, 0.8-1, 1-1.2, 1.2-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.5-8, 8-8.5, 8.5-9, 9-9.5, 9.5-10 mg, e.g., administered subcutaneously.

In embodiments, the MC4R agonist, e.g., setmelanotide, is administered, e.g., via one or multiple administrations, over a period of at least 3 weeks, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks or more, or at least 1, 2, 3, 4, 5, 6, 7, 8, 8, 9, 10, 11, or 12 months or more, or at least 1, 2, 3, 4 years or more. In embodiments, where multiple administrations are provided of the MC4R agonist, the time interval in between any two of the administrations is at least 6 hours, e.g., 6 h, 12 h, 24 h, 1 day, 2 days, 3 days, 4 days, 5 days, 6 days, 7 days, 1 week, 2 weeks, 3 weeks, 4 weeks, or more. In embodiments, the interval in between any two of the administrations is 1 day.

### Kits

A MC4R agonist described herein, e.g., setmelanotide, can be provided in a kit. The kit may include a MC4R agonist described herein and, optionally, a container, a pharmaceutically acceptable carrier and/or informational material. The informational material can be descriptive, instructional, marketing or other material that relates to the methods described herein and/or the use of the MC4R agonist for the methods described herein.

The informational material of the kits is not limited in its form. In one embodiment, the informational material can include information about production of the MC4R agonist, physical properties of the MC4R agonist, concentration, date of expiration, batch or production site information, and so forth. In one embodiment, the informational material relates to methods for administering the MC4R agonist, e.g., by a route of administration described herein and/or at a dose and/or dosing schedule described herein.

In one embodiment, the informational material can include instructions to administer an MC4R agonist described herein in a suitable manner to perform the methods described herein, e.g., in a suitable dose, dosage form, or mode of administration (e.g., a dose, dosage form, or mode of administration described herein). In another embodiment, the informational material can include instructions to administer an MC4R agonist to a suitable subject, e.g., a human, e.g., an obese human, e.g., severely obese human, e.g., having a disease or disorder associated with a gene in Table 1.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about an MC4R agonist described herein and/or its use in the methods described herein. The informational material can also be provided in any combination of formats.

In addition to an MC4R agonist, the composition of the kit can include other ingredients, such as a surfactant, a lyo-protectant or stabilizer, an antioxidant, an antibacterial agent, a bulking agent, a chelating agent, an inert gas, a tonicity agent and/or a viscosity agent, a solvent or buffer, a stabilizer, a preservative, a pharmaceutically acceptable carrier and/or a second agent for treating a condition or disorder described herein. Alternatively, the other ingredients can be included in the kit, but in different compositions or containers than an MC4R agonist described herein.

In some embodiments, a component of the kit is stored in a sealed vial, e.g., with a rubber or silicone closure (e.g., a polybutadiene or polyisoprene closure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

An MC4R agonist described herein can be provided in any form, e.g., liquid, frozen, dried or lyophilized form. It is preferred that a composition including the MC4R agonist described herein be substantially pure and/or sterile. When an MC4R agonist described herein such as setmelanotide is provided in a liquid solution, the liquid solution preferably is an aqueous solution, with a sterile aqueous solution being preferred. In one embodiment, the MC4R agonist is supplied with a diluents or instructions for dilution. The diluent can include for example, a salt or saline solution, e.g., a sodium chloride solution having a pH between 6 and 9, lactated Ringer's injection solution, D5W, or PLASMA-LYTE A Injection pH 7.4© (Baxter, Deerfield, IL).

The kit can include one or more containers for the composition containing an MC4R agonist described herein. In some embodiments, the kit contains separate containers, dividers or compartments for the composition and informational material. For example, the composition can be contained in a bottle, vial, IV admixture bag, IV infusion set, piggyback set or syringe (e.g., prefilled syringe), and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In embodiments, the composition is contained in an injector device, e.g., a pen-type injector. The containers of the kits can be airtight or waterproof (e.g., impermeable to changes in moisture or evaporation).

### ENUMERATED EMBODIMENTS

1. A method of treating a disease, disorder, or condition in in a subject comprising administering to the subject melanocortin-4 receptor (MC4R) agonist, wherein the disease, disorder, or condition is related to an MC4R pathway agonizable gene.
2. The method of embodiment 1, wherein the MC4R pathway agonizable gene is selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, TRPC5, PHIP, and MeCP2.
3. The method of any one of embodiments 1-2, wherein the disease, disorder, or condition is characterized by a mutation (e.g., a substitution mutation, a deletion mutation, or a polymorphism) in the MC4R pathway agonizable gene.
4. The method of embodiment 3, wherein the subject is homozygous or heterozygous for the mutation in the MC4R pathway agonizable gene.
5. The method of any one of embodiments 3-4, wherein the subject is or is identified as being a heterozygous carrier of the mutation(s), e.g., having one functional allele and one non-functional allele of the MC4R pathway agonizable gene.
6. The method of any one of embodiments 3-4, wherein the subject is or is identified as being a compound heterozygous carrier of the mutation(s), e.g., having two distinct non-functional alleles, e.g., having the MC4R pathway agonizable gene.
7. The method of any one of embodiments 3-4, wherein the subject is or is identified as being a homozygous carrier of the mutation(s), e.g., having a homozygous null genotype of the MC4R pathway agonizable gene.
8. The method of any one of embodiments 2-7, wherein the MC4R pathway agonizable gene is selected from RAI1 and SRC.
9. The method of any one of the preceding embodiments, wherein the disease, disorder, or condition is characterized by a plurality of mutations (e.g., at least 2, 3, 4, 5, 6, 7, 8, 9, or 10 mutations) in the MC4R pathway agonizable gene.
10. The method of embodiment 9, wherein the plurality of MC4R pathway agonizable genes comprises RAI1 and SRC.
11. The method of any one of embodiments 9-10, wherein the plurality of MC4R pathway agonizable genes further comprises SHH2.
12. The method of any one of the preceding embodiments, wherein the disease, disorder, or condition is characterized by modulation (e.g., upregulation or downregulation) of the MC4R pathway agonizable gene.
13. The method of any one of the preceding embodiments, wherein the disease, disorder, or condition comprises Bardet-Biedl syndrome, Alstrom Syndrome, Prader Willi syndrome, hypothalamic obesity, or Smith-Magenis syndrome.
14. The method of any one of the preceding embodiments, wherein the disease, disorder, or condition comprises Bardet-Biedl syndrome, Alstrom Syndrome, Prader Willi syndrome, or Smith-Magenis syndrome.
15. The method of any one of the preceding embodiments, wherein the disease, disorder, or condition comprises Bardet-Biedl syndrome.
16. The method of any one of the preceding embodiments, wherein the disease, disorder, or condition comprises Alstrom Syndrome.
17. The method of any one of the preceding embodiments, wherein the disease, disorder, or condition comprises Prader Willi syndrome.
18. The method of any one of the preceding embodiments, wherein the disease, disorder, or condition comprises Smith-Magenis syndrome.
19. The method of any one of the preceding embodiments, wherein the disease, disorder, or condition comprises hypothalamic obesity.
20. The method of any one of the preceding embodiments, wherein a symptom of the disease, disorder, or condition comprises obesity or hyperphagia.
21. The method of any one of the preceding embodiments, wherein the MC4R agonist is has the structure of formula (I):

(R²R³)-A¹-c(A²-A³-A⁴-A⁵-A⁶-A⁷-A⁸-A⁹)-A¹⁰-R¹ (I),
wherein:
A¹ is Acc, HN—(CH₂)ₘ—C(O), L- or D-amino acid, or deleted;
A² is Cys, D-Cys, hCys, D-hCys, Pen, D-Pen, Asp, or Glu;
A³ is Gly, Ala, β-Ala, Gaba, Aib, D-amino acid, or deleted;
A⁴ is His, 2-Pal, 3-Pal, 4-Pal, Taz, 2-Thi, 3-Thi, or (X¹, X², X³, X⁴, X⁵)Phe;
A⁵ is D-Phe, Phe, D-1-Nal, D-2-Nal, D-Trp, D-Bal, D-(X¹, X², X³, X⁴, X⁵)Phe, or D-(Et)Tyr;
A⁶ is Arg, hArg, Dab, Dap, Lys, Orn, or HN-CH((CH₂)ₙ—N(R⁴R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂)ₛ—C(O), or deleted;
A⁹ is Cys, D-Cys, hCys, D-hCys, Pen, D-Pen, Dab, Dap, Orn, or Lys;
A¹⁰ is Acc, HN—(CH₂)ᵣ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.
22. The method of embodiment 21, wherein A¹ is selected from Lys, D-Lys, Arg, and D-Arg.
23. The method of any one of embodiments 21-22, wherein A² and A⁹ are each independently selected from Cys, hCys, and Pen.
24. The method of any one of embodiments 21-23, wherein A³ is selected from Ala or D-Ala.
25. The method of any one of embodiments 21-24, wherein A⁴ is selected from His and D-His.
26. The method of any one of embodiments 21-25, wherein A⁵ is selected from Phe, D-Phe, D-1-Nal, and D-2-Nal.
27. The method of any one of embodiments 21-26, wherein A⁶ is Arg.
28. The method of any one of embodiments 21-27, wherein A⁷ is Trp.
29. The method of any one of embodiments 21-28, wherein A⁸ and/or A¹⁰ is deleted.
30. The method of any one of embodiments 21-29, wherein R¹ is NH₂.
31. The method of any one of embodiments 21-30, wherein one of R² and R³ is independently hydrogen and the other of R² and R³ is independently (C₁-C₃₀) acyl (e.g., acetyl).
32. The method of any of the preceding embodiments, wherein the MC4R agonist is Ac-Arg-c(Cys-D-Ala-His-D-Phe-Arg-Trp-Cys)-NH₂ (SEQ ID NO: 140).
33. The method of any one of the preceding embodiments, wherein the MC4R agonist is selected from:

(SEQ ID NO: 36)
Ac-Arg-cyclo[Cys-D-Ala-His(3-Me)-D-Phe-Arg-Trp-
Cys]-NH₂;
(SEQ ID NO: 37)
Ac-Arg-cyclo[Cys-D-Ala-His(1-Me)-D-Phe-Arg-Trp-
Cys]-NH₂;
(SEQ ID NO: 9)
Ac-Arg-cyclo[Cys-D-Ala-Trp-D-Phe-Arg-Trp-Cys]-NH₂;
(SEQ ID NO: 8)
Ac-Arg-cyclo[Cys-D-Ala-Gln-D-Phe-Arg-Trp-Cys]-NH₂;
(SEQ ID NO: 7)
Ac-Arg-cyclo[Cys-D-Ala-Asn-D-Phe-Arg-Trp-Cys]-NH₂;
(SEQ ID NO: 38)
Ac-Arg-cyclo[Cys-D-Ala-Arg-D-Phe-Arg-Trp-Cys]-NH₂;
(SEQ ID NO: 39)
Ac-Arg-cyclo[Cys-D-Ala-Tyr-D-Phe-Arg-Trp-Cys]-NH₂;
(SEQ ID NO: 40)
Ac-Arg-cyclo[Cys-D-Ala-D-Pro-D-Phe-Arg-Trp-Cys]-
NH₂;
(SEQ ID NO: 2)
Ac-Arg-cyclo[Cys-D-Ala-Pro-D-Phe-Arg-Trp-Cys]-NH₂;
(SEQ ID NO: 4)
Ac-Arg-cyclo[Cys-D-Ala-Pro-D-Phe(p-F)-Arg-Trp-
Cys]-NH₂;
(SEQ ID NO: 41)
Ac-Arg-cyclo[Cys-D-Ala-Atc-D-Phe-Arg-Trp-Cys]-NH₂;
(SEQ ID NO: 42)
Ac-Arg-cyclo[Cys-D-Ala-QAla-D-Phe-Arg-Trp-Cys]-
NH₂;
(SEQ ID NO: 43)
Ac-Arg-cyclo[Cys-D-Ala-sChp-D-Phe-Arg-Trp-Cys]-
NH₂;
(SEQ ID NO: 44)
Ac-Arg-cyclo[Cys-D-Ala-X-D-Phe-Arg-Trp-Cys]-NH₂,
(SEQ ID NO: 15)
Ac-Arg-cyclo[hCys-Ala-D-Phe-Arg-Trp-Cys]-NH₂;
(SEQ ID NO: 14)
Ac-Arg-cyclo[hCys-D-Ala-D-Phe-Arg-Trp-Cys]-NH₂;
(SEQ ID NO: 45)
Ac-Arg-cyclo[hCys-D-Ala-D-Phe-Arg-Trp-Pen]-NH₂;
(SEQ ID NO: 26)
Ac-Arg-cyclo[Glu-D-Ala-D-Phe-Arg-Trp-Dpr]-NH₂;
(SEQ ID NO: 27)
Ac-Arg-cyclo[Glu-Ala-D-Phe-Arg-Trp-Dpr]-NH₂;
(SEQ ID NO: 46)
Ac-Arg-cyclo[hCys-Aib-D-Phe-Arg-Trp-Cys]-NH₂;
(SEQ ID NO: 47)
Ac-Arg-cyclo[hCys-Sar-D-Phe-Arg-Trp-Cys]-NH₂;
(SEQ ID NO: 48)
Ac-Arg-cyclo[hCys-Val-D-Phe-Arg-Trp-Cys]-NH₂;
(SEQ ID NO: 49)
Ac-Arg-cyclo[hCys-D-Val-D-Phe-Arg-Trp-Cys]-NH₂;
(SEQ ID NO: 50)
Ac-Arg-cyclo[hCys-Gln-D-Phe-Arg-Trp-Cys]-NH₂;
(SEQ ID NO: 51)
Ac-Arg-cyclo[hCys-D-Gln-D-Phe-Arg-Trp-Cys]-NH₂;
(SEQ ID NO: 52)
Ac-Arg-cyclo[hCys-Ala-D-Phe-Arg-Trp-Pen]-NH₂;
(SEQ ID NO: 53)
Ac-Arg-cyclo[D-Pen-D-Ala-D-Phe-Arg-Trp-hCys]-NH₂;
(SEQ ID NO: 17)
Ac-Arg-cyclo[Cys-D-Ala-D-Phe-Arg-Trp-hCys]-NH₂;
(SEQ ID NO: 54)
Ac-Arg-cyclo[Pen-D-Ala-D-Phe-Arg-Trp-hCys]-NH₂;
(SEQ ID NO: 55)
Ac-Arg-cyclo[D-hCys-D-Ala-D-Phe-Arg-Trp-Cys]-NH₂;
(SEQ ID NO: 20)
Ac-Arg-cyclo[hCys-Pro-D-Phe-Arg-Trp-Cys]-NH₂;
or
(SEQ ID NO: 56)
Ac-Arg-cyclo[hCys-D-Pro-D-Phe-Arg-Trp-Cys]-NH₂,

or a pharmaceutically acceptable salt thereof.
34. The method of any one of the preceding embodiments, wherein the MC4R agonist is formulated as a pharmaceutical composition.
35. The method of embodiment 34, wherein the pharmaceutical composition further comprises a pharmaceutically acceptable excipient.
36. The method of any one of the embodiments 34-35, wherein the pharmaceutical composition comprises a polyethylene glycol (e.g., a modified polyethylene glycol, e.g., mPEG-DSPE, e.g., mPEG-2,000-DSPE)
37. The method of any one of the embodiments 34-36, wherein the pharmaceutical composition comprises a neutral lipid, a phospholipid, or an alcohol.
38. The method of embodiment 37, wherein the neutral lipid comprises a diacyl glycerol (e.g., glycerol dioleate (GDO)).
39. The method of any one of embodiments 30-31, wherein the phospholipid comprises phosphatidylcholine (e.g., soybean phosphatidylcholine).
40. The method of any one of embodiments 30-32, wherein the alcohol comprises ethanol.
41. The method of any one of the preceding embodiments, wherein the MC4R agonist is formulated as a unit dosage form.
42. The method of embodiment 41, wherein the dosage of the MC4R agonist is between about 0.01 mg to 100 mg.
43. The method of any one of embodiments 41-42, wherein the dosage of the MC4R agonist is between about 1 mg and 20 mg.
44. The method of any one of the preceding embodiments, wherein the MC4R agonist is administered daily, weekly, or monthly.
45. The method of any one of the preceding embodiments, wherein the MC4R agonist is administered 1, 2, or 3 times daily.
46. The method of any one of the preceding embodiments, wherein the MC4R agonist is administered 1, 2, 3 times weekly.
47. The method of any of the preceding embodiments, comprising administering the agonist in a unit dosage suitable for injection, e.g., subcutaneous injection, to the subject.
48. The method of any one of embodiments 41-47, wherein the unit dosage form is disposed within a delivery device, e.g., a syringe (e.g., prefilled syringe), an implantable device, a needleless hypodermic injection device, an infusion pump (e.g., implantable infusion pump), or an osmotic delivery system.
49. The method of any of the preceding embodiments, wherein the MC4R agonist is administered subcutaneously, e.g., by subcutaneous injection.
50. The method of any of the preceding embodiments, wherein the MC4R agonist is administered daily over a period of at least 3 weeks, e.g., at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 weeks or more, or at least 1, 2, 3, 4, 5, 6, 7, 8, 9, 10, 11, or 12 months or more, or at least 1, 2, 3, 4 years or more.
51. The method of any of the preceding embodiments, wherein the subject is obese, e.g., severely obese.
52. The method of any of the preceding embodiments, wherein the subject has early onset severe obesity.
53. The method of any of the preceding embodiments, wherein the subject is hyperphagic.
54. The method of any of the preceding embodiments, wherein the subject has a body mass index (BMI) greater than 25 kg/m² (e.g., ≥25, 30, 31, 32, 33, 34, 35, 36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration.
55. The method of any of the preceding embodiments, wherein the subject has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration.
56. The method of any of the preceding embodiments, wherein the subject has a body mass index (BMI) greater than 40 kg/m² (e.g., ≥41, 42, 43, 44, 45, 46, 47, 48, 49, 50, 51, 52, 53, 54, 55 kg/m² or greater) prior to administration of the MC4R agonist, e.g., at the time the MC4R agonist is prescribed, or at the time of the first administration.
57. The method of any of the preceding embodiments, wherein the subject has failed one or more previous therapies, e.g., exercise, diet, or behavioral therapies, prior to administration of the MC4R agonist, e.g., at the time the MC4R agonist is prescribed, or at the time of the first administration.
58. The method of any of the preceding embodiments, wherein the subject has a lower body weight after administration of the MC4R agonist than before administration of the MC4R agonist.
59. The method of any of the preceding embodiments, wherein administration of the MC4R agonist results in a reduction of weight in the subject compared to the weight of the subject before treatment of about 1 kg to 3 kg after 1 week of treatment, or about 1 kg to 6 kg after 2 weeks of treatment, or about 2 kg to 12 kg after 4 weeks of treatment, or about 4 kg to 24 kg after 8 weeks of treatment, or about 8 kg to 48 kg after 16 weeks of treatment.
60. The method of any of the preceding embodiments, wherein administration of the MC4R agonist results in weight loss in the subject at a rate of about 1-2 kg/week, e.g., about 2 kg/week, e.g., over a period of 1-2 weeks of treatment or longer, 2-4 weeks of treatment or longer, 4-8 weeks of treatment or longer, 8-16 weeks of treatment or longer, 16-32 weeks or longer, or 32-64 weeks or longer.
61. The method of any of the preceding embodiments, wherein administration of the MC4R agonist results in a reduction in hunger level (e.g., a lower score on the Likert hunger scale, e.g., a lower score by at least 1, 2, 3, 4, 5, 6, 7, 8, 9 or 10 points) in the subject compared to the hunger level of the subject before treatment, e.g., results in abolishment of hunger (e.g., a score of 0 on the Likert hunger scale) in the subject, e.g., after 1-2 weeks of treatment or longer, 2-4 weeks of treatment or longer, 4-8 weeks of treatment or longer, or 8-16 weeks of treatment or longer.
62. The method of any of the preceding embodiments, wherein administration of the MC4R agonist results in no detectable/significant decrease in resting energy expenditure (REE) in the subject, e.g., over a period of 24 hours, one week, or 30 days or longer, e.g., as compared to a control REE (e.g., the REE in the subject prior to treatment or a predetermined REE, e.g., in subjects of similar pre-treatment BMI, e.g., when expressed as REE per kg of lean body mass).
63. The method of embodiment 62, wherein administration of the MC4R agonist results in an increase in resting energy expenditure (REE) in the subject, e.g., over a period of 24 hours, one week, or 30 days, or longer e.g., as compared to a control REE (e.g., the REE in the subject prior to treatment or compared to a predetermined REE, e.g., in subjects of similar pre-treatment BMI, when expressed as REE per kg of lean body mass, e.g., after a similar level of weight loss has been attained by fasting).
64. The method of any of the preceding embodiments, wherein administration of the MC4R agonist results in a reduction in food intake by the subject compared to a control (e.g., the food intake of the subject prior to treatment), e.g., wherein the food intake is daily food intake or food intake over a period of 24 hours, or one week.
65. The method of embodiment 64, wherein administration of the MC4R agonist results in a reduction in food intake of at least 100 kilocalories, e.g., at least 100, 125, 150, 175, 200, 225, 250, 275, 300, 325, 350, 375, 400, 425, 450, 475, 500, 525, 550, 575, 600, 1000 kilocalories or more, compared to a control (e.g., the food intake of the subject prior to treatment or a predetermined food intake level), e.g., wherein the food intake is daily food intake or food intake over a period of 24, hours, or one week.
66. The method of any of the preceding embodiments, wherein administration of the MC4R agonist results in a reduction in waist circumference of the subject compared to a control (e.g., the waist circumference of the subject prior to treatment), as measured 1, 2, 3, 4, 5, 6, 7, 8, 9, 10 weeks or more after initiation of treatment.
67. The method of embodiment 66, wherein administration of the MC4R agonist results in a reduction in waist circumference of at least 2 cm (e.g., at least 2, 3, 4, 5, 6, 7, 8, 9, 10 cm or more) in the subject compared to a control (e.g., the waist circumference of the subject prior to treatment), as measured 1, 2, 3, 4, 5, 6, 7, 8, 9, 10 weeks or more after initiation of treatment.
68. The method of any of the preceding embodiments, wherein administration of the MC4R agonist results in no detectable increase in blood pressure (e.g., diastolic and/or systolic blood pressure) of the subject compared to the blood pressure of the subject prior to treatment, as measured 1, 2, 3, 4, 5, 6, 7, 8, 9, 10 weeks or more after initiation of treatment.
69. The method of any of the preceding embodiments, wherein administration of the MC4R agonist results in a reduction in blood pressure (e.g., diastolic and/or systolic blood pressure) of the subject compared to the blood pressure of the subject prior to treatment, as measured 1, 2, 3, 4, 5, 6, 7, 8, 9, 10 weeks or more after initiation of treatment.
70. The method of embodiment 69, wherein administration of the MC4R agonist results in a reduction in systolic blood of the subject of at least 3 mmHg (e.g., at least 3, 3.5, 4, 4.5, 5, 5.5, 6, 6.5, 7 mmHg or more) compared to the blood pressure of the subject prior to treatment, as measured 1, 2, 3, 4, 5, 6, 7, 8, 9, 10 weeks or more after initiation of treatment.
71. The method of claim 70, wherein administration of the MC4R agonist results in a reduction in diastolic blood pressure of the subject of at least 4 mmHg (e.g., at least 4, 7, 7.5, 8, 8.5, 9, 9.5, 10 mmHg or more) compared to the blood pressure of the subject prior to treatment, as measured 1, 2, 3, 4, 5, 6, 7, 8, 9, 10 weeks or more after initiation of treatment.
72. The method of any of the preceding embodiments, wherein administration of the MC4R agonist results in a reduction of hunger in the subject compared to the hunger of the subject prior to treatment, as determined 1, 2, 3, 4, 5, 6, 7, 8, 9, 10 weeks or more after initiation of treatment.
73. The method of any of the preceding embodiments, wherein the subject is a mammal, e.g., a human.
74. The method of any of the preceding embodiments, comprising acquiring knowledge of the genotype of the subject, e.g., acquiring knowledge of the genotype of the MC4R pathway agonizable gene.
75. The method of embodiment 74, wherein the MC4R agonist is administered in response to the detection of a predetermined sequence, e.g., a mutation, in the MC4R pathway agonizable gene.
76. A method of treating a disease, disorder, or condition in a subject in need thereof, comprising:
administering an agonist of the melanocortin-4 receptor (MC4R) having a structure of Formula (I):

(R²R³)-A¹-c(A²-A³-A⁴-A⁵-A⁶-A⁷-A⁸-A⁹)-A¹⁰-R¹ (I),

wherein:
A¹ is Acc, HN—(CH₂)ₘ—C(O), L- or D-amino acid, or deleted;
A² is Cys, D-Cys, hCys, D-hCys, Pen, D-Pen, Asp, or Glu;
A³ is Gly, Ala, β-Ala, Gaba, Aib, D-amino acid, or deleted;
A⁴ is H is, 2-Pal, 3-Pal, 4-Pal, Taz, 2-Thi, 3-Thi, or (X¹, X², X³, X⁴, X⁵)Phe;
A⁵ is D-Phe, D-1-Nal, D-2-Nal, D-Trp, D-Bal, D-(X¹, X², X³, X⁴, X⁵)Phe, L-Phe or D-(Et)Tyr;
A⁶ is Arg, hArg, Dab, Dap, Lys, Orn, or HN-CH((CH₂)ₙ—N(R⁴R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂)ₛ—C(O), or deleted;
A⁹ is Cys, D-Cys, hCys, D-hCys, Pen, D-Pen, Dab, Dap, Orn, or Lys;
A¹⁰ is Acc, HN—(CH₂)ᵣ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN,
wherein the subject has a mutation in a gene selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, TRPC5, PHIP, and MeCP2.
77. A method of treating a disease, disorder, or condition in a subject comprising administering to the subject melanocortin-4 receptor (MC4R) agonist, wherein the disease, disorder, or condition is related to an melanocortin-4 receptor (MC4R) agonizable.
78. The method of embodiment 77, wherein the PCSK1 mutation is N221D.
79. A composition for use treating a disease, disorder, or condition in a subject comprising administering to the subject melanocortin-4 receptor (MC4R) agonist, wherein the disease, disorder, or condition is related to an MC4R pathway agonizable gene.
80. The composition for use of embodiment 80, wherein the MC4R agonist is a compound described herein.
81. The composition for use of embodiments 80-81, wherein the MC4R pathway agonizable gene is a gene described herein.

### EXAMPLES

### Example 1: An Open-Label Study to Evaluate the Safety and Efficacy of Setmelanotide in Subjects with Hypothalamic Obesity

### Purpose

This example describes a phase 2 study that seeks to evaluate the change in body weight in response to setmelanotide administered subcutaneously (SC) daily in patients with hypothalamic obesity (HO). Additional objectives of this study include evaluation of changes in parameters of body weight, body mass index (BMI), waist circumference, and hunger in response to setmelanotide in patients with HO and to evaluate the safety and tolerability of setmelanotide in patients with HO. In addition, this study seeks to evaluate changes in weight and BMI in patients of different age groups and changes in metabolic parameters following treatment with setmelanotide.

### Investigational Study Drug

Setmelanotide, 10 mg/mL in a sterile solution for injection, will be provided at 1.0, 2.0, 3.0 mg QD for patients 6 to <16 years of age, and 2.0 to 3.0 mg QD for patients ≥16 years of age.

### Primary Endpoints

The primary endpoint of this study is to identify a proportion of patients with ≥5% reduction from baseline in BMI after 16 weeks of setmelanotide treatment compared to a historic control of <5% in this patient population.

### Secondary Endpoints

The secondary endpoints include determination of:
Composite proportion of patients aged ≥6 to ≤18 years with 0.2 reduction of BMI Z-score and patients aged ≥18 years with 5% reduction of body weight from baseline after 16 weeks of setmelanotide
Proportion of patients aged ≥6 to ≤18 years with ≥0.2 reduction of BMI Z-score from baseline after 16 weeks of setmelanotide
Proportion of patients aged ≥18 years with ≥5% reduction of body weight from baseline after 16 weeks of setmelanotide
Change from baseline in waist circumference in patients aged 18 years after 16 weeks of setmelanotide treatment.
Change in hunger in response to 16 weeks of setmelanotide treatment as measured by change from baseline in daily and global hunger scores.
Safety and tolerability assessed by the frequency and severity of AEs, vital signs, and laboratory evaluations.
Change from baseline in metabolic parameters following treatment with setmelanotide, including fasting glucose, HbA1c, lipid profiles (total-, high-density lipoprotein [HDL]-, and low-density lipoprotein [LDL]-cholesterol, triglycerides).
Proportion of patients, regardless of age, with ≥5% body weight loss from baseline after 16 weeks of setmelanotide treatment compared to a historic control of <5% in this patient population.
Change from baseline in BMI, weight, and waist circumference, regardless of age, after 16 weeks of setmelanotide treatment.
Change from baseline in BMI Z-score in patients aged 6 to 12 years and in those aged 6 to <18 years.

### Study Design Rationale

This study describes a Phase 2, multicenter, open-label, proof of concept study designed to assess the effect of setmelanotide on weight loss within a population affected by HO. Approximately 15 patients aged 6 to 40 years, inclusive, are planned to be enrolled across approximately 3-5 clinical sites in the United States (US). Setmelanotide is being evaluated as a potential treatment for obesity in rare mechanistically induced populations with hypothalamic injury and subsequent obesity. The open-label design is supported by the compelling efficacy results from earlier pivotal studies in patients with POMC and LEPR deficiency obesity that demonstrate that setmelanotide induces rapid and sustained significant and clinically meaningful weight loss accompanied by statistically significant and clinically meaningful reduction in hunger in these patient populations.

### Screening Period

Upon providing informed consent, patients will enter the Screening Period, during which they will be assessed for eligibility and complete all screening procedures.

### Treatment Period

Patients who are determined to be eligible, based on Screening assessments, will return to the clinic for the Baseline Visit (Visit 2) and receive the first setmelanotide dose. The starting setmelanotide dose is dependent on patient age; however, for all patients, the setmelanotide dose will be titrated to a final dose of 3.0 mg/day (initial titration phase), as follows

### Patients Aged 6 to <16 Years, Inclusive

Starting at Baseline (Day 1; Visit 2) through approximately Day 14, patients will receive setmelanotide 1.0 mg/day.
Starting at approximately Day 15 through 28, patients will receive setmelanotide 2.0 mg/day.
   Patients/caregivers will be contacted by study center personnel on Day 15 to ensure dose titration has occurred and to document any AEs.
Starting on approximately Day 29 (Visit 3), patients will receive setmelanotide 3.0 mg/day; patients will continue to receive setmelanotide 3.0 mg/day for 12 weeks.

### Patients Aged ≥16 Years, Inclusive

Starting at Baseline (Day 1; Visit 2) through approximately Day 14, patients will receive setmelanotide 2.0 mg/day.
Starting at approximately Day 15, patients will receive setmelanotide 3.0 mg/day; patients will continue to receive setmelanotide 3.0 mg/day for 14 weeks.
   Patients/caregivers will be contacted by study center personnel on Day 15 to ensure dose titration has occurred and to document any adverse events.
   Patients will return to the study center for Visits 3, 4, and 5 (Weeks 4, 8, and 12, respectively), with each of these visits conducted approximately 4 weeks apart. All patients will return to the study center at Week 16 (Visit 6) and receive the last setmelanotide injection. Study endpoints are analyzed at Visit 6. After completion of Visit 6, participation in the current study will then conclude in one of the following 2 ways:
Patients meeting the primary endpoint may be eligible to be enrolled in a separate extension study under which auspices the patient will continue to receive setmelanotide.
Patients who do not meet the primary endpoint or elect not to continue setmelanotide are to discontinue setmelanotide at Visit 6 and return for an End-of-Study (EOS) Visit (Visit 7) 4 weeks thereafter for a final safety review under the auspices of the current study

### Inclusion Criteria

Patients must meet the following criteria to be eligible for study participation:
1. Patient has documented evidence of HO, including:
   a. Recent (within the previous 8 months before Screening) evidence of hypothalamic injury on magnetic resonance imaging (MRI); and
   b. Diagnosis of craniopharyngioma or other non-malignant brain tumor affecting the hypothalamic region; and
   c. Has undergone surgery, or chemotherapy, or radiation ≥6 months and ≤15 years before screening.
2. Patient has either unilateral hypothalamic lesions (at least 6 patients) or bilateral hypothalamic lesions (at least 7 patients), as assessed by MRI.
3. Aged 6 to 40 years, inclusive, at time of enrollment.
4. Obesity, documented by a BMI ≥35 kg/m² for patients ≥18 years of age or BMI ≥95th percentile for age and gender for patients 6 to <18 years of age based on the US Centers for Disease Control and Prevention criteria.
5. Documented increase in BMI (change from pre-surgery baseline in BMI Z-score ≥0.2 for patients <18 years of age or BMI >5% for patients ≥18 years of age) either during the first 6 months following surgery or within 1 year before surgery and still present at Screening.
6. More than 6 months after the end of post-tumor treatment, including chemotherapy, surgery, or radiation.
7. Patient must meet one of the following contraception requirements:
   If a female of childbearing potential, defined as fertile, following menarche and until becoming post-menopausal unless permanently sterile (hysterectomy, bilateral salpingectomy, or bilateral oophorectomy), must use a highly effective form of contraception.
   If a female of non-childbearing potential, defined as permanently sterile (status post hysterectomy, bilateral oophorectomy, or bilateral salpingectomy) or post-menopausal for at least 12 months (and confirmed with a screening follicle-stimulating hormone [FSH] level in the post-menopausal laboratory range), contraception is not required during the study.
   Younger female patients who have not reached menarche upon study entry will be assessed for Tanner staging and at first menarche will be required to comply with contraception requirements and pregnancy testing as outlined in the protocol.
   If a male with female partner(s) of childbearing potential, must agree to a double barrier method if they become sexually active during the study. Furthermore, male patients must not donate sperm during and for 90 days following their participation in the study.
8. Ability to communicate well with the Investigator, understand and comply with the requirements of the study, and understand and sign the written informed consent, or, for patients aged <18 years, a parent/legal guardian that can sign.
9. If receiving hormone replacement therapy (i.e., thyroid hormones, glucocorticoids, growth hormone or other medications known to affect metabolism or weight/body composition), the dose of such therapy has remained stable for at least 2 months prior to Screening.

### Exclusion Criteria

Patients meeting any of the following criteria are not eligible for study participation:
1. Weight gain >5% in the previous 3 months.
2. Weight loss ≥2% in the previous 3 months.
   Note: Dietary and/or exercise regimens, with or without the use of medications, supplements or herbal treatments associated with weight loss (e.g., orlistat, lorcaserin, phentermine, topiramate, naltrexone, bupropion, glucagon-like peptide-1 [GLP-1] receptor agonists, etc.) are allowed if:
the regimen and/or dose has been stable for at least 3 months prior to randomization
the patient has not experienced weight loss ≥2% during the previous 3 months, and
the patient intends to keep the regimen and/or dose stable throughout the course of the study.
3. Bariatric surgery or procedure (e.g., gastric bypass/band/sleeve, duodenal switch, gastric balloon, intestinal barrier, etc.) within the last 6 months. All patients with a history of bariatric surgery or procedures must be discussed with and receive approval from the Sponsor prior to enrollment.
4. Diagnosis of severe psychiatric disorders (e.g., schizophrenia, bipolar disorder, personality disorder), or Major Depressive Disorder (MDD) within the previous 2 years, or Screening Patient Health Questionnaire (PHQ)-9/PHQ-A score ≥15, or any suicidal ideation of type 4 or 5 on the Columbia-Suicide Severity Rating Scale (C-SSRS) during Screening, or lifetime history of suicide attempts, or any suicidal behavior in the last month.
5. Glycated hemoglobin (HbA1c) ≥10.0% at Screening.
6. Current, clinically significant pulmonary, cardiac, or oncologic disease considered severe enough to interfere with the study and/or confound the results. Any patient with a potentially clinically significant disease should be reviewed with the Sponsor to determine eligibility.
7. Glomerular filtration rate (GFR)<30 mL/min/1.73 m² during Screening.
8. Significant dermatologic findings relating to melanoma or pre-melanoma skin lesions (excluding non-invasive basal or squamous cell lesion), determined as part of a comprehensive skin evaluation performed by the Investigator during Screening. Any concerning lesions identified during Screening will be biopsied and results known to be benign prior to enrollment. If the pre-treatment biopsy results are of significant concern, the patient is not eligible for study participation.
9. History or close family history (parents or siblings) of skin cancer or melanoma (not including noninvasive, infiltrative basal or squamous cell lesion), or patient history of ocular-cutaneous albinism.
10. Participation in any clinical study with an investigational drug/device within 3 months or 5 half-lives, whichever is longer, prior to the first setmelanotide dose.
11. Previously enrolled in a clinical study involving setmelanotide or any previous exposure to setmelanotide.
12. Inability to comply with once daily (QD) injection regimen.
13. Pregnant and/or breastfeeding or desiring to become pregnant during this trial.
14. Cognitive impairment that, in the Investigator's opinion, precludes participation to the study and completions of study procedures or questionnaires.
15. Patient is, in Investigator's opinion, otherwise not suitable to participate in the study.

### Duration of Treatment

All patients will receive study treatment for 16 weeks. Total study participation will last between 22 and 28 weeks, based on the variable length of the Screening Period

### Treatment

All patients will receive open-label setmelanotide in this study. Setmelanotide will be provided as a preserved, multidose solution for injection to be administered SC, QD. It is formulated as a 10 mg/mL sterile, preserved, clear to slightly opalescent, colorless to slightly colored solution, practically free of visible particles. The drug product solution (1 mL) is presented in a clear 2R glass vial with a rubber stopper. Packaging and labeling will be prepared to meet all regulatory requirements. Setmelanotide will be administered as SC injection QD.
All patients will receive setmelanotide in this study. The starting setmelanotide dose is dependent on patient age at the time of Visit 2 (Day 1); however, for all patients, the setmelanotide dose will be titrated to a final dose of 3.0 mg/day (initial titration phase), as follows:

### Patients Aged 6 to <16 Years

Starting at Baseline (Day 1; Visit 2) through approximately Day 14, patients will receive setmelanotide 1.0 mg/day
   Starting at approximately Day 15 through 28, patients will receive setmelanotide 2.0 mg/day.
Patients/caregivers will be contacted by study center personnel on Day 15 to ensure dose titration has occurred and to document any adverse events.
Starting on approximately Day 29 (Visit 3), patients will receive setmelanotide 3.0 mg/day; patients will continue to receive setmelanotide 3.0 mg/day for 12 weeks.

### Patients Aged ≥16 Years

Starting at Baseline (Day 1; Visit 2) through approximately Day 14, patients will receive setmelanotide 2.0 mg/day.
Starting at approximately Day 15, patients will receive setmelanotide 3.0 mg/day; patients will continue to receive setmelanotide 3.0 mg/day for 14 weeks.

### Concomitant Therapy

Any medication or vaccine (including over-the-counter or prescription medicines, vitamins, and/or herbal supplements) that the patient is receiving at the time of enrollment or receives during the study must be recorded along with:
Reason for use
Dates of administration including start and end dates
Dosage information including dose and frequency
   The Medical Monitor should be contacted if there are any questions regarding concomitant or prior therapy.

### Prohibited Medications

Unless concomitant medications are likely to present a strong potential safety concern, the general goal of this protocol is to allow as many patients with this rare condition to participate in the study as possible. However, patients are not permitted to enter the study if they have had weight gain >5% during the previous 3 months or >2% weight loss during the prior 3 months. It should be noted that dietary and/or exercise regimens, with or without the use of medications, supplements or herbal treatments associated with weight loss (e.g., orlistat, lorcaserin, phentermine, topiramate, naltrexone, bupropion, glucagon-like peptide1 [GLP1] receptor agonists, etc.) are allowed if:
the regimen and/or dose has been stable for at least 3 months prior to randomization
the patient has not experienced weight loss ≥2% during the previous 3 months, AND the patient intends to keep the regimen and/or dose stable throughout the course of the study.
   GLP-1 receptor agonists may be used up to the dose approved for the treatment of diabetes mellitus (e.g., liraglutide up to a daily dose of 1.8 mg) as long as (1) it is not being prescribed for the treatment of obesity, (2) the dose has been stable for at least 3 months prior to enrollment, (3) the patient has not experienced >3% weight loss during the previous 3 months, and (4) the patient intends to keep the dose stable throughout the course of the study. Other medications that may cause weight loss (e.g., stimulants) are allowed as long as the patient (1) has used a stable dose for at least 3 months prior to enrollment, (2) has not lost weight during the previous 3 months, and (3) intends to keep the dose of the medication stable through the course of the study.
   All concomitant medications should be kept at a stable dose throughout the course of the study, unless a dose change is necessary to treat an AE.
   Treatment after the End of the Study
   After the EOS Visit, no further treatment is planned under the auspices of this protocol. Patients who meet the primary endpoint of this study may be eligible to enroll in a separate long-term extension study under which auspices the patient would continue to receive setmelanotide.

### Discontinuation and Withdrawal Criteria

Given this rare patient population, every effort will be made to encourage and keep patients enrolled in the study until completion, unless there are any safety concerns necessitating withdrawal of the patient. If the patient withdraws consent for disclosure of future information, the Sponsor may retain and continue to use any data collected before such a withdrawal of consent. If a patient withdraws from the study, he/she may request destruction of any samples taken and not tested, and the Investigator must document this in the site study records. Patients will be informed that they have the right to withdraw from the study at any time for any reason, without prejudice to their medical care. The Investigator also has the right to withdraw patients from the study, after discussion with the Sponsor, for reasons such as:
adverse events, which justify treatment or study withdrawal.
Non-adherence to study drug regimen or protocol requirements.
Non-compliance with instructions or failure to return for follow-up

### Patient Assessments

All screening evaluations must be completed and reviewed to confirm that potential patients meet all eligibility criteria. Exemplary evaluations include a medical history review, physical examination, comprehensive skin examination, measurement of height, weight, waist circumference, body composition assessment, pregnancy test (if applicable), daily hunger questionnaires, global hunger assessment, and evaluation of PHQ-A or PHQ-9, C-SSRS, SF-12 or SF-10, IWQOL, FSH, HbA1c, and fasting lipid panel. Adjustments may be made depending upon specific circumstances and in consultation with the investigator. Immediate safety concerns should be discussed with the sponsor immediately upon occurrence or awareness to determine if the patient should continue or discontinue study treatment.

### Example 2: A Two-Stage (Open-Label Run-In Followed by Randomized Withdrawal) Study of Setmelanotide in Subjects with Specific Gene Defects in the MC4R Pathway

### Purpose

This example describes a Phase 2 open label study that seeks to evaluate the proportion of obese patients with genetic defects in the MC4R pathway who achieve a reduction in body weight in response to setmelanotide treatment. Additional objectives include evaluating the change in metabolic parameters in patients with genetic defects, as well as gaining a deeper understanding of the safety of setmelanotide treatment in patients with genetic defects.

### Investigational Study Drug

Setmelanotide, 10 mg/mL in a sterile solution for injection. For patients 12 years of age and older, setmelanotide will be provided at 2 mg once daily (QD) for approximately 14 days, then increased to setmelanotide 3 mg QD for the remainder of the study. For patents between 6 and less than 12 years of age, setmelanotide will be provided at 1 mg once daily (QD) for approximately 7 days, then increased to setmelanotide 2 mg QD for approximately 7 days, then increased to setmelanotide 3 mg QD for the remainder of the study.

### Endpoints

### Primary:

To evaluate the proportion of obese patients with genetic defects in the melanocortin-4 receptor (MC4R) pathway who achieve a clinically meaningful reduction in body weight in response to setmelanotide after an initial response to open-label treatment

### Secondary:

To evaluate the initial response to open-label treatment with setmelanotide in patients with genetic defects in the MC4R pathway
To evaluate change in weight, waist circumference, hunger and quality of life in response to setmelanotide in patients with genetic defects in the MC4R pathway
To evaluate the safety and tolerability of setmelanotide in patients with genetic defects in the MC4R pathway

### Exploratory:

To evaluate the change in metabolic parameters following treatment with setmelanotide
To evaluate by gene: the proportion of patients with a meaningful reduction in body weight (responders), mean change in body weight, and change in hunger score

### Study Design Rationale

### Overall Study Design

This is a 2-stage (open-label run-in followed by randomized withdrawal), double-blind, placebo-controlled, Phase 2 study of setmelanotide in obese patients with specific gene defects in the MC4R pathway. Approximately 130 patients between the ages of 6 and 65 years, inclusive, are planned to be enrolled in Stage 2 (randomized withdrawal) of the study with approximately 500 patients enrolled in Stage 1 (open-label run-in) to meet the Stage 2 target.

### Screening Period

Upon providing informed consent, patients will enter the Screening Period, during which they will be assessed for eligibility and complete all standard screening procedures. During the Screening Period, patients will undergo medical evaluation and training on injection of study medication and other study procedures. Patients will be issued an electronic diary to capture daily compliance with injections (post Enrollment) and hunger score assessments (starting during Screening).

### Stage 1 (Open-Label Run-In)

Stage 1 of the study begins with the enrollment visit (Study Day 1). During the enrollment visit, patients will undergo all screening procedures and it will be reconfirmed that the patient continues to meet the Inclusion and Exclusion criteria. At the enrollment visit, the study center must confirm that the patient completed the electronic diary at least 4 of 7 days prior to the enrollment visit. If the diary was not appropriately completed, the patient may not enter the study.
At the enrollment visit, patients will enter Stage 1 of the study. During this stage, the patient will self-inject setmelanotide on a daily basis for 16 weeks. During this period, the patient will have virtual visits using a validated Telehealth platform with the study center. During the virtual visits, the patient will record a body weight measurement and be assessed for compliance and adverse events (AEs). Any visit that is planned as a virtual visit may be converted to an in-person visit, at the discretion of the Primary Investigator. If more than 2 virtual visits are to be converted into in-person visits, Sponsor approval is required.
To be eligible to enter Stage 2 of the study, a patient ≥18 years old must have achieved a body weight of at least 5% less than the Baseline Weight at the end of Stage 1 and a patient <18 years old must have achieved a decrease in body mass index (BMI) Z-score of at least 0.10.
If the patient completed the full 16 weeks of Stage 1 and at the Day 112 visit (Week 16) the patient has not achieved the required change in body weight or BMI Z-score since the Baseline Visit, then instead of the Stage 2 Entry Visit, the site will perform the End-of-Study (EOS) Visit for this patient. These patients will end treatment with setmelanotide and continue to be monitored for weight and resolution of any ongoing AEs with virtual visits once every 4 weeks until all AEs have resolved.

### Stage 2 (Double-Blind, Placebo-Controlled Randomized Withdrawal)

Patients who enter Stage 2 will continue in the study for an additional 24 weeks. Stage 2 of the study will begin with the Stage 2 Entry Visit. During the Stage 2 Entry Visit, the patient will complete all assessments. The patient will have a body weight recorded. This measurement will be their Stage 2 Entry Weight Measurement.
At the Stage 2 Entry Visit, all patients will be randomized 2:1 to either continue daily setmelanotide or receive matching placebo. Stratification by gene will occur for specific, more prevalent genes being enrolled into this study.
Patients will continue to have virtual visits and in-person clinic visits as per the schedule of events.
At the Primary Investigator's discretion, either (1) additional virtual or in-person visits may be scheduled or (2) planned virtual visits may be converted to in-person visits. If more than 2 virtual visits are to be converted into in-person visits, Sponsor approval is required.

### End of Treatment

The End of Treatment Visit will occur as in-person clinic visit on Study Day 280, which is the final day of treatment with setmelanotide or placebo. A final EOS visit will occur on Study Day 308. The EOS Visit will be conducted via telephone. Patients who respond to setmelanotide may be offered the option of enrolling into a Long-Term Extension study.

### Rescue

During Stage 2 of the study, patient weight will be monitored. If during a visit, a patient's body weight has increased by at least 5% from the Stage 2 Entry Weight, that patient will be eligible for rescue. If the Primary Investigator believes that it is in the best medical interest of the patient to stop double-blind treatment and instead to re-start open-label setmelanotide, the patient may convert to open-label treatment with setmelanotide.
In this situation, a patient must be scheduled for an in-person Rescue Visit at the clinic. At the Rescue Visit, the patient's body weight will be recorded. For a patient who is rescued, they will be considered a “non-responder” in assessing the Primary Endpoint of the study. At this visit, the patient will return their double-blind study drug supply and be issued open-label setmelanotide. The patient may then continue in the study, completing all visits and assessments. The patient's initial assignment to either study drug or placebo will continue to be blinded to all parties, but the patient will be provided open-label setmelanotide for the reminder of the study.

### Ensuring Diversity of Genes

A patient must have a pre-identified genetic variant in an established MC4R pathway gene that contributes to obesity to enroll in this study. A list of genes that have variants that are eligible for enrollment into the study include LEP, ISL1, DNMT3A, TRPC5, PLCNA4, NRP1, SEMA3E, SEMA3F, MECP2, SEMA3A, SEMA3C, PHIP, NRP2, MRAP2, MC3R, CPE, SEMA4B, SEMA3D, SIM1, HTR2C, SEMA3G, KSR2, MC4R, MAGEL2, RPGRIP1L, TBX3, PLCNA1, CREBBP, PLXNA1, PLXNA2, TUB.
The goal of the study is to enroll approximately 30 patients with each gene into the study. Enrollment will be monitored by the Sponsor and further enrollment of patients with a genotype will be paused once 30 patients with that particular gene have been enrolled into the study.
Patient response to setmelanotide by gene will be monitored by the Sponsor during the open-label portion of the study in 2 ways: the rate of patients qualifying for Stage 2 of the study and the magnitude of response to setmelanotide. Additionally, after the last planned patient with a particular gene has completed the study, the data for patients with that gene will be unblinded and analyzed for efficacy and safety. Based on these emerging data, the sample size of the study and/or the number of patients enrolled with a particular gene (i.e. approximately 30) may be adjusted. The Sponsor may increase or decrease the number of patients enrolled with a particular gene or close the study prior to enrolling 500 patients. The total sample size of the study will not be increased

### Inclusion Criteria

1. Patients must have a pre-identified genetic variant in an established MC4R pathway gene that contributes to obesity.

### Note:

For a gene variant to be eligible for inclusion in the study, the variant must be categorized by aCLIA/CAP/ISO15189certified laboratory using American College of Medical Genetics (ACMG) criteria as (1) Pathogenic, (2) Likely Pathogenic, or (3) a Variant of Uncertain Significance (VUS). In the case where an investigator has genetic results on a patient who may be eligible for the study, but the genetics have not yet been categorized by a CLIA/CAP/ISO15189certified laboratory, then the Sponsor may provide testing and/or categorization through a third-party laboratory.
2. Patients between the ages of 6 and 65, inclusive, at the time of signing Informed Consentor Assentare eligible for the study.
3. Obese, defined as BMI≥40 kg/m² for patients ≥18 years of age or BMI ≥97th percentile for age and gender for patients 6 up to <18 years of age based on the United States (US)Centers for Disease Control and Prevention criteria.
4. Study participant and/or parent or guardian is able to communicate well with the Investigator, to understand and comply with the requirements of the study (including QD injection regimen and all other study procedures) and is able to understand and sign the written informed consent/assent. Patients who are unable to comply with all study procedures due to cognitive limitations or any other reason should not be enrolled into the study.
5. If male or a childbearing female, including pre-pubertal females if relevant, agrees to use a highly reliable form of contraception throughout the study and for 90 days following the study. Highly reliable acceptable forms of contraception include hormonal (i.e., oral, implantable, or injectable) AND single-barrier method (i.e., condom), or an intrauterine device (IUD) AND single-barrier method (i.e., condom) or vasectomy/vasectomized partner. True abstinence is acceptable only if it is the preferred and usual lifestyle of the patient. Female participants of non-childbearing potential, defined as surgically sterile (status post hysterectomy, bilateral oophorectomy, or bilateral tubal ligation), post-menopausal for at least 12months (and confirmed with a screening follicle-stimulating hormone [FSH] level in the post-menopausal lab range), and failure to have achieved menarche, do not require contraception during the study. Female patients must not become pregnant and male patients must not donate sperm during and for 90 days following their participation in the study.

### Exclusion Criteria:

1. Recent intensive (within 2months) diet and/or exercise regimen with or without the use of weight loss agents including herbal medications that has resulted in >3% weight loss.
2. Use of any medication that is approved to treat obesity within 3months of first dose of study drug (e.g., orlistat, lorcaserin, phentermine-topiramate, naltrexone-bupropion).
   Note: Glucagon-like peptide-1 (GLP-1) receptor agonists may be used up to the dose approved for the treatment of diabetes mellitus (e.g., liraglutide up to a daily dose of 1.8 mg) as long as (1) it is not being prescribed for the treatment of obesity, (2) the dose has been stable for at least 3months prior to enrollment, (3) the patient has not experienced weight loss during the previous 3months, AND (4) the patient intends to keep the doses table throughout the course of the study.
3. Bariatric surgery within the previous 6 months.
   Note: Patients with a history of gastric bypass surgery should have documented evidence of stable weight, defined as weight loss in the last 3months of less than 3% of body weight.
4. Diagnosis of schizophrenia, bipolar disorder, personality disorder, major depressive disorder, or other psychiatric disorder(s) that the Investigator believes will interfere significantly with study compliance.
5. Any suicidal ideation of type 4 or 5 on the Columbia-Suicide Severity Rating Scale (C-SSRS) during Screening, any suicide attempt in the past 20 years or any suicidal behavior in the last month.
6. Current, clinically significant pulmonary, cardiac, or oncologic disease considered severe enough to interfere with the study and/or confound the results. Any patient with a potentially clinically significant disease should be reviewed with the Sponsor to determine eligibility.
7. Has significant features of (or meets the diagnostic criteria for) a genetic syndrome that is associated with obesity, such as Tatton-Brown-Rahman syndrome (DNMT3A), Rett Syndrome (MECP2), Chung-Jansen syndrome (PHIP), Schaaf-Yang syndrome (MAGEL2), ulnar mammary syndrome (TBX3), or Rubinstein-Taybi syndrome (CREBBP).
   Note: Although some of the genetic variants that are eligible to be enrolled into this study are associated with specific syndromes, the intent of this study is not to enroll children with significant cognitive impairment or other significant co-morbidities.
   Patients with the correct genetic variants, but who otherwise do not exhibit the syndrome, are eligible for enrollment.
8. Glycated hemoglobin (HbA1C) >10.0% at Screening.
9. History of significant liver disease other than non-alcoholic fatty liver disease (NAFLD) or nonalcoholic steatohepatitis (NASH).
10. Glomerular filtration rate (GFR)<60 mL/min at Screening.
11. History or close family history (parents or siblings) of melanoma, or patient history of oculocutaneous albinism.
   Note: If the type of skin cancer is not known, then the patient should not be enrolled into the study.
12. Significant dermatologic findings relating to melanoma or pre-melanoma skin lesions (excluding non-invasive basal or squamous cell lesion), determined as part of a comprehensive skin evaluation performed by the Investigator during Screening. Any concerning lesions identified during Screening will be biopsied and results known to be benign prior to enrollment. If the pretreatment biopsy results are of concern, the patient may need to be excluded from the study.
13. Pateints, in the opinion of the Investigator, not suitable to participate in the study.
14. Participation in any clinical study with an investigational drug/device within 3months prior to the first day of dosing.
15. Patients previously enrolled in a clinical study involving setmelanotide or any previous exposure to setmelanotide.
16. Significant hypersensitivity to any excipient in the study drug.
17. Females who are breastfeeding or nursing.

### Duration of Treatment

All patients will receive either setmelanotide or matching placebo for up to 40 weeks. Total participation in the study will last up to 52 weeks.

### Treatment

Study medication will be administered as SC injection once daily (QD).
In patients 12 years of age and older, setmelanotide 2 mg QD will be administered for approximately the first 14 days, then increased to setmelanotide 3 mg QD for the remainder of the study. Dose escalation should occur at the study visit planned for Day 14 (±3 days) and should occur on the day of that visit.
In patients aged 6 to <12 years old, setmelanotide 1 mg QD will be administered for approximately the first 7 days, then increased to setmelanotide 2 mg for approximately 7 days, then increased to 3 mg QD for the remainder of the study. Dose escalation should occur during the phone call planned for Day 7 (±2 days) and at the study visit planned for Day 14 (±3 days) and should occur on the day of that visit.
Investigators may increase or decrease the dose, if necessary, to treat an AE, although a dose greater than 3 mg QD should not be used in this study. If a Primary Investigator (PI) feels that a dose adjustment is required for a reason other than an AE (e.g., exaggerated weight loss), the decision should be discussed with the Sponsor prior to changing the dose.
All changes in dose other than the per-protocol dose titration should be captured as a protocol violation, regardless of the rationale for the dose adjustment.
There will be extensive training of patients in drug administration including educational materials. Study-specific training materials will be provided to both the investigative staff and study participants and caregivers.

### Concomitant Medications

Any medication or vaccine (including over-the-counter or prescription medicines, vitamins, and/or herbal supplements) that the patient is receiving at the time of enrollment or receives during the study must be recorded along with:
Reason for use
Dates of administration including start and end dates
Dosage information including dose and frequency
   Patients should not make significant changes to their diet or exercise routines during the course of the study.

### Prohibited Medications

Medications that are approved to treat obesity (e.g., orlistat, lorcaserin, phentermine-topiramate, naltrexone-bupropion) are not allowed within 3 months of first dose of study medication (e.g., enrollment) and are prohibited during the course of the study.
GLP-1 receptor agonists may be used up to the dose approved for the treatment of diabetes mellitus (e.g., liraglutide up to a daily dose of 1.8 mg) as long as (1) it is not being prescribed for the treatment of obesity, (2) the dose has been stable for at least 3 months prior to enrollment, (3) the patient has not experienced weight loss during the previous 3 months, AND (4) the patient intends to keep the dose stable throughout the course of the study. All concomitant medications should be kept at a stable dose throughout the course of the study, unless a dose change is necessary to treat an AE.

### Patient Assessments

Adherence to the study design requirements is essential and required for study conduct. Exemplary patient evaluations include a medical history review, physical examination, comprehensive skin examination, measurement of height, weight, waist circumference, body composition assessment, pregnancy test (if applicable), daily hunger questionnaires, global hunger assessment, and evaluation of PHQ-A or PHQ-9, C-SSRS, SF-12 or SF-10, IWQOL, FSH, HbA1c, and fasting lipid panel.
All screening evaluations must be completed and reviewed to confirm that potential patients meet all eligibility criteria. The investigator will maintain a screening log to record details of all patients screened and to confirm eligibility or record reasons for screening failure, as applicable.
When scheduled at the same time point, the order of procedures should be as follows: obtain vital signs, perform 12-lead electrocardiogram (ECG), and perform blood draws (at the specified time point, if applicable). Adjustments may be made depending upon specific circumstances and in consultation with the Sponsor.
Immediate safety concerns should be discussed with the Sponsor immediately upon occurrence or awareness to determine if the patient should continue or discontinue study treatment.

### Example 3. A Randomized Study Capturing 5 Independent Sub-Studies of Setmelanotide in Subjects with POMC, PCSK1, LEPR, SRC1, SH2B1, and PCSK1 N221D Gene Defects

### Purpose

This study protocol describes 5 independent, randomized, double-blind, placebo-controlled, sub-studies of setmelanotide in obese patients with 6 specific gene defects in the MC4R pathway: POMC or PCKS1, LEPR, SRC1, SH2B1, and PCSK1 N221D.
These 5 sub-studies have high degree of similarities. The objectives and endpoints are identical for all 5 sub-studies in patients with POMC and/or PCSK1, LEPR, SRC1, SH2B, and PCSK1 N221D gene defects in the melanocortin-4 receptor (MC4R) pathway.

### Investigational Study Drug

Setmelanotide, 10 mg/mL in a sterile solution for injection, will be provided at 1.0, 2.0, 3.0 mg QD for patients 6 to <16 years of age, and 2.0 to 3.0 mg QD for patients ≥16 years of age.

### Objectives and Endpoints

### Primary (Identical for all 5 Sub-Studies)

Objective: To evaluate the proportion of obese patients who respond to setmelanotide at 52 weeks of treatment
Endpoints: Proportion of patients who are responders to active treatment compared to placebo:
For patients ≥18 years old at the time of enrollment, a responder is defined as achieving a ≥10% reduction in body weight at 52 weeks of treatment
For patients <18 years old at the time of enrollment, a responder is defined as reduction of body mass index (BMI) ≥5% at 52 weeks of treatment

### Secondary (Identical for all 5 Sub-Studies)

### Objectives:

i) To evaluate the change in body weight in response to setmelanotide from baseline to 52 weeks of treatment
ii) To evaluate changes in hunger score in response to setmelanotide from baseline to 52 weeks of treatment
iii) To evaluate change in BMI and waist circumference in response to setmelanotide from baseline to 52 weeks of treatment
iv) To evaluate the safety and tolerability of setmelanotide

### Endpoints:

i) Mean change and mean percent change in body weight in patients in response to setmelanotide at 52 weeks of treatment compared to placebo
ii) Change in % Centers for Disease Control and Prevention 95^{th} percentile in patients <18 with BMI>40 at 52 weeks of treatment compared to placebo
iii) Mean body weight loss, and % body weight loss in responders (defined as patients with >5% body weight loss (if >18 years of age), and a decrease in % BMI by 3% (if <18 years of age) after 12 weeks of therapy on 3.0 mg/day or maximally tolerated dose, as compared to placebo responders
iv) Mean percent change in the weekly average most hunger score at 52 weeks of setmelanotide treatment compared to placebo
v) Mean change in BMI and change in BMI Z-score and wait circumference at 52 weeks of setmelanotide treatment compared to placebo
vi) The overall safety and tolerability of setmelanotide in patients with genetic defects in the MC4R pathway

### Study Design Rationale

Five randomized, double-blind, placebo-controlled independent sub-studies will enroll obese patients (6 to 65 years of age) and with POMC and/or PCSK1, LEPR, SRC1, SH2B1, or PCSK1 N221D genetic defects in the MC4R pathway. Patients with heterozygous POMC and/or PCSK1 and LEPR genetic defects, will be stratified based on their genetic variant (pathogenic, likely pathogenic and VOUS) (per American College of Medical Genetics [ACMG] classification). The number of patients with a VOUS variant in each of these two studies will initially be capped at 50%. Patients with homozygous, heterozygous, or compound heterozygous SRC1 genetic defect will be enrolled in the appropriate sub studies.

### Screening

The Screening Period begins with signing the informed consent/assent form, and will last between 2 and 8 weeks (Day−14 to −56). During the Screening Period, patients will undergo all procedures as outlined in order to determine if they meet the Inclusion and Exclusion criteria of the study. During the Screening Period, patients or caregivers will undergo training on injection of study drug and other study procedures. Patients will be issued an electronic diary (e-diary) to capture daily compliance with injections (post enrollment) and hunger score assessments (starting Screening). Each sub-study will have the same Screening Period.

### Baseline

### At the Study Visit Day 1:

Patients will undergo all procedures and eligibility for the study will be reconfirmed.
   Patients who meet all screening criteria will be randomized 1:1 to receive daily setmelanotide or matching placebo.
Patients with POMC or PCSK1 or LEPR gene defects will also be stratified based on ACMG classification (P/LP: VOUS, ratio 1:1), in addition to the age classification.
Patients with SRC1, SH2B1, and PCSK1 N221D gene defects will be only stratified based on age.
All eligible patients will be stratified based on age group: −6 to <12 years of age-12 to <18 years of age-18 to ≤65 years of age
The study center must confirm that patients completed the electronic diary (e-diary) at least 4 of the 7 days prior to the enrollment visit. If thee-diary is not appropriately completed patients may not enter the study. The enrollment visit may be re-scheduled if the remaining visit window allows.
Patients will have body weight assessed; this will be recorded as the patient's baseline weight. Patients will be issued a scale and any additional required equipment to capture weight and vital signs measurements during telehealth visits.
Patients will inject subcutaneously (SC) their first dose of setmelanotide under the supervision of the study staff.

### Treatment Period (52 Weeks)

Following randomization, patients will self-inject (or the caregiver will inject the patient with) SC setmelanotide daily for approximately 52 weeks. During the study, patients will attend either in-person or telehealth visits. Any visit planned as a virtual visit may be converted to an in-person visit, at the discretion of the Primary Investigator. If more than 2 virtual visits are to be converted into in-person visits.
Patients will receive individualized counseling in healthy nutrition, based on the guidelines of the Obesity Society, American College of Cardiology and American Heart Association on every visit. Counselling will be performed by qualified personnel. Patients will be encouraged to perform 150 min moderate exercise per week.

### End of Treatment (EOT)

The EOT Visit will occur as in-person clinic visit at Week 52, which is the final day of treatment with setmelanotide or placebo.

### Post-Treatment Follow-Up

Patients will enter a LongTerm Extension Study (LTE). When the last patient, last visit occurs in the index sub-study, each patient eligibility in the LTE will be reassessed based on weight loss criteria. Patients who do not enroll into the LTE study will have an End of Treatment (EOT) Visit conducted via telephone at Week 56.

### Rescue Treatment

Patients will be offered to start a rescue treatment, if after 26 weeks treatment, and regardless of treatment assignment,

### Inclusion Criteria

### Inclusion Criteria:

1. Patients must have a pre-identified:
Heterozygous genetic variant in the POMC gene or PCSK1 gene
Heterozygous genetic variant in the LEPR gene
Homozygous or heterozygous variant, compound heterozygous in the SRC1,
Homozygous or heterozygous variant, compound heterozygous in SH2B1gene, or chromosomal 16p11.2 deletion encompassing the SH2B1gene
Heterozygous N221D variant in the PCSK1 gene. For POMC, PCSK1, LEPR, SRC1and SH2B1 gene variants, to be considered for inclusion, the variant must either:
be categorized by a CLIA/CAP/ISO15189-certified laboratory using ACMG criteria as a) Pathogenic; orb) Likely Pathogenic; orc) a Variant of Uncertain Significance (VOUS) or be an N221D variant of the PCSK1 gene. If a patient has composite heterozygous variants eligible for the study she/he will be accounted for the higher category based on ACMG classification. For example, if a patient is a composite heterozygous for POMC pathogenic variant, and LEPR VOUS, the patient will be categorized as POMC pathogenic (Sub-study 35a). If the 2 variants have the same ACMG classification, adjudication will be assigned to the less prevalent gene sub-study.
For example, if a patient is a composite heterozygous for LEPR VOUS and SRC1 VOUS, the patient will be categorized as SRC1 (Sub-study35c). The final Sub-study assignment will follow this hierarchy: deletion>pathogenic>likely pathogenic>VOUS>RISK. Patients that harbor RISK variants are not eligible, with the exception of PCSK1 N221D.
   If a patient has two variants with the same ACMG classification, adjudication will be assigned following this hierarchy: PCSK1>SRC1>SH2B1>LEPR>POMC>PCSK1 N221D.

### Exclusion Criteria:

If the investigator has genetics results on a patient who may been eligible for the study, but the genetics have not yet been categorized by a CLIA/CAP/ISO15189 certified laboratory, then Rhythm may provide testing and/or categorization through a third-party laboratory.
2. Between 6 and 65 years of age at the time of provision of informed consent/assent.
3. Obese, defined as BMI≥30 kg/m for patients ≥18 years of age or BMI ≥95^{th} percentile forage and gender for patients 5 up to 17 years of age, based on based on the United States (US)Centers for Disease Control and Prevention criteria.
4. Patient and/or parent or guardian is able to communicate well with the Investigator, understand and comply with the requirements of the study (including once daily [QD] injection regimen and all other study procedures), and is able to understand and sign the written informed consent/assent. Patients who are unable to comply with all study procedures due to cognitive limitations or any other reason should not be enrolled into the study.
5. Patient and/or parent or guardian reports that patient experienced childhood obesity, defined as the patient and/or parent or guardian reporting that the patient was significantly overweight during childhood.
6. For women of child-bearing potential (WOCBP), agrees to use a highly effective form of contraception throughout the study and for 90 days following the study.
Highly effective forms of contraception include:—Combined (estrogen and progestogen containing) hormonal contraception associated with inhibition of ovulation (oral, intravaginal, or transdermal)—Progestogen-only hormonal contraception associated with inhibition of ovulation (oral, injectable, or implantable)—Intrauterine device (IUD)
7. Sexual abstinence only if it is the preferred and usual lifestyle of the patient. Reported history of failed lifestyle intervention of diet and exercise.
8. Reported history of hyperphagia.

### Exclusion Criteria:

1. Recent intensive (within 2 months) diet and/or exercise regimen with or without the use of weight loss agents including herbal medications that has resulted in >2% weight loss.
2. Use of any medication that is approved to treat obesity within 3 months of first dose of study (e.g., or list at, phentermine-topiramate, naltrexone-bupropion, GLP-1 treatments) if >2% weight loss in the last 3months.
3. History of bariatric surgery with evidence of weight loss of >2% in the last 3 months.
4. Documented diagnosis of schizophrenia, bipolar disorder, personality disorder, major depressive disorder or other significant psychiatric disorder(s) that the Investigator believes will interfere significantly with study compliance.
5. Any suicidal ideation of type 4 or 5 on the Columbia Suicide Severity Rating Scale (C-SSRS) during Screening, any suicide attempt in the past 5 years, or any suicidal behavior in the last month.
6. Current, clinically significant pulmonary, cardiac or oncologic disease considered severe enough to interfere with the study and/or confound the results. Any patient with a potentially clinically significant disease should be reviewed.
7. Glycated hemoglobin (HbA1C) >10% at Screening.
8. History of significant liver disease other than non-alcoholic fatty liver disease (NAFLD) or nonalcoholic steatohepatitis (NASH).
9. Glomerular filtration rate (GFR)<30 mL/minb at Screening.
1O·History or close family history (parents or siblings) of melanoma, or patient history of oculocutaneous albinism.
11. Significant dermatologic findings relating to melanoma or pre-melanoma skin lesions (excluding non-invasive basal or squamous cell lesion), determined as part of a comprehensive skin evaluation performed by the Investigator during Screening. Any concerning lesions identified during Screening will be biopsied and results known to be benign prior to enrollment. If the pre-treatment biopsy results are of concern, the patient may need to be excluded from the study.
12. Pateints, in the opinion of the Study Investigator, not suitable to participate in the study.
13. Participation in any clinical study with an investigational drug/device within 3 months or 5half-lives, whichever is longer, prior to the first day of dosing.
14. Previously enrolled in a clinical study involving setmelanotide or any previous exposure to setmelanotide.
15. Significant hypersensitivity to any excipient in the study drug.
16. If female, pregnant or breastfeeding.

### Duration of Treatment

All patients will receive either setmelanotide or matching placebo for up to 40 weeks. Total participation in the study will last up to 52 weeks.

### Treatment

Study medication will be administered as SC injection once daily (QD).
In patients 12 years of age and older, setmelanotide 2 mg QD will be administered for approximately the first 14 days, then increased to setmelanotide 3 mg QD for the remainder of the study. Dose escalation should occur at the study visit planned for Day 14 (±3 days) and should occur on the day of that visit.
In patients aged 6 to <12 years old, setmelanotide 1 mg QD will be administered for approximately the first 7 days, then increased to setmelanotide 2 mg for approximately 7 days, then increased to 3 mg QD for the remainder of the study. Dose escalation should occur during the phone call planned for Day 7 (±2 days) and at the study visit planned for Day 14 (+3 days) and should occur on the day of that visit.
Investigators may increase or decrease the dose, if necessary, to treat an AE, although a dose greater than 3 mg QD should not be used in this study. If a Primary Investigator (PI) feels that a dose adjustment is required for a reason other than an AE (e.g., exaggerated weight loss), the decision should be discussed with the Sponsor prior to changing the dose.
All changes in dose other than the per-protocol dose titration should be captured as a protocol violation, regardless of the rationale for the dose adjustment.
There will be extensive training of patients in drug administration including educational materials. Study-specific training materials will be provided to both the investigative staff and study participants and caregivers.

### Patient Assessments

Adherence to the study design requirements is essential and required for study conduct. Exemplary patient evaluations include a medical history review, physical examination, comprehensive skin examination, measurement of height, weight, waist circumference, body composition assessment, pregnancy test (if applicable), daily hunger questionnaires, global hunger assessment, and evaluation of PHQ-A or PHQ-9, C-SSRS, SF-12 or SF-10, IWQOL, FSH, HbA1c, and fasting lipid panel.
All screening evaluations must be completed and reviewed to confirm that potential patients meet all eligibility criteria. The investigator will maintain a screening log to record details of all patients screened and to confirm eligibility or record reasons for screening failure, as applicable.
When scheduled at the same time point, the order of procedures should be as follows: obtain vital signs, perform 12-lead electrocardiogram (ECG), and perform blood draws (at the specified time point, if applicable). Adjustments may be made depending upon specific circumstances and in consultation with the Sponsor.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of treating a disease, disorder, or condition in a subject comprising administering to the subject a melanocortin-4 receptor (MC4R) agonist, wherein the disease, disorder, or condition is related to an MC4R pathway agonizable gene selected from ARL6, RAI1, SRC1, BBS19, BBS21, CEP290, IFT74, LZTFL1, MKS1, TRIM32, WDPCP, RPS6KA3, HTR2C, KSR2, PROK2, RAB23, MRAP2, AFF4, ADCY3, TUB, OTP, GPR101, TBX3, ACBD7, AGRP, CADM1, CADM2, CARTPT, CCDC28B, CCK, CNR1, CREBBP, CREBRF, CUL4B, DYRK1B, ENPP1, EP300, FMR1, FTO, GHRL, GIPR, GLP1R, INPP5E, INS, INSIG2, IRS1, IRS4, KCTD15, KIDINS220, MCHR1, MSRA, NDN, NEGR1, NLGN2, NPY, NR0B2, NTRK2, PCNT, PCSK2, PHF6, PMCH, PPARG, PYY, SDC3, SEC16B, SLC6A14, SNRPN, THRB, TMEM18, TMEM67, TRAPPC9, UCP1, UCP3, VPS13B, NRP1, NRP2, PLXNA1, PLXNA2, PLXNA3, PLXNA4, SEMA3A, SEMA3B, SEMA3D, SEMA3E, SEMA3F, SEMA3G, DNMT3A, RPGRIP1L, ISL1, TRPC5, PHIP, and MeCP2.

2. The method of claim 1, wherein the disease, disorder, or condition is characterized by a mutation (e.g., a substitution mutation, a deletion mutation, or a polymorphism) in the MC4R pathway agonizable gene.

3. The method of claim 2, wherein the subject is homozygous or heterozygous for the mutation in the MC4R pathway agonizable gene.

4. The method of claim 2, wherein the subject is or is identified as being a heterozygous carrier of the mutation(s), e.g., having one functional allele and one non-functional allele of the MC4R pathway agonizable gene.

5. The method of claim 2, wherein the subject is or is identified as being a compound heterozygous carrier of the mutation(s), e.g., having two distinct non-functional alleles, e.g., having the MC4R pathway agonizable gene.

6. The method of claim 2, wherein the subject is or is identified as being a homozygous carrier of the mutation(s), e.g., having a homozygous null genotype of the MC4R pathway agonizable gene.

7. The method of claim 1, wherein the MC4R pathway agonizable gene is selected from RAI1 and SRC.

8. The method of claim 7, wherein the MC4R pathway agonizable gene is RAI1.

9. The method of claim 7, wherein the MC4R pathway agonizable gene is SRC.

10. The method of claim 1, wherein the disease, disorder, or condition is characterized by a plurality of mutations (e.g., at least 2, 3, 4, 5, 6, 7, 8, 9, or 10 mutations) in the MC4R pathway agonizable gene.

11. The method of claim 10, wherein the plurality of MC4R pathway agonizable genes comprises RAI1 and SRC.

12. The method of claim 10, wherein the plurality of MC4R pathway agonizable genes further comprises SHH2.

13. The method of claim 1, wherein the disease, disorder, or condition is characterized by modulation (e.g., upregulation or downregulation) of the MC4R pathway agonizable gene.

14. The method of claim 1, wherein the disease, disorder, or condition comprises Bardet-Biedl syndrome, Alstrom Syndrome, Prader Willi syndrome, hypothalamic obesity, or Smith-Magenis syndrome.

15. The method of claim 1, wherein the disease, disorder, or condition comprises Bardet-Biedl syndrome.

16. The method of claim 1, wherein the disease, disorder, or condition comprises Alstrom Syndrome.

17. The method of claim 1, wherein the disease, disorder, or condition comprises Prader Willi syndrome.

18. The method of claim 1, wherein the disease, disorder, or condition comprises Smith-Magenis syndrome.

19. The method of claim 1, wherein the disease, disorder, or condition comprises hypothalamic obesity.

20. The method of claim 1, wherein a symptom of the disease, disorder, or condition comprises obesity or hyperphagia.

21. The method of claim 1, wherein the MC4R agonist is has the structure of formula (I):
(R²R³)-A¹-c(A²-A³-A⁴-A⁵-A⁶-A⁷-Ag-A⁹)-A¹⁰-R¹ (I),
wherein:
A¹ is Acc, HN—(CH₂)ₘ—C(O), L- or D-amino acid, or deleted;
A² is Cys, D-Cys, hCys, D-hCys, Pen, D-Pen, Asp, or Glu;
A³ is Gly, Ala, β-Ala, Gaba, Aib, D-amino acid, or deleted;
A⁴ is His, 2-Pal, 3-Pal, 4-Pal, Taz, 2-Thi, 3-Thi, or (X¹, X², X³, X⁴, X⁵)Phe;
A⁵ is D-Phe, Phe, D-1-Nal, D-2-Nal, D-Trp, D-Bal, D-(X¹, X², X³, X⁴, X⁵)Phe, or D-(Et)Tyr;
A⁶ is Arg, hArg, Dab, Dap, Lys, Orn, or HN-CH((CH₂)ₙ—N(R⁴R⁵))—C(O);
A⁷ is Trp, 1-Nal, 2-Nal, Bal, Bip, D-Trp, D-2-Nal, D-Bal or D-Bip;
A⁸ is Gly, D-Ala, Acc, Ala, 13-Ala, Gaba, Apn, Ahx, Aha, HN—(CH₂)ₛ—C(O), or deleted;
A⁹ is Cys, D-Cys, hCys, D-hCys, Pen, D-Pen, Dab, Dap, Orn, or Lys;
A¹⁰ is Acc, HN—(CH₂)ᵣ—C(O), L- or D-amino acid, or deleted;
R¹ is OH or NH₂;
each of R² and R³ is, independently for each occurrence, selected from the group consisting of H, (C₁-C₃₀)alkyl, (C₁-C₃₀)heteroalkyl, (C₁-C₃₀)acyl, (C₂-C₃₀)alkenyl, (C₂-C₃₀)alkynyl, aryl(C₁-C₃₀)alkyl, aryl(C₁-C₃₀)acyl, substituted (C₁-C₃₀)alkyl, substituted (C₁-C₃₀)heteroalkyl, substituted (C₁-C₃₀)acyl, substituted (C₂-C₃₀)alkenyl, substituted (C₂-C₃₀)alkynyl, substituted aryl(C₁-C₃₀)alkyl, and substituted aryl(C₁-C₃₀)acyl;
each of R⁴ and R⁵ is, independently for each occurrence, H, (C₁-C₄₀)alkyl, (C₁-C₄₀)heteroalkyl, (C₁-C₄₀)acyl, (C₂-C₄₀)alkenyl, (C₂-C₄₀)alkynyl, aryl(C₁-C₄₀)alkyl, aryl(C₁-C₄₀)acyl, substituted (C₁-C₄₀)alkyl, substituted (C₁-C₄₀)heteroalkyl, substituted (C₁-C₄₀)acyl, substituted (C₂-C₄₀)alkenyl, substituted (C₂-C₄₀)alkynyl, substituted aryl(C₁-C₄₀)alkyl, substituted aryl(C₁-C₄₀)acyl, (C₁-C₄₀)alkylsulfonyl, or —C(NH)—NH₂;
m is, independently for each occurrence, 1, 2, 3, 4, 5, 6 or 7;
n is, independently for each occurrence, 1, 2, 3, 4 or 5;
s is, independently for each occurrence, 1, 2, 3, 4, 5, 6, or 7;
t is, independently for each occurrence, 1, 2, 3, 4, 5, 6, or 7;
X′, X², X³, X⁴, and X⁸ each is, independently for each occurrence, H, F, Cl, Br, I, (C₁₋₁₀)alkyl, substituted (C₁₋₁₀)alkyl, (C₂₋₁₀)alkenyl, substituted (C₂₋₁₀)alkenyl, (C₂₋₁₀)alkynyl, substituted (C₂₋₁₀)alkynyl, aryl, substituted aryl, OH, NH₂, NO₂, or CN.

22. The method of claim 21, wherein A¹ is selected from Lys, D-Lys, Arg, and D-Arg.

23. The method of claim 21, wherein A² and A⁹ are each independently selected from Cys, hCys, and Pen.

24. The method of claim 21, wherein A³ is selected from Ala or D-Ala.

25. The method of claim 21, wherein A⁴ is selected from His and D-His.

26. The method of claim 21, wherein A⁵ is selected from Phe, D-Phe, D-1-Nal, and D-2-Nal.

27. The method of claim 21, wherein A⁶ is Arg.

28. The method of claim 21, wherein A⁷ is Trp.

29. The method of claim 21, wherein A⁸ and/or A¹⁰ is deleted.

30. The method of claim 21, wherein R¹ is NH₂.

31. The method of claim 21, wherein one of R² and R³ is independently hydrogen and the other of R² and R³ is independently (C₁-C₃₀) acyl (e.g., acetyl).

32. The method of claim 1, wherein the MC4R agonist is Ac-Arg-c(Cys-D-Ala-His-D-Phe-Arg-Trp-Cys)-NH₂ (SEQ ID NO: 140).

33. The method of claim 1, wherein the MC4R agonist is formulated as a pharmaceutical composition.

34. The method of claim 33, wherein the pharmaceutical composition further comprises a pharmaceutically acceptable excipient.

35. The method of claim 33, wherein the pharmaceutical composition comprises a polyethylene glycol (e.g., a modified polyethylene glycol, e.g., mPEG-DSPE, e.g., mPEG-2,000-DSPE)

36. The method of claim 1, comprising administering the MC4R agonist in a unit dosage suitable for injection, e.g., subcutaneous injection, to the subject.

37. The method of claim 36, wherein the unit dosage form is disposed within a delivery device, e.g., a syringe (e.g., prefilled syringe), an implantable device, a needleless hypodermic injection device, an infusion pump (e.g., implantable infusion pump), or an osmotic delivery system.

38. The method of claim 37, wherein the MC4R agonist is administered subcutaneously, e.g., by subcutaneous injection.

39. The method of claim 1, wherein the subject is obese, e.g., severely obese.

40. The method of claim 1, wherein the subject is hyperphagic.

41. The method of claim 1, wherein the subject has a body mass index (BMI) greater than 35 kg/m² (e.g., ≥36, 37, 38, 39, 40, 41, 42, 43, 44, 45, 46, 47, 48, 49, 50 kg/m² or greater) prior to administration of the MC4R agonist, e.g., at the time the MC4R agonist is prescribed, or at the time of the first administration.

42. The method of claim 1, wherein the subject has a body mass index (BMI) greater than 40 kg/m² (e.g., ≥41, 42, 43, 44, 45, 46, 47, 48, 49, 50, 51, 52, 53, 54, 55 kg/m² or greater) prior to administration of the MC4R agonist, e.g., at the time the MC4R agonist is prescribed, or at the time of the first administration.

43. The method of claim 1, wherein the subject has failed one or more previous therapies, e.g., exercise, diet, or behavioral therapies, prior to administration of the MC4R agonist, e.g., at the time the MC4R agonist is prescribed, or at the time of the first administration.

44. The method of claim 1, wherein the subject has a lower body weight after administration of the MC4R agonist than before administration of the MC4R agonist.
